(19) United States
(12) Patent Application Publication
(10) Pub. No.: US 2004/0152885 A1
(43) Pub. Date: Aug. 5, 2004

(54) Lp mammalian proteins; related reagents

(76) Inventors: Bernard Yaovi Amegadzie, Malvern, PA (US); Margaret Barbara Basinski, Indianapolis, IN (US); William L. Scott, Indianapolis, IN (US); Dayue Chen, Carmel, IN (US); Chongxi Huang, Indianapolis, IN (US); Gerald Patrick Keleher, Indianapolis, IN (US); Douglas Raymond Perkins, New Palestine, IN (US); Paul Robert Rosteck, Indianapolis, IN (US); Scott William Rowlinson, Indianapolis, IN (US); Patanjali Raghavac Sankhavaram, Carmel, IN (US); Eugene Thomas Seno, Weybridge, VT (US); Eric Wen Su, Carmel, IN (US); Yu Zhi, Indianapolis, IN (US)

Correspondence Address: Gerald P keleher, Eli Lilly & Company, Patent Division, PO Box 6288, Indianapolis, IN, 46206-6288, US

(21) Appl. No.: 10/480,172

(22) PCT Filed: Mar. 1, 2002
(86) PCT No.: PCT/US02/05093

(51) Int. Cl. C12Q 1/68; C07H 21/04

(52) U.S. Cl. 536/23.5

(57) ABSTRACT

Isolated nucleic acid molecules encoding polypeptides from a human, reagents related thereto (including purified polypeptides specific antibodies) are provided. Methods of using said reagents and diagnostic kits are also provided.

### FIELD OF THE INVENTION

The present invention generally relates to compositions related to proteins. In particular, it provides purified genes, polynucleotide sequences, proteins, polypeptides, antibodies, binding compositions, and related reagents useful, e.g., in the diagnosis, treatment, and prevention of cell proliferative, autoimmune/inflammatory, cardiovascular, neurological, and developmental disorders, and in the assessment of the effects of exogenous compounds on the expression of nucleic acid and amino acid sequences of such proteins.

### BACKGROUND OF THE INVENTION

Protein transport and secretion are essential for cellular function. Protein transport is mediated by a signal peptide located at the amino terminus of the protein to be transported or secreted. Proteins targeted to the ER may either proceed through the secretory pathway or remain in any of the secretory organelles such as the ER, Golgi apparatus, or lysosomes. Proteins that transit through the secretory pathway are either secreted into the extracellular space or retained in the plasma membrane. Proteins that are retained in the plasma membrane contain one or more transmembrane domains, each comprised of about 20 hydrophobic amino acid residues. Secreted proteins are generally synthesized as inactive precursors that are activated by post-translational processing events during transit through the secretory pathway. Such events include glycosylation, proteolysis, and removal of the signal peptide by a signal peptidase. Examples of secreted proteins with amino terminal signal peptides are discussed below and include proteins with important roles in cell-to-cell signaling. Such proteins include transmembrane receptors and cell surface markers, extracellular matrix molecules, cytokines, hormones, growth and differentiation factors, enzymes, neuropeptides, and vasomediators (reviewed in Alberts, et al. (1994) Molecular Biology of The Cell, Garland Publishing, New York, N.Y., pp. 557-560, 582-592). The discovery of new secreted proteins and the polynucleotides encoding them satisfies a need in the art by providing new compositions which are useful in the diagnosis, prevention, and treatment of cell proliferative, autoimmune/inflammatory, cardiovascular, neurological, and developmental disorders, and in the assessment of the effects of exogenous compounds on the expression of nucleic acid and amino acid sequences of secreted proteins.

### SUMMARY OF THE INVENTION

The present invention is based in part upon the discovery of LP (LP318a, LP318b, LP288, LP289, LP343, LP319a, LP319b, LP321, LP317, LP283, LP344, LP345, or LP346) proteins and/or polypeptides. The invention provides substantially pure, isolated, and/or recombinant LP protein or peptide (LP318a, LP318b, LP288, LP289, LP343, LP319a, LP319b, LP321, LP317, LP283, LP344, LP345, or LP346) exhibiting identity over a length of at least about 12 contiguous amino acids to a corresponding sequence of SEQ ID NO: Y; a natural sequence LP (LP318a, LP318b, LP288, LP289, LP343, LP319a, LP319b, LP321, LP317, LP283, LP344, LP345, or LP346) of SEQ ID NO: Y or Table 1, 2, 3, 4, 5, 6, 7 or 8; a fusion protein comprising LP (LP318a, LP318b, LP288, LP289, LP343, LP319a, LP319b, LP321, LP317, LP283, LP344, LP345, or LP346) sequence. In preferred embodiments, the portion is at least about 20, 25, 30, 35, 40, 45, 50, 55, 60, 65, 70, 75, 80, 85, 90, 95, or 100 contiguous amino acid residues in length. In other embodiments, the LP (LP318a, LP318b, LP288, LP289, LP343, LP319a, LP319b, LP321, LP317, LP283, LP344, LP345, or LP346): LP318a comprises a mature sequence of Table 1; LP318b comprises a mature sequence of Table 2; LP288 comprises a mature sequence of Table 3; LP289 or LP343 comprises a mature sequence of Table 4; LP319a or LP319b comprises a mature sequence of Table 5; LP321 comprises a mature sequence of Table 6; LP317 comprises a mature sequence of Table 7; and LP283 LP344, LP345, or LP346 comprises a mature sequence of Table 8; protein or peptide: is from a warm blooded animal selected from a mammal, including a primate; comprises at least one polypeptide segment of SEQ ID NO:Y exhibits a plurality of portions exhibiting the identity; is a natural allelic variant of the LP (LP318a, LP318b, LP288, LP289, LP343, LP319a, LP319b, LP321, LP317, LP283, LP344, LP345, or LP346) has a length at least about 30 amino acids; exhibits at least two non-overlapping epitopes which are specific for a mammalian LP(LP318a, LP318b, LP288, LP289, LP343, LP319a, LP319b, LP321, LP317, LP283, LP344, LP345, or LP346) exhibits identity over a length of at least about 20 amino acids to LP (LP318a, LP318b, LP288, LP289, LP343, LP319a, LP319b, LP321, LP317, LP283, LP344, LP345, or LP346) exhibits at least two non-overlapping epitopes which are specific for a LP (LP318a, LP318b, LP288, LP289, LP343, LP319a, LP319b, LP321, LP317, LP283, LP344, LP345, or LP346) exhibits identity over a length of at least about 25 amino acids to a primate LP (LP318a, LP318b, LP288, LP289, LP343, LP319a, LP319b, LP321, LP317, LP283, LP344, LP345, or LP346) is glycosylated; is a synthetic polypeptide; is attached to a solid substrate; is conjugated to another chemical moiety; is a 5-fold or less substitution from natural LP (LP318a, LP318b, LP288, LP289, LP343, LP319a, LP319b, LP321, LP317, LP283, LP344, LP345, or LP346) sequence; or is a deletion or insertion variant from a natural LP (LP318a, LP318b, LP288, LP289, LP343, LP319a, LP319b, LP321, LP317, LP283, LP344, LP345, or LP346) sequence. Various preferred embodiments include a composition comprising a sterile LP (LP318a, LP318b, LP288, LP289, LP343, LP319a, LP319b, LP321, LP317, LP283, LP344, LP345, or LP346) protein or peptide and a carrier, wherein the carrier is: an aqueous compound, including water, saline, and/or buffer; and/or formulated for oral, rectal, nasal, topical, or parenteral administration. The invention further provides a fusion protein, comprising: mature protein comprising sequence of Table 1, 2, 3, 4, 5, 6, 7 or 8 a detection or purification tag, including a FLAG, His6, or Ig sequence; or sequence of another LP LP318a, LP318b, LP288, LP289, LP343, LP319a, LP319b, LP321, LP317, LP283, LP344, LP345, or LP346) protein or peptide. These reagents also make available a kit comprising such an LP (LP318a, LP318b, LP288, LP289, LP343, LP319a, LP319b, LP321, LP317, LP283, LP344, LP345, or LP346) protein or polypeptide, and: a compartment comprising the protein or polypeptide; and/or instructions for use or disposal of reagents in the kit. Providing an antigen, the invention further provides a binding compound comprising an antigen binding portion from an antibody, which specifically binds to a natural LP (LP318a, LP318b, LP288, LP289, LP343, LP319a, LP319b, LP321, LP317, LP283, LP344, LP345, or LP346) protein or polypeptide, wherein: the protein or polypeptide is a primate protein; the binding compound is an Fv, Fab, or Fab2 fragment; the binding compound is conjugated to another chemical moiety; or the antibody: is raised against a peptide sequence of a mature polypeptide comprising sequence of Table 1, 2, 3, 4, 5, 6, 7, or 8 is raised against a mature LP (LP318a, LP318b, LP288, LP289, LP343, LP319a, LP319b, LP321, LP317, LP283, LP344, LP345, or LP346) is immunoselected; is a polyclonal antibody; binds to a denatured LP, LP1, LP2, LP3, LP4, LP5, LP6, LP7, LP8, LP9, or an LP of Table 1, 2, 3, 4, 5, 6, 7 or 8 exhibits a Kd to antigen of at least 30 μM; is attached to a solid substrate, including a bead or plastic membrane; is in a sterile composition; or is detectably labeled, including, for example, a radioactive, enzymatic, structural, or fluorescent label. Preferred kits include those containing the binding compound, and: a compartment comprising the binding compound; and/or instructions for use or disposal of reagents in the kit. Many of the kits will be used for making a qualitative or quantitative analysis. Other preferred compositions will be those comprising: a sterile binding compound, or the binding compound and a carrier, wherein the carrier is: an aqueous compound, including water, saline, and/or buffer; and/or formulated for oral, rectal, nasal, topical, or parenteral administration. The present invention further provides an isolated or recombinant LP nucleic acid encoding a protein or peptide or fusion protein described above, wherein: the LP protein and/or polypeptide is from a mammal, including a primate; or the LP nucleic acid encodes an antigenic peptide sequence from an LP (LP318a, LP318b, LP288, LP289, LP343, LP319a, LP319b, LP321, LP317, LP283, LP344, LP345, or LP346) of Table 1, 2, 3, 4, 5, 6, 7, or 8 encodes a plurality of antigenic peptide sequences from an LP (LP318a, LP318b, LP288, LP289, LP343, LP319a, LP319b, LP321, LP317, LP283, LP344, LP345, or LP346) of Table 1, 2, 3, 4, 5, 6, 7, or 8 exhibits identity to a natural cDNA encoding the segment; is an expression vector; further comprises an origin of replication; is from a natural source; comprises a detectable label; comprises synthetic nucleotide sequence; is less than 6 kb, preferably less than 3 kb; is from a mammal, including a primate; comprises a natural full length coding sequence; is a hybridization probe for a gene encoding an LP family protein; or is a PCR primer, PCR product, or mutagenesis primer. In certain embodiments, the invention provides a cell or tissue comprising such a recombinant LP nucleic acid. Preferred cells include: a prokaryotic cell; a eukaryotic cell; a bacterial cell; a yeast cell; an insect cell; a mammalian cell; a mouse cell; a primate cell; or a human cell. Other kit embodiments include a kit comprising the described LP nucleic acid, and: a compartment comprising the LP nucleic acid; a compartment further comprising an LP (LP318a, LP318b, LP288, LP289, LP343, LP319a, LP319b, LP321, LP317, LP283, LP344, LP345, or LP346) protein or polypeptide; and/or instructions for use or disposal of reagents in the kit. In many versions, the kit is capable of making a qualitative or quantitative analysis. Other LP nucleic acid embodiments include those which: hybridize under wash conditions of at least 42° C., 45° C., 47° C., 50° C., 55° C., 60° C., 65° C., or 70° C. and less than about 500 mM, 450 mM, 400 mM, 350 mM, 300 mM, 250 mM, 200 mM, 100 mM, to an LP of SEQ ID NO: X that exhibit identity over a stretch of at least about 30, 32, 34, 36, 38, 39, 40, 42, 44, 46, 48, 49, 50, 52, 54, 56, 58, 59, 75, or at least about 150 contiguous nucleotides to an LP (LP318a, LP318b, LP288, LP289, LP343, LP319a, LP319b, LP321, LP317, LP283, LP344, LP345, or LP346). In other embodiments: the wash conditions are at 55° C. and/or 300 mM salt; 60° C. and/or 150 mM salt; the identity is over a stretch is at least 55 or 75 nucleotides. In other embodiments, the invention provides a method of modulating physiology or development of a cell or tissue culture cells comprising introducing into such cell an agonist or antagonist of an LP (LP318a, LP318b, LP288, LP289, LP343, LP319a, LP319b, LP321, LP317, LP283, LP344, LP345, or LP346).

### DETAILED DESCRIPTION OF THE PREFERRED EMBODIMENTS

I. General

It is to be understood that this invention is not limited to the particular compositions, methods, and techniques described herein, as such compositions, methods, and techniques may, of course vary. It is also to be understood that the terminology used herein is for the purpose of de scribing particular embodiments only, and is not intended to limit the scope of the present invention which is only limited by the appended claims.

As used herein, including the appended claims, singular forms of words such as “a,” “an,” and “the” include, e.g., their corresponding plural referents unless the context clearly dictates otherwise. Thus, for example, reference to “an organism” includes, e.g., one or more different organisms, reference to “a cell” includes, e.g., one or more of such cells, and reference to “a method” include, e.g., reference to equivalent steps and methods known to a person of ordinary skill in the art, and so forth.

Unless otherwise defined, all technical and scientific terms used herein have the same meaning as commonly understood by a person of ordinary skill in the art to which this invention belongs. Although methods and materials similar or equivalent to those described herein can be used to practice or test the present invention, suitable methods and materials are described below. All publications, patent applications, patents, and other references discussed herein are provided solely for their disclosure before the filing date of the present application. Nothing herein is to be construed as an admission that the invention is not entitled to antedate any such disclosure by virtue of its prior invention. All publications, patent applications, patents, and other references mentioned herein are incorporated by reference in their entirety for the teachings for which they are cited (as the context clearly dictates), including all figures, drawings, pictures, graphs, hyperlinks, and other form of browser-executable code. Specifically applicants incorporate by reference Provisional Applications P14811, U.S. Serial No. 60/276596; filed Mar. 16, 2001; P14860, U.S. Serial No. 60/283654, filed Apr. 13, 2001; P14881, U.S. Serial No. 60/285238, filed Apr. 20, 2001; P14843, U.S. Serial No. 60/288548, filed May 3, 2001, and P15010, U.S. Serial No. 60/290351, filed May 11, 2001.

Polynucleotide sequences encoding an LP of the present invention are analyzed with respect to the tissue sources from which they were derived. Various cDNA library/tissue information described herein is found in the cDNA library/tissues of the LIFESEQ GOLD™ database (Incyte Genomics, Palo Alto Calif.) which corresponding information is incorporated herein by reference. Generally, in the LIFESEQ GOLD™ database a cDNA sequence is derived from a cDNA library constructed from a primate, (e.g., a human issue). Each tissue is generally classified into an organ/tissue category (such as, e.g., cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract). Typically, the number of libraries in each category is counted and divided by the total number of libraries across all categories. Results using the LIFESEQ GOLD™ database reflect the tissue-specific expression of cDNA encoding an LP of the present invention.

Additionally, each LP sequence of the invention is also searched via BLAST against the UniGene database. The UniGene database contains a non-redundant set of gene-oriented clusters. Each UniGene cluster theoretically contains sequences that represent a unique gene, as well as related information such as the tissue types in which the gene has been expressed and map location.

Particularly interesting portions, segments, or fragments of LP's of the present invention are discovered based on an analysis of hydrophobicity plots calculated via the “GREASE” application, which is a computer program implementation based on the Kyte-Doolittle algorithm (J. Mol. Biol. (1982) 157:105-132) that calculates a hydropathic index for each amino acid position in a polypeptide via a moving average of relative hydrophobicity. A hydrophilicity plot is determined based on a hydrophilicity scale derived from HPLC peptide retention times (see, e.g., Parker, et al., 1986 Biochemistry 25:5425-5431). Another hydrophobicity index is calculated based on the method of Cowan and Whittaker (Peptide Research 3:75-80; 1990). Antigenic features of LPs are calculated based on antigenicity plots (such as, e.g., via algorithms of Welling, et al. 1985 FEBS Lett. 188:215-218; the Hopp and Woods Antigenicity Prediction (Hopp & Woods, 1981 Proc. Natl. Acad. Sci., 78, 3824); the Parker Antigenicity Prediction (Parker, et al. 1986 Biochemistry, 25, 5425); the Protrusion Index (Thornton) Antigenicity Prediction (Thornton, et al. 1986 EMBO J., 5, 409); and the Welling Antigenicity Prediction (Welling, et al. 1985 FEBS Letters.188, 215)). Particularly interesting secondary LP structural features (e.g., such as a helix, a strand, or a coil) are discovered based on an application which is a computer implementation program based on the Predator (Frishman, and Argos, (1997) Proteins, 27, 329-335; and Frishman, D. and Argos, P. (1996) Prot. Eng., 9, 133-142); GOR IV (Methods in Enzymology 1996 R. F. Doolittle Ed., vol. 266, 540-553 Garnier, J, Gibrat J-F, Robson B); and Simpa96 (Levin, et al., J FEBS Lett 1986 Sep. 15;205(2):303-308) algorithms. One of skill in the art can use such programs to discover such secondary structural features without undue experimentation given the sequences supplied herein.

Features of LP No: 1 & 2 (LP318a(c16hDGL) & LP318b(c22hDGL))

LP318a(c16hDGL) (SEQ ID NO: 2) and LP318b(c16hDGL) (SEQ ID NO: 4) are novel human polypeptides. LP318a(c16hDGL) nucleic acid sequence was discovered using a normalized human brain cDNA Library whose construction is based generally on methods of Ko (1990) Nucleic Acids Res. 18(19): 5705-11, and Soares, et al (1994) Proc. Natl. Acad. Sci. 91:9228-9232. Briefly, tissues from twelve brain subregions (Hypothalamus, Thalamus, Amygdyla, Sensory Cortex, Motor Cortex, Hippocampus, Cerebellum, Pons and Locus Coeruleus, Caudate/Putamen/Nucleus Accumbens, Entero-Cortex and Anterior Hippocampus, Prefrontal cortex, Anterior Cingulate Cortex) were obtained from the Harvard Medical School Tissue Bank and used to make mRNAs aliquots that were used to generate cDNAs. The cDNAs were amplified using the polymerase chain reaction (PCR) and subsequently normalized by determining the ratio of high-, medium-, and low-abundance control genes. After normalization, a representative brain cDNA library was constructed from the normalized cDNAs. LP318a(c16hDGL) sequence was discovered using this library. Similarly, LP318b(c16hDGL) was discovered using nucleic acid sequence information obtained from LP318a(c16hDGL).

Sequence analysis of LP318a(c16hDGL) amino acid structure demonstrates that LP318a(c16hDGL) exhibits amino acid sequence similarity to a rodent (e.g., mouse) protein designated mDGL1. The gene (mdgl1) encoding the mDGL1 protein is located on a small segment of mouse chromosome 16, which is highly homologous to a segment of human chromosome 22. The mouse and human chromosomal regions on, respectively, chromosomes 16 and 22 may represent ortho- or paralog segments (particularly, the C22q11 region of human chromosome 22; see, e. g., Botta, et al., 1997 Mammalian Genome. 8(12): 890-5, 1997 [published erratum appears in Mamm Genome April 1998; 9(4):344]). Given, the relationship of mouse chromosome 16 to human chromosome 22, Applicants conducted a search for additional sequence similar to LP318a(c16hDGL) based on the homologous relationship between the rodent and primate chromosomal locations. Using LP318a(c16hDGL) sequence as bait, Applicants discovered the LP318b(c16hDGL) nucleic acid sequence and its interesting location on human chromosome 22.

LP318b(c16hDGL) nucleic acid sequence was localized to the C22q11 region of human chromosome 22. Moreover, the following diseases, conditions, syndromes, disorders, and/or pathological states have also been mapped to this and surrounding regions of human chromosome 22, such as, for example: CATCH 22 syndrome, which is a spectrum of human conditions collectively referred using this medical acronym to refer to the cardiac anomalies, abnormal facial features, thymic hypoplasias, cleft palate, hypocalcemias, and chromosome 22 microdeletions that are associated with it (see, e.g., Krahn, et al., 1998 Mayo Clinic Proceedings. 73(10):956-959; Vataja & Elomaa 1998 British Journal of Psychiatry 172: 518-520; Sergi, et al., 1999 Pathologica 91(3): 166-172; Momma, et al., 1999 Cardiology in the Young. 9(5):463-467; Ryan, et al., 1997 Journal of Medical Genetics. 34(10):798-804; Momma, et al., 1999 Pediatric Cardiology 20(2): 97-102; Kerstjens-Frederikse, et al., 1999 Journal of Medical Genetics 36(3): 221-224; Edelmann, et al., 1999 Human Molecular Genetics 8(7): 1157-1167; Ruangdaraganon, et al., 1999 Journal of the Medical Association of Thailand 82 Suppl 1:S179-185; Gaspar, et al., 1999 Genetic Counseling 10(1): 51-57; Yong, et al., 1999 European Journal of Pediatrics 158(7): 566-570; Bassett, et al., 1998 American Journal of Medical Genetics 81(4): 328-337; Karayiorgou, et al., 1998 Biological Psychiatry 43(6):425-431); conotruncal anomaly face syndrome (CTAFS) (see, e.g., Maeda, et al., 2000 Am J Med Genet Jun. 5;92(4):269-72; Matsuoka, et al., 1998 Hum Genet. 103(1):70-80), Cat eye syndrome (see, e.g., McTaggart, et al., 1998 Cytogenetics & Cell Genetics 81 (3-4):222-228; Momma, et al., 1997 Journal of the American College of Cardiology 30(4): 1067-1071; Craigen, et al., 1997 American Journal of Medical Genetics 72(1):63-65; Lewin, et al., 1997 American Journal of Cardiology 80(4): 493-497; Stratton, et al., 1997 American Journal of Medical Genetics 69(3): 287-289; Knoll, et al., 1995 Am J Med Genet 55(2):221-4); DiGeorge syndrome, in which individuals, have variable severity and combinations of e.g., conotruncal heart defects, abnormalities of the ear and palate, facial dysmorphism, and mental retardation as well as partial or complete aplasia/hypoplasia of the thymus and endocrine dysfunction, e.g. hypoparathyroidism. Patients with DiGeorge syndrome may present with impaired immune function, heart failure, hypocalcemia, facial dysmorphism, impaired hearing, and mental retardation. The syndrome, which is a significant cause of heart and craniofacial defects as well as mental retardation, is probably underdiagnosed and presents a large spectrum of presentation, from cases where the most prominent feature of the syndrome is hypocalcemia with hypoparathyroidism, to cases with asymptomatic, latent or late-onset hypocalcemia. Thus, it would be useful to have a genetic marker to identify individuals with this syndrome. For example, a recommendation to clinicians whom have patients presenting with late-onset or recurrent hypoparathyroidism is to perform a genetic analysis of the human 22q11 region to determine if the individual has a feature of DiGeorge syndrome (see, e.g., Hong R. 1998 Seminars in Hematology 35(4): 282-290; Sutherland, et al., 1998 Genomics 52(1): 37-43; Funke, et al., 1999 American Journal of Human Genetics 64(3): 747-58; Puech, et al., 1997 PNAS USA (18): 10090-10095; Kimber, et al., 1999 Human Molecular Genetics 8(12): 2229-2237; Lindsay, et al., 1999 Nature 401(6751): 379-383; Roberts, et al., 1997 Human Molecular Genetics 6(2): 237-245; Thomas, et al., 1997 Clinical Pediatrics 36(5): 253-266; Stoffel, et al., 1996 Human Genetics 98(1): 113-115; and Pizzuti, et al., 1996 American Journal of Human Genetics 58(4): 722-729); and velocardiofacial syndrome (VCFS) (see, e.g., Funke, et al., 1998 Genomics 53(2): 146-154; Saint-Jore, et al., 1998 Human Molecular Genetics 7(12): 1841-1849; Van Geet, et al., 1998 Pediatric Research 44(4): 607-611; Ford, et al., 2000 Laryngoscope 110(3 Pt 1): 362-367; Usiskin, et al., 1999 Journal of the American Academy of Child & Adolescent Psychiatry 38(12): 1536-1543; Olney, et al., 1998 Ear, Nose, & Throat Journal. 77(6): 460-461; Gothelf, et al., 1997 American Journal of Medical Genetics 72(4): 455-461; Sirotkin, et al., 1997 Genomics. 41(1): 75-83; Kumar, et al., 1997 American Journal of Cardiology. 79(3): 388-390).

Accordingly, an isolated and/or recombinant DNA molecule comprising LP318b(c16hDGL) nucleic acid sequence meets the statutory utility requirement of 35 U.S.C. §101 since it can be used to hybridize near sequence associated with one or more of the above stated diseases, conditions, syndromes, disorders, and/or pathological states and thus, LP22hDGL would serve as a marker for a such a disease, condition, syndrome, disorder, and/or pathological state. Additionally, compositions comprising LP318b(c16hDGL) polypeptides or polynucleotides (fragments thereof), LP318b(c16hDGL) agonists or antagonists, and/or binding compositions (e.g., LP318b(c16hDGL) antibodies) will also be useful for diagnosis, prognosis, treatment, amelioration, and/or intervention of such an above referenced disease, condition, or state.

Likewise, given the sequence similarity between LP318a(c16hDGL) and LP318b(c16hDGL) (see Table 1 below), it is likely that LP318a(c16hDGL) and LP318b(c16hDGL) are encoded by paralogous genes, which arose during some gene duplication event. Consequently, although LP318a(c16hDGL) nucleic acid sequence is not located on human chromosome 22, it also will be useful as a distinct marker for detecting, marking, associating with, and/or diagnosing individuals present with CTAFS, VCFS, DiGeorge, CATCH 22 or CTAFS-, VCFS-, DiGeorge-, or CATCH 22-like phenotypes. Such situations are not uncommon. Although, LP318a(c16hDGL) does not map to the chromosomal region deleted in, for example, CATCH 22 patients an LP318a(c16hDGL) mutein or variant could have evolved a degree of independence to modify such conditions or LP318a(c16hDGL) could affect other components of the same signaling pathway (see, e.g., a similar situation described by Clouthier, et al., 1998 Development 125: 813-824, where endothelin receptor dysfunction contributes to cranial and cardiac defects that mimic CATCH 22 phenotypes). Furthermore, Tsai, et al., (1999 Am J Med Genet 82(4): 336-339) describe a child with features consistent with having velocardiofacial syndrome (VCFS) such as congenital heart disease (atrial septal defect, ventricular septal defect, pulmonic stenosis), submucosal cleft palate, hypernasal speech, learning difficulties, and right fifth finger anomaly manifestations. All of these features are diagnostic for VCFS; however, upon genetic analysis of this patient a microdeletion in the 22q locations was not identified. Instead, a (4)(q34.2) deletion was discovered suggesting, similar to the situation of Clouthier, et al., that other components of the same signaling pathway could be involved or that multilocus effects may modify or affect the condition. Tsai, et al., suggest that this result emphasize the importance of searching for other karyotype abnormalities when a velocardiofacial syndrome-like phenotype is present. Consequently, given the relationship between LP318a(c16hDGL) and LP318b(c16hDGL), individuals presenting with CTAFS, VCFS, DiGeorge, CATCH 22 or CTAFS-, VCFS-, DiGeorge-, or CATCH 22-like phenotypes should be checked not only using a LP318b(c16hDGL) genetic analysis but also using an LP318a(c16hDGL) analysis since LP318a(c16hDGL) may also be involved in the organization of such phenotypes. Accordingly, an isolated and/or recombinant DNA molecule comprising LP318a(c16hDGL) nucleic acid sequence also meets the statutory utility requirement of 35 U.S.C. §101 since it can be used to hybridize near sequence associated with one or more of the above stated diseases, conditions, syndromes, disorders, and/or pathological states and thus serve as a marker for a such a disease, condition, syndrome, disorder, and/or pathological state.

The CTAFS, VCFS, DiGeorge, Cat Eye, and CATCH 22 syndromes are often associated with anomalous developmental characteristics of the cardiovascular and/or nervous systems, and/or anomalous development of the face and head (see, e.g., Momma, et al., 1999 Ped Cardio 20: 97-102; Hong, R., 1998, Seminars in Hematology, 35: 282-290). It has been suggested that the DiGeorge syndrome is associated with a basic embryological defect (e.g., inadequate development of the facial neural crest tissues) (see, e.g., Hong, 1998). Additional defects for the DiGeorge syndrome have been suggested to be in defects of the primitive aortic arches (see, e.g., Johnson, et al., 1995, Amer J of Cardio 76: 66-69). In addition, people with the CATCH 22 syndrome have an unexpectedly high incidence of behavioral disorders, e.g., major psychoses, including, e.g., schizophrenia and bipolar disorders (see, e.g., Vataja & Elomaa 1998 Brit J of Psychiatry 172: 518-520). Such behavioral abnormalities have been associated with defects in brain morphology (such as, e.g., defects in the midline structures of the brain) (see, e.g., Vataja & Elomaa 1998 Brit J of Psychiatry 172: 518-520). Recently, it has been suggested that genes encoding components of the nodal signaling pathway must be expressed only on one side of structures in the developing embryo to ensure correct placement and patterning. Errors in the nodal signaling pathway, for example, randomize the sidedness and morphology of the heart and other organs. Such findings present a way to reconcile the multiple phenotypic effects seen in CATCH 22 syndromes. For example, it has recently been demonstrated that nodal signaling is required for the proper development of laterally asymmetric structures in the brain (e.g., in the dorsal diencephalon, specifically, habenular nucleii and pineal structures)(see, e.g., Concha, et al., 2000 Neuron 28: 399-409; and Liang, et al., 2000 Development 127:5101-112). Currently, it is believed that genes involved in the nodal pathway (such as, e.g., squint, cyclops, lefty, antivin, and pitx2) are also responsible for localizing components of the dorsal diencephalon to the left side of the brain in vertebrates. According to this model, midline tissues in the developing vertebrate brain (where the nodal pathway is turned on) repress genes such as, e.g., cyclops and pitx2 on the right side of the developing diencephalon thus leading to morphological asymmetries of the nervous system. LP318b(c16hDGL) or LP318a(c16hDGL) may play a role in this system, for example, by having an effect on the nodal signaling system or by, e.g., modulating the pitx2 or cyclops effector portion of the system. The Drosophila protein with sequence similarity to LP318a(c16hDGL) and LP318b(c16hDGL) (see Table 1 below) is proposed to be a ligand protein further supporting such a ligand like function here for LP318a(c16hDGL) or LP318b(c16hDGL). Accordingly, for example, a two-hybrid type of system for identifying protein-protein interactions is encompassed herein to determine potential interactions of LP318b(c16hDGL) and/or LP318a(c16hDGL) with any of the currently described proteins known to influence morphological asymmetries (e.g., such as those described for the brain in Concha, et al., 2000 Neuron 28: 399-409; and Liang, et al., 2000 Development 127:5101-112). Such methods of determining protein-protein interactions are well known in the art (see, e.g., Fields and Song, 1989 Nature 340:245-6 for descriptions of the original yeast two-hybrid system design. Since then, modifications and improvements are well known in the art, see, e.g., Zhu, et al., 2000 Nature Genetics 26: 283-9 describing a method of analyzing hundreds or thousands of protein samples using a single protein chip technology that is applicable for a wide variety of assays, such as, e.g., ATP and GTP binding assays, protein kinase assays, nuclease assays, helicase assays, and protein-protein interaction assays. Similar technology could be employed here, without undue experimentation, to determine the characteristics of LP318b(c16hDGL) or LP318a(c16hDGL). Both these references are encompassed herein for their assay teachings.).

LP318a(c16hDGL)'s homology to proteins involved in blood coagulation (e.g., plasma kallikrein, coagulation factor XI, and plasminogen) and the possession of apple domains, which have been shown to be involved in binding other members of the coagulation cascade (such as, e.g., kininogen, and factor XIIa) suggest that LP318a(c16hDGL) may also be participate in the blood coagulation system. Furthermore, at least two additional pieces of evidence suggest that LP318a(c16hDGL) may also participate in inflammatory processes. One is based on the observation that after injury there is typically a simultaneous activation of the innate immune response and the coagulation system. This simultaneous activation has been discovered to be a phylogenetically-conserved, ancient and adaptive response that can be traced back to the early stages of eukaryotic evolution and which persists today so that the same proinflammatory stimuli that activate elements of the human clotting cascade also activate phagocytic effector cells (neutrophils, monocytes, and macrophages). Thus, a complex and highly integrated linkage between systemic inflammation and coagulation is maintained in all vertebrates (see, e.g., Opal S. M. 2000 Crit Care Med. (9 Suppl): S77-80). The second piece of evidence supporting this view is the expression data for LP318a(c16hDGL), which is primarily in IL-5 activated eosinophils, and eosinophils exhibiting hyper-eosinophilia, and in asthma patients. Thus, supporting the linkage between the immune system and the coagulation system.

It has been discovered that LP318a(c16hDGL) sequence (SEQ ID NO: 1) is expressed in the following number of LIFESEQ GOLD™ database tissue and cDNA libraries: Genitalia, Female 1/106; Genitalia, Male 4/114; Germ Cells 1/5; Hemic and Immune System 4/159; Musculoskeletal System 1/47; Respiratory System 1/93; and Nervous System 8/198.

Consequently, based on the expression pattern of LP318a(c16hDGL), its homology to proteins with known functions, and literature suggesting the role of such proteins in human conditions, diseases, syndromes, etc., it is likely that compositions comprising LP318a(c16hDGL) polypeptides (or fragments thereof), polynucleotides (or fragments thereof), and/or LP318a(c16hDGL) antibodies (or LP318a(c16hDGL) binding compositions), and related reagents are also useful for the diagnosis, prognosis, treatment, amelioration, and/or intervention of a disease, condition, or state including, but not limited to, e.g., cell proliferative, autoimmune/inflammatory, coagulative, cardiovascular, neurological, and developmental disorders.

TABLE 1
Primate, e.g., human, LP318a(c16hDGL) polynucleotide sequence (SEQ ID NO: 1) and
corresponding polypeptide (SEQ ID NO: 2). The nomenclature used for this LP reflects chromosomal
location and species status (c16 indicates LP318a(c16hDGL) sequence is found on human chromosome 16.
The lower case letter ‘h’ indicates a sequence is primate, e.g., human.). The ORF for
LP318a(c16hDGL) is 41-679 bp (with the start (ATG) and stop codons (TAG) identified in bold
typeface and underlined in case numbering is misidentified one skilled in the art could determine
the open reading frame without undue experimentation).

LP318a(c16hDGL) DNA Seguence (685 bp) (ORF = 41-679):
LP3l8a(c16hDGL) (start (atg) and stop (tga) codons are indicated in bold
typeface and underlined).
ATGAGGCTTCCCCCAAAGOTTATTTTCCTGCTTCGTTCCATTAGCAAAGCAGTTGCTGCTACAGACTGGGCA
CATTCGGGCCATCGGTGGGTTACTGGGTCAAGAACTTTTGACAGGAAGGCTATGGGCTGCCAGTGGCCTCTG
TGTCTTTGGGTGAGCCCCGGAGTCCAGGTGACCCTCAACCTTCACGGCGAGGCCTCCTACCTCCTCCAGGCA
CTGGGCTCCCTCTGCTCCCCGTGGGCCGCTCCCCGCGTGGGGCCACTGCCCCCGGCCCCCCCCATGGTGCGG
ATTTCAAAGCCCAAGACGTTTCAGGCCTACTTGGATGATTGTCACCGGAGGTATAGCTGTGCCCACTGCCGC
GCTCACCTGGCCAACCACGACGACCTCATCTCCAAGTCCTTCCAGGGCAGTCAGGGGCGTGCCTACCTCTTC
AACTCAGTGGTGAACGTGGGCTGCGGGCCAGCCGAGGAGCGGGTCCTGCTGACCGGCCTCCATGCTGTCGCC
GACATCCACTGCGAGAACTGCAAGACCACTTTGGGCTGGAAATATGAACAGGCCTTTGAGAGCAGCCAGAAG
TACAAAGAGGGGAAGTACATCATTGAACTCAACCACATGATCAAAGACAACGGCTGGGACTGA
LP318a(c16hDGL) Full-Length Seguence (212aa):
The underlined portion is a predicted signal sequence (Met-1 to Lys-40). A predicted SP cleavage site
is between Lys-40 and Ala-41 indicated as follows: 1
MRLPPKVIFLLRSISKAVAATDWAHSGHRWVTGSRTFDRK\hat{ }AMGCQ 45. An optional predicted signal
sequence MRLPPKVIFLLRSISKAVA (Met-1 to Ala-19) based on a different signal peptide analysis allocates
an alternative cleavage site between Ala-19 and Ala-20 indicated as follows: 1
MRLPPKVIFLLRSISKAVA\hat{ }ATDWA 24. Alternative cleavage points may represent alternative mature
LP318a(c16hDGL) variants (all of which are encompassed herein).
MRLPPKVIFLLRSISKAVAATDWAHSGHRWVTGSRTFDRKAMGCQWPLCLWVSPGVQVTLNLHGEASYLLQA
LGSLCSPWAAPRVGPLPPAPAMVRISKPKTFQAYLDDCHRRYSCAHCRAHLANHDDLISKSFQGSQGRAYLF
NSVVNVGCGPAEERVLLTGLHAVADIHCENCKTTLGWKYEQAFESSQKYKEGKYIIELNHMIKDNGWD
An LP318a(c16hDGL) Mature Sequence (172aa):
A predicted mature LP318a(c16hDGL) sequence is as follows:
AMGCQWPLCLWVSPGVQVTLNLHGEASYLLQALGSLCSPWAAPRVGPLPPAPAMVRISKPKTFQAYLDDCH
RRYSCAHCRAHLANHDDLISKSFQGSQGRAYLFNSVVNVGCGPAEERVLLTGLHAVADIHCENCKTTLGWK
YEQAFESSQKYKEGKYIIELNHMIKDNGWD
An additional LP318a(c16hDGL) Mature Sequence (193aa):
Another predicted mature LP318a(c16hDGL) sequence is as follows:
ATDWAHSGHRWVTGSRTFDRKAMGCQWPLCLWVSPGVQVTLNLHGEASYLLQALGSLCSPWAAPRVGPLPP
APAMVRISKPKTFQAYLDDCHRRYSCAHCRAHLANHDDLISKSFQGSQGRAYLFNSVVNVGCGPAEERVLL
TGLHAVADIHCENCKTTLGWKYEQAFESSQKYKEGKYIIELNHMIKDNGWD

TABLE 2

Primate, e.g., human, LP318b(c16hDGL) polynucleotide sequence (SEQ ID NO: 3) and
corresponding polypepude (SEQ ID NO: 4). The nomenclature used for this LP
reflects chromosomal location and species status (c22 indicates LP318b(c16hDGL)
sequence is found on human chromosome 22. The lower case letter ‘h’ indicates
a sequence is primate, e.g., human.). The ORF for LP318b(c16hDGL) is 41.
502 bp (with the start (ATG) and stop codons (TAG) identified in bold typeface and
underlined in case numbering is misidentified one skilled in die art could determine
the open reading frame without undue experimentation).

LP318b (c16hDGL) DNA Sequence (508 bp) (ORF = 41-502):
LP318b(c16hDGL) (start (atg) and stop (tga) codons are indicated in
bold typeface and underlined).
AGAATATATAGAGAATATAGATACATAGGTCCTTTATCCAATGGAGGAGCTGATGCTCAGTCGGAATGACA
GTGTTCTCCACCCATCCTCAGGTCATGACATCCCCCCAGCCTCAGGTCATGAACTCCCCGCATCCTCATAC
GTCATGACCACCGATCCTCAGCTAACAGCCGCGGGGCTGGACTCCCCTGGTTCTGGGCAGCACCGCTTCGT
TCCAGAAAGCACTCGCAGCCAATCTCACGGGACCTTCTTCTTTCAGTCCTTTCAGGGGAGCCAGGGACGCG
CCTACCTCTTCAATTCCGTGGTGAACGTGGGCTGCGGCCCTGCAGAGGAGAGGGTCCTTCTCACCGGGCTG
CATGCGCTTGCCGACATCTACTGCGAGAACTCCAAGACCACGCTCGGGTGCAAATACGAGCATGCCTTTGA
GAGCAGTCAGAAATATAAGGAAGGAAAATTCATCATTGAGCTTGCTCATATGATCAAAGACAATGGCTGGG
AGTAAAAAAAA
LP318b (c16hDGL) Full-Length Sequence (153aa):
MEELMLSRNDSVLHPSSGHDIPPASGHELPASSYVMTTDPQLTAAGLDSPGSGQHRFVPESTRSQSHGTFF
FQSFQGSQGRAYLFNSVVNVGCGPAEERVLLTGLHAVADIYCENCKTTLGWKYEHAFESSQKYKEGKFIIE
LAHMIKDNGWE

Interesting segments of LP318a(c16hDGL) are discovered fragments Leu-11 to Val-18; Ala-19 to Glu-45; Trp-46 to Leu-60; Leu-62 to Gly-74; Ser-75 to Pro-90; Ser-98 to Arg-113; Arg-113 to Ala-124; Asn-125 to Ser-137; Asn-149 to Leu-164; Thr-178 to Glu-195; Lys-197 to Leu-202; Leu-11 to Ala-18; Ala-19 to Phe-37; Gln-45 to Leu-62; Gly-74 to Ala-91; Pro-92 to Lys-101; Thr-102 to Trp-114; Trp-114 to Ala-124; Asn-125 to Gly-136; Ser-137 to Val-148; Asn-149 to His-165; Leu-179 to Glu-195; Lys-6 to Ser-13; Ile-14 to Arg-29; Val-31 to Cys-44; Gln-45 to Ser-53; Ile-97 to Trp-106; Leu-107 to His-122; Leu-123 to Ile-130; Lys-132 to Ala-141; Val-147 to Arg-158; His-165 to Gly-180; and Lys-182 to Lys-197, whose discovery was based on an analysis of hydrophobicity, hydropathicity, and hydrophilicity plots. Additional interesting sections of LP318a(c16hDGL) are the discovered portions of LP318a(c16hDGL) from Pro-4 to Arg-12; Ser-13 to His-28; Trp-30 to Thr-36; Arg-39 to Leu-50; Trp-51 to Thr-59; Leu-60 to Leu-73; Trp-106 to Cys-116; Ala-117 to Ile-130; Ser-131 to Gly-139; Phe-144 to Glu-156; Leu-161 to Asp-169; and Gly-196 to Asn-209. These fragments were discovered based on analysis of antigenicity plots. Further, particularly interesting LP318a(c16hDGL) structures (e.g., such as a helix, a strand, or a coil) that have been discovered are the following LP318a(c16hDGL) helix structures: Leu-11 to Val-18; Ser-67 to Ala-72; and Glu-184 to Phe-187. Particularly interesting discovered coil structures are Met-1 to Pro-4; His-25 to His-28; Gly-33 to Asp-38; Ala-41 to Trp-46; Ser-53 to Gly-55; Leu-62 to Glu-65; Gly-74 to Ala-93; Ser-98 to Pro-100; Cys-110 to Cys-110; His-126 to His-126; Phe-134 to Gly-139; Val-150 to Ala-155; Glu-173 to Lys-182; Glu-195 to Gly-196; and Asp-208 to Asp-212. Particularly interesting discovered strand structures are Leu-48 to Trp-51; Gln-57 to Leu-60; Met-94 to Arg-96; Ala-141 to Leu-143; and Tyr-197 to Glu-201. Further encompassed by the invention are contiguous amino acid residue combinations of any of the predicted secondary structures described above. For example, one coil-strand-coil-helix-coil-strand-coil motif of LP295 combines the Ser-53 to Gly-55 coil, with the Gln-57 to Leu-60 strand, the Leu-62 to Glu-65 coil, the Ser-67 to Ala-72 helix, the Gly-74 to Ala-93 coil, the Met-94 to Arg-96 strand, and the Ser-98 to Pro-100 to form an interesting fragment of contiguous amino acid residues from Ser-53 to Pro-100. Other combinations of contiguous amino acids are contemplated as can be easily determined.

Interesting segments of LP318b(c16hDGL) are discovered fragments Ser-11 to Ile-21; Pro-22 to Met-36; Gly-53 to Ser-66; His-67 to Gly-77; Val-91 to His-106; Thr-119 to Glu-136; Leu-13 to Ile-21; Ile-21 to Trp-34; Val-35 to Ala-44; Ala-45 to Phe-57; Val-58 to Gly-68; Phe-75 to Ala-82; Val-88 to Arg-99; Ala-107 to Gly-121; Lys-123 to Lys-138; Phe-139 to Met-146; Ser-25 to Trp-34; Val-35 to Ala-45; Gly-53 to Ser-66; Phe-75 to Trp-83; Val-88 to Glu-97; Ala-96 to Ala-107; Ala-107 to Asn-115; and Trp-122 to Lys-133, whose discovery was based on an analysis of hydrophobicity, hydropathicity, and hydrophilicity plots. Additional interesting sections of LP318b(c16hDGL) are the discovered portions of LP318b(c16hDGL) from Asn-8 to Ser-17; Ser-33 to Pro-40; His-67 to Glu-80; Phe-85 to Glu-97; Leu-102 to Ala-109;Asp-110 to Thr-119; Lys-123 to Ser-130; and Gly-137 to His-145. These fragments were discovered based on analysis of antigenicity plots. Further, particularly interesting LP318b(c16hDGL) structures (e.g., such as a helix, a strand, or a coil) that have been discovered are the following LP318b(c16hDGL) helix structures: Ile-4 to Leu-9; Phe-52 to Ala-61;Thr-91 to Gly-98;Thr-107 to Phe-115; His-117 to Arg-121; Ile-145 to Ala-164; Glu-171 to Asp-178; Ile-188 to Glu-196; Asn-205 to Leu-208; Gln-281 to Ser-285; Glu-290 to Lys-296; Pro-301 to Ile-304; Lys-330 to Ser-334; and Gln-352 to Lys-356. Particularly interesting discovered coil structures are Ile-14 to Pro-16; Asp-21 to Asp-27; Glu-37 to Arg-38; Ala-46 to Thr-50; Arg-63 to Asn-68; Leu-85 to Lys-88; Lys- 100 to Thr-104; Ser-123 to Asp-127; Asn-132 to Ser-135; Asp-140 to Glu-142; Lys-167 to Gln-168; Lys-212 to Thr-222; Thr-246 to Leu-255; Tyr-264 to Ala-268; Pro-275 to Gly-278; Arg-308 to Asp-317; Thr-343 to Asp-347; Val-361 to Thr-366; Gln-381 to Pro-390; Met-398 to Ser-400; and Ile-409 to Lys-413. Particularly interesting discovered strand structures are Ala-17 to Leu-19; Val-129 to Ile-131; Ala-270 to Ile-273: and Met-392 to Ile-395. Further encompassed by the invention are contiguous amino acid residue combinations of any of the predicted secondary structures described above. For example, one helix-coil-coil-strand-coil motif of LP318b(c16hDGL) combines the Gln-352 to Lys-356 helix, with the Val-361 to Thr-366 and Gln-381 to Pro-390 coils, and the Met-392 to Ile-395 strand to form an interesting fragment of contiguous amino acid residues from Gln-352 to Ile-395. Other combinations of contiguous amino acids are contemplated as can be easily determined.

Features of LP No: 3 (LP288)

LP288 is a novel primate (e.g., human) polypeptide (SEQ ID NO: 6), which is a newly discovered member of the LDL receptor family of proteins. Specifically, LP288 appears to be a member of the LDL receptor-related group of proteins (LRPs), which are found throughout the animal phyla ranging, for example, from invertebrates (such as, e.g., worms such as, e.g., Caenorhabditis elegans), to insects (such as, e.g., Drosophila melanogaster), to birds (e.g., chickens), to mammals (e.g., rodents), and to primates (e.g., humans). LDL receptor-related proteins (LRPs), exhibit typical ligand binding characteristics—high affinity and broad specificity or a “one-receptor-many-ligand” profile. Studies show that LRPs are multi-ligand receptors for lipoprotein remnants and many other physiologically important ligands (see, e.g., the review by Krieger & Herz 1994 Annu. Rev. Biochem 63:601-37). Until recently, LRPs were considered simply as cellular transporters for cholesterol and other lipids, however, this view has changed and other important functions exist for these proteins (e.g., such as regulators of developmental processes and participants in synaptic transmission)(see, e.g., Gotthardt, et al. 2000 J. Biol. Chem. 275:25616-25624). Accordingly, given the “one-receptor-many ligand” profile of LRPs, it is likely that LP288, like other LRPs, will also recognize non-lipoprotein ligands and function in a wide variety of biological processes. For example, recent findings show that an insect LRP (Drosophila arrow) binds non-lipoprotein members of the Wnt/Wng signaling pathway, a pathway that is involved in a variety of developmental and adult functions (see, e.g., Pandur & Kuhl, 2001 BioEssays 23:201-210). LP288 exhibits sequence similarity to both Arrow and another recently described insect LRP (designated CG8909) supporting the view that LP288 may also function in the vertebrate Wnt/Wng signaling pathway.

Typically, LRPs share common structures (such as e.g., amino acid motifs, modules, and/or domains) that are arranged in characteristic locations within an LRP (e.g., most easily visualized with respect to their positioning in a primary LRP amino acid sequence). For example, the following amino acid motifs, modules, and/or domains are routinely found in characteristic locations in LRPs.

At the N-terminal portion of a canonical LRP, a series (e.g., ranging from about two to about eight) of LRP ligand-binding-like domains (such as, e.g., the low-density lipoprotein receptor class A module) are typically found. Routinely, an LRP-A module or domain contains between 2-12 complement-type cysteine rich repeats. LP288 contains approximately eight LRP-A-like domains at its N-terminad most location. Following the LRP ligand-binding-like region (i.e., moving C-terminad along the primary amino acid structure of a typical LRP), one or several epidermal growth factor (EGF)-like domains are characteristically positioned. LP288 contains two such EGF-like domains at this location. Typically, following the N-terminal-most EGF-like domains, is a region characterized by the presence of multiple YWTD-like motifs that is flanked C-terminad by one or several EGF-like domains. Characteristically, in all LRPs reported to date, it has been shown (Springer 1998J. Mol. Biol. 283:837-62) that two YWTD containing regions are never contiguous but are always separated by EGF- or FN3-like domains. For example, in vertebrate LRP1 and LRP2 some YWTD regions are flanked by single EGF-like domains while other YWTD regions are flanked by multiple (e.g., up to 11 EGF-like domains in the C. elegans LRP1), however two YWTD regions are never adjacent). LP288 exhibits such an ordering so that each LP288 YWTD region is made up of multiple YWTD-like motifs that are not contiguous but are separated by one or more flanking EGF-like domains

Recently, these YWTD-containing regions of LRPs have themselves been more fully characterized and have now been shown to be more structured than had been previously realized (Springer 1998 J. Mol. Biol. 283:837-62). Using established three-dimensional structures of flanking EGF-domains as models, Springer demarcated the boundaries of LDR EGF-like domains as being approximately two or three amino acid residues before the first cysteine of the EGF-like domain and two or three residues after the last cysteine of the EGF-like domain. Using such EGF boundaries as markers, Springer analyzed YWTD-containing regions in a variety of proteins to establish that a consensus sequence existed for every YWTD region that was flanked by an EGF-like domain (see, e.g., Springer 1998 J. Mol. Biol. 283:837-62). Consequently, it was established that every LRP YWTD containing region is made up of six separate YWTD repeat sequences (each repeat sequence, designated as YWTD repeat Nos. 1-6, is approximately 40-44 residues in length). A set of six YWFD repeats make up a structural unit that Springer defines as a single YWTD domain (to avoid confusion, Applicants refer to similar LP288 regions as YWTD islands). The sequence and structure of the LP288 YWTD regions conform to such a model. Each YWTD region in LP288 can be further subdivided into a set of six individual YWTD-like repeats, which possess specific characteristic features. Every LP288 YWTD island is bounded by at least one EGF-like domain.

An individual YWTD island is predicted to fold into a higher order structure designated a six-bladed beta-propeller, which is composed of six similar subunits (see, e.g., Murzin, et al. 1996 J. Mol. Biol. 247:536-540). Each beta sheet of the beta propeller has an almost identical tertiary structure (but see below suggesting that the blade positions may be more conserved between than within propellers) and the beta sheets are radially arranged about a pseudosymmetrical axis ultimately yielding a compact higher order structure that is cylindrical or toroidal-like in shape and that brings neighboring modules (e.g., EGF-like domains) into close proximity. Using the LP288 sequence information provided herein, similar higher order structures can be predicted for the four LP288 YWTD islands by one of ordinary skill using common art known techniques (see, e.g., the descriptions in the methods section of Springer 1998 J. Mol. Biol. 283:837-62, and Fulop and Jones 1999, Curr. Opin. Struct. Biol. 9:715-721, incorporated herein by reference for these teachings).

As stated above, each YWTD island is characteristically made up of six individual YWTD repeats (designated, from the N- to C-terminad direction, as YWTD repeats Nos. 1-6). Each YWTD island is flanked by one or more EGF-like domains. After analyzing YWTD repeats from 89 such YWTD islands, Springer demonstrated that each YWTD repeat has within it a characteristic motif sequence (comprising about five contiguous amino acid residues), which is located at the beginning of the repeat (e.g., within the first 5-7 amino acids of the repeat sequence). For example, YWTD repeats Nos. 2-6 characteristically have a YWTD-like consensus motif (e.g., Tyr-Trp-Thr-Asp) located at the beginning of the repeat sequence. However, at repeat position No. 1, a different (though similar) motif is characteristically found. Instead of a YWTD-like motif, similar residues (such as, LFAN (Leu-Phe-Ala-Asn)) are found. The repeat sequences found at YWTD repeat position No. 1 (in a YWTD island) are similar in length to other YWTD repeat sequences and, except for the YWTD-like motif itself, sequences at the No. 1 repeat positions are as homologous to other repeats (e.g., those at positions Nos. 2-6) as those repeats are to themselves. Moreover, LFAN-like motifs have also been shown to be approximately equivalent to YWTD-like motifs (see, e.g., J. Mol. Biol (98) 283: 837-62), further supporting the idea that a repeat sequence at position No. 1 is, in fact, a YWTD repeat sequence like the repeat sequences at positions Nos. 2-6. Springer also suggests that another characteristic feature of YWTD repeat motifs is that the amino acid residue positioned before each YWTD-like or LFAN-like motif is occupied by a hydrophobic amino acid residue. This feature is typically found in every repeat throughout every YWTD island. Springer suggest that such YWTD repeat arrangements in LRPs have been maintained over evolutionary time—since the divergence of nematodes from chordates to maintain the resulting higher order structural unit-a beta propeller comprising six similar structural subunits. Analysis of the YWTD regions of LP288, demonstrates that these regions exhibit both the structural and organizational features that are characteristic of LRP YWTD islands (as characterized by Springer). Such evidence further supports the conclusion that LP288 is a novel LRP.

LP288 has four YWTD islands, each of which is flanked by at beast one EGF-like domain. Every LP288 YWTD island is made up of six individual YWTD repeats, which are approximately 40-43 contiguous amino acids in length. Moreover, each LP288 YWTD repeat contains an amino acid motif sequence, located at the beginning of the repeat, that has either a YWTD-like or LFAN-like motif depending upon which position the motif sequence occupies in the YWTD island (e.g., position No. 1, 2, 3, 4, 5, or 6). For instance, as predicted, all LP288 position No. 1 repeats contain an LFAN-like motif at the beginning of the repeat sequence. Futhermore, a hydrophobic amino acid residue occupies the position preceding every LP288 LFAN-like motif. For example, the following LP288 hLFAN-like motifs (where h is any hydrophobic amino acid residue) are found at the No. 1 repeat positions of each of the four LP288 YWTD domains: LLFAN (for LP288 YWTD domain 1), LLFAR (for LP288 YWTD domain 2), LIFAR (for LP288 YWTD domain 3), and LLFSS (for LP288 YWTD domain 4). Comparison of these four motifs results in a consensus LFAN-like motif for LP288 of “lFup.”

Similarly, all LP288 repeat sequences at positions Nos. 2-6 in the LP288 YWTD islands contain a YWTD-like motif preceded by a hydrophobic amino acid residue. For example, the following hYWTD-like motifs (where h is any hydrophobic amino acid residue) are found at the beginning of LP288 YWTD repeat positions Nos. 2-6. VFWSD, LYWTD, IYWTD, MYWVD, and LYWTD (for LP288 YWTD domain 1); VYWTD, LYWTD, MYWTD, LYWAD, and IYWTD (for LP288 YWTD domain 2); VYWSD, VYWTD, MYWTD, LYWAD, and IYWTD (for LP288 YWTD domain 3); and VYYTD LYWTD, LFWTD, IYWVD, and IYWTD (for LP288 YWTD domain 4). Comparison of these motifs results in consensus YWTD-like motifs for LP288 of “VaaoD,” “IYWTD,” “haWTD,” “hhWsD,” and “IYWTD” for, respectively, repeat positions Nos. 2-6.

Interestingly, a cladistic-like analysis comparing the sequences of every YWTD repeat in LP288 shows that a positional effect exists for the sequence of any particular YWTD repeat within any LP288 YWTD domain. So that, for instance, a repeat sequence at position No. 3 is more similar to any other repeat sequence at that same position (i.e. No. 3) in any other island than it is to other YWTD repeat sequences. For example, all four position No. 1 YWTD repeat sequences in LP288 are more closely similar (using sequence identity as a measure) to each other than to repeat sequences at other positions in the LP288 YWTD islands. Consequently, an analysis of all 24 LP288 YWTD repeat sequences (i.e., 6 repeats/island×four LP288 YWTD islands) produces a cladigram-like grouping that is arranged by position so that all YWTD repeats at position No. 1 are grouped as “sister” groups; all YWTD repeats at position No. 2 are grouped as “sister” groups; all YWTD repeats at position No. 3 are grouped as “sister” groups, and so on. An exception to this finding exists, however, at YWTD repeat position No. 5. There, the position No. 5 YWTD repeat sequence of LP288 YWTD domains 1 and 2 are more similar to each other while the No. 5 YWTD repeat sequences of LP288 YWTD domains 3 and 4 are more alike. Thus, a subdivision of sequence similarity exists at YWTD repeat position No. 5 so that the position No. 5 sequences of islands' 1 and 2 are most alike. The same pertains for sequences at position No. 5 in YWTD islands' 3 and 4.

Interestingly, LP288 YWTD islands' 1 and 2 are adjacently ordered followed by the subsequent adjacent ordering of islands 3 and 4. Without being bound by theory, such sequence similarity combined with such ordering suggests that the conservation of sequences within a particular positional YWTD repeat is maintained to functionally conserve the resulting higher order structures that make up the blades of the beta-propeller encoded by a YWTD island as each repeat in a YWTD island is used to construct subunits of the beta-propeller structure. Moreover, adjacent blades of separate beta-propeller structures have been proposed to interact to form higher order structures. Accordingly, it is possible that repeat position No. 5 in LP288 YWTD islands' 1 and 2 and/or repeat position sequences No. 5 of LP288 islands' 3 and 4 may interact to form higher order multi-beta-propeller structure/s (see, e.g., Fulop and Jones 1999, Curr. Opin. Struct. Biol. 9:715-721). Furthermore, it is possible that a particular position of an LP288 YWTD repeat within a YWTD island has a similar structural and/or functional role from one island to another thus compelling evolutionary selection to maintain sequence similarity at specific repeat positions between different islands. Such an analysis of LP288 is consistent with the interpretation of Springer (1998) who shows that other LRPs (e.g., vertebrate LRP1, and LRP2) also exhibit groupings of two or four YWTD islands that are separated from other such groups by only single EGF-like domains between the adjacent YWTD islands of a group. Springer suggests that the proximity of these LRP YWTD islands imply that they act in concert, for example, in binding a single ligand. The groupings in LP288 (of four YWTD domains) add further weight to the evidence that LP288 is an LRP. Moreover, it also suggests that the groupings of YWTD islands in LP288 may convey a similar function by promoting mutual interactions (e.g., to bind a single ligand).

The importance of YWTD islands in low-density lipoprotein receptors (LDLRs) has been demonstrated (in one manner) by the fact that a large proportion of mutations map to YWTD regions in families afflicted by hypercholesterolaemia. These YWTD mutations are particularly detrimental when they occur in YWTD repeat sequences at positions No. 1 and 2 (see, e.g., Hobbs, et al 1992 Annu Rev Genet 24: 133-170, Chae, et al. 1999 Clin Genet 55(5): 325-31, and Krawczak & Cooper, 1997 Trends Genet 13:121-122). An explanation of the consequence of such mutations has recently been advanced in light of the effect these YWTD mutations have on the formation or functioning of beta-propeller structures proposed to form from YWTD islands (see, e.g., Springer 1998 J. Mol. Biol. 283:837-62 and references cited therein). An additional characteristic feature of LRPs are that these receptors typically have a single transmembrane segment which is followed by a cytoplasmic tail containing characteristic intracellular binding motifs.

LP288 possesses both a single transmembrane segment and intracellular binding motifs that have been described as being characteristic for LRPs. For example, as reported to date, LDLRs (including LRPs) characteristically have at least one NPxY motif in their cytoplasmic tail (where N is asparagine; P is proline; Y is tyrosine; and x is any amino acid residue). These NPxY motifs have been reported to function as internalization signals (e.g., required for clustering of LRPs into coated pits). However, others have recently proposed that a cytoplasmic YxxL-like motif and di-leucine repeats (LL) rather than NpxY motif, serve as the dominant agents for LRP endocytosis (see, e.g., Li, et al. 2000 J. Biol. Chem 275: 17817-17194). LP288 contains both NPxY-like (e.g., NPSY), YxxL-like (e.g., YNQL, and WDDL), YxxP-like (e.g., YSNP), and di-leucine repeat (LL) (e.g., GLLR, and QLLQ) sequence motifs (among others) in its cytoplasmic portion.

The conflicting data regarding the role of the NPxY and YxxL motifs may be reconciled by a current proposal suggesting that LRPs may both endocytosis (e.g., through coated pits) and also function via non-internalized intracellular signaling pathways (e.g., MAP kinase pathways)(see, e.g., Gotthardt, et al. 2000 J. Biol. Chem 275 No. 33: 25616-25624, and Pandur & Kuhl, 2001 BioEssays 23:201-210). Not being bound by theory, the ability to direct an LRP toward one or another pathway (e.g. internatilized or non-internalized) may be mediated depending upon what particular binding partners are complexed with the cytoplasmic portion of an LRP. For example, DAB1 binding to LRP tails competes and prevents bound LRP from clustering into coated pits and thus, DAB1 prevents subsequent LRP endocytosis. This data suggests a mechanism by which alternative signaling through LDL receptor family members is accomplished.

In this mechanism, specific assemblies on a particular LRP cytoplasmic tail (e.g., via scaffold and adaptor proteins that may be engaged in intracellular signaling pathways) drive an LRP system towards, for example, an endocytotic pathway or an intracellular signaling pathway. Such a multi-functional LRP system would be dynamic depending on various factors controling it (such as, e.g., the number and kind of binding partners, the on-off rates of binding, binding partner local concentrations, the timing, and/or seeding of initial complex binding partners, binding constants and/or specificities or affinities to particular LRP cytoplasmic motifs, etc.). Given that it has been shown that the NPxY motif is also a substrate for various intracellular binding partners (such as, e.g., the Shc adaptor protein, which has the ability to interact with a large number of tyrosine-phosphorylated proteins), and that the NPxY motif also binds a PI/PTB domain, which itself has been identified as a binding motif in the Shc adaptor protein (Margolis 1996 J Lab Clin Med 128(3): 235-41), the NPxY motif may be a context-dependent motif that functions both in endocytosis or via an intracellular signaling pathway depending upon the interaction of other members of a binding complex on an LRP cytoplasmic tail. Such a system would resemble the combinatorial-like functioning features of DNA transcriptional regulator complexes (e.g., like those seen in “turning on” HOX genes).

Recently, however, an additional finding (see, e.g., Schaefer, et al. 1999 J. Biol. Chem. 274: 37965-37973) suggests that endocytotic internalization is required to activate an intracellular signaling pathway (e.g., the mitogen-activated protein kinase (MAPK) cascade). This data suggests that LP288 may also function similarly, so that internalization is required for and/or biases a particular intracellular signaling pathway. Thus, an LP288 variant designed to block its internalization (e.g., but mutating or abolishing an Lxxy or tSxV motif in its cytoplasmic portion) would be restricted in its signaling options and vice versa.

To allow diverse signals to be routed through a multi-ligand receptor such as an LRP, specificity of signal transduction is maintained through the assembly of multiprotein complexes. Thus, a given LRP receptor may bind multiple signaling molecules, yet activation of the appropriate intracellular signaling pathway would depend on the particular binding partner members of a complex, e.g. such as co-receptors (e.g., Wnt) for the extracellular portion of a complex and/or adaptor or scaffold proteins for the intracellular portion of a complex. These homo/hetero-multimeric complexes will likely be dynamic (e.g., such as, using the requisite formation of an extracellular complex to act as a temporary platform for a subsequent formation of an intracellular complex).

Applicants invention encompasses such complexes that are formed with LP288 (e.g., both extracellular, intracellular, and complexes including both intra- and extra-cellular complexes). In addition, Applicants invention encompasses LP288 variants whose bias drive pathway specificity for LP288 binding complexes, for example, by biasing the formation of particular LP288 complexes (both intra- and extra-cellularly). For example, to bias a particular pathway, LP288 variants are encompassed herein in which, for example, cytoplasmic-like motifs are removed, added, and/or mutated. Moreover, particular LP288 variants are encompassed herein that, e.g., bias which pathway an LP288 would take (e.g., endocytosis, or intracellular signaling). Non-limiting examples of such LP288 variants include those in which, for example, YxxL, NPxY, tSxV, or YxxP LP288 motifs have been removed, added, relocated, and/or mutated (e.g., such as by substituting a tyrosine residue (Y) for an X in an YxxP motif; or by adding a second terminal SxV motif to the end of an LP288 cytoplasmic tail to, e.g., increase the binding affinity for, e.g., PDZ domains).

The presence of characteristic intracellular binding motifs in LP288 further supports a view that LP288 can form multimeric complexes through interactions with various binding partners. Additionally, other studies (Gotthardt, et al. 2000 J. Biol. Chem 275 No. 33: 25616-25624, incorporated herein by reference) reveal that LRPs interact (e.g., through proteins having either PID or PDZ domains, with a much broader range of proteins than had previously been recognized (such as, e.g., cytoplasmic adaptor and scaffold proteins such as, e.g., SEMCAP-1, JIP-1, PSD-95, JIP-2, Talin homologue, OMP25, CAPON, DAB1, ICAP-1, MINT2, PIP4, 5-Kinase homologue, Sodium channel brain 3, and APC subunit 10). These molecules have known functions that relate to, for example, cell adhesion, cell activation, reorganization of the cytoskeleton, neurotransmission, regulation of synaptic transmission, activation and/or modulation of MAP kinase pathways, local organization of the cytoskeleton, cell adhesion, and endocytosis. Accordingly, it is likely that the LP288 (in conjunction with binding complexes formed with these and other similar molecules) will perform similar functions. For example, using a yeast two-hybrid screening assay, and/or an in vitro GST pull down assay, and/or similar such approaches, one of ordinary skill in the art could identify, without undue experimentation, binding partners of LP288 signaling complexes that form with a cytoplasmic portion of LP288 (such as, e.g., the binding partners SEMCAP-1, JIP-1, PSD-95, JIP-2, Talin homologue, OMP25, CAPON, DAB1, ICAP-1, MINT2, PIP4, 5-Kinase homologue, Sodium channel brain 3, and APC subunit 10). Given Applicants teachings regarding the LP288 sequence; the LP288 cytoplasmic portion; teachings of various LP288 domains, motifs, regions, and/or modules; the identification of particular binding motifs in an LP288 cytoplasmic domain; the knowledge in the art regarding methods of determining such binding partners (e.g., such as, two-hybrid assays and GST pull-down assays); and teachings in the art regarding performing such methods with LRP-like proteins (see, e.g., the methods described in Gotthardt, et al. 2000 J. Biol. Chem 275: 25616-25624 which are incorporated herein by reference for these teachings), one of ordinary skill in the art would not require undue experimentation in determining binding partners for LP288.

For example, recently it has been shown that an insect LDLR protein (Drosophila arrow, which is homologous to vertebrate LRP5 and LRP6) forms extracellular and intracellular heteromeric complexes with members of the Wnt/Wg signaling pathway, such as, for example the transmembrane proteins Frizzled (Fz) and Dfrizzled (DFz2) (see, e.g., Pandur & Kuhl, 2001 BioEssays 23:201-210). These data suggest that these cell surface molecules are essential co-receptors for Wnt ligands. Members of the Wnt/Wingless (Wg) family of secreted glycoproteins function in a variety of developmental processes including cell differentiation, cell polarity, cell migration, and cell proliferation. LP288 shows sequence similarity to both the Drosophila arrow protein and to another Drosophila LDRL-like protein related to arrow (designated CG8909). Both arrow and CG8909 are reported to be LDRL-like receptors exhibiting sequence similarity to primate, rodent (LRP5 and LRP6), fish, and worm LDRL-like receptor proteins (see, e.g., the report on CG8909 in the Drosophila Flybase located at http: //flybase.bio.indiana.edu). Accordingly, LP288 may also play a role in developmental events mediated by vertebrate homologs of the Wnt/Wg cascade of proteins (e.g., such as vertebrate homologs of Drosophila genes known to act downstream of wingless, such as, e.g., Adenomatous polypopsis coli tumor suppressor homolog 2, Apc, armadillo, arrow, Axin, decapentaplegic, dishevelled, engrailed, eyelid (antagonizes Wingless signaling), frizzled (receptor for Wingless), frizzled2 (receptor for Wingless), frizzled3 (antagonizes Wingless signaling), gooseberry distal, gooseberry proximal, lines, naked cuticle, pangolin, porcupine, shaggy/zeste white 3, and supernumerary limbs). Additionally, LP288 may form intracellular complexes with proteins known to be involved in binding intracellular motifs (e.g., such as PZD domains) of members of this signaling pathway (such as, e.g., vertebrate homologs of the binding partners of Frizzled proteins (e.g., disheveled (dsh), prickled, inturned, fuzzy, and multiple-wing-hair proteins) since it has also been shown that the terminal S/TxV motif of members of the frizzled gene family interact directly with PDZ domains found vertebrate intracellular molecules. For example, a Xenopus dsh protein, when co-expressed with rat frizzled family members in a Xenopus blastomer, translocates from a cytoplasmic pool to a membrane location, indicating both the conserved functional association of these proteins and their sequence conservation (since one protein is from an amphibian and the other is from a rodent). Furthermore, phenotypes resulting from mutations in a mouse homolog of arrow (LRP6) display a variety of defects such as, for example, midbrain/hindbrain morphogenetic defects, axis truncations, and limb patterning defects.

LP288 has five PxxP-like motif sequences (PDEP, PPAP, PVLP, PNTP, and PAAP), where “P” is Proline, and x is any amino acid residue. Typically, PxxP motifs are found intracellularly in proline rich regions, for example, such as in the cytoplasmic tails of receptors. The PxxP motif is known to bind with SH3 domains of various intracellular proteins (see, e.g., Kay, et at. 2000 FASEB J. 14:231-241). What is unusual in LP288 about the appearance of such a proline-rich region containing PxxP motifs, is that this region is located in the extracellular portion of LP288, just before the transmembrane domain. However, PxxP motifs are typically found in the cytoplasmic portion of receptor proteins where, upon binding the receptor's cognate ligand, intracellular proteins subsequently interact with the receptor's nascently-forming cytoplasmic binding complex to activate particular intracellular pathways and/or cascades. Accordingly, the presence of such a proline-rich PxxP-containing region at an extracellular location of LP288 is interesting. However, a recent finding (Schaefer, et al. 1999 J. Biol. Chem. 274:37965-37973) suggests a possible function for such a placement. Schaefer, et al. show that a neural cell adhesion molecule requires internalization (e.g., via endocytosis) to activate a particular signaling pathway (a mitogen-activated protein kinase (MAPK) cascade). Similarly, perhaps the extracellular presence of a proline-rich PxxP-containing region in LP288 is a mechanism to mask SH3-binding until an endocytotic pathway is realized for an LP288. Absent internalization, the intracellular binding sites in an LP288 would control the system (e.g., leading to activation of a different signaling cascade, such as, for example, a JAK/STAT pathway). However, if LP288 did become internalized (e.g., via binding of unique extracellular ligand/s or, e.g., by formation of a specific extracellular binding complex), then the previously sequestered PxxP motifs would now, in effect, become “unmasked” via internalization and be made available for interaction with, for example, SH3-like binding partners. Consequently, the presence of an extracellular proline-rich PxxP-containing region in LP288 suggests a previously unrecognized mechanism to insure its signal transduction specifity for a receptor having “high-affinity” and “broad-specificity” characteristics.

Given all the available evidence discussed herein, LP288 is a novel LRP member of the low-density lipoprotein receptor family. This is confirmed by LP288's possession of a repertoire of domains and modules, that have been shown to characteristic for the LDLR family (e.g., having multiple ligand binding domains, EGF modules, YWTD domain(s), a single transmembrane ‘domain’, and a cytoplasmic tail with characteristic sequence motifs).

Given the sequence information and knowledge of the secondary structural features of LRPs and how these features map onto the LP288 sequence presented herein (e.g., the relationship between the primary amino acid sequence of LP288 YWTD regions and higher order beta propeller-like structures or the relationship of EGF-like domains and their higher order structures) one of ordinary skill in the art would be able to design amino acid modifications of LP288 to affect LP288 function in such regions (e.g., like those found in the YWTD regions of individuals with lipid disorders). In fact, commercial services are available to rapidly produce three-dimensional configurations and higher order structures of known primary amino acid sequences thus avoiding undue experimentation when assessing higher order structures of a sequence of interest (see, e.g., Structural GenomiX, 10505 Roselle St., San Diego, Calif. 92121).

LP288 nucleic acid sequence (SEQ ID NO: 5) is expressed in the following number of LIFESEQ GOLD™ database tissue and cDNA libraries: Cardiovascular System 5/68; Connective Tissue 2/47; Digestive System 16/148; Embryonic Structures 3/21; Endocrine System 6/53; Exocrine Glands 5/64; Genitalia, Female 11/106; Genitalia, Male 13/114; Hemic and Immune System 19/159; Liver 5/35; Musculoskeletal System 3/47; Nervous System 65/198; Pancreas 2/24; Respiratory System 6/93; Skin 2/15; and Urinary Tract 7/64.

Consequently, based on the expression pattern of LP288, its homology to proteins with known functions, and literature suggesting the role of such proteins in human conditions, diseases, syndromes, etc., it is likely that compositions comprising LP288 polypeptides (or fragments thereof), polynucleotides (or fragments thereof), and/or LP288 antibodies (or LP288 binding compositions), and related reagents are also useful for the diagnosis, prognosis, treatment, amelioration, and/or intervention of a disease, condition, or state including, but not limited to, e.g., cell proliferative, autoimmune/inflammatory, coagulative, cardiovascular, neurological, and developmental disorders.

TABLE 3

Primate, e.g., human, LP288 polynucleotide sequence (SEQ ID NO: 5) and corresponding pnlypeptide.
(SEQ ID NO: 6). The ORF for LP288 is 142-5859 bp (with the start (ATG) and stop codons (TAA)
identified in bold typeface and underlined in case numbering is misidentified one skilled in the art
could determine the open reading frame without undue experimentation).

LP288 DNA sequence: (7974bp) (ORF = 142-5859):
ATGAGGCGGCAGTGGGGCGCGCTGCTGCTTGGCGCCCTGCTCTGCGCACACGGCCTGGCCAGCAGCCCCGA
GTGTGCTTGTGGTCGGAGCCACTTCACATGTGCAGTGAGTGCTCTTGGAGAGTGTACCTGCATCCCTGCCC
AGTGGCAGTGTGATGGAGACAATGACTGCGGGGACCACAGCGATCAGGATGGATGTATACTACCTACCTGT
TCCCCTCTTGACTTTCACTGTGACAATGGCAAGTGCATCCGCCGCTCCTGGGTGTGTGACGGGGACAACGA
CTGTGAGGATGACTCGGATGAGCAGGACTGTCCCCCCCGGGAGTGTGAGGAGGACGAGTTTCCCTGCCAGA
ATGGCTACTGCATCCGGAGTCTGTGGCACTGCGATGGTGACAATGACTGTGGCGACAACAGCGATGAGCAG
TGTGACATGCGCAAGTGCTCCGACAAGGAGTTCCGCTGTAGTGACGGAAGCTGCATTGCTGAGCATTGGTA
CTGCGACGGTGACACCGACTGCAAAGATGGCTCCGATGAGGAGAACTGTCCCTCAGCACTGCCAGCGCCCC
CCTGCAACCTGGAGGAGTTCCAGTGTGCCTATGGACGCTGCATCCTCGACATCTACCACTGCGATGGCGAG
GATGACTGTGGAGACTGGTCAGACGAGTCTGACTGCTCCTCCCACCAGCCCTGCCGCTCTGGGGAGTTCAT
GTGTGACAGTGGCCTGTGCATCAATGCAGGCTGGCGCTGCGATGGTGACGCGGACTGTGATGACCAGTCTG
ATGAGCGCAACTGCACCACCTCCATGTGTACGGCAGAACAGTTCCGCTGTCACTCAGGCCGCTGTGTCCGC
CTGTCCTGGCGCTGTGATGGGGAGGACGACTGTGCAGACAACAGCGATGAAGAGAACTGTGAGAATACAGG
AAGCCCCCAATGTGCCTTGGACCAGTTCCTGTGTTGGAATGGGCGCTGCATTGGGCAGAGGAACCTGTGCA
ACGGGGTCAACGACTGTCGTGACAACAGCGACCAAAGCCCACAGCAGAATTGCCGGCCCCGGACGGGTGAG
GAGAACTGCAATGTTAACAACGGTGGCTGTGCCCAGAAGTGCCAGATGGTGCGGGGGGCAGTGCAGTGTAC
CTGCCACACAGGCTACCGGCTCACAGAGGATGGGCACACGTGCCAAGATGTGAATGAATGTGCCCAGGAGG
GGTATTGCAGCCAGGGCTGCACCAACAGCGAAGGGGCTTTCCAATGCTGCTGTCAAACAGGCTATGAACTA
CGGCCCGACCGGCGCAGCTGCAAGGCTCTGGGGCCAGAGCCTGTGCTGCTCTTCCCCAATCGCATCGACAT
CCGGCAGGTGCTGCCACACCGCTCTGAGTACACACTGCTGCTTAACAACCTGGAGAATGCCATTGCCCTTG
ATTTCCACCACCGCCGCGAGCTTGTCTTCTOGTCAGATGtCACCCTGGACCGCATCCTCCGTGCCAACCTC
AACGGCAGCAACGTGGAGGAGGTTGTGTCTACTGGGCTGGAGACCCCAGGGGGCCTGGCTGTGGATTGGGT
CCATGACAAACTCTACTGGACCGACTCAGGCACCTCGAGGATTGAGGTGOCCAATCTGGACGGGGCCCACC
GGAAGGTGTTGCTGTGGCAGAACCTGCAGAAGCCCCGGGCCATTGCCTTGCATCCCATGGAGGGTACCATT
TACTGGACAGACTGGGGCAACACCCCCCGTATTGAGGCCTCCAGCATGGATGGCTCTGOACGCCGCATCAT
TGCCGATACCCATCTCTTCTGGCCCAATGGCCTCACCATCGACTATGCCGGGCGCCGTATGTACTGGGTGG
ATGCTAAGCACCATGTCATCGAGAGGGCCAATCTGGATGGGAGTCACCGTAAGGCTGTCATTAGCCAGGGC
CTCCCGCATCCCTTCGCCATCACAGTGTTTGAAGACAGCCTGTACTGGACAGACTGGCACACCAAGAGCAT
CAATAGCGCTAACAAATTTACGGGGAAGAACCAGGAAATCATTCGCAACAAACTCCACTTCCCTATGGACA
TCCACACCTTGCACCCCCAGCGCCAACCTGCAGGGAAAAACCGCTCTGGGGACAACAACGCACGCTGCACG
CACCTGTGTCTGCCCAGTGGCCAGAACTACACCTGTGCCTGCCCCACTGGCTTCCGCAAGATCAGCAGCCA
CGCCTGTGCCCAGAGTCTTGACAAGTTCCTGCTTTTTGCCCGAAGGATGGACATCCOTCGAATCAGCTTTG
ACACAOAGGACCTGTCTGATGATGTCATCCCACTGGCTGACGTGCGCAGTGCTGTGGCCCTTGACTGGGAC
TCCCGGGATGACCACGTGTACTGGACAGATGTCAGCACTGATACCATCAGCAGGGCCAAGPGGGATCCAAC
AGGACAGGAGGTGGTAGTGGATACCAGTTTGGAGAGCCCAGCTGGCCTGGCCATTCATTGGGTCACCAACA
AACTGTACTGGACAGATGCAGGTACAGACCGGATTGAAGTAGCCAACACAGATGGCAGCATGAGAACAGTA
CTCATCTGGGAGAACCTTGATCGTCCTCGGGACATCGTGGTGGAACCCATGGGCGGGTACATCTATTGGAC
TGACTGGGGTGCGAGCCCCAAGATTGAACGAGCTGGCATGGATGCCTCAGGCCGCCAAGTCATTATCTCTT
CTAATCTGACCTGGCCTAATGGGTTAGCTATTGATTATGGCTCCCAGCGTCTATACTGGGCTGACGCCGGC
ATGAAGACAATTGAATTTGCTGGACTGGATGGCAGTAAGAGGAAGGTGCTGATTGGAAGCCAGCTCCCCCA
CCCATTTGGGCTGACCCTCTATGGAGAGCGCATCTATTGGACTGACTGGCAGACCAAGAGCATACAGAGCG
CTGACCGGCTGACAGGCCTGGACCGGGAGACTCTGCAGCAGAACCTGOAAAACCTAATGGACATCCATGTC
TTCCACCGCCGCCGGCCCCCAGTGTCTACACCATGTGCTATGGAGAATGGCGGCTGTAGCCACCTGTGTCT
TAGGTCCCCAAATCCAAGCGGATTCAGCTGTACCTGCCCCACAGGCATCAACCTGCTGTCTGATGGCAAGA
CCTGCTCACCAGGCATGAACAGTTTCCTCATCTTCGCCAGGAGGATAGACATTCGCATCGTCTCCCTGGAC
ATCCCTTATTTTCCTGATGTGGTGGTACCAATCAACATTACCATCAACAACACCATTGCCGTTGGAGTAGA
CCCCCAGGAAGGAAAGGTGTACTGGTCTGACAGCACACTGCACAGGATCAGTCGTGCCAATCTGGATGGCT
CACAGCATOAGGACATCATCACCACAGGGCTACAGACCACAGATGGGCTCGCGCTTGATGCCATTGGCCGG
AAAGTATACTGGACAGACACGGGAACAAACCGGATTGAAGTGCGCAACCTGGACGGGTCCATGCGGAAAGT
GTTGGTGTGGCAGAACCTTGACAGTCCCCGGGCCATCGTACTGTACCATGACATGGGGTTTATGTACTGGA
CAGACTGGGGGGAGAATGCCAAGTTAGAGCGGTCCGGAATGCATGGCTCAGACCGCGCGGTGCTCATCAAC
AACAACCTAGGATGGCCCAATGGACTGACTGTGGACAAGGCCACCTCCCAACTGCTATGGGCCGATGCCCA
CACCGAGCGAATTGAGGCTGCTGACCTGAATGGTGCCAATCGGCATACATTGGTGTCACCGGTGCACCACC
CATATGGCCTCACCCTGCTCGACTCCTATATCTACTGGACTGACTGGCAGACTCGCAGCATCCACCGTGCT
GACAAGGGTACTGGCAGCAATGTCATCCTCGTGAGGTCCAACCTCCCAGGCCTCAPGGACATGCAGGCTGT
GGACCGGGCACAGCCACTAGGTTTTAACAAGTGCGGCTCGAGAAATGGCGGCTGCTCCCACCTCTGCTTGC
CTCGGCCTTCTGGCTTCTCCTGTGCCTGCCCCACTGGCATCCAGCTGAAGGGAGATGGGAAGACCTGTGAT
CCCTCTCCTGAGACCTACCTGCTCTTCTCCAGCCGTGGCTCCATCCGGCGTATCTCACTGGACACCAGTGA
CCACACCGATGTGCATGTCCCTGTTCCTGAGCTCAACAATGTCATCTCCCTGGACTATGACAGCGTGGATG
GAAAGGTCTATTACACAGATGTGTTCCTCGATGTTATCAGGCGAGCAGACCTGAACGGCAGCAACATCCAG
ACAGTGATCGGGCGAGGGCTGAAGACCACTGACCGGCTGGCAGTGGACTGGGTCGCCAGGAACCTGTACTG
GACAGACACAGGTCGAAATACCATTGAGGCGTCCAGCCTCGATCGTTCCTGCCCCAAAGTACTGATCAACA
ATAGCCTGGATGAGCCCCGGGCCATTGCTGTTTTCCCCAGGAAGGGGTACCTCTTCTGGACAGACTCGGGC
CACATTGCCAAGATCGAACGGGCAAACTTGGATGGTTCTGAGCGGAAGGTCCTCATCAACACAGACCTGGG
TTGGCCCAATGGCCTTACCCTGGACTATGATACCCGCAGGATCTACTGGGTGGATGCGCATCTGGACCGCA
TCGAGACTGCTGACCTCAATGGGAAACTGCGGCAGGTCTTGGTCGGCCATGTGTCCCACCCCTTTGCCCTC
ACACAGCAAGACAGGTGGATCTACTGGACAGACTGGCAGACCAAGTCAATCCAGCGTGTTGACAAATACTC
ACGCCGGAACAAGGAGACAGTGCTGGCAAATGTGGAAGGACTCATGGATATCATCGTGGTTTCCCCTCAGC
GGCAGACAGGGACCAATGCCTGTGGTGTGAACAATGGTGGCTGCACCCACCTCTGCTTTGCCAGAGCCTCG
CACTTCGTATGTCCCTGTCCTGACCAACCTGATAGCCAGCCCTGCTCCCTTGTGCCTGGCCTGGTACCACC
AGCTCCTAGGGCTACTGGCATCAGTGAAAAGAGCCCAGTGCTACCCAACACACCACCTACCACCTTGTATT
CTTCAACCACCCGGACCCGCACGTCTCTGGAGGAGGTGGAAGGAAGATGCTCTGAAAGCGATGCCACGCTG
GGCCTCTGTGCACGTTCCAATGACGCTGTTCCTGCTGCTCCAGGGGAAGGACTTCATATCAGCTACGCCAT
TGGTGGACTCCTCAGTATTCTGCTGATTTTGGTGCTGATTGCAGCTTTGATGCTGTACAGACACAAAAAAT
CCAAGTTCACTGATCCTGGAATGGGGAACCTCACCTACAGCAACCCCTCCTACCGAACATCCACACAGGAA
GTGAAGATTGAAGCAATCCCCAAACCAGCCATGTACAACCAGCTGTGCTATAAGAAAGAGGGAGGGCCTGA
CCATAACTACACCAAGGAGAAGATCAAGATCGTAGAGGGAATCTGCCTCCTGTCTGGGGATGATGCTCAGT
GGGATGACCTCAAGCAACTGCGAAGCTCACGGGGGGGCCTCCTCCGGGATCATGTATGCATGAAGACAGAC
ACGGTGTCCATCCAGGCCAGCTCTGGCTCCCTGGATGACACAGAGATGGAGCAGCTGTTACAGGAAGAGCA
GTCTGAGTGTAGCAGCGTCCATACTGCAGCCACTCCAGAAAGACGAGGCTCTCTGCCAGACACGGGCTGGA
AACATGAACGCAAGCTCTCCTCAGAGAGCCAGGTCTAA
LP288 Full-Length sequence (1905aa):
(SEQ ID NO: 6). The underlined portion is a predicted signal sequence (Met-1 to Ala-20). A predictcd SP
cleavage site is between Ala-20 and Ser-21 indicated as follows: 1
MRRQWGALLLGALLCAHGLA\hat{ }SSPE 24.
MRRQWGALLLGALLCAHGLASSPECACGRSHFTCAVSALGECTCIPAQWQCDGDNDCGDHSDEDGCILPTC
SPLDFHCDNGKCIRRSWVCDGDNDCEDDSDEQDCPPRECEEDEFPCQNGYCIRSLWHCDGDNDCGDNSDEQ
CDMRKCSDKEFRCSDGSCIAEHWYCDGDTDCKDGSDEENCPSAVPAPPCNLEEFQCAYGRCILDIYHCDGD
DDCGDWSDESDCSSHQPCRSGEFMCDSGLCINAGWRCDGDADCDDQSDERNCTTSMCTAEQFRCHSGRCVR
LSWRCDGEDDCADNSDEENCENTGSPQCALDQFLCWNGRCIGQRKLCNGVNDCGDNSDESPQQNCRPRTGE
ENCNVNNCGCAQKCQMVRGAVQCTCHTGYRLTEDGHTCQDVNECAEEGYCSQGCTNSEGAFQCWCETGYEL
RPDRRSCKALGPEPVLLFANRIDIRQVLPHRSEYTLLLNNLENAIALDFHHRRELVFWSDVTLDRILRANL
NCSNVEEVVSTGLESPGGLAVDWVHDKLYWTDSGTSRIEVANLDGAHRKVLLWQNLEKPRAIALHPMEGTI
YWTDWGNTPRIEASSMDGSGRRIIADTHLFWPNGLTIDYAGRRMYWVDAKHHVIERANLDGSHRKAVISQG
LPHPFAITVFEDSLYWTDWHTKSINSANKFTGKNQEIIRNKLHFPMDIHTLHPQRQPAGKNRCCDNNGGCT
HLCLPSGQNYTCACPTGFRKISSHACAQSLDKFLLFARRMDIRRISFDTEDLSDDVIPLADVRSAVALDWD
SRDDHVYWTDVSTDTISRAKWDGTGQEVVVDTSLESPAGLAIDWVTNKLYWTDAGTDRIEVANTDGSMRTV
LIWENLDRPRDIVVEPMGGYMYWTDWGASPKIERAGMDASGRQVIISSNLTWPNGLAIDYGSQRLYWADAG
MKTIEFAGLDGSKRKVLIGSQLPHPFGLTLYGERIYTDWQTKSIQSADRLTGLDRYETLQENLENLMDIHV
FHRRRPPVSTPCAMENGGCSHLCLRSPNPSGFSCTCPTGINLLSDGKTCSPGMNSPLIFARRIDIRNVSLD
IPYFADVWPINITMKNTIAVGVDPQEGKVYWJSDSTLHRISRANLDCSQHEDIITTGLQTTDGLAVDATGR
KVYWTDTGTNRIEVGNLDGSMRKVLVWQNLDSPRAIVLYHEMGFMYWTDWGENAKLERSGMDGSDRAVLIN
NNLGWPNGLTVDKASSQLLWADAHTERIEAADLNGANRHTLVSPVQHPYGLTLLDSYIYWTDWQTRSIHRA
DKGTGSNVILVRSNLPGLMDMQAVDRAQPLGFNKCGSRNGGCSHLCLPRPSGFSCACPTGIQLKGDGKTCD
PSPETYLLFSSRGSIRRISLDTSDHTDVHVPVPELNNVISLDYDSVDGKVYYTDVFLDVIRRADLNGSNME
TVIGRGLKTTDGLAVDWVARNLYWTDTGRNTIEASRLDGSCRKVLINNSLDEPRAIAVFPRKGYLFWTDWG
HIAKIERANLDGSERKVLINTDLGWPNDLTLDYDTRRIYWVDAHLDRIESADLNGKLRQVLVGHVSHPFAL
TQQDRWIYWTDWQTKSIQRVDKYSCRNKETVLANVEGLMDIIVVSPQRQTGTNACGVNNGGCTHLCFARAS
DFVCACPDEPDSQPCSLVPGLVPPAFRATGMSEKSPVLPNTPPTTLYSSTTRTRTSLEEVEGRCSERDARL
GLCARSNDAVPAAPCEGLHISYAIGGLLSILLIINVIAALMLYRHKKSKFTDPGMGNLTYSNPSYRTSTQE
VKIEAIPKPAMYNQLCYKKEGGPDHNYTKEKIKIVEGICLLSGDDAEWDDLKQLRSSRGGLLRDHVCMKTD
TVSIQASSCSLDDTEMEQLLQEEQSECSSVHTAATPERRGSLPDTGWKHERKLSSESQV*
An LP288 Variant (1631aa):
The following is an LP288 variant encompassed by the present invention (comprising generally the
extracellular portion of a mature LP288). Such an LP288 variant could be used as a competitive binding
agent for various LP288 ligands.
SSPECACGRSHFTCAVSALGECTCIPAQWQCDGDNDCGDHSDEDGCILPTCSPLDFHCDNGKCIRRSWVCD
GDNDCEDDSDEQDCPPRECEEDEFPCQNGYCIRSLWHCDGDNDCGDNSDEQCDMRKCSDKEFRCSDGSCIA
EHWYCDGDTDCKDGSDEENCPSAVPAPPCNLEEFQCAYGRCILDIYHCDGDDDCGDWSDESDCSSHQPCRS
GEFMCDSGLCINAGWRCDGDADCDDQSDERNCTTSMCTAEQFRCHSGRCVRLSWRCDGEDDCADNSDEENC
ENTGSPQCALDQFLCWNGRCIGQRKLCNGVNDCGDNSDESPQQNCRPRTGEENCNVNNGGCAQKCQMVRGA
VQCTCHTGYRLTEDGHTCQDVNECAEEGYCSQGCTNSEGAFQCWCETGYELRPDRRSCKALGPEPVLLFAN
RIDIRQVLPHRSEYTLLLNNLENAIALDFHHRRELVFWSDVTLDRILRANLNGSNVEEVVSTGLESPGGLA
VDWVHDKLYWTDSGTSRIEVANLDGAHRKVLLWQNLEKPRAIALHPMEGTIYWTDWGNTPRIEASSMDGSG
RRIIADTHLFWPNGLTIDYAGRRMYWVDAXHHVIERANLDGSHRKAVISQGLPHPFAITVFEDSLYWTDWH
TKSINSANKFTGKNQEIIRNKLHFPMDIHTLHPQRQPAGKNRCGDNNGGCTHLCLPSGQNYTCACPTGFRK
ISSHACAQSLDKFLLFARRNDIRRISFDTEDLSDDVIPLAnVRSAVALDWDSRDDHVYWIDVSTDTISRAK
WDGTGQEVVVDTSLESPAGLAIDWVTNKLYWTDAGTDRIEVANTDGSMRTVLIWENLDRPRDIVVEPMGGY
MYWTDWGASPKIERAGMDASGRQVIISSNLTWPNGLAIDYGSQRLYWADAGMKTIEFAGLDGSKRKVLIGS
QLPHPFGLTLYGERIYWTDWQTKSQSADRLTGLDRETLQENELENLMDIHVFHRRRPPVSTPCAMENGGCS
HLCLRSPNPSGFSCTCPTGINLLDGKTCSPGSMNSFLIFARRIDIPJSLDIPYFADVVVPINITMCJJTIA
VGVDPQEGKWSDSTLHRISRAIQDGSQHEDIITTGLQTTDGLAVDAIGRKVYWTDTGTNRIFEVRFNLDGS
MRKVLVWQNLDSPRAIVLYHEMGHMYWTDWGENAKLERSGMDGSDRAVLINNNLGWPNGLTVDKASSQLLW
ADAHTERIEAADLNGANRHTLVSPVQHPYGLTLLDSYIYWTDWQTRSIHRADKCTGSNVILVRSNLPGLMD
MQAVDRAQPLGFNKCGSRNGGCSHSCLPRPSGFSCACPTGIQLKGDGKTCDPSPETYLLFSSRGSIRRISL
DTSDHTDVHVPVPELNNVISLDYLSVDGKVYYTDVFLDVIRRADLNGSNMETVIGRGLKTTDGLAVDWVAR
NLYWTDTGRNTIEASRLDGSCRKVLINNSLDEPRAIAVFPRKGYLFWTDWGHIAKIERANLDGSERKVLIN
TDLGWPNGLTLDYDTRRIYWVDAHLDRIESADLNGKLRQVLVGHVSHPFALTQQDRWIYWTDIWQTSIQRV
DKYSGRNKETVLANVEGLMDIIVVSPQRQTGTNACGVNNGGCTHLCFARASDFVCACPDEPDSQPCSLV*
An LP288 Variant (154aa):
The following is an LP288 variant encompassed by the present invention (comprising generally the
intracellular portion of LP288). Such an LP288 variant could be used as an agonist or antagonist for
LP288 intracellular signaling.
SKFTDPGMGNLTYSNPSYRTSTQHVKIEAIPKPAMYNQLCYKKEGGPDHNYTKEKIKIVEGICLLSGDDAE
WDDLKQLRSSRGGLLRDHVCMKTLTVSIQASSGSLDDTEMEQLLQEEQSECSSVHTAATPERRGSLPDTGW
KHERKLSSESQV

Interesting portions of LP288 are the segments: Cys-378 to Cys-393 (CTCHTGYRLTEDGHTC), Cys-418 to Cys-433 (CWCETGYELRPDRRSC), and Cys-722 to Cys-736 (CACPTGFRKISSHAC) which have been discovered to be EGF-like domain signatures. Typically, an EGF domain includes six cysteine residues (here, they would be LP288 cysteines: C378, C393, C418, C433, C722, and C736). An additionally interesting segment of LP281 is the segment Asp-395 to Cys-418 (DVNECAEEGYCSQGCTNSEGAFQC), which has been discovered to be a calcium-binding EGF-like domain. An additionally interesting portion of LP288 is a segment identified as a potential aspartic acid and asparagine hydroxylation site (CTNSEGAFQCWC; from Cys-409 to Cys-420). Additionally, interesting portions of LP288 are segments: Cys-44 to Cys-66 (CIPAQWQCDGDNDCGDHSDEDGC), Cys-83 to Cys-105 (CIRRSWVCDGDNDCEDDSDEQDC), Cys-122 to Cys-143 (CIRSLWHCDGDNDCGDNSDEQC), Cys-160 to Cys-182 (CIAEHWYCDGDTDCKDGSDEENC), Cys-203 to Cys-225 (CILDIYHCDGDDDCGDWSDESDC), Cys-243 to Cys-265 (CINAGWRCDGDADCDDQSDERNC), Cys-282 to Cys-304 (CVRLSWRCDGEDDCADNSDEENC), and Cys-324 to Cys-349 (CIGQRKLCNGVNDCGDNSDESPQQNC), which have been discovered to exhibit an LDL-receptor class A (LDLRA) module profile (module assignments for LP288 were made, among other factors, on, for example, database sequences, related annotations, and publications (see, e.g., Bork, et al., 1996 Quart. Rev. Biophys 29:119-167, and Bork & Bairoch 1995 Trends Biochem Sci 2 (Suppl)). Alternative boundary markings for LDLRA modules of LP288 are segments: Cys-42 to Glu-63 (CTCIPAQWQCDGDNDCGDHSDE), Cys-83 to Cys-105 (CIRRSWVCDGDNDCEDDSDEQDC), Cys-122 to Cys-143 (CIRSLWHCDGDNDCGDNSDEQC), Cys-160 to Cys-182 (CIAEHWYCDGDTDCKDGSDEENC), Cys-203 to Cys-225 (CILDIYHCDGDDDCGDWSDESDC), Cys-243 to Cys-265 (CINAGWRCDGDADCDDQSDERNC), Cys-282 to Cys-304 (CVRLSWRCDGEDDCADNSDEENC), and Cys-324 to Cys-349 (CIGQRKLCNGVNDCGDNSDESPQQNC). Similar LDLRA modules have been found in other receptor related to the LDL receptor, for example: vertebrate low-density lipoprotein receptor-related protein 1 (LRP1) (reviewed in Krieger & Herz 1994 Annu. Rev. Biochem. 63:601-637) and vertebrate low-density lipoprotein receptor-related protein 2 (LRP2) (also known as gp330 or megalin), contain LDLRA modules. Additionally, interesting portions of LP288 are respectively, LP288 YWTD islands 1-4 are approximately:

(GPEPVLLFANRIDIRQVLPHRSHYTLLLNNLENAIALDFHHRRELVFWSDVTLDRILRANLNGSNVEEVVS
TGLESPGGLAVDWVHDKLYWTDSGTSRIEVANLDGAHRKVLLWQNLEKPRAIALHPMEGTIYWTDWGNTPRI
EASSMDGSGRRIIADTHLFWPNGLTIDYAGRRMYWVDAKHHVIERANLDGSHRKAVISQGLPHPFAITVFED
SLYWTDWHTKSINSANKFTGKNQEIIRNKLHFPMDIHTLHPQR);
(LDKFLLFARRMDIRRISFDTEDLSDDVIPLADVRSAVALDWDSRDDHVYWTDVSTDTISRAKWDGTGQEVV
VDTSLESPAGLAIDWVTNKLYWTDAGTDRIEVANTDGSMRTVLIWENLDRPRDIVVEPMGGYMYWTDWGASP
KIERAGMDASGRQVIISSNLTWPNGLAIDYGSQRLYWADAGMKTIEFAGLDGSKRKVLIGSQLPHPFGLTLY
GERIYWTDWQTKSIQSADRLTGLDRETLQENLENLMDIHVFHRRRPPV);
(NSFLIFARRIDIRMVSLDIPYFADVVVPINITMKNTIAVGVDPQEGKVYWSDSTLHRISRANLDGSQHEDT
ITTGLQTTDGLAVDAIGRKVYWTDTGTNRIEVGNLDGSMRKVLVWQNLDSPRAIVLYHEMGFMYWTDWGENA
KLERSGMDGSDRAVLINNNLGWPNGLTVDKASSQLLWADAHTERIEAADLNGANRHTLVSPVQHPYGLTLLD
SYIYWTDWQTRSIHRADKGTGSNVILVRSNLPGLMDMQAVDRAQPL);
and
(ETYLLFSSRGSIRRISLDTSDHTDVHVPVPELNNVISLDYDSVDGKVYYTDVFLDVIRRADLNGSNMETVI
GRGLKTTDGLAVDWVARNLYWTDPGRNTIEASRLDGSCRKVLINNSLDEPRAIAVFPRKGYLFWTDWGHIAK
IERANLDGSERKVLINTDLGWPNGLTLDYDTRRIYWVDAHLDRIESADLNGKLRQVLVGHVSHPFALTQQDR
WIYWTDWQTKSIQRVDKYSGRNKETVLANVEGLMDIIVVSPQRQ).

Additionally, interesting portions of LP288 are LP288 YWTD repeats (Nos. 1-6) for LP288 YWTD island No. 1 are approximately:

(ELVFWSDVTLDRILRANLNGSNVEEVVSTGLESPGGLAVDWVH),
(DKLYWTDSGTSRIEVANLDGAHRKVLLWQNLEKPRAIALHPM),
(GTIYWTDWGNTPRIEASSMDGSGRRIIADTHLFWPNGLTIDYA),
(RRMYWVDAKHHVIERANLDGSHRKAVISQGLPHPFAITVFED),
and
(SLYWTDWHTKSINSANKFTGKNQEIIRNKLHFPMDIHTLHPQR).

Additionally, interesting portions of LP288 are LP288 YWTD repeats (Nos. 1-6) for LP288 YWTD island No. 2 are approximately:

(DHVYWTDVSTDTISRAKWDGTGQEVVVDTSLESPAGLAIDWVTN),
(KLYWTDAGTDRIEVANTDGSMRTVLIWENLDRPRDIVVEPMGGY),
(MYWTDWGASPKIERAGMDASGRQVIISSNLTWPNGLAIDYGSQR),
(LYWADAGMKTIEFAGLDGSKRKVLIGSQLPHPFGLTLYGERI),
and
(YWTDWQTKSIQSADRLTGLDRETLQENLENLMDIHVFHRRRPPV).

Additionally, interesting portions of LP288 are LP288 YWTD repeats (Nos. 1-6) for LP288 YWTD island No. 3 are approximately:

(FLIFARRIDIAMVSLDIPYFADVVVPINITMKNTIAVGVDPQEG),
(KVYWSDSTLHRISRANLDGSQHEDIITTGLQTTDGLAVDAIGR),
(KVYWTDTGTNRIEVGNLDGSMRKVLVWQNLDSPRAIVLYHEMG),
(FMYWTDWGENAKLERSGMDGSDRAVLINNNLGWPNGLTVDKASS),
(QLLWADAHTERIEAADLNGANRHTLVSPVQHPYGLTLLDSY),
and
(IYWTDWQTRSIHRADKGTGSNVILVRSNLPGLMDMQAVDRAQPL).

Additionally, interesting portions of LP288 are LP288 YWTD repeats (Nos. 1-6) for LP288 YWTD island No. 4 are approximately:

(YLLFSSRGSIRRISLDTSDHTDVHVPVPELNNVISLDYDSVDG),
(KVYYTDVFLDVIRRADLNGSNMHTVIGRGLKTTDGLAVDWVAR),
(NLYWTDTGRNTIEASRLDGSCREVLINNSLDEPRAIAVFPRK),
(GYLFWTDWGHIAKIERANLDGSERKVLINTDLGWPNGLTLDYDT),
(RRIYWVDAHLDRIESADLNGKLRQVLVGHVSHPFALTQQDR),
and
(WIYWTDWQTKSIQRVDKYSGRNHETVLANVEGLMDIIVVSPQRQ).

Additionally, interesting portions of LP288 are the LP288 proline-rich region containing PxxP motif sequences is approximately, (PGLVPPAPRATGMSEKSPVLPNTPPTTLYSSTTRTRTSLEEVEGRCSERDARLGLCARSNDAVPAAP) from about Pro-1652 to about Pro-1718. Additionally, interesting portions of LP288 are the LP288 cytoplasmic domain is approximately,

(FTDPGMGNLTYSNPSYRTSTQEVKIEAIPKPAMYNQLCYKKEGGPDHNYTKEKIKIVEGICLLSGDDAEWD
DLKQLRSSRGGLLRDHVCMKTDTVSIQASSGSLDDTEMEQLLQEEQSECSSVHTAATPERRGSLPDTGWKHE
RKLSSESQV)

from about Phe-1754 to about Val-1905. The seven-amino acid, C-terminal domain of LP288 (LSSESQV) contains a terminal tSxV motif (where S is serine, x is any amino acid, and V is valine). This motif has been suggested to interact with PDZ domains of various intracellular proteins.

Other interesting segments of LP288 are discovered fragments Gly-11 to Cys-255; Gly-28 to Gln-50; Asp-64 to Cys-83; Arg-85 to Glu-97; Asp-98 to Cys-110; Glu-112 to Leu-126; Asn-133 to Ser-156; Asn-181 to Asp-214; Cys-216 to Ser-233; Ser-233 to Gly-252; Gly-252 to Cys-277; Cys-282 to Gly-308; Ser-309 to Arg-328; Leu-330 to Cys-365; Cys-433 to Pro-455; Val-506 to Ser-530; Lys-555 to Ile-579; Arg-589 to Gly-609; Val-615 to Val-635; Ser-637 to Asp-657; Trp-658 to Asn-679; His-688 to Asn-705; Thr-759 to Asp-781; Val-809 to Ala-835; Ile-854 to Ile-884; Gln-895 to Gln-915; Arg-916 to Glu-928; Ile-941 to Asp-962; Phe-1053 to Ala-1085; Tyr-1096 to Ile-1118; Thr-1121 to Thr-1143; Trp-1163 to Asp-1185; Arg-1202 to Ser-1222; Val-1249 to Trp-1270; Gln-1271 to Asn-1285; Ile-1287 to Glu-1306; Cys-1324 to Thr-1337; Pro-1382 to Thr-1402; Gly-1424 to Thr-1447; Ile-1466 to Gly-1491; Leu-1509 to Ala-1534; Thr-1512 to Thr-1592; Leu-1594 to Asn-1615; Leu-1627 to Thr-1674; Glu-1720 to Lys-1751; Cys-1791 to Lys-1804; Glu-1811 to Trp-1823; Glu-1883 to Arg-1897; Trp-5 to Ala-16; Gly-18 to Ala-35; Trp-49 to Ile-67; Asp-91 to Glu-114; His-128 to Met-145, Lys-147 to Ile-161; Cys-167 to Ala-188; His-209 to Phe-236; Trp-248 to Phe-275; Arg-288 to Asp-315; Asn-332 to Cys-349; Pro-351 to Met-371; Glu-388 to Gly-414; Thr-422 to Glu-439; Leu-497 to Leu-516; Thr-528 to Lys-546; Trp-573 to Ile-592; Val-620 to Leu-640; Trp-658 to Asn-679; Pro-692 to Thr-710; Gly-717 to Ser-739; Trp-789 to Gly-820; Trp-832 to Thr-851; Asp-876 to Val-896; Trp-963 to Glu-987; Arg-998 to Ser-1014; Arg-1019 to Ile-1034; ala-1085 to Arg-1104; Ser-1106 to Thr-1120; Thr-1120 to Val-1138; Asp-1142 to Lys-1159; Asp-1185 to Val-1204; Asn-1214 to Glu-1224; Asp-1229 to Leu-1248; Asp-1269 to Asn-1285; Asp-1298 to Leu-1308; Asn-1311 to Leu-1323; Pro-1328 to Gln-1340; Lys-1342 to Ser-1355; Ser-1360 to Val-1379; Leu-1390 to Thr-1402; Arg-1410 to Ile-1423; Trp-1444 to Arg-1462; Lys-1495 to Val-1508; Thr-1512 to Arg-1527; His-1535 to Arg-1549; Asp-1573 to Gly-1599; Ser-1607 to Thr-1625; Ala-1632 to Ser-1649; Pro-1657 to Val-1670; Ser-1682 to Arg-1703; Cys-1707 to Leu-1722; Ile-1728 to Tyr-1747; His-1749 to Leu-1762; Tyr-1764 to Ile-1778; Leu-1790 to Lys-1808; Ser-1817 to Leu-1836; Cys-1842 to Gln-1864; Glu-1868 to Thr-1878; Thr-1878 to Thr-1891; Leu-9 to Cys-25; Cys-25 to Ile-45; Ile-45 to Pro-69; Val-89 to Cys-117; Cys-129 to Lys-151; Cys-167 to Pro-189; Leu-205 to Ser-233; Arg-249 to Thr-271; Val-283 to Ala-313; Lys-329 to Gly-363; His-381 to Cys-405; Ala-415 to Glu-439; Asp-519 to Ala-543; Leu-561 to Ser-587; Arg-611 to His-631; His-659 to Ile-677; Leu-681 to His-711; Asp-781 to Val-809; Lys-829 to Thr-845; Lys-953 to Asn-985; Phe-1053 to Lys-1081; Asp-1089 to Thr-1121; Gly-1135 to Lys-1159; Met-1181 to Leu-1205; Ser-1223 to Val-1249; Ser-1263 to Asn-1285; Met-1299 to Gly-1319; Ile-1339 to Ser-1359; Leu-1433 to Cys-1461; Trp-1487 to Gly-1515;Thr-1521 to Asp-1543; Thr-1563 to Val-1593; Gly-1663 to Arg-1709; His-1723 to Tyr-1747; Lys-1753 to Glu-1779; Tyr-1787 to Ile-1807; Asp-1819 to Val-1841; and Gly-1855 to His-1895, whose discovery was based on an analysis of hydrophobicity, hydropathicity, and hydrophilicity plots. Additional interesting sections of LP288 are the discovered portions of LP288 from Leu-9 to Ala-20; Gly-40 to Asp-56; Asp-64 to Pro-73; Gly-82 to Asp-95; Gln-103 to Leu-126; Asn-133 to Lys-147; Cys-155 to His-164; Pro-189 to Ile-207; Asp-213 to Asp-224; Arg-233 to Cys-250; Glu-262 to Gln-274; Asp-300 to Gln-311; Phe-317 to Lys-329; Pro-351 to Gln-367; Asp-395 to Leu-426; Leu-473 to Leu-481; Asn-498 to Val-518; Thr-528 to Asn-539; Leu-540 to Trp-550; His-562 to Pro-577; Ile-579 to Asp-594; Trp-599 to Val-615; Asn-700 to His-711; Ser-716 to Lys-730; Ser-732 to Phe-746; Ala-747 to Glu-760; Asp-771 to Trp-789; Gly-847 to Leu-858; Arg-862 to Gly-879; Asp-921 to Leu-932; Leu-982 to His-993; Val-1002 to Ser-1014; Pro-1021 to Leu-1037; Thr-1042 to Arg-1055; Ile-1057 to Phe-1069; Val-1073 to Gly-1087; His-1116 to Thr-1125; Thr-1141 to Val-1160; Tyr-1175 to Asn-1189; Val-1204 to Leu-1216; Val-1218 to Thr-1232; Ser-1263 to Arg-1273; Thr-1282 to Arg-1290; Asn-1292 to Val-1302; Leu-1306 to His-1322; Arg-1327 to Lys-1342; Ser-1360 to Asp-1370; Thr-1371 to Val-1381; Asn-1415 to Leu-1427; Trp-1444 to Ser-1455; Val-1508 to Thr-1521; Asn-1596 to Pro-1608; Gln-1609 to His-1626; Tyr-1726 to Val-1740; Met-1746 to Phe-1754; Thr-1755 to Pro-1767; Ile-1807 to Ser-1817; Lys-1844 to Ser-1854; Asp-1858 to Leu-1866; and Gly-1892 to Ser-1901. These fragments were discovered based on analysis of antigenicity plots. Further, particularly interesting LP288 structures (e.g., such as a helix, a strand, or a coil) that have been discovered are the following LP288 helix structures: Gln-4 to Leu-8; Leu-464 to Asn-466; Asn-469 to Ile-471; Leu-489 to Leu-493; Gln-738 to Arg-748; Arg-979 to Asn-988; Gln-1300 to Arg-1304; Leu-1406 to Arg-1411; Ile-1493 to Arg-1498; Arg-1538 to Ser-1541; Arg-1700 to Arg-1703; Ala-1742 to Tyr-1747; Trp-1823 to Ser-1831; and Thr-1860 to Glu-1868. Particularly interesting discovered coil structures are Met-1 to Arg-2; Gly-18 to His-31; Leu-39 to Thr-43; Asp-52 to Gly-65; Thr-70 to Leu-74; Cys-78 to Lys-82; Asp-91 to Tyr-121; Cys-129 to Asp-140; Cys-148 to Ser-159; Cys-167 to Asn-159; Cys-167 to Asn-192; Gly-201 to Gly-201; Cys-210 to Leu-242; Ala-246 to Met-269; Cys-277 to Arg-281; Cys-289 to Gln-316; Trp-320 to Glu-355; Asn-360 to Ala-365; His-380 to Gly-383; Glu-388 to Asp-395; Gly-403 to Ala-415; Cys-419 to Pro-440; Leu-454 to Ser-458; Ser-485 to Ser-485; Asn-496 to Ser-500; Gly-509 to Gly-515; Thr-528 to Thr-532; Leu-540 to Ala-543; Gln-551 to Arg-557; His-562 to Gly-566; Asp-572 to Pro-577; Ser-583 to Gly-588; Trp-599 to Leu-603, Ala-608 to Arg-610; Ala-617 to Lys-618; Asn-626 to Arg-632; Gly-639 to Phe-644; Glu-650 to Leu-653; Asp-657 to Lys-672; Leu-681 to Asp-686; Leu-690 to Lys-669; Gly-703 to Gly-708; Leu-714 to Asn-719; Cys-724 to Arg-729; Ser-732 to Ser-732; Phe-757 to Asp-765; Trp-780 to Asp-785; Val-792 to Thr-796; Asp-803 to Gln-807; Leu-815 to Gly-820; Asn-828 to Asn-828; Asp-834 to Asp-838; Asn-844 to Met-849; Asn-857 to Arg-862; Pro-868 to Gly-871; Asp-877 to Glu-885; Gly-888 to Arg-894; Asn-901 to Gly-907; Asp-911 to Gln-915; Asp-921 to Met-924; Gly-931 to Lys-936; Ser-943 to Gly-950; Gly-955 to Glu-956; Asp-962 to Ser-967; Thr-975 to Leu-977; Arg-997 to Thr-1004; Met-1008 to Ser-1014; Ser-1020 to Gly-1033; Ser-1038 to Asn-1048; Leu-1064 to Ile-1066; Asp-1089 to Gly-1093; Asp-1099 to Thr-1101; Asn-1109 to Glu-1116; Gly-1122 to Gly-1128; Ile-1134 to Arg-1136; Asp-1142 to Asn-1146; Asn-1152 to Met-1157; Asn-1165 to Pro-1169; Glu-1177 to Gly-1179; Thr-1184 to Asn-1189; Ser-1195 to Asp-1201; Asn-1208 to Gly-1215; Asp-1219 to Ala-1221; Ala-1230 to His-1231; Asp-1239 to Arg-1245; Pro-1251 to Leu-1258; Asp-1269 to Thr-1272; Ala-1278 to Ser-1284; Ser-1291 to Leu-1296; Gln-1306 to Cys-1320; Leu-1325 to Phe-1331; Cys-1335 to Gly-1338; Gly-1343 to Glu-1353; Ser-1360 to Ser-1363; Asp-1370 to Thr-1375; Pro-1382 to Asn-1385; Tyr-1392 to Gly-1397; Asp-1413 to Asn-1418; Arg-1425 to Gly-1432; Thr-1445 to Asn-1450; Arg-1456 to Ser-1460; Asn-1468 to Arg-1474; Phe-1479 to Tyr-1484; Asp-1489 to Gly-1491; Asn-1500 to Ser-1504; Asp-1513 to Leu-1520; Asp-1523 to Arg-1527; Leu-1544 to Asn-1545; His-1555 to Asp-1566; Asp-1573 to Lys-1577; Lys-1584 to Lys-1590; Ser-1607 to Thr-1614; Asn-1620 to Cys-1624; Ala-1632 to Ser-1633; Cys-1639 to Cys-1648; Pro-1652 to Thr-1677; Ser-1682 to Thr-1683; Arg-1696 to Cys-1697; Arg-1709 to Gly-1721; Ile-1728 to Gly-1729; Lys-1750 to Gly-1760; Ser-1765 to Arg-1770; Ile-1781 to Pro-1784; Lys-1794 to Thr-1803; Ser-1817 to Ala-1821; Ser-1832 to Leu-1836; Thr-1845 to Thr-1845; Ser-1853 to Leu-1859; Ser-1874 to Ser-1874; Ala-1880 to Trp-1893; and Ser-1903 to Val-1905. Particularly interesting discovered strand structures are Thr-33 to Cys-34; Arg-85 to Val-89; Leu-126 to Trp-127; Ile-161 to Tyr-166; Leu-205 to Tyr-208; Cys-243 to Ile-244; Val-283 to Trp-287; Val-375 to Cys-379; Arg-385 to Leu-386; Val-441 to Phe-444; Glu-503 to Ser-507; Ala-517 to Trp-520; Leu-525 to Trp-527; Arg-534 to Ala-538 to Ala-558 to Leu-561; Ile-579 to Ala-581; Arg-590 to Ala-593; Thr-604 to Asp-606; Met-612 to Val-615; Val-621 to Arg-624; Lys-633 to Ile-636; Ala-645 to Val-648; His-711 to Leu-712; Thr-721 to Cys-722; Tyr-788 to Thr-790; Glu-808 to Asp-812; Ala-822 to Val-826; Leu-830- Trp-832; Arg-839 to Ala-843; Arg-850 to Trp-855; Asp-863 to Val-866; Tyr-872 to Thr-876; Gln-895 to Ser-899; Thr-926 to Ala-930; Lys-938 to Ile-941: Arg-957 to Trp-960; Leu-1016 to Leu-1018; Ile-1034 to Leu-1037; Arg-1060 to Ser-1063; Val-1072 to Val-1074; Ala-1085 to Val-1086; Lys-1094 to Tyr-1096; Asp-1117 to Thr-1120; Ala-1130 to Val-1131; Lys-1137 to Thr-1141; Arg-1147 to Val-1150; Arg-1158 to Trp-1163; Met-1181 to Trp-1183; Arg-1202 to Ile-1206; His-1246 to Val-1249; Tyr-1264 to Trp-1267; Ile-1275 to His-1276; Val-1286 to Arg-1290; Thr-1354 to Phe-1358; Arg-1365 to Ile-1367; Val-1377 to His-1378; Val-1387 to Ser-1389; Lys-1398 to Tyr-1401; Glu-1420 to Ile-1423; Thr-1451 to Ala-1454; Arg-1462 to Ile-1466; ala-1475 to Val-1478; Leu-1485 to Trp-1487; Lys-1507 to Asn-1511; Ile-1529 to Trp-1531; Val-1551 to Val-1553; Trp-1568 to Trp-1571; Asp-1602 to Val-1606; His-1626 to Phe-1629; Val-1636 to Cys-1637; His-1723 to Ala-1727; Leu-1790 to Tyr-1792; Lys-1808 to Val-1810; Ile-1813 to Leu-1815; Val-1841 to Cys-1842; Thr-1847 to Gln-1851; and Val-1876 to His-1877. Further encompassed by the invention are contiguous amino acid residue combinations of any of the predicted secondary structures described above. For example, one helix-coil- strand-coil motif of LP288 combines the Leu-1406 to Arg-1411 helix, with the Asp-1413 to Asn-1418 coil, the Glu-1420 to Ile-1423 strand, and the Arg-1425 to Gly-1432 coil form an interesting fragment of contiguous amino acid residues from Leu-1406 to Gly-1432. Other combinations of contiguous amino acids are contemplated as can be easily determined.

LP288 Variants

Encompassed herein are LP288 variants, such as, e.g., variants comprising only an extracellular region of LP288. Other LP288 variants encompassed by the present invention are LP288 variants that have mutated, truncated, and or missing cytoplasmic portion/s (e.g., a mutant LP288 lacking the carboxyl intracellular domain). For example, an LP288 variant lacking a carboxyl intracellular domain may act as an antagonist to vertebrate members of the Wnt signaling group. Additionally, the cytoplasmic portion of LP288 contains a tetra-amino-acid motif NPxY, which also plays a central role in mediating the interaction of receptor tails with endocytotic machinery, and also serves as a docking site for cytoplasmic adaptor and scaffold proteins. Mutations in this region of an LDL receptor result in impaired endocytosis of LDL by, e.g., the liver, which in turn leads to elevated plasma cholesterol concentrations and coronary artery disease. Accordingly, further LP288 variants encompassed herein include those involving modifications to LxxY, tSxV an NPxY sequence of LP288. For example, addition of an NPxY sequence to LP288 cytoplasmic portion is contemplated so that, e.g., increased internalization of lipoprotein-like molecules would occur thereby treating, e.g., an atherosclerotic-like condition.

LP288 FUNCTIONS: Given the teachings supplied herein, for example, of: LP288 primary amino acid, LP288 higher order structures; the relationship of LP288 amino acid sequence to higher order structural features; the comparability of LP288 sequence and/or LP288 higher order structure with known LRPs (such as, e.g., members of the low density lipoprotein-related protein family, such as, e.g., LRP1, alpha-2-macroglobin receptor, LRP2, LRP5, and LRP6); and the relationship of higher order structural features of LRPs with their known functions; it is likely that an LP288 or an LP288 variant plays similar roles in a variety of physiological processes. Some non-limiting functions an LP288 or LP288 variant is likely to participate in, modulate, maintain, effect, or regulate are those such as, for example: ligand endocytosis; maintenance of plasma cholesterol concentrations; modulation of lipases; lipoprotein processing; protein and/or remnant scavenging; mediation of the cellular uptake of apoE-containing, remnant-like lipoproteins; processing apolipoproteins (e.g., such as ApoE, ApoE-4, etc.); lipid metabolism, catabolism, clearance, and/or recycling, amyloid clearance (e.g., from the nervous system, e.g., from the CNS, e.g., from the brain), liver function; plasma clearance of chylomicron remnants; plasma clearance of activated alpha 2-macroglobulin; local modulation of complexes between plasminogen activators and their endogenous inhibitors (e.g., hypoxic conditions generated as a result of tumors has been shown to induce expression of plasminogen activator inhibitor-1 (PAI-1) and LRP suggesting that cancerous conditions associated with tumorogenesis require the activity of proteases, protease inhibitors, their complexes, and LRP receptors to act as ligands for them. Accordingly, prevention of uptake of such entities may modulate tumorogenesis); prevention of atherosclerotic disease; maintenance of protease homeostasis; modulation of local serpin concentrations; regulation of protease-serpin complexes; coronary artery diseases, conditions, and/or syndromes; cell adhesion; cell migration (e.g., receptor (uPAR)-bound urokinase (uPA) binds its inhibitor PAI-1, which is localized in an extracellular matrix, and the resulting complex is internalized by endocytotic receptor activity of LRPs. Recently, it has been shown that interference with LRP binding of this type of complex significantly decreases cell motility via suppression of filopodia and membrane ruffling activity (see, e.g., Chazaud, et al. 2000 Exp. Cell Res. 258:237-214, incorporated herein for the assay methods described therein). These results suggest that such complexes (uPAR:uPA;PAI-1:LPR) support the membrane ruffling activity involved in the guidance of migrating cells); cell motility associated with cell activation; local reorganization of cytoskeletal elements; uterine implantation during embryonic development; neurotransmission; long-term potentiation (LTP) such as, e.g., in the hippocampus; brain development (e.g., such as in defects of CNS development, such as, e.g. holoprosencephaly); embryogenesis (e.g., such as axis formation); induction of receptor clustering (such as, e.g., inducing clustering of NMDA receptors thereby stimulating local Ca⁺² influx); protection against endotoxins; blood disorders (e.g., factor VIII metabolism); neuronal apoptosis; neuronal survival; cell proliferation conditions (e.g., recently, it has been reported that an LRP with similarity to LRP1 is inactivated in at least 40% of non-small cell lung cancer cell lines (NSCLC) suggesting that LRPs, particularly LRP1-like proteins may play a role in tumorogenesis of NSCLs (see, e.g., Liu, et al., 2000 Cancer Research, 60:1961-1967, incorporated herein for the assay methods described therein)); autoimmune disease (e.g., active Heymann nephritis of rat, an autoimmune glomerular disease, is a clinical model of human membranous glomerulonephritis. Recently, it has been shown that an N-terminal portion of LRP2 is the autoantigenic target responsible for the generation of this rodent disease suggesting that a similar LRP2-like region in other proteins (such as LP288) may also generate similar human conditions or disorders (see, e.g., Oleinikov, et al. 2000 J. Amer Soc Neph 11:57-64, incorporated herein for the assay methods described therein). Given LP288's similarity to LRP2, similar N-terminal cysteine-like regions in LP288 may also be autoantigenic thus leading to similar autoimmune disorders, conditions, or syndromes that would affect e.g., the kidney); diabetic-like conditions and/or obesity-like conditions (recently, it has been shown that insulin activity regulates LRP presentation in adipocytes and can be inhibited by phosphatidylinositide 3-kinase treatment (see, e.g., Ko, et al., 2001 Biochemistry 40:752-759, incorporated herein for the assay methods described therein)); aging and/or insulin resistance (e.g., insulin resistance and chylomicron clearance is significantly reduced in aged humans and rodents. Recent data on the insulin regulation of LRP presentation suggests that age related insulin resistance may lead to detrimental plasma lipoprotein profila due to the reduced expression of LRPs. Similarly, age related insulin resistance may account for aberrant LRP expression on adipocytes leading to disorders associated with obesity); vitamin metabolism; organ development; neuronal degeneration; neuronal pathfinding; axon guidance; regulation of synaptic transmission; protein conformational disorders or diseases; Alzheimer or Alzheimer-like conditions; activation and/or modulation of MAP kinase pathways; local organization of the cytoskeleton; cell adhesion; and endocytosis of various LDL-like ligands.

Non-limiting examples of ligands for LP288 include, for instance, lipoproteins containing ApoE, lipases, proteases (such as, e.g., PA, alpha2M, or PAI-1) and/or protease/serpin inhibitor complexes, heparin-binding growth factors (e.g., midkine (MK)), and signaling proteins (such as, e.g., Reln or Reln-like proteins, vertebrate Wnts or vertebrate Wnt-like proteins, or TSP-1 or TSP-1-like proteins). Non-limiting examples of cytoplasmic binding partners and/or effectors of ligand binding of LP288 are, e.g. endocytotic machinery proteins; Dab1; X11; Fe65; Jip1; Jip2; PIP 4,5 kinase; PSD95; SEMCAP1; OMP25; ICAP-1; and Capon (among others).

LP288 & TOXINS: It has recently been reported that LRPs serve as gateways for the entry cellular of exotoxin A from Pseudomonas aeruginosa (PEA) (see, e.g., Kounnas et al. 1992a J. Biol. Chem. 267:12420-12423). Pseudomonas aeruginosa, an increasingly prevalent opportunistic human pathogen, is the most common gram-negative bacterium found in nosocomial infections. An LP288 variant (such as, e.g., an LP288 variant comprising only an extracellular portion of a mature complete LP288) may play a protective role by acting as a competitive “secreted” binding agent for released exotoxin A thus preventing it from being taken up into cells were its subsequent detrimental effects are realized. Consequently, LP288 would have a specific utility in currently available form as a means to treat a known biological poison. To test the role of LP288 in toxin uptake (such as, e.g., exotoxin A from Pseudomonas aerugionosa (PEA)) one can adapt art known techniques such as, e.g., the methods described in Willnow & Herz 1994 J. Cell. Sci 107:719-726, incorporated herein for these assay teachings). For example, by generating LRP deficient cells that can be subsequently transfected with various LP288 constructs (using ordinary genetic engineering techniques).

LP288 & LIPIDS: Evidence suggests that LRPs have the ability to mediate cellular uptake of lipophilic molecules, such as, for example, apoE-containing and remnant-like lipoproteins. To examine the role of LP288 in the regulation of lipoproteins many common art-existing methods can be used (see, e.g., the various methods and techniques discussed in, for example, Krieger & Herz 1994 Annu Rev Biochem 63:601-37, which is incorporated herein by reference for the methods and techniques described therein). A non-limiting example of such a technique to employ are the methods described in Sugiyama, et al. 2000 Biochemistry 39:15817-15825, which is incorporated herein by reference for the assay techniques described therein.

Features of LP No: 4 (LP289)

LP289 is a novel primate (e.g., human) polypeptide (SEQ ID NO: 8), which is a newly discovered member of the immunoglobulin superfamily (IgSF) of proteins. Specifically, LP288 is a novel member of the IgLON family, which includes, e.g., such proteins as LAMP, OBCAM, Neurotrimin (NtM), CEPU, GP50, KILON, and GP55. Characteristic features of members of the IgLON family include the presence of conserved cysteines as well as a number of Asn-linked potential glycosylation sites. Sequence similarity (at the amino acid level) with other known IgLON proteins suggests that LP289 is involved in cell recognition, cell adhesion, and/or opiod-type receptor functioning. Typically, among the IgLONs described to date, common structures (such as e.g., amino acid motifs, modules, and/or domains), arranged in characteristic locations within an IgLON protein are found. For example, the following amino acid motifs, modules, and/or domains are routinely found in characteristic locations in proteins that are members of the IgLON grouping.

At the N-terminal portion of a typical IgLON (for example, such as OBCAM), a series (e.g., typically three) of immunoglobulin-like C2-type, domains (IgC2) are found. Each immunoglobulin-like C2-type domain characteristically contains two conserved cysteine residues in the domain sequence that form disulfide bonds with each other (when drawn in cartoon form the Ig-C2-like domains resemble a series of loops, in which each loop is closed by disulfide bridges formed between the two conserved cysteine residues of the Ig-like-C2-type domain; see, e.g., Fig. 2C in Funatsu, et al. 1999 J. Bio. Chem. 274:8224-8230). Additionally, a number of N-linked glycosylation sites are found. LP289 follows this pattern by possessing three immunoglobulin-like C2-type domains, each of which has two conserved cysteine residues moreover, LP289 has a number of putative N-linked glycosylation sites. Following the immunoglobulin-like C2-type domains (i.e., moving C-terminad along the primary amino acid structure of a typical IgLON), most IgLONs possess a GPI-anchor like motif followed by a hydrophilic spacer region and a C-terminal signal sequence of predominately hydrophobic amino acids. LP289 also exhibits this pattern since it does not possess a membrane-spanning domain but contains a C-terminal hydrophobic sequence characteristic of a GPI anchor site membrane by attachment via a phosphatidylinositol linkage.

LP289 exhibits sequence identity/similarity at the amino acid level to members of the immunoglobulin protein superfamily, most notably with the various IgLON cell-adhesion molecules known as (see, e.g., Schofield, et al. 1989 EMBO J. 8:489-495, Hachisuka, et al., 2000 Dev. Brain Res. 122:183-191, Funatsu et al., 2000 J Comp Neurology 424:74-85). LP289 shares sequence homology with: (1) opiod-binding protein cell adhesion molecule (OBCAM) also designated OPCML, a protein that binds opioid alkaloids in the presence of acidic lipids, exhibiting selectivity for mu ligands. In the adult brain OBCAM is principally expressed in the gray matter in a pattern that suggests OBCAM plays a role in the synaptic machinery of the nervous system (such as, e.g., modulating opiod receptor functioning) (see, e.g., Loh and Smith 1996 “Regulation of Acute and Chronic Opiod Receptor Functions by OBCAM a cell Adhesion-like Molecule” in NIDA Research Monograph 161 titled, “Molecular Approaches to Drug Abuse Research: Vol. III: Recent Advances and Emerging Strategies”; Hachisuka, et al. 1999 Brain Res. 842:482-486; Hachisuka, et al. 2000 Dev. Brain Res. 122:183-191), specifically OBCAM has been reported to be expressed in the hypothalamic supraoptic (SON) and paraventricular nuclei (PVN) of the hypothalamus. In particular, OBCAM expression is observed primarily on dendrites of AVP-secreting magnocellular neurons while KILON is expressed mainly on dendrites of AVP secreting neurons and occasionally on OXT-secreting neurons suggesting that KILON and OBCAM confer the ability of magnocellular neurons of the hypothalamus to rearrange synaptic connectivity (see, e.g., Miyata, et al., 2000 Jour. Comp. Neuro 424:74-85); (2) Neurotrimin (Ntm), a subfamily of differentially expressed neural cell adhesion molecules that have been shown to regulate the development of neuronal projections via attractive and repulsive mechanisms that are cell type specific and are mediated by homophilic and heterophilic interactions (see, e.g., Struyk, et al. 1995 J. Neurosci. 15:2141-2156, Gil, et al., 1999 J. Neurosci 18:9312-9325); (3) CEPUS, a molecule that provides a favorable route for migrating neurons to generate a neuron-specific guidance in developing neurons in vivo (see, e.g., Kim, et al.1999 Mol Cells 9(3):270-276); (4) KILON, an IgLON member specifically expressed in the dentate gyrus (DG) of the adult rat that is involved in neurite outgrowth and capable of interacting with LAMP (see, e.g., Funatsu, et al. 1999 J. Bio. Chem. 274:8224-8230) and; (5) limbic associated membrane protein (LAMP), which is an Ig superfamily member that mediates selective neuronal growth and axon targeting (see, e.g., Pimenta, et al. 1995b Neuron 15:287-297). LAMP is a self-binding, antibody-like cell surface adhesion protein involved in formation of connections between adjacent neurons. The cDNA cloning and structural analysis of human LAMP is described in Gene 1996 170:189-195. (LAMP; PCT Publication W09630052-A1, published 03 Oct. 1996). LAMP protein, and by analogy LP289, may be important in nerve growth and differentiation, epilepsy, Alzheimer's disease, and schizophrenia or schizophrenic-like conditions. LAMP contributes to the guidance of developing axons and remodeling of mature circuits in the limbic system. The LAMP protein is essential for normal growth of the hippocampal mossy fiber projection. LAMP is attached to the membrane by a GPI-Anchor. It is expressed on limbic neurons and fiber tracts as well as in single layers of the superior colliculus, spinal chord, and cerebellum.

LP289 nucleic acid also has similarities to additional nucleic acids, described as having similar or analogous properties, including (1) chicken mRNA for CEPU-1, an immunoglobulin superfamily molecule expressed by developing cerebellar Purkinje cells (Spaltmann and Brummendor Neurosci. 16 (5), 1770-1779 (1996)); (2) chicken CEPU gene identified as a neural secreted glycoprotein belonging to the immunoglobulin-like opioid binding cell adhesion molecule (OBCAM) subfamily, (Kim et al., 1999 Mol. Cells 9 (3), 270-276); and (3) Bovine mRNA for opioid binding protein/cell adhesion molecule OBCAM.

Performing studies with phosphatidyl inositol (“PI”) specific phospholipase C can confirm the suggestion that LP289 is bound to the cell membrane via a PI linkage (such techniques are commonly used and known in the art and would not require undue experimentation, see, e.g., the methods described in Hachisuka, et al. 1996 Neurochem. Int. 28:373-379). The immunoglobulin domains of LP289 make up the binding or self-adhesion domains of LP289. It is likely that LP289 is developmentally regulated, for example, other IgLON members show such regulation, e.g., during early development (E16, embryonic day 16) OBCAM is found on post mitotic neurons and in fiber tracts in the CNS that contain expanding axons suggesting the OBCAM functions in axonal outgrowth.

The chromosomal location of LP289 genomic sequence has currently been established to reside on chromosome 19. It has been discovered that LP289 sequence (SEQ ID NO: 7) is expressed in the following number of LIFESEQ GOLD™ database tissue and cDNA libraries: Digestive System 1/151; Genitalia, Male 2/118; Germ Cells 1/5; Hemic and Immune System 3/166; Liver 1/34; Respiratory System 1/95; Sense Organs 1/10; and Nervous System 17/221.

TABLE 4

Primate, e.g., human, LP289 polynucleotide sequence (SEQ ID NO: 7) and corresponding
polypeptide (SEQ ID NO: 8). The ORF for LP289 is 17-1027 bp (with the start (ATG) and stop codons
(TAG) identified in bold typeface and underlined). In case the numbering used herein is misidentified
one skilled in the art could determine the open reading frame without undue experimentation using the
described translation and figures listed herein.

LP289 Nucleic Acid Sequence (2653 bp) (ORF = 17-1027)
LP289 start (atg) and stop (tga) codons are indicated in bold typeface
and underlined.
ATGCCCCCCCCTGCGCCCGGGGCCCGGCTCCGGCTTCTCGCCGCCGCCGCCCTGGCCGGCTTGGCCGTCAT
CAGCCGAGGGCTGCTCTCCCAGAGCCTGGAGTTCAACTCTCCTGCCGACAACTACACAGTGTGTGAAGGTG
ACAACGCCACCCTCAGCTGCTTCATCGACGAGCACGTGACCCGCGTGGCCTGGCTGAACCGCTCCAACATC
CTGTATGCCGGCAATGACCGCTGGACCAGCGACCCGCGGGTGCGGCTGCTCATCAACACCCCCGAGGAGTT
CTCCATCCTCATCACCGAGGTGGGGCTCGGCGACGAGGGCCPCTACACCTGCTCCTTCCAGACCCGCCACC
AGCCGTACACCACTCAGGTCTACCTCATTGTCCACGTCCCTGCCCGCATTGTGAACATCTCGTCGCCTGTG
ACGGTGAATGAGGGGGGCAATGTGAACCTGCTTTGCCTGGCCGTGGGGCGGCCAGAGCCCACGGTCACCTG
GAGACAGCTCCGAGACGGCTTCACCTCGGAGGGAGAGATCCTGGAGATCTCTGACATCCAGCGGGGCCAGG
CCGGGGAGTATGAGTGCGTGACTCACAACGGGGTTAACTCGGCGCCCGACAGCCGCCGCGTGCTGGTCACA
CTCAACTATCCTCCGACCATCACGGACGTGACCAGCGCCCGCACCGCGCTGGGCCGGGCCGCCCTCCTGCG
CTGCGAAGCCATGGCGGTTCCCCCCGCGGATTTCCAGTGGTACAAGGATGACAGACTGCTGAGCAGCGGCA
CGGCCGAAGGCCTGAAGGTGCAGACGGAGCGCACCCGCTCGATGCTTCTCTTTGCCAACGTGAGCGCCCGG
CATTACGGCAACTATACGTGTCGCGCCGCCAACCGACTGGGAGCGTCCAGCGCCTCCATGCGGCTCCTGCG
CCCAGGATCCCTGGAGAACTCAGCCCCGAGGCCCCCAGGGCTCCTGGCCCTCCTCTCCGCCCTGGGCTGGC
TGTGGTOGAGAATGTAG
LP289 Full length Protein sequence (336aa)
(SEQ ID NO:8) The underlined portion is a predicted signal sequence (Met-1 to Ser-30). A predicted SP
cleavage site between Ser-30 and Gln-31 is indicated as follows: 1
MPPPAPGARLRLLAAAALAGLAVISRGLLS\hat{ }QS 32. An alternative predicted cleavage sire (based on a
differernt signal peptide analysis) is between Ala-22 and Val-23 and indicated as follows: 1
MPPPAPGARLRLLAAAALAGLA\hat{ }VI24. A resulting LP289 polypeptide (in either instance) is encompassed
herein.
LP289 has a hydrophobic C-terminal sequence consistent with that found on other GPI-linked proteins;
typically, this type of sequence is cleaved during post-translational processing, such as, e.g., when
a protein is inserted into a membrane via a GPI linkage (see, e.g., Cross, Ann. Rev. Cell Biol.
6:1-39, 1990; Ferguson and Williams, Ann. Rev. Biochem. 57:285.320,1988; Gerber et al.,J. Biol.
Chem. 267:18168. 12173, 1992). A putative GPI anchor attachment point in LP289 is indicated (by double
underlining) at or near (e.g., within 1, 2, 3, 4, or 5, amino acid residues either C-terminad or N-
terminad to the indicated asparagine), the asparagine (N) in the following LP289 sequence (SLENSAP).
The prediction for an LP GPI anchor site herein is based on the w, w+2 rule for predicting the
site of GPI anchor addition in proteins from the method of Udenfriend and Kodukula, 1995b Methods
Enzymol. 250,571-582. Other rules and/or algorithms for predicting the site of a GPI anchor
addition in proteins have been devised based on experimental observations (see, e.g., Udenfriend
and Kodukula, 1995b; Eisenhaber et al., 1999); howvever, such predictions are not perfect. Accordingly,
Applicants invention encompasses 1, 2, 3, 4, or 5, amino acid residues either and/or both C-terminad
and/or N-terminad to a predicted GPI site described herein. Furthermore, Applicants invention
encompasses LP variants in which LP289 alterations prevent a typical GPI modification thereby
resulting in a soluble/secreted LP289 variant. Sequence C-terminad to such an LP289 putative GPI
anchor sire is typically removed during processing to a mature LP289 form. LP289 sequence that is
typically removed during process of LP289 is indicated below by waved underling
(SAPRPPGLLALLSALGWLWWRM). LP289 polypeptides encompassed herein include full-length forms encoded by
an ORF disclosed herein, as well as any mature forms therefrom. Such a mature LP289 could be formed,
for example, by the removal of a signal peptide. Further as used herein, a “mature” LP encompass,
e.g., post-translational modifications other than proteolytic cleavages (such as, e.g., by way of a
non-limiting example, glycosylations, myristylations, phosphorylations, prenylations, acylations, and
sulfations). Such variants are also encompassed by an LP of the present invention. Further, an LP of
the invention encompass all fragments, analogs, homologs. and derivatives of an LP described herein,
thus the invention encompasses both LP precursors and any modified versions (such as, e.g., by post-
translational modification) of an LP encoded by an LP nucleic acid sequence described herein.).
MPPPAPGARLRLLAAAALAGLAVISRGLLSQSLEFNSPADNYTVCEGDNATLSCFIDEHVTRVAWLNRSNIL
YAGNDRWTSDPRVRLLINTPEEFSILITEVGLGDEGLYTCSFQTRHQPYTTQVYLIVHVPARIVNISSPVTV
NEGGNVNLLCLAVGRPEPTVTWRQLRDGFTSEGEILEISDIQRGQAGEYECVTHNGVNSAPDSRRVLVTVNY
PPTITDVTSARTALGRAALLRCEAMAVPPADFQWYKDDRLLSSGTAEGLKVQTERTRSMLLFANVSARHYGN
YTCRAANRLGASSASMRLLRPGSLENSAPRPPGLLALLSALGWLWWRM*
An LP289 Mature Sequence (306aa):
A predicted mature LP289 sequence is as follows:
QSLEFNSPADNYTVCEGDNATLSCFIDEHVTRVAWLNRSNILYAGNDRWTSDPRVRLLINTPEEFSILITEV
GLGDEGLYTCSFQTRHQPYTTQVYLIVHVPARIVNISSPVTVNEGGNVNLLCLAVGRPEPTVTWRQLRDGFT
SEGEILEISDIQRGQAGEYECVTHNGVNSAPDSRRVLVTVNYPPTITDVTSARTALGRAALLRCEAMAVPPA
DFQWYKDDRLLSSGTAEGLKVQTERTRSMLLFANVSARHYGNYTCRAAVRLGASSASMRLLRPGSLENSAPR
PPGLLALLSALGWLWWRM*
An Alternate LP289 Mature Sequence (314aa):
An alternate predicted mature LP289 sequence is as follows:
VISRGLLSQSLEFNSPADNYTVCEGDNATLSCFIDEHVTRVAWLNRSNILYAGNDRWTSDPRVRLLINTPEE
FSILITEVGLGDEGLYTCSFQTRHQPYTTQVYLIVHVPARIVNISSPVTVNEGGNVNLLCLAVGRPEPTVTW
RQLRDGFTSEGEILEISDIQRGQAGEYECVTHNGVNSAPDSRRVLVTVYPPTITDVTSARTAGLGRAALLRC
EAMAVPPADFQWYKDDRLLSSGTAEGLKVQTERTRSMLLFANVSARHYGNYTCRAANRLGASSASMRLLRPG
SLENSAPRPPGLLALLSALGWLWWRM*
An Alternate LP289 Mature Sequence (284aa):
A further alternate predicted mature LP289 sequence is as follows:
QSLEFNSPADNYTVCEGDNATLSCFIDEHVTRVAWLNRSNILYAGNDRWTSDPRVRLLINTPEEFSILITEV
GLGDEGLYTCSFQTRHQPYTTQVYLIVHVPARIVNISSPVTVNEGGNVNLLCLAVGRPEPTVTWRQLRDGFT
SEGEILEISDIQRGQAGEYECVTHNGVNSAPDSRRVLVTVNYPPTITDVTSARTALGRAALLRCEAMAVPPA
DFQWYKDDRLLSSGTAEGLKVQTERTRSMLLFANVSARHYGNYTCRAANRLGASSASMRLLRPGSLEN*
An Alternate LP289 Mature Sequence (292aa):
A further alternate predicted mature LP289 sequence is as follows:
VISRGLLSQSLEFNSPADNYTVCEGDNATLSCFIDEHVTRVAWLNRSNILYAGNDRWTSDPRVRLLINTPEE
FSILITEVGLGDEGLYTCSFQTRHQPYTTQVYLIVHVPARIVNISSPVTVNEGGNVNLLCLAVGRPEPTVTW
RQLRDGFTSEGEILEISDIQRGQAGEYECVTHNGVNSAPDSRRVLVTVNYPPTITDVTSARTALGRAALLRC
EAMAVPPADFQWYKDDRLLSSGTAEGLKVQTERTRSMLLFANVSARHYGNYTCRAANRLGASSASMRLLRPG
SLEN*
A Variant LP289 (291aa):
The structural features of an LP289 variant represent a soluble counterpart to a non-soluble LP289
version encompassed herein, wherein a difference of a soluble LP289 is at the LP289 C-terminus in
which a GPI-anchored binding site is absent. For example, a non-limiting example of such a variant
LP289 sequence encompassed herein is as follows:
VISRGLLSQSLEFNSPADNYTVCEGDNATLSCFIDEHVTRVAWLNRSNILYAGNDRWTSDPRVRLLINTPEE
FSILITEVGLGDEGLYTCSFQTRHQPYTTQVYLIVHVPARIVNISSPVTNEGGNVNLLECLAVGRPEPTVTW
RQLRDGFTSEGEILEISDIQRGQAGEYECVTHNGVNSAPDSRRVLVTVNYPPTITDVTSARTALGRAALLRC
EAMAVPPADFQWYKDDRLLSSGTAEGLKVQTERTRSMLLFANVSARHYGNYTCRAANRLGASSASMRLLRPG
SLE*

Analysis of the primary amino acid structure of LP289 demonstrates that LP289 possesses typical IgLON characteristics, including homology to known IgLON members and IgLON-like motifs. Particularly interesting portions or fragments of a full length LP289 polypeptide include, e.g., a discovered putative signal peptide-like sequence from Met-1 to Ser-30 (MPPPAPGARLRLLAAAALAGLAVISRGLLS). An alternative predicted cleavage site (based on a different signal peptide analysis) results in an alternate putative signal peptide-like sequence from Met-1 to Ala-22 (MPPPAPGARLRLLAAALAGLA). Based on the teachings supplied herein (e.g., the LP289 sequence and its relationship with the domains, motifs, and signatures of other known IgLONs) and those known in the art (e.g., assay methods to determine binding activities of suspected IgLONs such as neurite outgrowth, homo- or heterophilic binding, axonal pathfinding, opiod-like binding, see, e.g., the assays described in, e.g., Hachisuka, et al. 1996 Neurochem. Int. 28:373-379 and others listed herein), one skilled in the art would be able to test LP289 for IgLON-like activities without undue experimentation (e.g., using common assay techniques and commercially available reagents).

Additionally interesting portions of LP289 are three immunoglobulin-like domains: from about Gly-47 to about Phe-114: (GDNATLSCFIDEHVTRVAWLNRSNILYAGNDRWTSDPRVRLLINTPEEFSILITEVGLGDEGLYTCSF); from about Gly-147 to about Thr-197: (GGNVNLLCLAVGRPEPTVTWRQLRDGFTSEGEILEISDIQRGQAGEYECVT); and from about Gly-231 to about Ala-293 (GRAALLRCEAMAVPPADFQWYKDDRLLSSGTAEGLKVQTERTRSMLLFANVSARHYGNYTCRA). Additionally interesting segments of LP289 are discovered fragments Arg-11 to Ser-30; Ser-32 to Asn-49; Arg-68 to Leu-88; Val-125 to Val-144; Pro-1 60 to Leu-180; Gly-188 to Val-210; Pro-245 to Ala-262; Arg-11 to Ala-22; Val-23 to Asn-36; Ser-3⁷ to Thr-51; Ser-53 to Ala-64; Ala-65 to Ala-74; Pro-83 to Phe-95; Ser-96 to Thr-111; Tyr-121 to Pro-141; Val-150 to Pro-162; Asp-184 to Cys-195; Val-196 to Arg-208; Arg-209 to Asp-222; Val-223 to Ala-234; Met-241 to Leu-257; Ser-259 to Thr-272; Arg-273 to Ser-282; Arg-283 to Ser-303; Arg-318 to Ser-327; Arg-18 to Leu-28; Leu-29 to Cys-45; Glu-46 to Ala-64; Asn-67 to Leu-88; Glu-101 to Tyr-110; Thr-111 to Gln-124; Val-125 to Val-136; Asn-137 to Leu-152; Gly-158 to Ile-179; Glu-181 to Thr-197; Asp-198 to Thr-213; Asn-215 to Leu-235; Cys-238 to Arg-255; Leu-256 to Met-275; Val-281 to Arg-305; Arg-308 to Pro-320; whose discovery was based on an analysis of hydrophobicity, hydropathicity, and hydrophilicity plots of LP289. Additional interesting sections of LP289 are the discovered portions of LP289 from about Ala-8 to about Ala-22 from about Phe-35 to about Asp-48 from about Asn-49 to about Thr-61 from about Leu-66 to about Asp-77; Pro-83 to about Phe-95 from about Ser-96 to about Glu-107 from about Gly-119 to about Gln-119 from about Val-129 to about Ser-140 from about Pro-141 to about Asn-152 from about Leu-152 to about Val-164 from about Trp-166 to about Glu-176 from about Gly-177 to about Gly-188 from about Gln-189 to about Val-201 from about Thr-213 to about Ala-226 from about Arg-227 to about Leu-236 from about Phe-248 to about Ser-259 from about Thr-269 to about Phe-278 from about Ser-303 to about Leu-312. These fragments were discovered based on analysis of antigenicity plots of LP289. Further, particularly interesting LP 289 secondary structures (e.g., such as a helix, a strand, or a coil) are the following LP289 coil structures: from about Met-1 to about Pro-6; from about Gly-27 to about Asp-40; from about Glu-46 to about Asn-49; from about Asn-67 to about Ser-69; from about Ala-74 to about Arg-84; from about Asn-90 to about Glu-94; from about Gly-103 to about Gly-108; from about Thr-116 to about Thr-122; from about Pro-132 to about Ala-133; from about Ile-138 to about Pro-141; from about Glu-146 to about Asn-149; from about Val-157 to about Thr-163; from about Asp-171 to about Gly-177; from about Gly-188 to about Glu-192; from about His-198 to about Ser-207; from about Asn-215 to about Val-223; from about Ala-242 to about Phe-248; from about Tyr-251 to about Asp-254; from about Ser-258 to about Gly-264; from about Ala-283 to about Asn-288; Leu-297 to about Ser-300; from about Arg-308 to about Gly-321; and from about Met-336 to about Met-336. Particularly interesting LP289 helix structures are: from about Leu-12- to about Gly-20; from about Arg-227 to about Glu-239; from about Ala-302 to about Arg-305; and from about Leu-326 to about Ala-328. Particularly interesting strands are from about Tyr-42 to about Cys-45; from about Leu-52 to about Phe-55; from about Ala-64 to about Ala-64; from about Ile-71 to about Tyr-73; from about Val-85 to about Leu-87; from about Ser-96 to about Glu-101; from about Tyr-110 to about Cys-112; from about Gln-124 to about Val-129; from about Ile-135 to about Asn-137; from about Thr-143 to about Val-144; from about Asn-151 to about Leu-155; from about Val-164 to about Trp-166; from about Tyr-193 to about Thr-197; and from about Arg-209 to about Val-214. Further encompassed by the invention are contiguous amino acid residue combinations of any of the predicted secondary structures described above. For example, one strand-coil-helix-coil motif of LP289 combines the Arg-209 to Val-214 strand; and the Asn-215 to Val-223 coil; and the Arg-227 to Glu-239 helix; with the Ala-242 to Phe-248 coil to form an interesting fragment of contiguous amino acid residues from about Arg-209 to about Phe-248. Other combinations of contiguous amino acids contemplated are also encompassed as can be easily determined.

Further encompassed herein are LP289 splice variants, such as, e.g., splice variants in which alterations in the processing of an LP289 mRNA results in a soluble/secreted LP289 that is not anchored to a cell membrane. In one such embodiment, such a full-length LP289 splice variant is from about Met-1 to about Thr-33 (MPPPAPGARLRLLAAAALAGLAVISRAASSTST), with a predicted cleavage site as indicated as follows: MPPPAPGARLRLLAAAALAGLA\hat{ }VISRAASSTST. The resulting mature LP289 splice variant sequence (also known as LP343; see below) is from about Val-23 to about Thr-33 (VISRAASSTST). This LP289 splice variant sequence was discovered from a brain cDNA library further supporting the role of this LP289 variant in neural functions described herein.

```
Alternatively soliced LP289:
Analysis of non-splice variant LP289 nucleic acid and the splice
variant nucleic acid (whose polypeptide product is designated as
LP343 (SEQ ID NO: 9)), indicates that during mRNA proccesing the
parent of LP289 nucleic acid sequence the 2nd exon is skipped in
the LP289 splice variant (see below). The resulting LP289 splice
variant created is a small peptide (approximately 11 amino acid
residues in length after removal of a signal sequence).
                                                                                      exon 1|exon 2
LP289 GCCGCCCTGGCCGGCTTGGCCGTCATCAGCCGAGGGCTGCTCTCCCAGAGCCTGGAGTTC
LP343 GCCGCCCTGGCCGGCTTGGCCGTCATCAGCCGAG--------------------------
            *********************************
                                                                                                              exon 2|exon 3
LP289 AACTCTCCTGCCGACAACTACACAGTGTGTGAAGGTGACAACGCCACCCTCAGCTGCTTC
LP343 -----------------------------------------------------CTGCTTC
                                                                                                                      *******
L2289 ATCGACGAGCACGTGACCCGCGTGGCCTGGCTGAACCGCTCCAACATCCTGTATGCCGGC
LP343 ATCGACGAGCACGTGACCCGCGTGGCCTGGCTGAACCGCTCCAACATCCTGTATGCCGGC
            ***********************************************************
L2289 Splice Variant Nucleic Acid Sequence (754 bp) (ORF = 19-120):
LP289 splice variant (LP343) start (atg) and stop (tga) codons are
indicated in bold typeface and underlined. >ds42802 Nucleic acid
sequence is:
ATGCCCCCCCCTGCGCCCGGGGCCCGGCTCCGGCTTCTCGCCGCCGCCGCCCTGGCCGGCTTGGC
CGTCATCAGCCGAGCTGCTTCATCGACGAGCACGTGAC
Alignment of the splice variant polypeptide (LP343) with LP289:
LP343 MPPPAPGARLRLLAAAALAGLAVISRAASSTST---------------------------
LP289 MPPPAPGARLRLLAAAALAGLAVISRGLLSQSLEFNSPADNYTVCEGDNATLSCFIDEHV
            *************************** * *
L2343 ------------------------------------------------------------
LP289 TRVAWLNRSNILYACNDRWTSDPRVRLLINTPEEFSILITEVGLGDEGLYTCSFQTRHQP
L2343 ------------------------------------------------------------
L2289 YTTQVYLIVHVPARIVNISSPVTVNEGGNVNLLCLAVGRPEPTVTWRQLRDGFTSEGEIL
LP343 ------------------------------------------------------------
LP289 EISDIQRGQAGEYECVTHNGVNSAPDSRRVLVTVNYPPTITDVTSARTALGRAALLRCEA
LP343 ------------------------------------------------------------
L2289 MAVPPADFQWYKDDRLLSSGTAEGLKVQTERTRSMLLFANVSARHYGNYTCRAANRLGAS
LP343 ------------------------------------------------------------
LP289 SASMRLLRPGSLENSAPRPPGLLALLSALCWLWWRM
```

This LP289 splice variant (LP343; SEQ ID NO: 10) may be useful as a therapeutic peptide to treat nervous system diseases. For example, LP343 (LP289 splice variant) may cross the blood-brain barrier since it is a small amino-acid peptide after cleavage of its predicted signal peptide. Additionally, the LP289 splice variant (LP343) may also self-interact with the parent LP289 to modulate for example, parent LP289 expression, LP289 binding characteristics, LP2289 placement in the cell. Alternatively, LP343 may act as a secreted factor that can function as, e.g., a growth factor in the establishment of neural circuitry that has been established, maintained, or remodelled by, e.g., parent LP289 or another IgLON (as described in detail herein). Furthermore, LP343 may act as a ligand for the parent LP289 or other IgLON member. Such splicing interactions are not unheard of, for example, Karpa, et al. 2000 Mol Pharmacol 58(4):677-683 show that the truncated dopamine D3 receptor splice variant (D3nf) inteacts with the parent D3 receptor to cause it to relocate. Furthermore, Elmhurst, et al. 2000 Brain Res Mol Brain Res 80(1):63-74, demonstrate that the D3nf splice variant reduces ligand binding to the D3 dopamine receptor. Accordingly, discovery of the LP343 splice variant strongly suggests its role in modulating and/or interacting with a parent LP289 function.

Given the teachings supplied herein of: LP289 primary amino acid and higher order structures, the relationship of the LP289 amino acid sequence and higher order structural features compared with known IgSF members (e.g., such as IgLON members (such as, e.g., LAMP, OBCAM, Ntm, CEPU, GP50, KILON, and GP55) and their higher order structural features (including the known functions of these IgLON members and their higher order structures), it is likely that an LP289 or an LP289 variant play similar roles in a variety of physiological processes. Some non-limiting examples of functions an LP289, LP289 variant, or an LP289 binding agent (e.g., such as an LP289 antibody (or fragment thereof)) is likely to participate in are, for example, those such as: neuorogenesis; the formation, development, and/or modification of regional centers in the brain such as, for example, nuclei of, for example, the forebrain: such as, for example, the olfactory bulb and cortex; the neocortex; the striatum, the nucleus accumbens; the basal forebrain; the limbic circuit; the thalamus (including, for example, reticular thalamic nucleus, dorso-caudal nucleus, dorso-intermedial nucleus, dorso-orales nucleus, ventral-caudal nucleus, ventral-intermediate nucleus, ventral-oralis posterior nucleus, ventral-oralis anterior nucleus, sub-thalamic nucleus, and substantia nigra); the hypothalamus: (including, for example, anterior lobe of the pituitary (adenohypophysis), posterior lobe of the pituitary (neurohypophysis), optic chiasm, preoptic nucleus, anterior nucleus, dorsomedial nucleus, ventromedial nucleus, posterior nucleus, mammiliary body, hypothalamic supraoptic nuclei (SON), and paraventricular nuclei (PVN)); the Midbrain, such as, for example, the tectum (superior and inferior colliculi) or the tegmentum; the Hindbrain; the Pons and/or the Cerebellum; and the Medulla; the formation, development, and/or modification of neural architecture such as, e.g., during the formation of the dendritic tree of e.g., Purkinje cells in the cerebellum; modulation of axonal targeting, neurite pathfinding; the formation, development, and/or modification of neural circuitry; the formation, development, maintenance, and/or modification of distinct neuronal systems; the formation, maintenance, and/or modification of neural connections; cell adhesion; neurite outgrowth; regulation of the development of neuronal projections via cell specific attractive and/or repulsive mechanisms; the formation, maintenance, and/or modification of neural circuitry; the formation, maintenance, and/or modulation of axo-dendritic, dendro-dendritic, axo-axonal, dendro-axonal, axo-somata, dendro-somata, and somata-somata, interactions during the formation, maintenance, and/or modification of neuronal projections; cell-cell interactions in the nervous system; the formation, promotion, maintenance and/or remodeling of fiber fasciculation on neural cells (such as, e.g., axonal surfaces of developing or remodeling neural surfaces (e.g., by either homophilic or heterophilic interactions) for example, such as, targeting and/or maintenance of brain nucleii projections (e.g., such as targeting of thalamocortical axons to their appropriate cortical area during development); modulate adhesion of neurons to a substrate; modulate and/or regulate binding of an opiod and/or opiod-like ligand; specification of neuronal connectivity; modulation of an opiod and/or opiod-like nociception state, condition, or syndrome; modulation of an opiod and/or opiod-like antinociception state, condition, or syndrome; facilitation, and/or modulation of opiod and/or opiod-like signal transduction; facilitation, and/or modulation of opiod and/or opiod-like signal transduction, wherein the opiod or opiod-like composition is a mu or mu-like opiod; facilitation, and/or modulation of opiod and/or opiod-like signal transduction, wherein the opiod or opiod-like composition is a kappa or kappa-like opiod; facilitation, and/or modulation of opiod and/or opiod-like signal transduction, wherein the opiod or opiod-like composition is a delta or delta-like opiod; modulate the formation of an opiod or opiod-like receptor coupling to an intracellular scaffold and/or adaptor protein/s; facilitate and/or modulate an opiod receptor/G-protein coupling; modulate, treat, and/or diagnose, and/or prognose an opiod or opiod-like tolerance state, condition, or syndrome; modulate, treat, and/or diagnose, and/or prognose an opiod or opiod-like dependence state, condition, or syndrome; modulate, treat, and/or diagnose, and/or prognose an opiod or opiod-like dependence state, condition, or syndrome, wherein the opiod or opiod-like dependence state, condition, and/or syndrome is related to a delta or delta-type opiod or opiod-like composition; modulate, treat, and/or diagnose, and/or prognose an opiod or opiod-like dependence state, condition, or syndrome, wherein the opiod or opiod-like dependence state, condition, and/or syndrome is related to a mu or mu-type opiod or opiod-like composition; modulate, treat, and/or diagnose, and/or prognose an opiod or opiod-like dependence state, condition, or syndrome, wherein the opiod or opiod-like dependence state, condition, and/or syndrome is related to a kappa or kappa-type opiod or opiod-like composition; treatment and/or modulation of a disease, condition, syndrome, or a state of respiratory control; treatment and/or modulation of a disease, condition, syndrome, or a state of appetite control; modulate the formation of an opiod receptor-G-protein binding complex; modulate an opiod receptor conformational transformation; the formation, promotion, maintenance and/or remodeling of synaptic connectivity; the promotion, stimulation, maintenance, and/or inhibition of neurite outgrowth; growth cone guidance; inhibition and/or promotion of a regeneration of CNS or PNS neurons after damage; stimulation of embryonic neurons; inhibition of mature neurons; a neuro-attractant; and a neuro-repellant.

In one embodiment, LP289 nucleic acids of the invention can be used to create LP289-derived polypeptides that interact with native LP289 located, for example, at a neural cell surface either to stimulate the growth and differentiation activities of LP289 or to inhibit those activities. Particularly preferred are such polypeptides that are soluble LP289 analogs having binding domains effective to bind LP289. The LP289-inhibitory polypeptides that are encoded by these nucleic acids can be used to treat diseases characterized by abnormal growth and functioning of neurons, such as, for example, neurons of the central nervous system, such as those involved in conditions of epilepsy, Alzheimer's disease, and schizophrenia-like diseases states or conditions. Antisense strategies to inhibit the expression of LP289 can also be used to treat these diseases. Another use for LP289 nucleic acids of the invention is to create targeting polypeptides for directing the delivery of biological agents to locations within the nervous system, such as, for example, neurons involved in brain circuits associated with analgesia. LP289 polypeptides of the invention are useful targeting agents because they bind LP289 found at the cell surfaces in the nervous system, for example, such as cells in the limbic system. Such targeting agents encompassed herein are bound covalently or noncovalently to a biological agent or a vehicle for delivering biological agents such as, for example, delivery methods described herein or otherwise art known. Biological agents are those that can act on a cell, organ or organism, including, but not limited to, pharmaceutical agents and gene delivery agents. Numerous targetable delivery vehicles are known, including liposomes, ghost cells and polypeptide matrices (see, for example, Huang et al., Proc. Natl. Acad. Sci. USA, 84, 7851-7855, 1987; Kreuter, Infection 19 Supp. 4, 224-228, 1991; or Michel et al., Research in Virology, 144, 263-267, 1993).

In another embodiment, LP289 nucleic acids can also be used to transform stem cells to program their development as neural system neurons. These replacement neurons can be implanted to treat neuropathologies by reconnecting circuits involved in cognition, mood, memory and learning, and cardiovascular regulation, providing therapies for diseases, conditions, syndromes, etc., such as, for example, dementia (including without limitation Alzheimer's disease, multi-infarct dementia, dementia associated with Parkinson's disease), all forms of epilepsy, major depression, anxiety (including, without limitation, manic-depressive illness, generalized anxiety, obsessive-compulsive disorders, panic disorder and others), schizophrenia, and schizophrenaform disorders (including without limitation schizoaffecto disorder), cerebral palsy and hypertension. Non-limiting examples of stem cells that are useful in neural stem cell replacement therapy include human cortical and subcortical fetal brain cells, porcine fetal brain cells, human subventricular zone cells, and glial progenitor cells, including 02A cells (which are progenitors for all glial cell types, including astrocytes and oligodendrocytes).

The LP289 nucleic acids of the invention can be used to create LP289-derived polypeptides that interact with LP289 located at a neuron cell surface either to stimulate the growth and differentiation activities of LP289 or to inhibit those activities. Particularly preferred are such polypeptides that are soluble LP289 analogs having binding domains effective to bind LP289. An LP289-inhibitory polypeptide can be used to treat diseases characterized by abnormal growth and functioning of neurons, such as epilepsy, Alzheimer's disease, and schizophrenia. Antisense strategies to inhibit the expression of LP289 can also be used to treat these diseases. Another use for the nucleic acids of the invention is to create targeting polypeptides for directing the delivery of biological agents to the nervous system where LP289 is expressed (for example, such as, the limbic system, the dentate gyrus, the forebrain, the thalamus, the midbrain, etc.). The LP289 polypeptides are useful targeting agents because they bind to LP289 found at the cell surface of neuronal cells such as, for example, in the CNS, such as, for example, in the limbic system. Such targeting agents are bound covalently or noncovalently to a biological agent or a vehicle for delivering biological agents that can act on a cell, organ, or organism, including, but not limited to, pharmaceutical agents and gene delivery agents. Non-limiting examples of biological agents that can be usefully targeted to, e.g., the limbic system include, neurotransmitter biosynthetic enzymes (such as tyrosine hydroxylase), neurotransmitter transporters (such as the GABA transporter), neurotransmitter receptors (such as type la, Ib, II or III dopamine receptors, a and (3 adrenergic receptors and 5-HT receptors), neurotrophic and growth factors (such as NGF, BDNF, NT-3, NT-4, NT-5, TGF13, basic FGF and GDNF), neurotrophic factor receptors, protein kinases (such as MAP kinases and protein kinase C) and protein phosphatases. Further agents include, without limitation, antidepressants, neuroleptics, anti-epileptics and antagonists of neurotransmitter receptors (such as type la, 1b, II or III dopamine receptors, A and B adrenergic receptors and 5-HT receptors).

The ability of LP289 expression to modulate growth and/or differentiation of various embryonic cell populations, such as, for example, various neuronal embryonic cell populations (such as, e.g., neurons from the hippocampus, the perirhinal cortex, the olfactory bulb, and the visual cortex) can be tested using a substrata of CHO cells, which have been transfected and/or transformed by LP289 (with controls of CHO cells transfected and/or transformed with a vector only).

For example, cells from various CNS areas are tested such as, for example, cells from hippocampal and perirhinal cortex in comparison to cells from non-LP289 expressing areas, such as, for example, cardiovascular or connective tissue system cells. Primary neurons from rodent E16 embryos are prepared as outlined by Ferri and Levitt, 1993 Cerebral Cortex 3; 187-198. In some experiments, cells are marked by adding lipophilic dye PKH26 (Sigma Chemical Co., St. Louis, Mo.); if they are not so marked, an antibody stain is used later in the experiment to identify neural cells. The cells are plated in DMEM/10% FCS at a density of 5×103 cells/ml per cm2, onto coverslips on which there are monolayers of transformed CHO cells. After 48 hours in culture, the cells attached to the coverslips are fixed with formaldehyde and, if the neural cells are not dye-marked, stained for neural cells with anti-MAP2, as described in Ferri and Levitt, 1993 Cerebral Cortex 3: 187-198. For each experiment, six coverslips are examined and the longest neuron in a randomly selected field of 10-15 process-bearing cells is measured. The plated cells are examined within 24 hours to determine the presence or absence of: neurite growth, a well-differentiated neural morphology, cyto-architecture, and arborization. Cells can also be examined for these criteria when they are pre-treated with LP289 antibody, or soluble LP289 to determine if the length and/or number of neurites are significantly modified.

LP289 and Neural Development and Neural Circuits: Based on an analysis of LP289, such as, e.g., its homology to other IgLON members, LP289 is likely to be involved in the regional specification of the central nervous system, e.g., regional specification of the brain, such as, e.g., the formation, maintenance, and/or modulation of neural circuits in the brain. For example, individual members of the IgLON family are expressed on distinct populations of neurons that, for the most part, form functional circuits in the nervous system, such as, e.g., in the brain. For instance, the IgLON member Neurotrimin (Ntm) has an expression pattern that is largely complementary to that of LAMP, with the highest expression of Ntm in the sensorimotor cortex, Neurotrimin's expression in layers IV, V, and VI of the cortex, the subplate, and the rostral lateral thalamus as well as in the pontine nucleus and cerebellum suggests a potential role in the development of thalamocortical and pontocerebellar projections, respectively (Struyk et al., 1995). The IgLON member OBCAM has a more restricted distribution, with highest expression in the cortical plate and hippocampus (Struyk et al., 1995 J Neurosci 15:2141-2156) another member of the IgLON family of proteins.

The IgLON member LAMP is expressed by cortical and subcortical neurons of the limbic system (Levitt, 1984 Science 223:299-301) and has been strongly implicated in the development of projections in this system (Pimenta et al., 1995 Neuron 15:287-297; Zhukareva and Levitt, 1995 Mol Cell Neurosci 10:43-55). LAMP has been shown to play a role in specifying a subset of thalamocortical projections, which at early developmental stages are selectively expressed in the perirhinal and frontal limbic cortex and medial limbic thalamic nuclei (Levitt, 1984 Science 223:299-301; Horton and Levitt, 1988; Pimenta et al., 1996). LAMP acts homophilicaly to promote adhesion and growth of limbic axons (Pimenta et al., 1995 Neuron 15:287-297; Zhukareva and Levitt, 1995 Mol Cell Neurosci 10:43-55), and antibody perturbation studies show that LAMP can regulate the formation of septohippocampal and intrahippocampal circuits (Keller and Levitt, Neuroscience 28: 455-474, 1989; Pimenta et al., 1995 Neuron 15:287-297). Furthermore, for limbic thalamic axons, LAMP acts as an attractive guidance signal that also induces branch formation while nonlimbic thalamic fibers are deflected and axonal branching is inhibited by LAMP. For instance, neocortical and limbic striatal dopamine circuitry (such as, e.g., circuitry of dopamine receptor neurons in the substantia nigra (SN) whose axons connect to neostriatal (caudate-putamen) nucleii or dopamine receptor neurons in the ventral tegmental area (VTA) whose axons connect with limbic or ventral striatum, including nucleus accumbens nucleii,) are postulated to affect information transfer from cerebral cortex, amygdala, and hippocampus to brain areas that effect thought, action, and emotions. Furthermore, current evidence strongly indicates that the ventral tegmental and the nucleus accumbens regions are involved in addiction, with the neurotransmitter dopamine playing a crucial role. Thus, stimulation of the dopaminergic pathway from the VTA to the NA is highly rewarding, and several addictive drugs, including cocaine, amphetamine, heroin, and nicotine, lead either to increased dopamine release or increased dopamine efficacy in the NA. Moreover, more than 90% of cerebral dopamine receptors are located in such striatal nuclei and dopamine and/or dopamine-related brain circuitry is known to play a role in schizophrenic, or schizophrenic-like conditions. Consequently, experiments can be carried out to determine if LP289 plays a role in the formation, maintenance, and/or such remodeling of CNS neural circuits by e.g., promoting the adhesion and/or growth of developing neural extensions (such as, for example, developing neurites). In this view, LP289 expression in the formation, maintenance, and/or modulation of such circuits would have important consequences for diseases, syndromes, or conditions of mood, thought, appetite, addiction, and/or emotion. Consequently, it would be useful to examine the role of LP289 in such neural circuits.

One means to expense LP298's role in the nervous system is to determine if an LP289 binding agent, such as, for example, an antibody or antibody binding fragment directed against an LP289 polypeptide (or fragment thereof) interferes in the post natal development of a neural circuit of the central nervous system, e.g., such as an hippocampal circuit, the following or similar experiments can be carried out (other known neural circuits and their corresponding neuronal architecture can be examined in a similar fashion using similar methods): Newborn Sprague-Dawley rats are injected intraventricularly with Fab fragments of anti-LP289, control anti-paramyosin IgG, and anti-L1. Anti-L1, which binds to developing axons, is as described by Sweadner, J. Neurosci. 3: 2504-2517, 1983. All antisera are purified on a protein A column using a protein A affinity enhancement buffer (the MAPSII buffer system used as recommended by the supplier: Biorad Labs, Hercules, Calif.). Fab fragments are prepared from the antisera by digestion with immobilized papain (Pierce, Rockford, Ill.) and purified by protein-A affinity chromatography. The Fab fragments (10/cg in 10 ul of saline) are injected on postnatal day 0, 2, 4, and 6 into the cisterna magna using a 35-32 gauge needle. On day 9, the animals are sacrificed by transcardial perfusion with 4.9% sodium sulfide in 0.1 M phosphate buffer (pH 7.4). Brains are fixed in Carnoy's solution together with 1.2% sodium sulfide. Paraffin sections of the brains are prepared (in this instance) for mossy fiber staining using the Timm method (see, Haug, Adv. Anat. Embryol. Cell Biol. 47: 1-71, 1973) although other methods of examining neural arthitecture in other region of the brain can also be used. Subfields are analyzed for density of innervation using the Bioquant OS/2 image analysis system (R & M Biometrics, Nashville, Tenn.) to examine neural architecture, for example, such as the mossy fiber projection of granule cells to pyramidal neurons of the hippocampus express. Results are examined to determine if anti-LP289 treatment, but not the other antibody treatments, results in an uncharacteristic neuronal architecture, such as, for example, a diffuse pattern of mossy fiber projections indicating misdirected fibers. Quantitative effects can also be determined, for example, a positive effect of LP289 treatment should result in a statistically significant increase in the area occupied by, for example, mossy fiber projections.

Spinal Cord Regeneration Model: To evaluate the role LP289 in a spinal cord regeneration response (based on the methods of O'Hara, and Chernoff 1994 Tissue and Cell, 26: 599-611; Chernoff, et al. 1998 Wound Rep. Reg. 6: 435-444; Chernoff, et al, 2000 Wound Rep. Reg. 8: 282-291, which are incorporated herein for these teachings) a tissue culture system using axolotl spinal cord ependymal cells is used to test the effects of LP289. Additionally, using other techniques (see, e.g., Itasaki, et al, 1999 Nature Cell Biology Dec;1(8):E203-207; Momose, et al., 1999 Develop. Growth Differ. 41:335-344; and Atkins, et al., 2000 Biotechniques 28: 94-96, 98, 100; which are incorporated herein for these teachings) one can conduct localized transfection studies of LP289 constructs to LP289 constructs in frog limb cultures and frog spinal cord. Although these methods were developed first in the chick, they can be used in a frog limb system for example, to examine the role of LP289 in, for example, regeneration.

Features of LP No: 5 & 6 (LP319a & LP319b)

LP319a & LP319b are a novel primate (e.g., human) polypeptides (SEQ ID NO: 12 & 14), which are a newly discovered variant members of the IgLON family, which includes, e.g., such proteins as LAMP, OBCAM, Ntm (neurotrimin), CEPU, GP50, KILON, and GP55. See above for a description of IgLON sequence and structures. LP319a & LP319b as opposed to LP289, however, are not canonical IgLON members since they display features at the amino acid level that are different from a typical IgLON, e.g., LP319a has one, not two, conserved cysteines in the most N-terminad Ig-like C2-type domain; however, the second LP319a Ig-like C2-type domain (moving C-terminad) resembles a typical IgLON Ig-like C2-type domain, while the third Ig-like C2-type domain contains only one cysteine and appears truncated (in comparison to other IgLONs). Additionally, LP319a contains no GPI-anchor-like motif followed by a hydrophilic spacer region and a C-terminal signal sequence of predominately hydrophobic amino acids. Accordingly, native LP319a is likely to be a secreted IgLON similar to CEPUS, a soluble counterpart to the cerebellar Purkinje cell specific antigen, CEPU-1, which is a secreted IgLON member that is believed to provide a favorable route for migrating CEPU-positive population of neurons to generate a neuron-specific guidance in developing neurons in vivo. It is likely that native LP319a performs a similar role as CEPUS. Applicants invention encompasses, however, variant LP319s such as, e.g., variants in which a C-terminad GPI anchor sequence and/or an Ig-C2 like domain is fused to a native LP319a sequence.

LP319b is also not a typical IgLON in that it displays features at the amino acid level which differ from other IgLONs. Specifically, LP319b does not exhibit three typical IgLON Ig-like-C2-type domains. The carboxy-most Ig-like-C2-type domain of LP319b contains only one conserved cysteine and appears truncated in comparison to other IgLONs. Additionally, LP319b contains no GPI-anchor-like motif followed by a hydrophilic spacer region and a C-terminal signal sequence of predominately hydrophobic amino acids. Accordingly, native LP319b is likely to be a secreted IgLON similar to CEPUS, a soluble counterpart to the cerebellar Purkinje cell specific antigen, CEPU-1.

The chromosomal locations of LP319a and LP319b genomic sequences have been established to reside on chromosome 7. LP319a sequence (SEQ ID NO: 11) is expressed in the following number of LIFESEQ GOLD™ database tissue and cDNA libraries: Digestive System 1/151; Embryonic Structures 3/23; Genitalia, Male 2/118; Germ Cells 1/5; Hemic and Immune System 3/166; Liver 1/34; Respiratory System 1/95; Sense Organs 1/10; and Nervous System 17/221. LP319b nucleic acid sequence (SEQ ID NO: 13) is expressed in the following number of LIFESEQ GOLD™ database tissue and cDNA libraries: Digestive System 1/151; Embryonic Structures 3/23; Genitalia, Male 2/118; Germ Cells 1/5; Hemic and Immune System 3/166; Liver 1/34; Respiratory System 1/95; Sense Organs 1/10; and Nervous System 17/221.

TABLE 5

Primate, e.g., human, LP319a polynucleotide sequence (SEQ ID NO: 11 and corresponding
polypeptide (SEQ ID NO: 12). The ORF for LP319a is 62-787 bp (with the starr (ATG) and stop codons
(TAG) identified in bold typeface and underlined. In case the numbering used herein is misidentified
one skilled in the art could determine the open reading frame without undue experimentation using the
described translation and figures listed herein.

LP319a Nucleic Acid Sequence (2597 bp) (ORF = 62-787)
LP319a start (atg) and stop (tga) codons are indicated in bold typeface and
underlined.
ATGCCCCCCGCTGCGC4CGGGGCCCGGCTCCGGCTTCTCGCCGCCGCCGCCCTGGCCGGCTTGGCCGTCATC
AGCCGGGGGCTGCTCTCCCAGAGGCTGGAGTTCAACTCTCCTGCCGACAACTACACACATGTGACCCGCGTG
GCCTGGCTGAACCGCTCCAACATCCTGTACGCCGGCAACGACCGCAGGACCAGGGACCCGCGGGTGCGGCTG
CTCATCAACACCtCCGAGGAGTTCTCCATCCTCGTCACCGAGGTGGGGCTCGGCGACGAGGGCCTCTACACC
TGCTCCTTCCAGACCCGCCACCAGCCGTACACCACTCAGGTCTACCTCATTGTCCACGTCCCTGCCCGCGTT
GTGAACATCTCGTCGCCTGTGATGGTGAATGAGGGAGGTAATGTGAACCTGCTTTGCCTGGCCGTGGGGCGG
CCAGAGCCCACGGTCACCTGGAGACAGCTCCGAGACGGCTTCACCTCGGAGGGAGAGATCCTGGAGATCTCT
GACATCCTGCCGGGCCAGGCCGGCCAGTATGAGTGCGTGACTCACAACGGGGTTAACTCGGCGCCCGACAGC
CGCCGCGTGCTGGTCACAGTCAACTATCCTCCGACCATCACGGACGTGACCAGCGCCCGCACCGCGCTGGGC
CGGGCCGCCTACTGCGCTCCGAAGCCATGGCGGTTTCCCCCGCGGATTTCCAGTGGTATAAGGATGACAGAC
TACTGA
LP319a Full-length Sequence (241aa):
(SEQ ID NO: 12) The underlined portion is a predicted signal sequence (Met-1 to Ser-30). A predicted SP
cleavage site is between Ser-30 and Gln-31 indicated as follows: 1
MPPAAPGARLRLLAAAALAGLAVISRGLLS\hat{ }QR 32. An alternative predicted cleavage site (based on a
different signal peptide analysis) is between Ala-22 And Val-23 indicated as follows: 1
MPPAAPGARLRLLAAAALAGLA\hat{ }VI 24. A resulting LP319a (in either instance) is encompassed herein.
MPPAAPGARLRLLAAAALAGLAVISRGLLSQRLEFNSPADNYTHVTRVAWLNRSNILYAGNDRRTRDPRVRI
LINTSEEFSILNTEVGLGDEGLYTCSFQTRHQPYTTQVYLIVHVPARVVNISSPVMVNEGGNVNLLCLAVCR
PEPTVTWRQLRDGFTSEGEILEISDILRGQAGEYECVTHNGVNSAPDSRRVLVTVNYPPTITDVTSARTALG
RAAYCAAKPWRFPPRISSGIPMTDY*
An LP319a Mature Sequence (211aa):
A predicted mature LP319a sequence is as follows:
QRLEFNSPADNYTHVTRVAWLNRSNILYAONDRRTRDPRVRLLINTSEEFSILVTEVGLGDEGLYTCSFQTR
HQPYTTQVYLIVHVPARVVNISSPVMVNEGGNVMLLCLAVGRPEPTVTWRQLRDGFTSEGEILEISDILRGQ
AGEYECVTHNGVNSAPDSRRVLVTVNYPPTITDVTSARTALGRAAYCAAKPWRFPPRISSGIRNTDY*
An Alternate LP319a Mature Sequence (222aa):
An alternate predicted mature LP319a sequence is as follows:
GLAVISRGLLSQRLEFNSPADNYTHVTRVAWLNRSNILYAGNDRRTRDPRVRLLINTSEEFSILVTEVGLGD
EGLYTCSFQTRHQPYTTQVYLIVNVPARVVNISSPVMVNEGGNVNLLCLAVGRPEPTVTWRQLRDGFTSEGE
ILEISDILRGQAGEYECVTHNGVNSAPDSRRVLVTVNYPPTITDVTSARTALGRAAYCAAKPWRPPPRISSG
IRMTDY
LP319b Nucleic Acid Sequence (2601 bp) (ORF = 21-791):
LP319b (SEQ ID NO: 13) start (atg) and stop (tga) codons are indicated
in bold typeface and underlined. Note, the “T” at position 274 can
also be “C”, therefore, the corresponding amino acid residue can either
be Pro (ccc) or Ser (tcc).
ATGCCCCCCGCTGCGCCCGGGCCCCGGCTCCGGCTTCTCGCCGCCGCCGCCCTGGCCGGCTTGGCCGTCATC
AGCCGGGGGCTGCTCTCCCAGAGGCTGGAGTTCAACTCTCCTGCCGACAACTACACACTGTGTGAAGGTGAC
AACGCCACCCTCAGCTOCTTCATGGACCAGCATGTGACCCGCGTGGCCTGGCTGAACCGCTCCAACATCCTG
TACGCCGGCAACGACCGCAGGACCAGGGACCCGCGGGTGCGGCTGCTCATCAACACCTCCGAGGAGTTCTCC
ATCCTCGTCACCGAGGTGGGGCTCGGCGACGAGCGCCTCTACACCTGCTCCTTCCAGACCCGCCACCACCCG
TACACCACTCAGGTCTACCTCATTGTCCACGTCCCTGCCCGCGTTGTGAACATCTCGTCGCCTGTGATGGTG
AATGACGGAGGTAATGTGAACCTGCTTTGCCTGGCCGTGGGGCGGCCAGAGCCCACGGTCACCTGGAGACAG
CTCCGAGACGGCTTCACCTCGGAGGGAGAGATCCTGGAOATCTCTGACATCCTGCGGGGCCAGGCCGGGGAG
TATGAGTGCGTGACTCACAACGGGGTTAACTCGGCGCCCGACAGCCGCCGCGTGCTGGTCACAGTCAACTAT
CCTCCGACCATCACGGACGTGACCAGCGCCCGCACCGCGCTGGGCCGGGCCGCCTACTGCGCTGCGAAGCCA
TGGCGGTTTCCCCCGCGGATTTCCAGTGGTATAAGGATGACAGACTACTGA
LP319b Full-length Sequence (256aa):
LP319b (SEQ ID NO: 14) The underlined portion is a predicted signal
sequence (Met-1 to Ser-30) . A predicted SP cleavage site is between
Ser-30 and Gln-31 indicated as follows: 1
MPPAAPGARLRLLAAAALAGLAVISRGLLS\hat{ }QR 32. An alternate predicted SP
cleavage site is between Ala-22 and Val-23 indicated as follows: 1
MPPAAPGARLRLLAAAALAGLA\hat{ }VI 24. Both mature LP319b versions are
encompassed herein.
MPPAAPGARLRLLAAAALAGLAVISRGLLSQRLEFNSPADNYTVCEGDNATLSCFMDEHVTRVAWLNRSNIL
YAGNDRRTRDPRVRLLINTSEEFSILVTEVGLGDEGLYTCSFQTRHQPYTTQVYLIVHVPARVVNISSPVMV
NEGGNVNLLCLAVGRPEPTVTWRQLRDGFTSEGEILEISDILRGQAGEYECVTHNGVNSAPDSRRVLVTVNY
PPTITDVTSARTALGRAAYCAAKPWRFPPRISSGIRNTDY*
An LP319b Mature Sequence (226aa):
A predicted mature LP3l9b sequence is as follows:
QRLEFNSPADNYTVCEGDNATLSCFMDEHVTRVAWLNRSNILYAGNDRRTRDPRVRLLINTSEEFSILVTEV
GLGDEGLYTCSFQTRHQPYTTQVYLIVHVPARVVNISSPVMVNEGGNVNLLCLAVGRPEPTVTWRQLRDGFT
SEGEILEISDILRGQAGEYECVTHNGVNSAPDSRRVLVTVNYPPTITDVTSARTALGRAAYCAAKPWRFPPR
ISSGIRMTDY
An Alternate LP319b Mature Sequence (234aa):
An alternate predicted mature LP3l9b sequence is as follows:
VISRGLLSQRLEFNSPADNYTVCEGDNATLSCFMDEHVTRVAWLNRSNILYAGNDRRTRDPRVRLLINTSEE
FSILVTEVGLGDEGLYTCSFQTRHQPYTTQVYLIVHVPARVVNISSPVMVNEGGNVNLLCLAVGRPEPTVTW
RQLRDGFTSEGEILEISDILRGQAGEYECVTHNGVNSAPDSRRVLVTVNYPPTITDVTSARTALGRAAYCAA
KPWRFPPRISSGIRNTDY
An LP319b Variant Sequence (286aa):
A variant LP319b sequence with a fusion of LP289 carboxy amino acid
sequence resulting in a complete third Ig-like C2 domain and a GPI
anchor sequence. The added sequence is indicated by underlining.
QRLEFNSPADNYTVCEGDNATLSCFMDEHVTRVAWLNRSNILYAGNDRRTRDPRVRLLINTSEEFSILVTEV
GLGDEGLYTCSFQTRHQPYTTQVYLIVHVPARVVNISSPVMVNEGGNVNLLCLAVGRPEPTVTWRQLRDGPT
SEGEILEISDILRGQAGEYECVTHNGVNSAPDSRRVLVTVNYPPTITDVTSARTALGRAAYCAAKPWRFPPR
ISSGIRNTDYLSSGTAEGHYGNYTCRAANRLGASSASMRLLRPGSLENSAPRPPGLLALLSALGWLWWRM
An LP319b Variant Sequence (294aa):
Another variant LP319b sequence with a fusion of LP289 carboxy sequence
resulting in a complete third Ig-like C2 domain and a GPI anchor
sequence. The added sequence is indicated by underlining.
VISRGLLSQRLEFNSPADNYTVCEGDNATLSCFNDEHVTRVAWLNRSNILYAGNDRRTRDPRVRLLINTSEE
FSILVTEVGLGDEGLYTCSFQTRHQPYTTQVYLIVHVPARVVNISSPVMVNEGGNVNLLCLAVGRPEPTVTW
RQLRDGFTSEGEILEISDILRGQAGEYECVTHNGVNSAPDSRRVLVTVNYPPTITDVTSARTALGRAAYCAA
KPWRFPPRISSGIRMTDYLSSGTAEGHYGNYTCRAANRLGASSASMRLLRPGSLENSAPRPPGLLALLSALG
WLWWRM

Analysis of the primary amino acid structure of LP319a & LP319b demonstrates that they possess typical IgLON characteristics, including homology to known IgLON members and IgLON-like motifs. Based on the teachings supplied herein (e.g., the LP319a & LP319b sequence and their relationship with the domains, motifs, and signatures of other known IgLONs) and those known in the art (e.g., assay methods to determine binding activities of suspected IgLONs such as neurite outgrowth, homo- or heterophilic binding, axonal pathfinding, opiod-like binding, e.g., the assays described in, e.g., Hachisuka, et al. 1996 Neurochem. Int. 28:373-379 such as which is incorporated by reference herein for such assay teachings), one skilled in the art would be able to test LP319a or LP319b for IgLON-like activities without undue experimentation (e.g., using common assay techniques and commercially available reagents). Some non-limiting examples of functions an LP319a/b, LP319a/b variant, or an LP319a/b binding agent (e.g., such as an LP319a/b antibody (or fragment thereof)) is likely to participate in are, for example, those such as: neuorogenesis; the formation, development, and/or modification of regional centers in the brain such as, for example, nuclei of, for example, the forebrain: such as, for example, the olfactory bulb and cortex; the neocortex; the striatum, the nucleus accumbens; the basal forebrain; the limbic circuit; the thalamus (including, for example, reticular thalamic nucleus, dorso-caudal nucleus, dorso-intermedial nucleus, dorso-orales nucleus, ventral-caudal nucleus, ventral-intermediate nucleus, ventral-oralis posterior nucleus, ventral-oralis anterior nucleus, sub-thalamic nucleus, and substantia nigra); the hypothalamus: (including, for example, anterior lobe of the pituitary (adenohypophysis), posterior lobe of the pituitary (neurohypophysis), optic chiasm, preoptic nucleus, anterior nucleus, dorsomedial nucleus, ventromedial nucleus, posterior nucleus, mammillary body, hypothalamic supraoptic nuclei (SON), and paraventricular nuclei (PVN)); the Midbrain, such as, for example, the tectum (superior and inferior colliculi) or the tegmentum; the Hindbrain; the Pons and/or the Cerebellum; and the Medulla; the formation, development, and/or modification of neural architecture such as, e.g., during the formation of the dendritic tree of e.g., Purkinje cells in the cerebellum; modulation of axonal targeting, neurite pathfinding; the formation, development, and/or modification of neural circuitry; the formation, development, maintenance, and/or modification of distinct neuronal systems; the formation, maintenance, and/or modification of neural connections; cell adhesion; neurite outgrowth; regulation of the development of neuronal projections via cell specific attractive and/or repulsive mechanisms; the formation, maintenance, and/or modification of neural circuitry; the formation, maintenance, and/or modulation of axo-dendritic, dendro-dendritic, axo-axonal, dendro-axonal, axo-somata, dendro-somata, and somata-somata, interactions during the formation, maintenance, and/or modification of neuronal projections; cell-cell interactions in the nervous system; the formation, promotion, maintenance and/or remodeling of fiber fasciculation on neural cells (such as, e.g., axonal surfaces of developing or remodeling neural surfaces (e.g., by either homophilic or heterophilic interactions) for example, such as, targeting and/or maintenance of brain nucleii projections (e.g., such as targeting of thalamocortical axons to their appropriate cortical area during development); modulation of adhesion to a substrate; modulation and/or regulation of action of neural binding of an opiod and/or opiod-like ligand; specification of neuronal connectivity; modulation of an opiod and/or opiod-like nociception state, condition, or syndrome; modulation of an opiod and/or opiod-like antinociception state, condition, or syndrome; facilitation, and/or modulation of opiod and/or opiod-like signal transduction; facilitation, and/or modulation of opiod and/or opiod-like signal transduction, wherein the opiod or opiod-like composition is a mu or mu-like opiod; facilitation, and/or modulation of opiod and/or opiod-like signal transduction, wherein the opiod or opiod-like composition is a kappa or kappa-like opiod; facilitation, and/or modulation of opiod and/or opiod-like signal transduction, wherein the opiod or opiod-like composition is a delta or delta-like opiod; modulate the formation of an opiod or opiod-like receptor coupling to an intracellular scaffold and/or adaptor protein/s; facilitate and/or modulate an opiod receptor/G-protein coupling; modulate, treat, and/or diagnose, and/or prognose an opiod or opiod-like tolerance state, condition, or syndrome; modulate, treat, and/or diagnose, and/or prognose an opiod or opiod-like dependence state, condition, or syndrome; modulate, treat, and/or diagnose, and/or prognose an opiod or opiod-like dependence state, condition, or syndrome, wherein the opiod or opiod-like dependence state, condition, and/or syndrome is related to a delta or delta-type opiod or opiod-like composition; modulate, treat, and/or diagnose, and/or prognose an opiod or opiod-like dependence state, condition, or syndrome, wherein the opiod or opiod-like dependence state, condition, and/or syndrome is related to a mu or mu-type opiod or opiod-like composition; modulate, treat, and/or diagnose, and/or prognose an opiod or opiod-like dependence state, condition, or syndrome, wherein the opiod or opiod-like dependence state, condition, and/or syndrome is related to a kappa or kappa-type opiod or opiod-like composition; treatment and/or modulation of a disease, condition, syndrome, or a state of respiratory control; treatment and/or modulation of a disease, condition, syndrome, or a state of appetite control; modulate the formation of an opiod receptor-G-protein binding complex; modulate an opiod receptor conformational transformation; the formation, promotion, maintenance and/or remodeling of synaptic connectivity; the promotion, stimulation, maintenance, and/or inhibition of neurite outgrowth; growth cone guidance; inhibition and/or promotion of a regeneration of CNS or PNS neurons after damage; stimulation of embryonic neurons; inhibition of mature neurons; a neuro-attractant; and a neuro-repellant.

Particularly interesting portions or fragments of the full length LP319a polypeptide (SEQ ID NO: 12) include, e.g., are two immunoglobulin-like domains: the first from about Ser-30 to about Phe-99: (SQRLEFNSPADNYTHVTRVAWLNRSNILYAGNDRRTRDPRVRLLINTSEEFSILVTEVGLGDEGLYTCSF) and the second from about Gly-132 to about Thr-182: (GGNVNLLCLAVGRPEPTVTWRQLRDGFTSEGEILEISDILRGQAGEYECVT). The LP319b immunoglobulin-like domains are from about Gly-47 to about Phe-114: (GDNATLSCFMDEHVTRVAWLNRSNILYAGNDRRTRDPRVRLLINTSEEFSILVTEVGLGDEGLYTCSF); and from about Gly-147 to about Val-197 (GGNVNLLCLAVGRPEPTVTWRQLRDGFTSEGEILEISDILRGQAGEYECV). A further, interesting portion of LP319a is the discovered heavy metal-associated-like domain at the C-terminus of LP319a from about Val-197 to about Lys-224 (VTVNYPPTITDVTSARTALCRAAYCAAK). The heavy metal associated domain of LP319b is from about Val-212 to about Lys-239 (VTVNYPPTITDVTSARTALGRAAYCAAK). Additionally interesting segments of LP319a are discovered fragments from about Arg-11 to about Leu-29; from about Gln-31 to about Trp-50; from about Asn-52 to about Ile-74; from about Thr-76 to about Asp-91; from about Glu-92 to about Gln-109; from about Val-110 to about Val-129; from about Gly-132 to about Arg-144; from about Pro-145 to about Glu-163; from about Leu-171 to about Val-181; from about Thr-182 to about Val-195; from about Ala-222 to about Arg-231; from about Arg-11 to about Leu-28; from about Ser-30 to about Ala-49; from about Arg-53 to about Leu-73; from about Val-84 to about Tyr-95; from about Thr-96 to about Gln-109; from about Val-110 to about Val-120; from about Val-121- Asn-136; from about Val-142 to about Ile-164; from about Ile-170 to about Leu-196; from about Pro-203 to about Pro-225; from about Leu-18 to about Phe-35; from about Thr-46 to about Ala-59; from about Arg-69 to about Glu-78; from about Glu-79 to about Gly-88; from about Thr-107 to about Ala-118; from about Arg-119 to about Met-128; from about Gly-162 to about Leu-171; from about Arg-172 to about Val-181; from about Thr-182 to about Arg-193; from about Arg-194 to about Thr-206; from about Val-208 to about Ala-223; from about Pro-225 to about Ser-234 whose discover is were based on an analysis of, hydropathicity, hydrophilicity and hydrophobicity plots. Additional interesting sections of LP319a are the discovered portions of LP319a from about Arg-8 to about Val-23; from about Asn-41 to about Trp-50, from about Leu-51 to about Asp-62 (LNRSNILYAGND); from about Val-70 to about Val-84; from about Val-84 to about Phe-99; from about Pro-117 to about Glu-131; from about Gly-132 to about Arg-144; from about Trp-151 co about Ile-164; from about Glu-166 to about Glu-177; from about Thr-198 to about Ala-211; from about Arg-212 to about Pro-225; from about Trp-226 to about Ile-236. These fragments were discovered based on analysis of antigenicity plots. Further, particularly interesting LP319a secondary structures (e.g., such as a helix, a strand, or a coil) that have been discovered are the following LP319a helix structures: from about Leu-13 to about Ala-17; and from about Arg-212 to about Leu-215. Particularly interesting discovered coil structures are from about Met-1 to about Pro-6; from about Asn-36 to about Tyr-42; from about Asn-52 to about Ser-54; from about Ala-59 to about Asp-67; from about Asn-75 to about Glu-79; from about Gly-88 to about Gly-93; from about Arg-102 to about Thr-107, from about Pro-117 to about Ala-118; from about Ile-123 to about Ser-125; from about Glu-131 to about Asn-134; from about Val-142 to about Thr-148; from about Leu-154 to about Gly-162; from about Arg-172 to about Glu-177; from about Asn-184 to about Ser-192; from about Asn-200 to about Ile-205; from about Ala-223 to about Gly-235; from about and Thr-239 to about Tyr-241. Particularly interesting discovered strand structures are from about Ala-49 to about Trp-50; from about Ile-56 to about Tyr-58; from about Ser-81 to about Glu-86; from about Tyr-95 to about Cys-97; from about Gln-109 to about Val-114; from about Val-120 to about Asn-122; from about Asn-136 to about Leu-140; from about Val-149 to about Arg-152; from about Ile-164 to about Leu-165; from about Tyr-178 to about Thr-182; and from about Arg-194 to about Val-199. Further encompassed by the invention are contiguous amino acid residue combinations of any of the predicted secondary structures described above. For example, one coil-strand-coil-strand-coil motif of LP319a combines an Arg-102 to Thr-107 coil, with an Gln-109 to Val-114 strand, with an Pro-117 to Ala-118 coil, with an Val-120 to Asn-122 strand, and an Ile-123 to Ser-125 coil to form an interesting fragment of contiguous amino acid residues from about Arg-102 to about Ser-125. Other such combinations of contiguous amino acids are contemplated as can be easily determined.

Interesting segments of LP319b are discovered fragments from about Leu-18 to about Phe-35; from about Asn-36 to about Thr-51; from about Arg-62 to about Ala-74; from about Arg-84 to about Glu-93; from about Glu-94 to about Gly-103; from about Glu-122 to about Ala-133; from about Arg-134 to about Met-143; from about Gly-177 to about Leu-186; from about Gly-187 to about Val-196; from about Thr-197 to about Gly-208; from about Arg-209 to about Thr-221; from about Asp-222 to about Ala-238; from about Pro 240 to about Ser-249; from about Arg-1 1 to about Leu-29; from about Ser-30 to about Asp-40; from about Asn-41 to about Ala-50; from about Glu-58 to about Asp-77; from about Arg-78 to about Ile-89; from about Arg-91 to about Asp-106; from about Glu-107 to about Gln-124; from about Val-125 to about Arg-134; from about Val-135 to about Glu-146; from about Glu-147 to about Arg-159; from about Pro-160 to about Glu-178; from about Leu-186 to about Val-196; from about Thr-197 to about Val-210; from about Thr-237 to about Val-246; from about Arg-11 to about Val-29; from about Ser-30 to about Val-44; from about Cys-45 to about Ala-64; from about Cys-68 to about Ala-78; from about Arg-79 to about Ile-89; from about Asn-90 to about Gly-108; from about Leu-109 to about Gln-124; from about Val-125 to about Val-135; from about Val-136 to about Leu-152; from about Val-157 to about Trp-166; from about Arg-167 to about Ile-182; from about Ser-186 to about Asn-199; from about Gly-200 to about Val-212; from about Val-214 to about Ser-225; and from about Arg-226 to about Arg-242, whose discovery is based on an analysis of hydropathicity, hydrophilicity, and hydrophobicity plots. Additional interesting sections of LP319b are the discovered portions of LP319b from about Arg-8 to about Val-23; from about Phe-35 to about Cys-45; from about Glu-46 to about Arg-62; from about Trp-65 to about Asp-77; from about Val-85 to about Phe-95; from about Ser-96 to about Thr-111; from about Ser-137 to about Thr-150 from about Asn-151 to about Val-164 from about Thr-165 to about Glu-181 from about Ile-182 to about Gly-191 from about Glu-192 to about Val-201 from about Val-212 to about Ala-226 from about Arg-227 to about Pro-240; and from about Trp-241 to about Gly-250. These fragments were discovered based on an analysis of antigenicity plots. Further, particularly interesting LP319b secondary structures (e.g., such as a helix, a strand, or a coil) that have been discovered are the following LP319b helix structures: from about Leu-13 to about Ala-17; and from about Arg-227 to about Leu-230. Particularly interesting discovered coil structures are from about Met-1 to about Pro-6; from about Gly-27 to about Gly-27; from about Asn-36 to about Asp-39; from about Glu-46 to about Cys-49; from about Asn-67 to about Ser-69; from about Ala-74 to about Asp-82; from about Asn-90; from about Glu-94 to about Gly-103 to about Gly-108; from about Thr-116 to about Thr-122; from about Pro-132 to about Ala-133; from about Ile-138 to about Ser-140; from about Glu-146 to about Asn-149; from about Val-157 to about Thr-163; from about Leu-169 to about Gly-177; from about Arg-187 to about Glu-192; from about Asn-199 to about Ser-207; from about Asn-215 to about Ile-220; from about Ala-238 to about Gly-25; and from about Thr-254 to about Tyr-256. Particularly interesting discovered strand structures are from about Try-42 to about Cys-45; from about Ala-64 to about Trp-65; from about Ile-71 to about Tyr-73; from about Ser-96 to about Glu-101; from about Tyr-110 to about Cys-112; from about Gln-124 to about Val-129; from about Val-164 to about Arg-167; from about Ile-179 to about Leu-180; from about Tyr-193 to about Thr-197; and from about Arg-209 to about Val-214.

Features of LP No: 7 (LP321)

LP321 is a novel primate (e.g., human) polypeptide (SEQ ID NO: 16) that is a newly discovered member of the defensin family of antimicrobial peptides, which are generally recognized a having antibiotic, antifungal, and antiviral activities. Specifically, LP321 exhibits sequence homology to enteric alpha defensins known as cryptdins. Defensins are a family of structurally related cysteine-rich peptides active against many Gram-negative and Gram-positive bacteria, fungi, and viruses (see, e.g., Lehrer, et al. Cell 64:229-230; Kagan, et al. 1994 Toxicology 87:131-149; Lehrer, et al. 1993 Annu. Rev. Immunol. 11:105-128; and White, et al. 1995 Curr. Opin. Struct. Biol. 5:521-527). Some defensins are also called corticostatins (CS) because they inhibit corticotropin-stimulated corticosteroid production. Defensins can kill cells by forming voltage-regulated multimeric channels in the membrane of the susceptible cell. Defensins play a significant role in innate immunity to infection and neoplasia. Antimicrobial peptides are a prevalent mechanism of host defense found throughout nature (Kaiser & Diamond 2000 J Leukoc Biol 6:779-84). In mammals, defensins are among the most abundant of these broad-spectrum antibiotics, and are expressed in epithelial and hematopoietic cells among others. The defensin peptides are especially abundant in neutrophils. In epithelial cells, defensins are found both as constitutively expressed and inducible genes. Induction has been observed in vitro by stimulation with bacterial lipopolysaccharide (LPs) as well as inflammatory mediators. In vivo, up-regulation of several defensin genes occurs in both infectious and inflammatory states. Gene regulation occurs via signal transduction pathways common to other innate immune responses, using transcription factors such as nuclear factor (NF)-kappa beta and NF interleukin-6. All together, these data suggest a broad-based innate host defense whereby potent antimicrobial peptides prevent colonization by pathogenic microorganisms. In addition, the recognition of bacteria coupled with a nascent inflammatory response can bolster this defense by a coordinated up-regulation of the peptides. Some peptides known to belong to the defensin family include, e.g.: Rabbit defensins and corticostatins: CS-I (NP-3A), CS-II (NP-3B), CS-III, (MCP-1), CS-IV (MCP-2), NP-4, and NP-5; Guinea-pig neutrophil defensin (GPNP); Human neutrophil defensins 1 to 4 and intestinal defensins 5 and 6; Mouse small bowel cryptdins 1 to 5 and; Rat NP-1 to NP-4. All these peptides range in length from approximately 29 to about 35 amino acids and typically, at the primary amino acid sequence level, they possess invariant cysteine residues that are involved in intrachain disulfide bonding. Before the characterization of mouse intestinal defensin cDNA, expression of defensins was thought to be limited to professional phagocytes, such as, for example, neutrophils and macrophages. The presence of high levels of cryptdin mRNA in Paneth cells led to the hypothesis that defensins synthesized in intestinal epithelium contribute to antimicrobial barrier function in the small bowel (Ouellette et al., 1989a J. Cell Biol. 108:1687-1695). Isolation and characterization of six mouse cryptdin peptides, two rat cryptdin peptides and 2 human cryptdin peptides, and the demonstration of antimicrobial activity of various cryptdin peptides indicate that the cryptdin defensins have an antimicrobial role in the small intestine. The immunohistochemical localization of cryptdins to Paneth cells is consistent with previous in situ hybridization analysis and suggests that defensins produced by these cells may restrict colonization and invasion of the small bowel by bacteria.

LP321 nucleic acid sequence (SEQ ID NO: 15) is expressed in the following LIFESEQ GOLD™ database tissue and cDNA libraries: Respiratory System 1/95. This LP321 expression pattern is commensurate with reports indicating that cryptdins are also found in lung marcrophages (see, e.g., Shirafuji, et al. 1999 Clin. & Diagnos. Lab. Immun 6:336-340).

TABLE 6

Primate, e.g., human, LP321 polynucleotide sequence (SEQ ID NO: 15) and corresponding
polypeptide (SEQ ID NO: 16). The ORF for LP321 is 75-338 bp (with the start (ATG) and stop codons
(TAA) identified in bold typeface and underlined. In the event that the numbering is misidentified,
one skilled in the art could still determine the open reading frame without undue experimentation).

LP321 DNA sequence (480 bp) (ORF = 75-338):
ATGAAGACACTAGTCCTCCTCTCTGCTCTTGTCCTGCTCGCCTTGCAGGTCCAGGCTGATCCCATTCAAGA
GGCAGAGGAAGAGACTAAAACTGAGGAGCAGCCAGCAGATGAGGACCAGGATGTGTCTGTCTCCTTTGAAG
GCCCAGAAGCCTCTGCTGTTCAAGATTTACGCGTAAGAAGGACCTTGCAGTGCAGTTGCAGAAGAGTCTGC
AGAAATACGTGTAGCTGCATTCGGCTATCAAGGTCCACATATGCATCATAA
LP321 Full-Length Sequence (87aa):
>LP321 (SEQ ID NO: 16). The underlined portion is a predicted signal sequence (Met-1 to Ala-19). A
predicted SP cleavage site is between Ala-20 and Asp.21 indicated as follows: 1
MKTLVLLSALVLLALQVQA\hat{ }DP 21. An alternate predicted SP cleavage site is between Gln-16 and Val 17
indicated as follows: 1 MKTLVLLSALVLLALQ\hat{ }VQ 18. Both mature LP321 versions arc encompassed
herein. LP321 polypeptides encompassed herein include full-length forms encoded by an ORF disclosed
herein, as well as any mature forms therefrom. Such a mature LP321 could be formed, for example, by
aminopeptidase modification, or by the removal of a signal peptide. Further as used herein, a “mature”
LP encompass, e.g., post-translational modifications other than proteolytic cleavages (such as, e.g., by way of a
non-limiting example, glycosylations, myristylations, phosphorylations, prenylations, acylations, and
sulfations). Any such variant is also encompassed by an LP of the present invcntion. Further, an LP of
the invention encompass all fragments, analogs, homologs, and derivatives of an LP described herein,
thus the invention encompasses both LP precursors and any modified versions (such as, e.g., by post-
translational modification) of an LP encoded by an LP nucleic acid sequence described herein.
Moreover, it has also been shown that cryptdin precursors can be processed and activated (e.g., in
Paneth cells) by the metalloproteinase matrilysin (MMP-7). Moreover, paneth cells of MMP-7 null mice
(MAT-/-) do not process procryptdin precursors, resulting in a lack of mature cryptdins (Wilson, et
al. 1999 Science 286,113-117). Accordingly, such post-translational processing is also encompassed
by use of the term a “mature” LP321 or a mature LP231 variant sequence of the invention.
MKTLVLLSALVLLALQVQADPIQEAEEETKTEEQPADEDQDVSVSFEGPEASAVQDLRVRRTLQCSCRRVCR
NTCSCIRLSRSTYAS*
An LP321 Mature Sequence (68aa):
A predicted mature LP321 sequence is as follows:
DPIQEAEEETKTEEQPADEDQDVSVSFEGPEASAVQDLRVRRTLQCSCRRVCRNTCSCIRLSRSTYAS
An LP321 Variant Sequence (71aa):
An alternate LP321 mature sequence is as follows:
VQADPIQEAEEETKTEEQPADEDQDVSVSFEGPEASAVQDLRVRRTLQCSCRRVCRTCSCIRLSRSTYAS
An LP321 Variant Sequence (87aa):
An alternate LP321. The underlined portion is a predicted propeptide
segment from about Met-1 to about Ser-52
(MKTLVLLSALVLLALQVQADPIQEAEEETKTEEQPADEDQDVSVSFEGPEAS). A predicted
cleavage site is between Ser-52 and Ala-53 indicated as follows: 1
MKTLVLLSALVLLALQVQADPIQEAEEETKTEEQPADEDQDVSVSFEGPEAS\hat{ }AVQD 56.
MKTLVLLSALVLLALQVQADPIQEAEEETKTEEQPADEDQDVSVSFEGPEASAVQDLRVRRTLQCSCRRVCR
NTCSCIRLSRSTYAS
An LP321 Variant Sequence (35aa):
An alternate LP321 mature sequence with a propeptide segment removed is:
AVQDLRVRRTLQCSCRRVCRNTCSCIRLSRSTYAS
An LP321 Variant Sequence (32aa):
Another alternate LP321 mature sequence with the N terminal portion
before the first cysteine shortened is:
DLRVRRTLQCSCRRVCRNTCSCIRLSRSTYAS
An LP321 Variant Sequence (28aa):
A further alternate L2321 mature sequence comprising an alternate N
terminal portion before the first cysteine is:
LRDLVCSCRRVCRNTCSCIRLSRSTYAS
An LP321 Variant Sequence (26aa):
Still another alternate LP321 mature sequence comprising an alternate N
terminal portion before the first cysteine is:
GLLCSCRRVCRNTCSCIRLSRSTYAS

An interesting segment of LP321 is the segment from about Met-1 to about Ser-52, which has been discovered to be a defensin propeptide-like domain. Other interesting segments of LP321 are the discovered portions of LP321 from about Ser-8 to about Ala-25; from about Asp-20 to about Phe-46; from about Ala-51 to about Arg-60; from about Arg-61 to about Cys-71; from about Ser-8 to about Ile-22; from about Gln-23 to about Glu-38, from about Asp-41 to about Ala-53; from about Arg-61 to about Arg-72, from about Cys-71 to about Leu-80; from about Val-17 to about Glu-27; from about Ile-22 to about Pro-35; from about Pro-35 to about Val-44; from about Glu-47 to about Asp-56; from about Val-59 to about Val-70; and from about Gln-64 to about Cys-77; whose discoveries were based on an analysis of hydrophobicity, hydropathicity, and hydrophilicity plots. Additional interesting sections of LP321 are the discovered portions of LP321 from about Val-5 to about Gln-18; from about Ala-19 to about Glu-27; from about Tyr-29 to about Pro-35; from about Ser-43 to about Ala-51; from about Gln-64 to about Ser-81; and from about Ser-52 to about Arg-61. These fragments were discovered based on analysis of antigenicity plots. Further, particularly interesting LP321 structures (e.g., such as a helix, a strand, or a coil) that have been discovered are the following LP321 helix structures: from about Leu-6 to about Leu-14; from about Ile-22 to about Glu-28; and from about Ala-53 to about Leu-57. Particularly interesting discovered coil structures are from about Glu-6 to about Gln-14; from about Glu-47 to about Glu-50; from about Cys-71 to about Cys-75; and from about Leu-53 to about Ser-57. Particularly interesting discovered strand structures are from about Val-42 to about Ser-45; and from about Cys-77 to about Arg-79. Further encompassed by the invention are contiguous amino acid residue combinations of any of the predicted secondary structures described above. For example, one helix-coil-strand-coil motif of LP321 combines the helix from about Ile-22 to about Glu-28, with the coil about Glu-6 to about Gln-14, the strand from about Val-42 to about Ser-45, and the coil from about Glu-47 to about Glu-50 to form an interesting fragment of contiguous amino acid residues from about Ile-22 to about Glu-50. Other combinations of contiguous amino acids are contemplated as can be easily determined.

LP321 Functions: Given the teachings supplied herein, for example, of: an LP321 primary amino acid, LP321 higher order structures, the relationship of an LP321 amino acid sequence to higher order structural features, the comparability of an LP321 sequence and/or an LP321 higher order structure with a known defensin (such as, e.g., members of the cryptdin protein family, such as, e.g., mouse cryptdins 1-6), and the relationship of higher order structural features of a cryptdin with their known functions, it is likely that an LP321, an LP321 variant, an LP321 hetero-or an LP321 homomultimer, and/or an LP321 binding agent (e.g., such as an LP321 antibody (or fragment thereof)) plays a similar role/s in a variety of physiological processes. For instance, some non-limiting examples of functions an LP321, an LP321 variant, an LP321 hetero- or an LP321 homomultimer, or an LP321 binding agent is likely to have and/or participate in are, for example, those such as: mucosal immunity (such as, e.g., mucosal surfaces in, e.g., epithelia in an airway, skin, oropharynx, a gingival crevice, and an urogenital surface); host defense; anti-microbial activity against (e.g., bacteria such as, e.g., Gram-positive bacteria, Gram-negative bacteria, S. typhimurim, S. typhimurium, S. typhimurium phoP- mutant, Staphylococcus aureus, Streptococcus pyogenes, Esherichia coli, and Listeria moncyotgenes), protists (such as, e.g., G. lamblia), fungi, and viruses (such as, e.g., enveloped viruses); inhibiting the colonization of an epithelium by a pathogenic microorganism; protection of developing spermatids; regulation of cell volume; chemotaxis; mitogenicity; inhibition of natural killer cell activity; modulation of ion flow (such as, e.g., calcium, sodium, potassium, or chloride ions); modulation of an ionic flow through a membrane and/or a pore, wherein said membrane is on a epithelial cell, modulation of an ionic flow on a membrane and/or a pore of a microbe; mediation of innate immunity; modulation of chloride ion flow; creation of an ionic pore in a membrane; create an ionic pore in a membrane, wherein said membrane is of an epithelium, wherein said epithelium is a broncho epithelium; wherein said epithelium is in a lung; creating an ionic pore in a membrane, wherein said membrane is of a microbe; mount and maintain a defense against a luminal floral pathogen such as, e.g., an enteric bacteria; prevention of sepsis; protection of stem cell integrity; and protection of stem cell integrity in a lumen, wherein said lumen is an intestinal lumen, wherein said lumen is a oropharyngeal lumen, or wherein said lumen is a urogenital lumen. The invention further provides a method for detecting an inflammatory pathology in a subject by determining the amount of LP321 in a biological sample from the subject and comparing that amount to an amount present in a normal subject. Such a method can be used to determine the presence of an inflammatory pathology such as an inflammatory bowel disease, pancreatitis, a malignant condition, an infection, or an ileititic condition. The invention also provides a method for treating an inflammatory sydrome, condition, state or disease in a subject by administering an LP321, an LP321 variant, an LP321 pharmaceutical composition, an LP321 binding agent, or an LP321 hetero- or an LP321 homomultimer to a subject having such a condition, state or disease. The invention also provides a method for treating a biological surface with an LP321, an LP321 variant, an LP321 hetero-or an LP321 homomultimer and/or an LP321 binding agent, wherein said surface is a mucosal surface; wherein said surface an epithelia in an airway; wherein said surface is skin, wherein said surface is a surface in the oropharynx; wherein said surface is a gingival crevice; and wherein said surface is a urogenital surface. Such treatment is particularly advantageous in subjects that are immunocompromised due, such as, for example, to: malnutrition, radiation, burns, immunosuppressive infections or conditions, autoimmune disease, neonatality, bone marrow transplantation, and/or chemotherapy. Non-limiting examples of how an LP321 of the invention can be administered are: orally, by nasogastric intubation, by transabdominal catheter, intravenously, or by aerosol inhalation. When administered orally, an LP composition of the invention is preferably in a delayed release formulation designed to permit release in, e.g., the intestinum carcum, the intestinum crassum, the intestinum ileum, the intestinum jejunum, the intestinum rectum, the intestinum tenue, or the intestinum mesenteriale. An LP321 of the invention can be administered as a composition with a physiologically acceptable medium, and can be administered in combination with other agents such as, for example, a cryptdin, a defensin, a thionin, or it can be administered simultaneously, or sequentially with any of the former. In another embodiment, an LP321, or an LP321 variant is administered in concert with a granulocyte colony-stimulating factor (G-CSF), or a G-CSF composition, (such as in the range of about 1.0 to about 10.0 ug/kg weight/day for about 1 to about 10 days in a subject, such as, e.g., an immunocompromised subject, such as, e.g., a subject who has neutropenia or a neutropenic-like condition). Cryptdins or cryptdin-like compositions exhibit antimicrobial activity against enteric microorganisms, which can become blood-borne pathogens if an epithelial layer is breached, for example, such as the epithelial layer in the intestines or an epithelium of the airway, such as, e.g., in the lungs (such as, e.g., the alveoli or an aveolar sac). Cryptdins or a cryptdin-like molecule can be secreted from a cell in which it is produced (Satoh, 1988Cell Tiss Res. 251:87-93; Satoh et al. 1988Acta Histochem 83:185-188).

It should be appreciated that various modifications can be made to an LP321 amino acid sequence without diminishing the antimicrobial activity of an LP peptide of the invention. It is intended that LP peptides exhibiting such modifications (including, e.g., amino acid additions, deletions and/or substitutions) are all within the scope of the term “LP321” and, therefore, within the scope of the invention. For example, LP321 variants, which are devoid of one or more amino acids located N-terminal to the first cysteine residue in the primary structure, are all within the scope of the present invention. Such an LP321 analog or variant can be synthesized using art-known methods or those described or referenced herein. Further, included herein are methods of prognosing, diagnosing, and/or treating a microbial infection, condition, disease, or state such as, e.g., otitis media using an LP321, an LP321 variant, or an LP321 binding agent of the invention. Further encompassed here are methods of prognosing, diagnosing, and/or treating a microbial infection, condition, disease, or state using an LP321 or an LP321 variant of the invention in combination with a cryptdin, such as, for example, cyrptdin 1, cyrptdin 2, cyrptdin 3, cyrptdin 4, cyrptdin 5, cyrptdin 6, or any combination thereof; or with a defensin, such as, for example, HD-1, HD-2, HD-3, HD-4, HD-5, HD-6, HNP-1, HNP-2, HNP-3 (or any combination thereof); or a thionin; or any combination thereof.

Typically, an antimicrobial activity of a cryptdin, or a cryptdin-like peptide can be determined against various pathogens. As disclosed herein, various microorganisms can be grown to an appropriate concentration, mixed with an appropriate medium (such as, for example, an agarose-trypticase soy mediums), and contacted with an LP321 or a cryptdin, or a defensin to assess an antimicrobial activity. An antimicrobial activity is apparent, for example, from clear zones that typically surround a cryptdin or cryptdin-like composition (e.g., such as an LP321) that is placed in an agar for a diffusion assay. Characteristically, an area of a clear zone is dependent on the concentration of the cryptdin or cryptdin-like molecule. Anti-LP321 binding agents (e.g., such as an LP321 antibody) can be used to determine the presence of an LP321 or an LP321 variant in a biological sample such as, e.g., a histological sample, or a lavage product, blood, an exudate or another biological sample. For example, a section of a small intestine is fixed by art-known means and incubated with anti-LP321 antibodies such as, e.g., an IgG fraction of LP antiserum. If desired, the anti-LP321 antibody is detectably labeled or an appropriate detectable second antibody is used to identify the presence of the primary antibody attached to an LP321 or an LP321 variant. Alternative methods of determining the presence of an LP321, or an LP321 variant in a biological sample obtained, for example, by intestinal lavage or by disrupting cells or tissues can be useful to determine the presence of an inflammatory process such as, for example, colitis, Crohns disease, inflammatory bowel syndrome, pancreatitis, a malignancy, an infection, or an ileititic condition, etc. In an inflammatory state, or condition a concentration of an LP321, or an LP321 variant is significantly altered from a concentration found in a normal condition or state. For example, a deviation from a normal level of an LP321 or an LP321 variant by about one to about two standard deviations from an established baseline control is typically indicative of an inflammatory condition and/or state. Non-limiting examples of such an inflammatory state or condition include, for example, colitis, Crohns disease, inflammatory bowel syndrome, pancreatitis, a malignancy, an infection, or an ileititic condition. Because of their broad range of antimicrobial activity and their ability to function within an intestinal epithelium or lumen, an LP321, or an LP321 variant, is a therapeutic agent for an infection of, e.g., the intestine, the lung, or a biological surface, wherein said surface is a mucosal surface; wherein said surface an epithelium in an airway; wherein said surface is an epithelia surface of skin, wherein said surface is a surface in an oropharyn geal lumen; wherein said surface is a gingival crevice; or wherein said surface is a urogenital surface. In particular, an LP321, or an LP321 variant of the invention is useful where a subject is immunocompromised due, for example, to: malignancy, malnutrition, chemotherapy, radiation, immunosuppressive viruses, autoimmune disease, or neonatality. In addition, an LP321, or an LP321 variant of the invention is useful in a surgical prophylaxis, for example, by functioning to help sterilize the small bowel. Thus, an LP of the invention can be useful as a medicament for treating a subject having a pathology characterized, in part, by an inflammatory process and/or condition, e.g., such as an inflammatory process state, state or condition described herein. In another embodiment, an LP of the invention is useful in a pharmaceutical composition for a topical application. In another embodiment, an LP of the invention is useful in a propeptide form. In another embodiment, an LP of the invention is useful as being sequestered in a first form (such as, e.g., a propeptide form) and a second composition (having the capacity to cleave a prosegment of an LP321 or an LP321 variant) is also sequestered (such as, e.g., a second composition like a matrilysin, or matrilysin-like composition) from the first form propeptide composition, wherein the second composition and the first propeptide form are brought together at a location in a subject to form an active LP321 or an active LP321 variant, for example, such as in the lung, the alveoli, the intestinum carcum, the intestinum crassum, the intestinum ileum, the intestinum jejunum, the intestinum rectum, the intestinum tenue, or the intestinum messenteriale, at the site of an infection, the semineferous tublules, near a Sertoli cell, in a Paneth cell, in the distal small bowel, in an airway cell, in an intestinal crypt, etc. A composition of the invention encompasses an LP of the invention that form multimeric complexes. In a particular embodiment, an LP321 or an LP321 variant forms an multimeric LP321 complex that is capable of forming a pore in a membrane, such as a lipid membrane, such as a lipid bilayer. An LP of the invention (or variant thereof), either purified from natural sources or synthetic, can be administered to a subject (in need of treatment) by various means, including orally, preferably in a slow-release type formulation that will avoid release within the stomach. Alternatively, an LP321 can be administered through nasogastric intubation, trans-abdominal catheter, by injection intravenously, or by aerosol administration. Furthermore, other LP321 variants encompassed herein are cyclic LP321 variants that are produced by ligation of two truncated LP321s or LP321 variants by adapting the method of Tang, et al. 1999 Science 286:498-502 (which is incorporated herein by reference for its techniques regarding head-to-tail ligation of truncated defensins).

An LP of the invention can be administered alone or in combination with other agents (such as, e.g., a defensin or a cryptdin known in the art). An LP of the invention administered in combination can be administered simultaneously or sequentially and can be repeated as necessary.

LP321 Antimicrobial Assays: The antimicrobial activity of a purified LP321 or LP321 variant is tested against wild type and phoP mutant S. typhimurium by means of a modified plate diffusion assay (Lehrer, et al. 1991b J. Immunol. Methods 137:167-173, which is incorporated herein by reference for its assay methods) using wild type S. typhimurium (ATCC 10428) or an isogenic phoP mutant of S. typhimurium (strain CS015 phoP102::Tn10d-Cam, Miller et al., supra, 1989). Note, the phoP locus is a two-component regulatory locus essential to S. typhimurium virulence and survival within macrophages (Fields et al., Science 243:1059-1062 (1989); Miller et al. Proc. Natl. Acad. Sci. USA 86:5054-5058 (1989), each of which is incorporated herein by reference). Mutants in the phoP locus are particularly sensitive to rabbit defensins NP-1 and NP-2 when compared to wild type parent strains (Fields et al., supra, 1989; Miller et al., Infect. Immun. 58:3706-3710, (1990), which is incorporated herein by reference).

Cells are grown to log phase in trypticase soy broth at 37° C., harvested by centrifugation and resuspended to approximately 10 million colony forming units (CFU) per ml in 10 mM sodium phosphate buffer (pH 7.4). A 100 ul aliquot of each organism is mixed with 10 ml 1.0% agarose in 0.03% (w/v) trypticase soy medium, 10 mM sodium phosphate (pH 7.4) at 42° C. Five ul samples of peptide solution are pipetted into 3 mm diameter wells formed in agarose with a sterile punch. After 3 hr at 37° C., the inoculated agarose plate is overlaid with 1.0% agarose containing 6.0% trypticase soy medium. After 12-16 hr, antimicrobial activity is demonstrated by clear zones surrounding wells loaded with antibacterial samples; the areas of the clear zones are typically concentration-dependent.

A cryptdin or cryptdin-like composition's antimicrobial activity in vitro is substantially enhanced in piperazine-N, N′-bis (2-ethane 5-sulfonic acid) (PIPES) or in N-2-hydroxyethylpiperazine-N′-2-ethanesulfonic acid (HEPES) as compared to its activity in sodium phosphate. Purified LP peptides of the invention are dissolved at various concentrations (such as, e.g., from about 0.1 to about 300 ug/ml) in 0.01% acetic acid and activity is examined against E. coli ML35 (ATCC). In a radial diffusion assay, 5 ul of peptide solution is transferred into wells formed in plates of 1% agarose buffered with 10 mM PIPES (pH 7.4) and containing 1×106 log-phase bacteria grown in trypticase soy broth. After 3 hr at 37° C., the plates are overlaid with 0.8% agarose containing 2× trypticase soy broth and incubated overnight.

Antibacterial activities of LP peptides are compared with, e.g., antibacterial activities of rabbit neutrophil defensin NP-1, which is purified from peritoneal exudates as described by Selsted, et al. 1985 J. Biol. Chem. 260:4579-4584 (incorporated herein by reference for such assay teachings) or with a known cryptdin sequence. Antibacterial activity is determined by measuring the diameter of clearing around each well and plotted as a function of peptide concentration. Positive results will typically produce a dose-dependent zone of clearing that demonstrates an inhibition of microbial growth. Potencies of an LP321 or an LP321 variant may vary depending on dosage and modification. For instance, an LP321 or LP321 variant may be more active than rabbi: NP-1 at a concentration above 100 ug/ml or more active than NP-1 when compared at 100 ug/ml and 300 ug/ml. Higher concentrations may he more effective than the same concentration of NP-1 at inhibiting the growth of S. aureus and of wild type and mutant strains of S. typhimurium. An inhibition of S. aureus is interpreted as indicating that an LP321 or LP321 variant peptide inhibits bacterial growth.

To determine if an effect of an LP321 or an LP321 variant peptide against E. coli is bacteriostatic or bacteriocidal, bacterial killing is quantitated as a function of time. Bactericidal assays are performed by incubating approximately 1-2×106 log-phase bacteria in 10 mM PIPES containing from about 0.1 to about 10 ug peptide/ml. After incubation for 15 or 30 min at 37° C., aliquots are removed, serially diluted, and plated on trypticase soy agar. Bactericidal activity is quantitated by counting colonies after overnight incubation at 37° C.

The bactericidal activity of, e.g., cryptdin 1 can be compared with an LP321 or LP321 variant peptide of the invention to compare microbial activity. Briefly, E. coli ML35 cells, S. aureus 502A cells or mutant or wild type S. typhimurium cells are incubated with various concentrations of rat cryptdin 1 or rabbit NP-1. Ten ug/ml rat cryptdin 1 has been reported to kill about 90% of the S. aureus cells and greater than 99% of the E. coli and mutant S. typhimurium cells, but is relatively ineffective in killing wild type S. typhimurium. Rat cryptdin 1 has been reported to be more effective than NP-1 in killing E. coli and mutant S. typhimurium cells, whereas NP-1 has been reported to be more effective in killing S. aureus. Such results can be compared to an LP321 or an LP321 variant of the invention to assess the relative microbiocidal strength of an LP composition described herein.

The effect of mouse cryptdins 1-3 and 6 at inhibiting the growth of the protozoan, Giardia lamblia (which is the most common cause of protozoan disease in the human small intestine) can also be examined in comparison with an LP321 or an LP321 variant. Briefly, trophozoites of the C6 clone of Giardia lamblia WB (ATCC 30957) are grown to late log phase in TYI-S-33 medium containing bovine bile. Free-swimming trophozoites are discarded and tubes with attached trophozoites are refilled with warm Dulbecco's PBS. Trophozoites are detached by chilling 10 min on ice, then harvested by centrifugation, resuspended at 2×107/ml in 25 mM HEPES (pH 7.5) containing 9.0% (isotonic) sucrose and incubated for 2 hr at 37° C. with various concentrations of, e.g., mouse cryptdins 1-3 or 6 and an LP peptide. Following incubation, trophozoite viability is determined and compared (depending on treatment) by trypan blue exclusion to determine how an LP321 or LP321 variant peptide or mouse cryptdins 1-3 or 6 kill Giardia trophozoites in a dose-dependent manner (e.g., it has been reported that 20 ug/ml of cryptdin 2.or cryptdin 3 reduces Giardia growth by greater than 2 orders of magnitude thus, indicating that such cryptdins are active against a variety of microorganisms see, WO 96/16075 for cryptdin assay methods (WO96/16075 is incorporated herein by reference for such assay methods).

Other assays for microbicidal activities can also be employed to test an LP321 or an LP321 variant of the invention. Such assays are commonly art known, and could be adapted for use to test an LP321, an LP321 variant or an LP321 binding agent without undue experimentation, (see, e.g., Selsted, M. E. (1993) in Investigational Approaches for Studying the Structures and Biological Functions of Myeloid Antimicrobial Peptides, ed. Setlow, J. K. (Plenum, N.Y.), Vol. 15, pp. 131-147; which is incorporated herein for such method techniques). For example, the assay teachings of Ouellette, et al 1994 Infect. Immun. 62, 5040-5047; Selsted, et al. 1992 J. Cell Biol. 118, 929-936; Eisenhauer, et al. 1992 Infect. Immun. 60, 3556-3565; and Ayabe, et al. 2000 Nature Immunology 1:113-118 are all incorporated herein by reference for their assay teachings. To investigate the ability of an LP321 or an LP321 variant of the invention to create an ionic current in a cell or a membrane, such as, e.g., a cell membrane, experiments are carried out based on the methods of Lencer, et al. 1997 Proc. Natl. Acad. Sci. USA Vol. 94:8585-8589, which is incorporated herein by reference for these assay teachings. Briefly, human intestinal T84 cells obtained from the American Type Culture Collection are cultured and passaged as described by Dharmsathaphorn & Madara 1990 Methods Enzymol. 192:354-359, which is incorporated herein by reference for these assay teachings. When grown on permeable supports, T84 cells form confluent monolayers of columnar epithelia that display polarized apical and basolateral membranes, high transepithelial resistances, and a regulated Cl2 secretory pathway analogous to that found in native crypt epithelium (Dharmsathaphorn & Madara 1990 Methods Enzymol. 192:354-359). Cl2 secretion is assessed as a short circuit current (Isc) using standard electrophysiologic techniques (Lencer, et al. 1992 J. Cell Biol. 117, 1197-1209, which is incorporated herein by reference for these method teachings). cAMP and cGMP are assessed in ethanol extracts of T84 cell monolayers by radioimmune assay kit (NEN). Hanks' balanced salt solution (HBSS; containing 1.67 mM CaCl2 0.8 mM MgSO4 5 mM KCl 0.45 mM KH2PO4 137 mM NaCl 0.33 mM Na2HPO4 5 mM glucose 10 mM Hepes, pH 7.4) is used for all assays unless otherwise stated. Abbreviations: HBSS, Hanks' balanced salt solution; Isc, short circuit current; BCECF-acid, 29,79-bis-(2-carboxyethyl)-5-(and-6)-carboxy-fluorescein.

Peptide Purification and Synthesis. To compare a cryptdin with an LP321 or an LP321 variant of the invention one can use any standard recombinant method employing a published cryptdin sequence to generate a cryptdin peptide of interest, or one can use a low molecular weight peptide fraction (P-60 cryptdin fraction; from which all known cryptdins to date have been purified) to purify a cryptdin of interest. Briefly, the peptide fraction is prepared by Biogel P-60 gel chromatography of an acid extract homogenate of adult outbred Swiss Webster mouse small intestine using a method of Selsted, et al 1992 J. Cell Biol. 118, 929-936, which is incorporated herein by reference for these assay method teachings. Further purification by reversed-phase HPLC yields a cryptdin-enriched pool (HPLC cryptdin pool) that contains mouse cryptdins 1-6 as well as a number of other non-cryptdin proteins. From this fraction, mouse cryptdins 1-6 are purified to homogeneity by HPLC using the methods of Selsted, supra; or Ouellette, et al. 1992 FEBS Lett. 304, 146-148 (which is incorporated herein by reference for these assay method teachings). Some studies can also be carried out using synthetic, folded, and oxidized cryptdin 3, prepared as described for cryptdin 1 (Selsted, et al 1992 J. Cell Biol. 118, 929-936). Synthetic and natural cryptdin 3 peptides have been shown to have identical physicochemical and antimicrobial characteristics (see, Selsted, M. E. (1993) in Investigational Approaches for Studying the Structures and Biological Functions of Myeloid Antimicrobial Peptides, ed. Sedow, J. K. (Plenum, N.Y.), Vol.15, pp.131-147).

Induced Pore Formation. Nonpolarized T84 cells (grown on glass coverslips) or polarized monolayers (grown on filter supports) are incubated at 37° C. for 30 min in HBSS containing the membrane impermeant fluorophore 29,79-bis-(2-carboxyethyl)-5-(and-6)-carboxyfluorescein (BCECF-acid, 0.1 uM) with or without the addition of an LP321, LP321 variant, or a cryptdin of interest (10-600 ug/ml). At the end of the exposure, coverslips or monolayers on their filter supports are washed in fresh HBSS containing 0.1 uM BCECF at 37° C. to remove the peptide. After an additional 10 min, coverslips or monolayers are washed again in fresh HBSS and examined by epifluorescence (490 nM excitation, 520 emission) and bright field microscopy using Nomarski optics for the presence of the fluorophore outside of the cells of interest.

To obtain direct evidence that an LP321-induced response in T84 cells is due to the formation of cryptdin-like-based channels in a membrane, the effect of an LP321 or an LP321 variant on the permeability of T84 cell plasma membranes to the impermeant fluorophore BCECF-acid (450 Da) is examined as described in Lencer, et al. 1997. For purposes of comparison, it has been shown that cryptdin 3 markedly increases membrane permeability of nonpolarized T84 cells to this organic acid.

Features of LP No: 8 (LP317)

LP317 is a novel primate (e.g., human) polypeptide (SEQ ID NO: 18) that exhibits similarity to a defensin family of proteins. Specifically, LP317 is a novel member of the gamma-thionin family of proteins. These defensins exhibit remarkable structural similarity to scorpion neurotoxins and insect defensins, which are generally recognized a having antibiotic, antifungal, antitumor, antineoplastic and antiviral activities. Additionally, gamma-thionins have been recently shown to rapidly and reversibly inhibit l(Na) without changing the kinetics or voltage dependence of activation or inactivation (Kushmerick, et al. 1998 FEBS Lett 440(3):302-306). Accordingly, an LP317, or an LP317 variant can function as a new class of sodium channel blockers. Furthermore, LP317 shares sequence similarity with amylase inhibitors. Elevation of serum amylase is associated with lung cancer (Grove, A. 1994 APMIS 102(2):135-44), myeloma (Fujii, et al. 1991 Arch Pathol Lab Med 115(9):952-956), and pancreatitis (Vissers, et al. 1999 J Emerg Med 17(6):1027-1037). Amylase also plays a role in dental plaque and caries formation (Scannapieco, et al. 1993 Crit Rev Oral Biol Med 4(3-4):301-307). Accordingly, compositions comprising LP317 polypeptides, polynucleotides, its agonists/antagonists and/or antibodies are useful for diagnosis, treatment and intervention of cancer, pancreatitis, and tooth decay. LP317 is also expressed in prostate stroma. Accordingly, compositions comprising LP317 polypeptides, polynucleotides, its agonists/antagonists and/or antibodies are also useful for the treatment of defects in or wounds to prostate. Defensins are a family of structurally related cysteine-rich peptides active against many Gram-negative and Gram-positive bacteria, fungi, and viruses (see, e.g., Lehrer, et al. Cell 64:229-230; Kagan, et al. 1994 Toxicology 87:131-149; Lehrer, et al. 1993 Annu. Rev. Immunol. 11:105-128; and White, et al. 1995 Curr. Opin. Struct. Biol. 5:521-527). Some defensins are also called corticostatins (CS) because they inhibit corticotropin-stimulated corticosteroid production. Defensins can kill cells by forming voltage-regulated multimeric channels in the membrane of the susceptible cell. Defensins play a significant role in innate immunity to infection and neoplasia. Antimicrobial peptides are a prevalent mechanism of host defense found throughout nature (Kaiser & Diamond 2000 J Leukoc Biol 6:779-84). In mammals, defensins are among the most abundant of these broad-spectrum antibiotics, and are expressed in epithelial and hematopoietic cells. The defensin peptides are especially abundant in neutrophils; however, gene expression is limited to the promyelocyte stage. In epithelial cells, defensin genes are found as both constitutively expressed and inducible. Induction has been observed in vitro by stimulation with bacterial lipopolysaccharide as well as inflammatory mediators. In vivo, up-regulation of several defensin genes occurs in both infectious and inflammatory states. Gene regulation occurs via signal transduction pathways common to other innate immune responses, using transcription factors such as nuclear factor (NF)-kappa beta and NF interleukin-6. Together, the data suggest a broad-based innate host defense whereby potent antimicrobial peptides are present to prevent initial colonization by pathogenic microorganisms. In addition, the recognition of bacteria coupled with a nascent inflammatory response can bolster this defense by a coordinated up-regulation of the peptides. Some peptides known to belong to the defensin family include, e.g.: Rabbit defensins and corticostatins: CS-I (NP-3A), CS-II (NP-3B), CS-III, (MCP-1), CS-IV (MCP-2), NP-4, and NP-5; Guinea-pig neutrophil defensin (GPNP); Human neutrophil defensins 1 to 4 and intestinal defensins 5 and 6; Mouse small bowel cryptdins 1 to 5 and; Rat NP-1 to NP-4. All these peptides range in length from approximately 29 to about 35 amino acids and typically, at the primary amino acid sequence level, they possess invariant cysteine residues that are involved in intrachain disulfide bonding.

Before the characterization of mouse intestinal defensin cDNA, expression of defensins was thought to be limited to professional phagocytes, such as, for example, neutrophils and macrophages. The presence of high levels of cryptdin mRNA in Paneth cells led to the hypothesis that defensins synthesized in intestinal epithelium contribute to antimicrobial barrier function in the small bowel (Ouellette et al., 1989a J. Cell Biol. 108:1687-1695). Isolation and characterization of six mouse cryptdin peptides, two rat cryptdin peptides and 2 human cryptdin peptides, and the demonstration of antimicrobial activity of various cryptdin peptides indicates that the cryptdins have an antimicrobial role in the small intestine. The immunohistochemical localization of cryptdin(s) to Paneth cells is consistent with previous in situ hybridization analysis and suggests that defensins produced by these cells may contribute to restricting the colonization and invasion of the small bowel by bacteria.

LP317 nucleic acid sequence (SEQ ID NO: 17) is only found in a human brain and prostate stroma of a LIFESEQ GOLD™ database tissue and cDNA library.

TABLE 7

Primate, e.g., human, LP317 polynucleotide sequence (SEQ ID NO: 17) and corresponding
polypeptide (SEQ ID NO: 18). The ORF for LP317 is 97-345 bp with the start (ATG) and stop Codons
(TAA) identified in bold typeface and underlined. In the event that the numbering is misidentified,
one skilled in the art could determine the open reading frame without undue experimentation.

LP317 DNA sequence: (540 bp) (ORF = 97-345):
LP317 (start (atg) and stop (tga) codons are indicated in bold typeface
and underlined).
ATGGAGCTCATCAAGTCCAGGGCGACCGTGTGCGCGCTCCTCCTGGCGCTGCTCCTGCTCTCGCACTACGA
CGGCGGGACGACGACGACGATGGTGGCGGAGGCCCGGGTGTGCATGGGCAAGAGCCAGCACCACTCGTTCC
CCTGCATCTCCGACCGCCTCTGCAGCAACGAGTGCGTCAAGGAGGACGGCGGGTGGACCGCCGGCTACTGC
CACCTCCGCTACTGCAGGTGCCAGAAGGCGTGCTAA
LP317 Full-Length Sequence (82aa):
(SEQ ID NO: 18) The underlined portion is a predicted signal sequence (Met-1 to Gly-25). A predicted SP
cleavage site is between Gly-25 and Gly-26 indicated as follows: 1 MELIKSRATVCALLLALLLLSHYDG\hat{ }GT
27. LP317 polypeptides encompassed herein include full-length forms encoded by an ORF disclosed herein, as
well as any mature forms therefrom. Such a mature LP317 could be formed, for example, by the removal of a
signal peptide or an aminopeptidase modification. Further as used herein, a “mature” LP encompass,
e.g., post-translational modifications other than proteolytic cleavages (such as, e.g., by way of a non-limiting
example, glycosylations, myristylations, phosphorylations, prenylations, acylations, and sulfations).
All such variants are also encompassed by an LP of the present invention. Further, an LP of the
invention encompass all fragments, analogs, homologs, and derivatives of an LP described herein, thus
the invention encompasses both LP precursors and any modified versions (such as, e.g., by post-
translational modification) of an LP encoded by an LP nucleic acid sequence described herein.
MELIKSRATVCALLLALLLLSHYDGGTTTMVAEARVCMGKSQHHSFPCISDRLCSNECVK
EDGGWTAGYCHLRYCRCQKAC
An LP317 Mature Sequence (57aa):
A predicted mature L2317 sequence is as follows:
GTTTTMVAEARVCMGKSQHHSFPCISDRLCSNECVKEDGGWTAGYCHLRYCRCQKAC
An LP317 Variant Sequence (47aa):
An alternate LP3l7 mature.
RVCMGKSQHHSFPCISDRLCSNECVKEDGGWTACYCHLRYCRCQKAC
An LP317 Variant Sequence (56aa):
An alternate LP317 mature sequence with the N terminal portion before
the first cysteine shortened.
TTTTMVAEARVCMGKSQHHSFPCISDRLCSNECVKEDGGWTAGYCHLRYCRCQKAC
An LP317 Variant Sequence (55aa):
An alternate LP317 mature sequence with the N terminal portion before
the first cysteine shortened.
TTTMVAEARVCMGKSQHHSFPCISDRLCSNECVKEDGGWTAGYCHLRYCRCQKAC
An LP317 Variant Sequence (53aa):
An alternate LP317 mature sequence with the N terminal portion before
the first cysteine shortened.
TMVAEARVCMGKSQHHSFPCISDRLCSNECVKEDGGWTAGYCHLRYCRCQKAC
An LP317 Variant Sequence (52aa):
An alternate LP317 mature sequence with the N terminal portion before
the first cysteine shortened.
MVAEARVCMGKSQHHSFPCISDRLCSNECVKEDGGWTAGYCHLRYCRCQKAC
An LP317 Variant Sequence (51aa):
An alternate LP317 mature sequence with the N terminal portion before
the first cysteine shortened.
VAEARVCMGKSQHHSFPCISDRLCSNECVKEDGGWTAGYCHLRYCRCQKAC
An LP317 Variant Sequence (50aa):
An alternate LP317 mature sequence with the N terminal portion before
the first cysteine shortened.
AEARVCMGKSQHHSFPCISDRLCSNECVKEDGGWTAGYCHLRYCRCQKAC
An LP317 Variant Sequence (49aa):
An alternate LP317 mature sequence with the N terminal portion before
the first cysteine shortened.
EARVCMGKSQHHSFPCISDRLCSNECVKEDGGWTAGYCHLRYCRCQKAC
An LP317 Variant Sequence (48aa):
An alternate LP317 mature sequence with the N terminal portion before
the first cysteine shortened.
ARVCMGKSQHHSFPCISDRLCSNECVKEDGGWTAGYCHLRYCRCQKAC

Interesting segments of LP317 are the segments from about Arg-36 to about Cys-82 (RVCMGKSQHHSFPCISDRLCSNECVKEDGGWTAGYCHLRYCRCQKAC), and from about Arg-36 to about Cys-59 (RVCMGKSQHHSFPCISDRLCSNEC), which have been discovered to exhibit gamma-thionin-like domain signatures. Other interesting segments of LP317 are the segments from about Val-37 to about Asp-52 (VCMGKSQHHSFPCISD), and from about Gly-69 to about Cys-82 (GYCHLRYCRCQKAC), which have been discovered to exhibit a purothionin-like signature. Gamma-purothionin inhibits protein translation in cell-free systems. A further interesting segment of LP317 is the segment from about Cys-49 to about Cys-78 (CISDRLCSNECVKEDGGWTAGYCHLRYCRC), which has been discovered to exhibit a scorpion-short-toxin-like signature. Other interesting segments of LP317 are discovered fragments are the discovered portions of LP317 from about Ser-6 to about Ala-16; from about Leu-13 to about Tyr-23; from about Cys-38 to about Cys-49; from about Ser-56 to about Thr-67; from about Gly-65 to about Tyr-75; from about Cys-38 to about Cys-49, from about Asp-63 to about Cys-71; from about Cys-11 to about Ser-21, from about His-22 to about Val-32; from about Met-31 to about Gly-40; from about Val-37 to about Ser-46, from about Pro-49 to about Val-71, from about Leu-54 to about Thr-67; and from about Val-60 to about Cys-71; whose discoveries were based on an analysis of hydrophobicity, hydropathicity, and hydrophilicity plots. Additional interesting sections of LP317 are the discovered portions of LP317 from about Ala-8 to Tyr-23; from about Asp-24 to about Glu-34; from about Arg-36 to about Lys-41; from about Pro-48 to about Cys-59; from about Cys-59 to about Gly-69; and from about Ser-70 to about Arg-80. These fragments were discovered based on analysis of antigenicity plots. Further, particularly interesting LP317 structures (e.g., such as a helix, a strand, or a coil) that have been discovered is the following LP317 helix structure: from about Thr-9 to about Leu-17. Particularly interesting discovered coil structures are from about His-22 to about Thr-27; from about Gly-40 to about Ile-50; from about Ser-51 to about Gly-69; and from about Gln-79 to about Cys-82. A particularly interesting discovered strand structure is from about Thr-30 to about Val-32 (TMV). Further encompassed by the invention are contiguous amino acid residue combinations of any of the predicted secondary structures described above. For example, one coil-strand-coil-coil motif of LP317 combines the coil from about His-22 to about Thr-27, the strand from about Thr-30 to about Val-32, the coil from about Gly-40 to about Ile-50, and the coil from about Ser-51 to about Gly-69 to form an interesting fragment of contiguous amino acid residues from about His-22 to about to about Gly-69. Other combinations of contiguous amino acids are contemplated as can be easily determined.

The use of the term LP peptide herein encompasses cationic peptide LP variants which, as defined herein, refer to an LP peptide of the invention with a net positive charge within the pH range of from about pH4.0 to about p10.0, including pH values of 3.7, 3.8, 3.9, 4.1, 4.2, 4.3, 4.4, 4.5, 4.6, 4.7, 4.8, 4.9, 5.0, 5.1, 5.2, 5.3, 5.5, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.7, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.9, 9.5, 9.6, 9.7, 9.8, 9.9, 10.0, 10.1, 10.2, 10.3, and 10.4. A cationic LP peptide variant is at least five contiguous amino acids in length of an LP peptide described herein, and has at least one basic amino acid (e.g., arginine, lysine, histidine). A cationic LP peptide variant typically does not have more than about 25, about 27, about 30, about 35, about 40, about 45, about 50, about 55 or about 60 amino acids, and typically has about 12, 13, 14, 15, 16, 17, 18, 19, amino acid residues; more preferably at least about: 20, 21, 22, 24, 26, or 29 amino acid residues, favorably at least about: 30, 31, 32, 33, 34, 35, 36, 37, 38, or 39 amino acid residues, more preferably, at least about: 40, 41, 42, 43, 44, 45, 46, 47, 48, or 49 amino acid residues; desirably at least about: 50, 51, 52, 53, 54, 55, 56, 57, 58, or 59 nucleotides, particularly at least about 60, 61, 62, 63, 64, 65, 66, 67, 68, or 69 amino acid residues; more particularly at least about 70, 71, 72, 73, 74, 75, 76, 77, 78, or 79; predominantly at least about 80, 81, 82, 83, 84, 85, 86, 87, 88, or 89; and even more favorably at least about 90, 91, 92, 93, 94, 95, 96, 97, 98, 99, or 100 amino acid residues.

Similar cationic-like peptide examples include, for instanced, vertebrate defensins, such as human neutrophil defensins [HNP 1-4], paneth cell defensins of mouse and human small intestine (Oulette and Selsted, FASEB J. 10:1280, 1996; Porter et al., Infect. Immun. 65:2396,1997), vertebrate defensins, such as HBD-1 of human epithelial cells (Zhao et al., FEBS Lett. 368:331, 1995), HBD-2 of inflamed human skin (Harder et al., Nature 387:861, 1997), bovine [defensins] (Russell et al., Infect. Immun. 64:1565, 1996), plant defensins, such as Rs-AFP 1 of radish seeds (Fehlbaum et al., J. Biol. Chem. 269:33159, 1994), alpha- and beta-thionins (Stuart et al., Cereal Chem. 19:288, 1942; Bohlmann and Apel, Annu. Rev. Physiol. Plant Mol. Biol. 42:227, 1991), and gamma-thionins (Broekaert et al., Plant Physiol. 108:1353, 1995). Yet another type of LP cationic peptide variant encompassed by the term LP peptide is a cationic peptide that has been conjugated with a bioactive agent, such as a one described herein. Additional cationic peptide variants encompassed by the term LP peptides are peptides that have one or more amino acids altered to a corresponding D-amino acid. For example, the N-terminal and/or C-terminal amino acid can be a D-amino acid. Certain cationic peptide variants are acetylated at the N-terminal amino acid, and/or amidated (or esterified) at the C-terminal amino acid. Moreover, a cationic peptide variant encompassed by the invention can be modified by incorporation of homoserine/homoserine lactone at the C-terminal amino acid. Typically, an LP cationic peptide variant encompassed herein exhibits at least 50%, and preferably, greater than 60, 70, 80, 85, 87, or 90% of an activity of a corresponding naturally occurring LP peptide of the invention as determined by any art known assay or an assay described or referenced herein. The antibiotic activity of such LP analogs or variants can be determined using any art known method, such as an assay described herein. As an illustration, an in vivo assay to measure anti-microbial activity is used as described herein. An in vivo assay can also be used to evaluate the activity of a cationic peptide analog or variant for treatment of tumors. Alternatively, in vitro assays can provide a simple test for anti-nepotistic LP analogs or anti-nepotistic variant LP peptides, such as the methylthiazoltetrazolium (MTT) or the lactate dehydrogenase (LDH) assay. The MTT assay is a tetrazolium dye colormetric assay that measures cell viability, while the LDH assay measures cell cytotoxicity.

LP317 Functions: Given the teachings supplied herein, for example, of: LP317 primary amino acid, LP317 higher order structures, the relationship of LP317 amino acid sequence to higher order structural features of thionins and short scorpion toxins, the comparability of LP317 sequence and/or LP317 higher order structure with known thionins and short scorpion toxins, and the relationship of higher order structural features of such proteins with their known functions, it is likely that an LP317, an LP317 variant, and/or in LP317 binding agent (e.g., such as an LP317 antibody (or fragment thereof)) plays a similar role/s in a variety of physiological processes. Some non-limiting examples of functions an LP317, an LP317 variant, an LP317 hetero- or an LP317 homomultimer, or an LP317 binding agent or LP binding compound is likely to participate in are, for example, those such as mucosal immunity (such as, e.g., mucosal surfaces in, e.g., epithelia in the airway, skin, oropharynx, gingival crevice, and urogenital); host defense; anti-microbial activity (such as, e.g., against bacteria; (such as, e.g., Gram-positive, Gram-negative, S. typhimurim, S. typhimurium, S. typhimurium phoP- mutant, Staphylococcus aureus, Streptococcus pyogenes, Esherichia coli, and Listeria moncyotgenes) protists (such as, e.g., G. lamblia); fungi; and viruses (such as, e.g., enveloped viruses); inhibiting the colonization of an epithelium by a pathogenic microorganism; protection of developing spermatids; regulation of cell volume; an anti-neoplastic agent; anti-tumorogenic effect; chemotaxis; mitogenicity; inhibition of natural killer cell activity; modulation of ion flow (such as, e.g., calcium, sodium, potassium, or chloride ions); modulation of an ionic flow through a membrane, wherein said mediation of innate immunity; modulate chloride ion flow; create an ionic pore in a membrane; creating an ionic pore in a membrane, wherein said membrane is in an epithelium, wherein said epithelium is a broncho epithelium; wherein said epithelium is in a lung; creating an ionic pore in a membrane, wherein said membrane is in a microbe; mounting and maintaining defense against a luminal floral pathogen such as, e.g., enteric bacteria; prevention of sepsis; protection of stem cell integrity; protection of stem cell integrity in a lumen, wherein said lumen is an intestinal lumen, wherein said lumen is a oropharyngeal lumen, or wherein said lumen is a urogenital lumen.

LP Antimicrobial Assays: The antimicrobial activity of an isolated or recombinant LP317 or LP variant of the invention is tested against wild type and phoP mutant S. typhimurium by means of a modified plate diffusion assay (Lehrer, et al. 1991 b J. Immunol. Methods 137:167-173, which is incorporated herein by reference for these assay methods) using wild type S. typhimurium (ATCC 10428) or an isogenic phoP mutant of S. typhimurium (strain CS015 phoP102::Tn10d-Cam, Miller et al., supra, 1989). The phoP locus is a two-component regulatory locus essential to S. typhimurium virulence and survival within macrophages (Fields et al., Science 243:1059-1062 (1989); Miller et al. Proc. Natl. Acad. Sci. USA 86:5054-5058 (1989), each of which is incorporated herein by reference). Other assays for microbicidal activities could also be employed to test an LP of the invention, such assays are commonly used (see, e.g., Selsted, M. E. (1993) in Investigational Approaches for Studying the Structures and Biological Functions of Myeloid Antimicrobial Peptides, ed. Setlow, J. K. (Plenum, N.Y.), Vol. 15, pp. 131-147; which is incorporated herein for such method techniques), art known, and could be adapted for use to rest for example an LP317, LP317 variants or LP317 binding agents without undue experimentation. For example, the assay teachings of Ouellette, et al 1994 Infect. Immmun. 62, 5040-5047; Selsted, et al. 1992 J. Cell Biol. 118, 929-936; Eisenhauer, et al. 1992 Infect. Immun. 60, 3556-3565; and Ayabe, et al. 2000 Nature Immunology 1:113-118 are incorporated herein by reference for their assay teachings.

Ionic Permeabilization Assays: To investigate the ability of an LP317 or an LP317 variant of the invention to create ionic currents in cells, experiments are carried out based on the methods of Lencer, et al. 1997 Proc. Natl. Acad. Sci. USA Vol. 94:8585-8589, which is incorporated herein by reference for these assay teachings.

Features of LP No: 9 (LP283, LP344, LP345, & LP346)

LP283 and its splice variants (LP344, LP345, & LP346) are novel primate (e.g., human) polypeptide (SEQ ID NO: 20, 21, 22, & 23) members of the epidermal growth factor (EGF) superfamily. The EGF superfamily comprises a diverse group of proteins that function as secreted signaling molecules, growth factors, and components of the extracellular matrix involved in, for example, cell-cell, and/or cell-matrix adhesion. Many members of this group play a role in vertebrate development, such as for example in the development, establishment, remodeling, and/or maintenance of various organ or organ systems, such as, e.g., the nervous system, the reproductive system, the urogenital system, etc.

LP283 exhibits a unique domain architecture having an N-terminal signal peptide sequence, a series of tandem-like EGF-like repeats (approximately nine) and a C-terminal CUB-like domain. The CUB domain (Complement subcomponents Clr/Cls, Uegf, Bmp1; see, Bork & Beckman 1993 J. Mol. Biol. 231:539-45) is a domain spanning approximately 100-110 amino acid residues, which were first reported in the complement subcomponents Clr/Cls, epidermal-growth-factor-related sea urchin protein and bone morphogenetic protein 1. CUB domains are involved in protein-protein and glycosaminoglycan interactions. A number of proteins have been identified that contain both EGF and CUB domains, including Drosophila tolloid, the mammalian tolloid-related genes BMP1 and mTll, fibropellin I and III from sea urchin, and the serum glycoprotein attractin. Each of these proteins is implicated in the regulation of extracellular processes such as communication, adhesion, and guidance. Based on sequence and domain architecture similarity between LP283 and such proteins it is likely that LP283 or an LP283 variant (or a fragment thereof) will also function in such a role/s. More specifically, the domain architecture and sequence of LP283 shows similarity at the amino acid sequence level to a mammalian gene family that is defined by two proteins designated SCUBE1 and SCUBE2 (signal peptide-CUB domain-EGF-related 1, and 2; see, Grimmond, et al. 2000 Genomics 70:74-81; Grimmond, et al. 2001 Mech Dev 102:209-211) and to the fibropellins (Bisgrove, et al. 1995 J. Mol. Evol 41:34-45).

Other proteins similar to LP283 are the fibropellins. These are secreted glycoproteins that form physical associations to provide a protein substratum of the apical lamina, a component of the hyaline layer that surrounds sea urchin embryos (Delgadillo-Reynoso et al. 1989 J. Mol. Evol. 29:314-327; Burke, et al. 1998 Cell Adhes Commun 5:97-108). Expression studies suggest that the fibropellins function during developmental periods when the organization of the sea urchin embryo is changing rapidly due to mesenchymal cell ingression, gastrulation, and larval morphogenesis, all of which are processes that are believed to involve the interaction of migrating cells and ECM components (Bisgrove and Raff 1993 Dev Biol 157:526-538). Thus, fibropellins function by mediating cell movements. Both EGF and CUB domains are implicated in the physical association of the fibropellins with ECM proteins. Given the relationship of LP283 sequence and the LP283 domain architecture to proteins such as SCUBE and fibropellin, it is likely that, among other things, LP283, LP283 variants, or LP283 fragments will play a role in the development of vertebrate organs or organ systems, such as, for example, the central nervous system, the reproductive system, the urogenital system, and/or the development of the limbs.

LP283 nucleic acid sequence (SEQ ID NO: 19) is expressed in the following LIFESEQ GOLD™ database tissue and cDNA libraries: Embryonic Structures 1/23; Endocrine System 3/63; Genitalia, Female 2/113; Hemic and Immune System 3/166; Musculoskeletal System 1/50; Nervous System 4/221; Sense Organs 1/10; and Urinary Tract 2/66. Sequence encoding LP283 and its splice variants (LP344, LP345, & LP346) has been localized to human chromosome region 6p21.1-21.33. Moreover, the following diseases, conditions, syndromes, disorders, or pathological states have also been mapped to this region of the human chromosome: psoriasis (Balendran, et al. 1999 J. Invest Dermatol 113(3):322-328), autosomal recessive polycystic kidney disease (Besbas, et al., 1998 Turk J Pediatr 40(2):245-7, and Mucher, et al., 1998 Genomics 48(1):40-45); autosomal recessive retinitis pigmentosa (Banerjee, et al., 1998 Genomics 48(2):171-177), Hamartoma of the breast (Dal Cin, et al., 1997 Genes Chromosomes Cancer (1):90-92), juvenile myoclonic epilepsy (Liu, et al., 1995 Am J Hum Genet 57(2):368-381), and hypotrichosis simplex of the scalp (Betz, et al., 2000 Am J Hum Genet 66(6):1979-1983). Accordingly, isolated and/or recombinant LP283, LP344, LP345, & LP346 (or a fragment thereof) meets the statutory utility requirement of 35 U.S.C. §101 since LP283, LP344, LP345,& LP346 nucleic acid sequence (or portions thereof) can be used to hybridize near one or more of the above stated diseases and thus serves as a useful new marker for such a disease gene. Accordingly, LP283, LP283 variants (e.g., LP344, LP345, & LP346) or fragments thereof have both specific and general utility. Compositions comprising P283, LP344, LP345, & LP346 polypeptides or polynucleotides, (fragments thereof), P283, LP344, LP345, & LP346 agonists or antagonists, and/or binding compositions (e.g., P283, LP344, LP345, & LP346 antibodies) will also be useful for diagnosis, and/or prognosis, of such a disease, condition, syndrome, or state.

TABLE 8

Primate, e.g., human, LP283 polynucleotide sequence (SEQ ID NO: 19) and corresponding
polypeptide (SEQ ID NO: 20). The ORF for LP283 is 100-3,129 bp (with the start (ATG) and stop codons
(TAA) identified in bold typeface and underlined. In the event that the numbering is misidentified,
one skilled in the art could determine the open reading frame without undue experimentation).

```
LP283 DNA sequence (3,183 bp) (ORF = 100-3,129):
ATGGGCTCOOGGCCCGTACCCGGGCTCTGCCTGCTTGTCCTGCTGGTCCACCCCCGCGCCGCCCAGTACAG
CAAAGCCGCGCAAGATGTGGATGAGTGTGTGGAGGGGACTGACAACTGCCACATCGATGCTATCTGCCAGA
ACACCCCGAGGTCATACAGTGCATCTGCAAGTCTGGGCTACACAGGGGACGGCAAACACTGCAAAGACGTG
GATGAGTGCGAGCGAGAGGATAATGCAGGTTGTGTGCATGACTGTGTCAACATCCCTGGCAATTACCGGTG
TACCTGCTATGATGGATTCCACCTGGCACATGACGGACACAACTGTCTGGATGTGGACGAGTGTGCCGAGG
GCAACGGCGGCTGTCAGCAGAGCTGTGTCAACATGATGGGCAGCTATGAGTGCCACTGCCGGGAAGGCTTC
TTCCTCAGCGACAACCAGCATACCTGTATCCAGCGGCCAGAAGAAGGAATGAATTGCATGAACAAGAACCA
CGGCTGTGCCCACATTTGCCGGGAGACACCCAAGGGGGGTATTGCCTGTGAATGCCGTCCTGGCTTTGAGC
TTACCAAGAACCAACGGGACTGTAAATTGACATGCAACTATGGTAACGGCGGCTGCCAGCACACGTGTGAT
GACACAGAGCAGGGTCCCCGGTGCGGCTGCCATATCAAGTTTGTGCTCCATACCGACGGGAAGACATCCAT
CGGGGAAAGGCGGCTAGAGCAGCACATCCCCACTCAAGCCGTTTCTAATGAGACCTGTGCTGTCAACAACG
GGGGCTGTCGCAGTAAGTGCCATGATGCAGCGACTGGTGTCCACTGCACCTGCCCTGTGGGCTTCATGCTG
CAGCCAGAAGGAAGACGTGCAAAGATATAGATGAGTGCCGCTTAAACAACGGCGGGCTGTGACCATATTTG
CCGCAACACAGTGGGCAGCTTCGAATGCAGTTGCAAGAAAGGCTATAAGCTTCTCATCAATGAGAGGAACT
GCCAGGATATAGACGAGTGTTCCTTTGATCGAACCTGTGACCACATATGTGTCAACACACCAGGAAGCTTC
CAGTGTCTCTGCCATCGTGGCTACCTGTTGTATGGTATCACCCACTGTGGGGATGTGGATGAATGCAGCAT
CAACCGGGGAGGTTGCCGTTTGGCTGCATCAACACTCCTGGCAGCTACCAGTGTACCTGCCCAAGCAGGCC
AGGGTCGGCTGCACTGGAATGGCAAAGATTGCACAGAGCCACTGAAGTGTCAGGGCAGTCCTGGGGCCTCG
AAAGCCATGCTCAGCTGCAACCGGTCTGGCAAGAAGGACACCTGTGCCCTGACCTGTCCCTCCAGGGCCCG
ATTTTTGCCAGAGTCTGAGAATGGCTTCACGGTGAGCTGTGGGACCCCCAGCCCCAGGGCTGCTCCAGCCC
GAGCTGGCCACAATGGGAACAGCACCAACTCCAACCACTGCCATGAGGCTGCAGTGCTGTCCATTAAACAA
CGGGCCTCCTTCAAGATCAAGGATGCCAAATGCCGTTTGCACCTGCGAAACAAAGGCAAAACAGAGGAGGC
TGGCAGAATCACAGGGCCAGGTGGTGCCCCCTGCTCTGAATGCCAGGTCACCTTCATCCACCTTAAGTGTG
ACTCCTCTCGGAAGGGCAAGGGCCGACGGGCCCGGACCCCTCCAGGCAAAGAGGTCACAAGGCTCACCCTG
GAACTGGAGGCAGAGGTCAGAGCCGAAGAAACCACAGCCAGCTGTGGGCTGCCCTGCCTCCGACAGCGAAT
GGAACGGCGGCTGAAAGGATCCCTGAAGATCCTCAGAAATCCATCAACCAGGACCGCTTCCTGCGTGCGCC
TGGCAGGCCTTGATTATGAGCTGGCCCACAAGCCGGGCCTGGTAGCCGGGGAGCGAGCAGAGCCGATGGAG
TCCTGTAGGCCCCGGGCAGCACCGTGCTGGGACCAAGTGTGTCAGCTGCCGCAGGGAACGTATTACCACGG
CCAGACGGAGCAGTGTGTGCCATGCCAGCGGGCACCTTCCAGGAGAGAGAAGGGCAGCTCTCCTGCCGACC
TTTGCCCTGGGAGTGATGCCCACGGGCCTCTTGGAGCCACCAACGTCACCACGTGTGCAGGTCAGTGCCCA
CCTGGCCAACACTCTGTAGATGGGTTCAAGCCCTGTCAGCCATGCCCACGTGGCACCTACCAACCTGAAGC
AGGACGGACCCTATGCTTCCCTTGTGGTGGGGGCCTCACCACCAAGCATGAAGGGGCCATTTCCTTCCAAG
ACTGTGACACCAAAGTCCAGTGCTCCCCAGGGCACTACTACAACACCAGCATCCACCGCTGTATTCGCTGT
GCCATGGGCTCCTATCAGCCCGACTTCCGTCAGAACTTCTGCAGCCGCTGTCCAGGAAACACAAGCACAGA
CTTTGATGGCTCTACCAGTGTGGCCCAATGCAAGAATCGTCAGTGTGGTGGGGAGCTGGGTGAGTTCACTG
GCTATATTGAGTCCCCCAACTACCCGGGCAACTACCCAGCTGGTGTGGAGTGCATCTGGAACATCAACCCC
CCACCCAAGCGCAAGATCCTTATCGTGGTACCAGAGATCTTCCTGCCATCTGAGGATGAGTGTGGGGACGT
CCTCGTCATGAGAAAGAACTCATCCCCATCCTCCATTACCACTTATGAGACCTGCCAGACCTACGAGCGTC
CCATTGCCTTCACTGCCCGTTCCAGGAAGCTCTGGATCAACTTCAAGACAAGCGAGGCCAACAGCGCCCGT
GGCTTCCAGATTCCCTATGTTACCTATGATGAGGACTATGAGCAGCTGGTAGAAGACATTGTGCGAGATGG
CCGGCTCTATGCCTCTGAAAACCACCAGGAGATTTTAAAGGACAAGAAGCTCATCAAGGCCTTCTTTGAGG
TGCTAGCCCACCCCCAGAACTACTTCAAGTACACAGAGAAACACAAGGAGATGCTGCCAAAATCCTTCATC
AAGCTGCTCCGCTCCAAAGTTTCCAGCTTCCTGAGGCCCTACAAATAG
LP283 Full-Length Sequence (1,009aa):
The underlined portion is a predicted signal sequence (Met-1 to Ala-27). A predicted SP cleavage site
is between Ala-20 and Gln-23 indicated as follows: 1 MGSGRVPGLCLLVLLVHARA\hat{ }AQ 22. An
alternate predicted SP cleavage site is between Cys-40 and Thr-43 indicated as follows: 1
MGSGRVPGLCLLVLLVHARAAQYSKAAQDVDECVEGTDNC\hat{ }ILT 43. Each mature LP283 version is
encompassed herein. An LP encompassed herein includcs full-length forms encoded by an ORF disclosed
herein, as well as any mature forms therefrom. Such a mature LP could be formed, for example, by the
removal oFa signal peptide and/or by aminopeptidase modification. Further, as used herein, a
“mature” LP encompasses, e.g., post-translational modifications other than proreolytic
cleavages (such as, e.g.. by way of a non-limiting example, glycosylations, mytistylations,
phosphorylations, prenylations, acylarions, and sulfations). Such variants are also encompassed by an
LP of the present invention. Further, an LP of the invention encompasses all fragments, analogs,
homologs, and derivatives of an LP described herein, thus the invention encompasses both LP precursors
and any modified versions (such as, e.g., by post-translational modification) of an LP encoded by an
LP nucleic acid sequence described herein.
MGSGRVPGLCLLVLLVHARAAQYSKAAQDVDECVEGTDNCHIDAICQNTPRSYKCICKSGYTGDGKHCKDVD
ECEREDNAGCVHDCVNIPGNYRCTCYDGFHLAHDGHNCLDVDECAEGNGGCQQSVNMSMGSYECHCREGFFL
SDNQHTCIQRPEEGMNCMNKNHGCAHICRETPKGIACECRPGFRLTKNQRDCKLTCNYGSNGGCQHTCDDTE
QGPRCGCHIKFVLHTDGKTCIGERRLEQHIPTQAVSNETCAVNNGGCDSKCHDAATGVHCTCPVGFMLQPDR
KTCKDIDECRLNNGGCDHICRNTVGSFECSCKKGYKLLINERNCQDIDECSFDRTCDHICVNTPGSFQCLCH
RGYLLYGITHCGDVDECSINRGGCRFGCINTPGSYQCTCPAGQGRLHWNGKDCTEPLKCQGSPGASKAMLSC
NRSGKKDTCALTCPSRARFLPESENGFTVSCGTPSPRAAPARAGHNGNSTNSNHCHEAAVLSIKQRASFKIK
DAKCRLHLRNKGKTEEAGRITGPGGAPCSECQVTFIHLKCDSSRKGKGRRARTPPGKEVTRLTLELEAEVRA
EETTASCGLPCLRQRMERRLKGSLKMLRKSINQDRFLLRLAGLDYELAHKPGLVAGERAEPMESCRPGQHRA
GTKCVSCPQGTYYHGQTEQCVPCPAGTFQEREGQLSCKLCPGSADHGPLGATNVTTCAGQCPPGQHSVDGFK
PCQPCPRGTYQPEAGRTLCFPCGGGLTTKHEGAISFQDCDTKVQCSPGHYYNTSIHRCIRCAMGSYQPDFRQ
NFCSRCPGNTSTDFDGSTSVAQCKNRQCGGELGEFTGYIESPNYPGNYPAGVECIWNINPPPKRKILIVVPE
IFLPSEDECGDVLVMRKNSSPSSITTYETCQTYERPIAFTARSRKLWINFKTSEANSARGFQIPYVTYDEDY
EQLVEDIVRDGRLYASENHQEILKDKKLIKAFFEVLAHPQNYFKYTEKHKEMPKSFIKLLRSKVDSSFLRPY
K
An LP283 Mature Sequence (989aa):
A predicted mature LP283 sequence is as follows:
AQYSKAAQDVDECVEGTDNCHIDAICQNTPRSYKCICKSGYTGDGKHCKDVDECEREDNAGCVHDCVNIPG
NYRCTCYDGFHLAHDGHNCLDVDECAEGNGGCQQSCVNMMGSYECHCREGFFLSDNQHTCIQRPEEGMNCM
NKNHGCAHICRETPKGGIACECRPGFELTKNQRDCKLTCNYGNGGCQHTCDDTEQGPRCGCHIKFVLHTDG
KTCIGERRLEQHIPTQAVSNETCAVNNGGCDSKCHDAATGVHCTCPVGFMLQPDRKTCKDIDECRLNNGGC
DHICRNTVGSFECSCKKGYKLLINERNCQDIDECSFDRTCDHICVNTPGSFQCLCHRGYLLYGITHCGDVD
ECSINRGGCRFGCINTPGSYQCTCPAGQGRLHWNGKDCTEPLKCQGSPGASKAMLSCNRSGKKDTCALTCP
SRARFLPESENGFTVSCGTPSPRAAPARAGHNGNSTNSNHCHEAAVLSIKQRASFKIKDAKCRLHLRNKGK
TEEAGRITGPGGAPCSECQVTFIHLKCDSSRKGKGRRARTPPGKEVTRLTLELEAEVRAEETTASCGLPCL
RQRMERRLKGSLKMLRKSINQDRFLLRLAGLDYELAHKPGLVAGERAEPMESCRPGQHRAGTKCVSCPQGT
YYHGQTEQCVPCPAGTFQEREGQLSCDLCPGSDAHGPLGATNVTTCAGQCPPGQHSVDGFKPCQPCPRGTY
QPEAGRTLCFPCGGGLTTKHEGAISFQDCDTKVQCSPGHYYNTSIHRCIRCAMGSYQPDFRQNFCSRCPGN
TSTDFDGSTSVAQCKNRQCGGELGEFTGYIESPNYPGNYPAGVECIWNINPPPKRKILIVVPEIFLPSEDE
CGDVLMRKNSSPSSITTYETCQTYERPIAFTARSRKLWINFKTSEANSARGFQIPYVTYDEDYEQLVSEDI
VRDGRLYASENQEILKDKKLIKAFFEVLAHPQNYFKYTEKHKEMLPKSFIKLLRSKVSSFLRPYK
An Alternate LP283 Mature Sequence (993aa):
Encompassed herein are LP283 splice variants. One such variant is listed below. Applicants discovered
that this LP283 variant (also known as LP344 (SEQ ID NO: 21)) is the result of loss of processing of
exon 7 from genomic LP283 sequence (see below). Exon 7 normally encodes the LP283 portion from about
Gly-238 to about Asn-253 (GERRLEQHIPTQAVSN). The alternate predicted mature LP283 sequence (LP344) is
as follows:
MGSGRVPGLCLLVLLVHARAAQYSKAAQDVDECVEGTDNCHIDAICQNTPRSYKCICKSGYTGDGKHCKDVD
ECEREDNAGCVHDCVNIPGNYRCTCYDGFHLAHDGHNCLDVDECAEGNGGCQQSCVNMMGSYECHCREGFFL
SDNQHTCIQRPEEGMNCMNKNHGCAHICRETPKGGIACECRPGFELTKNQRDCKLTCNYGNGGCQHTCDDTE
QGPRCGCHIKFVLHTDGKTCIETCAVNNGGCDSKCHDAATGVHCTCPVGFMLQPDRKTCKDIDECRLNNGGC
DHICRNTVGSFECSCKKGYKLLINERNCQDIDECSFDRTCDHICVNTPGSFQCLCHRGYLLYGITHCGDVDE
CSINRGGCRFGCINTPGSYQCTCPAGQGRLHWNGKDCTEPLKCQGSPAGSKAMLSCNRSGKKDTCALTCPSR
ARFLPESENGFTVSCGTPSPRAAPARAGHNGNSTNSNHCHEAAVLSIKQRASFKIKDADCRLHLRNKGKTEE
AGRITGPGGAPCSECQVTFIHLKCDSSRKGKGRRARTPPGKEVTRLTLELEAEVRAEETTASCGLPCLRQRM
ERRLKGSLKMLRKSINQDRFLLRLAGLDYELAHKPGLVAGERAEPMESCRPGQHRAGTKCVSCPQGTYYHGQ
TEQCVPCPAGTFQEREGQLSCDLCPGSDAHGPLGATNVTTCAGQCPPGQHSVDGFKPCQPCPRGTYQPEACR
TLCFPCGGGLTTKHEGAISFQDCDTKVQCSPGHYYNTSIHRCIRCAMGSYQPDFRQNFCSRCPGNTSTDFDG
STSVAQCKNRQCGGELGEFTGYIESPNYPGNYPAGVECIWNINPPPKRKILIVVPEIFLPSEDECGDVLVMR
KNSSPSSITTYETCQTYERPIAFTARSRKLWINFKTSEANSARGFQIPYVTYDEDYEQLVEDIVRDGRLYAS
ENHQEILKDDDLIKAFFEVLAHPQNFKYTEKHKEMLPKSFIKLLRSKVSSFLRPYK
            The 7th exon below is skipped in LP344 (SCUBE1h2)
LP283 479 ------GGGAAAGGCGGCTAGAGCAGCACATCCCCACTCAAGCCGTTTCTAATG------
Genomic 22120 AAACAGGGGAAAGGCGGCTAGAGCAGCACATCCCCACTCAAGCCGTTTCTAATGGTAAAT
An Alternate LP283 Mature Sequence (939aa):
Encompassed herein are LP283 splice variants. Another such LP variant is listed below. Applicants
discovered that this LP283 variant (also known as LP345, (SEQ ID NO: 22)) is the result of loss of
processing of exon 7 and exon 16 from genomic LP283 sequence (see below). Exon 7 normally encodes the
LP283 portion from about Gly-238 to about Asn-253 (GERRLEQHIPTQAVSN). Exon 16 normally encodes the
LP283 pottion from about Ser-654 to about Ala-706
(SCPQGTYYHGQTEQCVPCPAGTFQEREGQLSCDLCPGSDAHGPLGATNVTTCA). The alternate
predicted mature LP283 sequence (LP345,) is as follows:
MGSGRVPGLCLLVLLVHARAAQYSKAAQDVDECVEGTDNCHIDAICQNTPRSYKCICKSGYTGDGKHCKDVD
ECEREDNAGCVHDCVNIPGNYRCTCYDGFHLAHDGHNCLDVDECAEGNGGCQQSCVNMMGSYECHCREGFFL
SDNQHTCIQRPEEGMNCMNKNHGCAHICRETPKGGIACECRPGFELTKNQRDCKLTCNYGNGGCQHTCDDTE
QGPRCGCHIKFVLHTDGKTCIETCAVNNGGCKSKCHDAATGVHCTCPVGFMLQPDRKTCKDIDECRLNNGGC
DHICRNTVGSFECSCKKGYKLLINERNCQDIDECSFDRTCDHICVNTPGSFQCLCHRGYLLYGITHCGDVDE
CSINRGGCRFGCINTPGSYQCTCPAGQGRLHWNGKDCTEPLKCQGSPGASKAMLSCNRSGKKDTCALTCPSR
ARFLPESENGFTVSCGTPSPRAAPARAGHNGNSTNSNHCHEAAVLSIKQRASFKIKDAKCRLHLRNGKGTEE
AGRITGPGGAPCSECQVTFIHLKCDSSRKGKGRRARTPPGKEVTRLTLELEAEVRAEETTASCGLPCLRQRM
ERRLKGSLKMLRKSINQDRFLLRLAGLDYELAHKPGLVAGERAEPMESCRPGQHRAGTKCGQCPPGQHSVDG
FKPCQPCPRGTYQPEAGRTLCFPCGGGLTTKHEGAISFQCDCTKVQCSPGHYYNTSIHRCIRCAMGSYQPDF
RQNFCSRCPGNTSTDFDGSTSVAQCKNRQCGGELGEFTGYIESPNYPGNYPAGVECIWNINPPPKRKILIVV
PEIFLPSEDECGDVLVMRKNSSPSSITTYETCQTYERPIAFTARSRKLWINFKTSEANSARGFQIPYVTYDE
SYEQLVEDIVRDGRLYASENHQEILKDKKLIKAFFEVLAHPQNYFKYTEKHKEMPKSFIKDLLRSKVSSFLR
PYK
Exon 7 (see above) and exon 16 are missing in LP346. Exon 16 in LP344
is missing in LP345.
ds42405-LP345  GGGCAGCACCGTGCTGGGACCAAGTGTG---------------------------------
ds42406-LP344 GGGCAGCACCGTGCTGGGACCAAGTGTGTCAGCTGCCCGCAGGGAACGTATTACCACGGC
                            ****************************|exon 16 →
ds42405 ------------------------------------------------------------
ds42406 CAGACGGAGCAGTGTGTGCCATGCCCAGCGGGCACCTTCCAGGAGAGAGAAGGGCAGCTC
ds42405 ------------------------------------------------------------
ds42406 TCCTGCGACCTTTGCCCTGGGAGTGATGCCCACGGGCCTCTTGGAGCCAGGAACGTCACC
ds42405 ----------GTCAGTGCCCACCTGGCCAACACTCTGTAGATGGGTTCAAGCCCTGTCAG
ds42406 ACGTGTGCAGGTCAGTGCCCACCTGGCCAACACTCTGTAGATGGGTTCAAGCCCTGTCAG
                ←exon 16|**************************************************
An Alternate LP283 Mature Sequence (265aa):
Encompassed herein are LP283 splice variants. Still another such LP variant is listed below. Applicants
discovered that this LP283 variant (also known as LP346 (SEQ ID NO: 23)) is the result of loss of
processing of an alternative form of exon 7 and the loss of exon 17 from genomic LP283 sequence (see
below). Exon 7 normally encodes the LP283 portion from about Gly-238 to about Asn-253
(GERRLEQHIPTQAVSN). Loss of the alternative form of exon 7 and of exon 17 leads to a truncated LP283
variant and an altered sequence C-terminad to the normal exon 7 amino acid sequence so that the EGF-
like domain sequence
CAVNNGGCDSKCHDAATGVHCTCPVGFMLQPDRKTC is changed to
SGTPSQLHQQPCFFLTNSSLPSLTLI. The alternate predicted mature LP283 sequence (LP346) is as follows:
MGSGRVPGLCLLVLLVHARAAQYSKAAQDVDECVEGTDNCHIDAICQNTPRSYKCICKSGYTGDGKHCKDVD
ECEREDNAGCVHDCVNIPGNYRCTCYDGFHLAHDGHNCLDVDECAEGNGGCQQSCVNMMGSYECHCREGFFL
SDNQHTCIQRPEEGMNCMNKNHGCAHICRETPKGGIACECRPGFELTKNQRDCKLTCNYGNGGCQHTCDDTE
QGPRCGCHIKFVLHTDGKTCIDASGTPSQLHQQPCFFLTNSSPSLTLI
An alternative form of exon 7 (see above) and the reqular form of exon
17 are missing in LP346.
(exon 7)
SCUBE1h4ds  505 ----------------------------------ATGCCAGTGGTACTCCCTCTCAGCTC
Hs6_7350ch 20560 CACCTTACCCCCCATTTCCTTCTCTCTCCTCCAGATGCCAGTGGTACTCCCTCTCAGCTC
                                                                                                      **************************
SCUBE1h4ds  531 CACCAGCAACCCTGTTTCTTCCTCACCAACTCCAGCCTTCCATCTCTTACCTTGATTTGA
Ha6_7350ch 20620 CACCAGCAACCCTGTTTCTTCCTCACCAACTCCAGCCTTCCATCTCTTACCTTGATTTGA
                                  ************************************************************
SCUBE1h4ds  591 GGTCCTCTTAATACCTGGATCCCTCTTCCTGAATTCTTAGGCCTTATCTCACATATTTTC
Hs6_7350ch 20680 GGTCCTCTTAATACCTGGATCCCTCTTCCTGAATTCTTAGGCCTTATCTCACATATTTTC
                                  ************************************************************
SCUBE1h4ds  651 AG**********************************************************
Hs6_7350ch 20740 AGGTACCCTAGATGAGTTTAATTCCTTAAAGTTAATTCCTAATTCCTTTAGCCTTTAGGT
                                    **
cDNA-genomic DNA alignment for exon 7 and 17:
(exon 17)
SCUBE1h4ds  1720 ---------------------------------------------------------CT
Hs6_7350ch 30220 CTGGGTGGTGGGAATGCGGGGGTGGGTGGCTAGCGCGGCCGACTCTCCCTCAGTCAGCT
                                                                                                      **
SCUBE1h4ds  1722 GCCCGCAGGGAACGTATTACCACGGCCAGACGGAGCAGTGTGTGCCATGCCCAGCGGGCA
Hs6_7350ch 30280 GCCCGCAGGCAACGTATTACCACGGCCAGACGGAGCAGTGTGTGCCATGCCCAGCGGGCA
                                  ************************************************************
SCUBE1h4ds  1782 CCTTCCAGGAGAGAGAAGGGCAGCTCTCCTGCGACCTTTGCCCTGGGAGTGATGCCCACG
Hs6_7350ch 30340 CCTTCCAGGAGAGAGAAGGGCAGCTCTCCTGCGACCTTTGCCCTGGGAGTGATGCCCACG
                                  ************************************************************
SCUBE1h4ds  1842 GGCCTCTTGGAGCCACCAACGTCACCACGTGTGCAG------------------------
Hs6_7350ch 30400 GGCCTCTTGGAGCCACCAACGTCACCACGTGTGCAGGTGCCAGGGGAACAAACAATACAG
                                  ************************************
```

Interesting segments of LP283 are the following segments: from about Cys-33 to about Cys-68, from about Cys-74 to about Cys-110, from about Cys-116 to about Cys-151, from about Cys-201 to about Cys-236, from about Cys-256 to about Cys-291, from about Cys-297 to about Cys-332, from about Cys-338 to about Cys-371, from about Cys-377 to about Cys-413, from about Cys-55 to about Cys-68, from about Cys-95 to about Cys-110, from about Cys-136 to about Cys-151, from about Cys-182 to about Cys-197, from about Cys-276 to about Cys-291, from about Cys-317 to about Cys-332, from about Cys-357 to about Cys-371, and from about Cys-161 to about Cys-197, which have been discovered to be EGF-like domains. Additionally interesting segments of LP283 are the segments: from about Asp-29 to about Cys-55, from about Asp-70 to about Cys-95, from about Asp-112 to about Cys-136, from about Asp-293 to about Cys-317, from about Asp-334 to about Cys-357, and from about Asp-151 to about Arg-166 which are all identified as a calcium-binding EGF-like domains. Additional interesting segments of LP283 are: from about Cys-46 to about Cys-57, from about Cys-86 to about Cys-97, from about Cys-127 to about Cys-138, from about Cys-308 to about Cys-319, from about Cys-348 to about Cys-359, and from about Cys-388 to about Cys-399), which have been discovered to be aspartic acid and/or asparagine hydroxylation-like sites. A further interesting segment of LP283 is from about Cys-95 to about Thr-231, which has been discovered to be a keratin B2-like domain. A further interesting segment of LP283 is from about Cys-392 to about Thr-465, which has been discovered to be a metallothionein-like domain. Accordingly, one could test An LP283 or LP283 variant for possible metalloproteinase activity using any standard method in the art without requiring undue experimentation. For example, commercially available kits can be purchased which test for specific matrix metalloproteinase activity (see, e.g., Biotrack MMP Bioassays from Amersham Pharmacia Biotech Limited) or the method of Hojima, et al. (1985 J. Biol. Chem 260:15996-16003; incorporated herein for its assay methods) can be adapted for use with an LP of the invention to test for enzymatic activity, however, other methods are also known and can be adapted for use here given the teachings supplied herein of the LP283 sequence. A further interesting segment of LP283 is from about Cys-820 to about Tyr-929, which has been discovered to be a CUB-like domain. Other interesting segments of LP283 are discovered portions of LP283 from about Leu-11 to about Ser-24; from about Lys-55 to about Asn-79; from about Arg-154 to about Pro-176; from about Lys-192 to about Lys-226; from about His-230 to about Val-251; from about Asn-253 to about Ala-271; from about Thr-272 to about Thr-290; from about Cys-291 to about Cys-308; from about Gly-353 to about Asp-375; from about Cys-377 to about Cys-399; from about Trp-408 to about Gly-421; from about Lys-427 to about Ser-447; from about Ala-449 to about Pro-466; from about Ala-470 to about Ala-499; from about Phe-501 to about Gly-526; from about Pro-527 to about Thr-538; from about Phe-539 to about Val-563; from about Thr-564 to about Gly-584; from about Asn-608 to about Glu-633; from about Ala-635 to about Tyr-660; from about Ala-673 to about Leu-683; from about Cys-688 to about Thr-703; from about Gly-728 to about Glu-751; from about Asp-805 to about Cys-820; from about Phe-827 to about Ile-847; from about Asn-849 to about Val-876; from about Gln-926 to about Val-944; from about Pro-975 to about Ile-994; from about Leu-11 to about Gln-22; from about Lys-25 to about Glu-35; from about Thr-37 to about Arg-51; from about Ser-52 to about Gly-65; from about Gly-65 to about Gly-81; from about Gly-81 to about Gly-91; from about His-137 to about Lys-177; from about Gly-179 to about Phe-227; from about Leu-229 to about Ala-271; from about Phe-282 to about Glu-316; from about Ser-318 to about Ile-347; from about Cys-397 to about Gly-421; from about Gly-424 to about Arg-450; from about Ala-470 to about Ala-499; from about Lys-502 to about Gly-529; from about Phe-539 to about Val-574; from about Thr-579 to about Asn-608; from about His-625 to about Gln-645; from about Gly-644 to about Gly-663; from about Gln-664 to about Glu-678; from about Arg-679 to about Ser-691; from about Ala-706 to about Gly-743; from about His-750 to about Ser-774; from about Ile-775 to about Ser-803; from about Phe-806 to about Gly-821; from about Gly-822 to about Ile-850; from about Lys-881 to about Thr-896; from about Tyr-897 to about Ile-912; from about Asn-920 to about Glu-934; from about Glu-934 to about Leu-949; from about Tyr-950 to about Ala-967; from about Glu-970 to about Lys-995; from about Gly-8 to about Ala-18; from about Arg-19 to about Asp-43; from about Ile-45 to about Ile-56; from about Cys-57 to about His-84; from about Ala-104 to about Asn-129; from about Ser-145 to about His-170; from about Cys-172 to about Glu-189; from about Glu-189 to about Cys-201; from about Asn-202 to about Cys-223; from about His-230 to about Ile-246; from about Pro-247 to about Cys-278; from about Gln-285 to about His-306; from about Cys-308 to about Lys-324; from about Asn-328 to about Leu-358; from about Thr-369 to about Gly-387; from about Asn-390 to about Leu-406; from about Asn-409 to about Met-429; from about Ser-431 to about Pro-446; from about Ala-473 to about Val-492; from about Ser-494 to about Leu-510; from about Leu-512 to about Val-537; from about Cys-544 to about Arg-565; from about Leu-570 to about Leu-585; from about Leu-588 to about Lys-601; from about Leu-629 to about Cys-652; from about Ser-654 to about Cys-671; from about Ala-673 to about Asp-686; from about Leu-687 to about Ala-699; from about Thr-700 to about Phe-719 from about Lys-720 to about Cys-739; from about Pro-741 to about Ser-755; from about Phe-756 to about Arg-777; from about Ile-779 to about Cys-795; from about Cys-795 to about Ala-813; from about Ala-813 to about Thr-828; from about Gly-829 to about Glu-845; from about Lys-857 to about Leu-867; from about Pro-868 to about Leu-877; from about Met-879 to about Pro-900; from about Lys-915 to about Gln-926; from about Val-930 to about Ile-943; from about Val-944 to about Lys-963; from about Leu-964 to about Ala-973; and from about His-974 to about Pro-990; whose discoveries were based on an analysis of hydrophobicity, hydropathicity, and hydrophilicity plots. Additional interesting sections of LP283 are the discovered portions of LP283 from about Leu-12 to about Val-30; from about Asp-31 to about Ile-45; from about Cys-46 to about Tyr-61; from about Thr-62 to about Asp-72; from about Cys-82 to about Tyr-98; from about Asp-99 to about Cys-110; from about Cys-114 to about Val-128; from about Val-129 to about Leu-144; from about Ser-145 to about Gly-167; from about Ile-171 to about Lys-192: from about Lys-198 to about Asp-214; from about Asp-232 to about Glu-244; from about Thr-248 to about Asp-264; from about Ser-265 to about Val-274; from about His-275 to about Asp-287; from about Asp-295 to about His-306; from about Ile-307 to about Cys-319; from about Leu-326 to about Phe-340 from about Arg-342 to about Cys-359; from about Tyr-366 to about Arg-381; from about Ser-378 to about Gly-393; from about Thr-391 to about Arg-405; from about Arg-448 to about Pro-468; from about Asn-485 to about Gln-497; from about Ser-500 to about Gly-516; from about Glu-519 to about Ile-540; from about Glu-578 to about Arg-591; from about Leu-613 to about Val-630; from about Gly-632 to about Gln-645; from about Cys-652 to about Thr-665; from about Glu-666 to about Gln-682; from about Ala-699 to about Asp-717; from about Lys-720 to about Ala-734; from about Ala-734 to about Gly-752; from about Ala-753 to about Val-763; from about Thr773 to about Arg-791; from about Glu-792 to about Ala-813; from about Lys-816 to about Pro-837; from about Gly-838 to about Arg-856; from about Leu-859 to about Glu-872; from about Glu-872 to about Pro-885; from about Ser-886 to about Phe-903; from about Ala-919to about Asp-933; from about Glu-938 to about Arg-948; from about His-955 to about Leu-972; from about Pro-975 to about Pro-990; and from about Ile-994 to about Phe-1004. These fragments were discovered based on analysis of antigenicity plots. Further, particularly interesting LP 283 segments are LP secondary structures (e.g., such as a helix, a strand, or a coil). Particularly interesting LP283 coil structures are the following: from about Met-1 to about Pro-7; from about Glu-35 to about Cys-40; from about Asn-48 to about Ser-52; from about Ser-59 to about Asp-70; from about Arg-76 to about Cys-82; from about Asn-88 to about Asn-92; from about Tyr-98 to about Phe-101 from about His-105 to about Cys-110; from about Glu-118 to about Cys-123; from about Met-131 to about Ser-133; from about Cys-138 to about Gly-141; from about Ser-145 to about His-149; from about Gln-153 to about Asn-160; from about Asn-163 to about Gly-167; from about Glu-174 to about Gly-179; from about Cys-184 to about Asp-196; from about Tyr-203 to about Cys-208; from about Cys-212 to about Gly-222; from about Thr-231 to about Lys-234; from about His-245 to about Asn-253; from about Asn-259 to about Asp-264; from about His-268 to about Gly-273; from about Cys-278 to about Val-280; from about Gln-285 to about Asp-295; from about Asn-300 to about Asp-305; from about Asn-310 to about Ser-314; from about Cys-319 to about Tyr-323; from about Glu-329 to about Asl-345; from about Thr-351 to about Ser-354; from about His-360 to about Tyr-363; from about Thr-369 to about Val-375; from about Ile-379 to about Cys-384; from about Asn-390 to about Ser-394; from about Cys-399 to about Gln-403; from about Asn-409 to about Pro-416; from about Gln-420 to about Ser-426; from about Asn-433 to about Asp-439 to about Thr-444 from about Gly-458; from about Cys-463 to about Asn-485; from about Asn-514 to about Thr-518; from about Thr-525 to about Glu-534; from about Asp-545 to about Gly-552; from about Thr-557 to about Lys-561; from about Cys-583 to about Gly-584; from about Gly-618 to about Gly-628; from about Glu-633 to about Thr-650; from about Cys-655 to about Thr-659; from about His-662 to about Gln-664; from about Val-669 to about Gln-582; from about Leu-687 to about Gly-698; from about Ala-706 to about Arg-736; from about Pro-741 to about Leu-746; from about Glu-751 to about Gly-752; from about Asp-758 to about Thr-761; from about Ser-766 to about Ile-775; from about Gly-784 to about Arg-791; from about Arg-797 to about Thr-810; from about 817 to about Glu-826; from about Ser-833 to about Gly-843; from about Ile-850 to about Lys-855; from about Phe-866 to about Gly-874; from about Asn-882 to about Ser-887; from about Acy-894 to about Pro-900, from about Thr-916 to about Gly-924; from about Tyr-932 to about Asp-933; from about Asp-946 to about Arg-948; from about Ser-952 to about Glu-953; from about Ala-973 to about Gln-976; and from about Pro-1007 to about Lys-1009. Particularly interesting helix structures are from about His-17 to about Ala-27; from about Leu-242 to about Glu-243; from about Leu-570 to about Glu-571; from about Arg-589 to about Leu-596; from about Leu-600 to about Lys-605; from about Tyr-936 to about Arg-945; from about Gln-956 to about Phe-969; and from about Ser-992 to about Leu-997. Particularly interesting strand structures are the following: from about Lys-54 to about Cys-57; from about Cys-95 to about Cys-97; from about Leu-111 to about Val-113; from about Ile-180 to about Cys-182; from about Ile-225 to about Leu-229; from about Cys-256 to about Ala-257; from about His-275 to about Cys-276; from about Cys-297 to about Arg-298; from about His-306 to about Cys-308; from about Leu-325 to about Ile-327; from about His-346 to about Val-349; from about Gln-356 to about Leu-358; from about Leu-364 to about Tyr-366; from about Tyr-395 to about Cys-397; from about Ala-428 to about Leu-430; from about Thr-460 to about Ser-462; from about Val-537 to about Leu-542; from about Cys-652 to about Val-653; from about Val-702 to about Thr-704; from about Thr-637 to about Phe-640; from about Ile-754 to about Phe-756; from about Val-812 to about Ala-813; from about Tyr-830 to about Ile-831 from about Glu-845 to about Trp-848; from about Lys-857 to about Val-861; from about Val-876 to about Arg-880; from about Phe-903 to about Phe-903; and from about Ile-927 to about Val-930. Further encompassed by the invention are contiguous amino acid residue combinations of any of the predicted secondary structures described above. For example, one coil-strand-coil-helix-coil motif of LP283 combines the Cys-212 to about Gly-222 coil; with the Ile-225 to about Leu-229 strand; with the Thr-231 to about Lys-234 coil; with the Leu-242 to about Glu-243 helix; with the His-245 to about Asn 253 coil to form an interesting fragment of contiguous amino acid residues from Cys-212 to Asn-253. Other combinations of contiguous amino acids are contemplated as can be easily determined.

Interesting segments of LP344 are the following segments: from about Cys-33 to about Cys-68, from about Cys-74 to about Cys-110, from about Cys-116 to about Cys-151, from about Cys-201 to about Cys-236, from about Cys-240 to about Cys-275, from about Cys-281 to about Cys-316, from about Cys-322 to about Cys-355, from about Cys-366 to about Cys-397, from about Cys-55 to about Cys-68, from about Cys-95 to about Cys-110, from about Cys-136 to about Cys-151, from about Cys-182 to about Cys-197, from about Cys-260 to about Cys-275, from about Cys-301 to about Cys-316, from about Cys-341 to about Cys-355, and from about Cys-161 to about Cys-197, which have been discovered to be EGF-like domains. Additionally interesting segments of LP344 are the segments: from about Asp-29 to about Cys-55, from about Asp -70 to about Cys-95, from about Asp-112 to about Cys-136, from about Asp-318 to about Cys-341, and from about Asp-151 to about Arg-166 which are all identified as a calcium-binding EGF-like domains. Additional interesting segments of LP344 are: from about Cys-46 to about Cys-57, from about Cys-86 to about Cys-97, from about Cys-127 to about Cys-138, from about Cys-308 to about Cys-319, from about Cys-348 to about Cys-359, and from about Cys-388 to about Cys-399, which have been discovered to be aspartic acid and/or asparagine hydroxylation-like sites. A further interesting segment of LP344 is from about Cys-95 to about Thr-231, which has been discovered to be a keratin B2-like domain. A further interesting segment of LP344 is from about Cys-376 to about Thr-449, which has been discovered to be a metallothionein-like domain. A further interesting segment of LP344 is from about Cys-804 to about Tyr-913, which has been discovered to be a CUB-like domain. Other interesting segments of LP344 are discovered portions of LP344 from about Leu-11 to about Ser-24; from about Lys-55 to about Asn-79; from about Arg-154 to about Pro- 176; from about Lys-192 to about Lys-226; from about Asn-238 to about Ala-255; from about Thr-256 to about Thr-274; from about Cys-275 to about Cys-382; from about Gly-337 to about Asp-359; from about Cys-361 to about Cys-383; from about Trp-492 to about Gly-405; from about Lys-411 to about Ser-431; from about Ala-433 to about Pro-450; from about Ala-454 to about Ala-483; from about Phe-585 to about Gly-510; from about Pro-511 to about Thr-512; from about Phe-522 to about Val-557; from about Thr-558 to about Gly-568; from about Asn-692 to about Glu-617; from about Ala-619 to about Tyr-644; from about Ala-657 to about Leu-667; from about Cys-672 to about Thr-787; from about Gly-712 to about Glu-735; from about Asp-799 to about Cys-804; from about Phe-811 to about Ile-831; from about Asn-833 to about Val-860; from about Gln-901 to about Val-928; from about Pro-959 to about Ile-988; from about Leu-11 to about Gln-22; from about Lys-25 to about Glu-35; from about Thr-37 to about Arg-51; from about Ser-52 to about Gly-65; from about Gly-65 to about Gly-81; from about Gly-81 to about Gly-91; from about His-137 to about Lys-177; from about Gly-179 to about Phe-227; from about Phe-266 to about Glu-300; from about Ser-302 to about Ile-331; from about Cys-381 to about Gly-405; from about Gly-408 to about Arg-434; from about Ala-454 to about Ala-483; from about Lys-487 to about Gly-513; from about Phe-523 to about Val-558; from about Thr-553 to about Asn-592; from about His-609 to about Gln-629; from about Gly-628 to about Gly-647; from about Gln-648 to about Glu-662; from about Arg-663 to about Ser-675; from about Ala-680 to about Gly-727; from about His-734 to about Ser-787; from about Ile-759 to about Ser-787; from about Phe-790 to about Gly-805; from about Gly-806 to about Ile-834; from about Lys-865 to about Thr-880; from about Tyr-881 to about Ile-896; from about Asn-904 to about Glu-918; from about Glu-918 to about Leu-923; from about Tyr-934 to about Ala-951; from about Glu-954 to about Lys-979; from about Gly-8 to about Ala-18; from about Arg-19 to about Asp-43; from about Ile-45 to about Ile-56; from about Cys-57 to about His-84; from about Ala-104 to about Asn-129; from about Ser-145 to about His-170; from about Cys-172 to about Glu-189; from about Glu-189 to about Cys-201; from about Asn-202 to about Cys-223; from about Pro-221 to about Cys-212; from about Gln-269 to about His-290; from about Cys-292 to about Lys-308; from about Asn-312 to about Leu-342; from about Thr-353 to about Gly-321; from about Asn-374 to about Leu-390; from about Asn-393 to about Met-413; from about Ser-415 to about Pro-430; from about Ala-457 to about Val-476; from about Ser-478 to about Leu-494; from about Leu-494 to about Val-521; from about Cys-538 to about Arg-549; from about Leu-554 to about Leu-569; from about Leu-572 to about Lys-585; from about Leu-613 to about Cys-636; from about Ser-638 to about Cys-655; from about Ala-657 to about Asp-670; from about Leu-671 to about Ala-683; from about Thr-684 to about Phe-703; from about Lys-704 to about Cys-713; from about Pro-725 to about Ser-739, from about Phe-740 to about Arg-761; from about Ile-713 to about Cys-789; from about Cys-779 to about Ala-797; from about Ala-797 to about Thr-812; from about Gly-813 to about Glu-829; from about Lys-831 to about Leu-851; from about Pro-852 to about Leu-861; from about Met-863 to about Pro-884; from about Lys-899 to about Gln-910; from about Val-914 to about Ile-927; from about Val-928 to about Lys-947; from about Leu-948 to about Ala-957; and from about His-958 to about Pro-974; whose discoveries were based on an analysis of hydrophobicity, hydropathicity, and hydrophilicity plots. Additional interesting sections of LP344 are the discovered portions of LP344 from about Leu-12 to about Val-30; from about Asp-31 to about Ile-45; from about Cys-46 to about Tyr-61; from about Thr-62 to about Asp-72; from about Cys-82 to about Tyr-98; from about Asp-99 to about Cys-110; from about Cys-114 to about Val-128; from about Val-129 to about Leu-144; from about Ser-145 to about Gly-167; from about Ile-171 to about Lys-192: from about Lys-198 to about Asp-214; from about Thr-232 to about Asp-248; from about Ser-249 to about Val-258; from about His-259 to about Asp-271; from about Asp-279 to about His-290; from about Ile-291 to about Cys-303; from about Leu-280 to about Phe-324; from about Arg-325 to about Cys-343; from about Tyr-350 to about Arg-365; from about Ser-362 to about Gly-367; from about Thr-375 to about Arg-389; from about Arg-432 to about Pro-452; from about Asn-469 to about Gln-481; from about Ser-484 to about Gly-500; from about Glu-503 to about Ile-524; from about Glu-562 to about Arg-575; from about Leu-597 to about Val-614; from about Gly-616 to about Gln-629; from about Cys-636 to about Thr-649; from about Glu-650 to about Gln-666; from about Ala-683 to about Asp-701; from about Lys-704 to about Ala-728; from about Ala-718 to about Gly-736; from about Ala-737 to about Val-747; from about Thr-757 to about Arg-775; from about Glu-776 to about Ala-797; from about Lys-800 to about Pro-821; from about Gly-822 to about Arg-840; from about Leu-843 to about Glu-856; from about Glu-856 to about Pro-869; from about Ser-870 to about Phe-887; from about Ala-903 to about Asp-917; from about Glu-922 to about Arg-932; from about His-939 to about Leu-955; from about Pro-959 to about Pro-974; and from about Ile-978 to about Phe-988. These fragments were discovered based on analysis of antigenicity plots. Further, particularly interesting LP 283 segments are LP secondary structures (e.g., such as a helix, a strand, or a coil). Particularly interesting LP344 coil structures are the following: from about Met-1 to about Pro-7; from about Glu-35 to about Cys-40; from about Asn-48 to about Ser-52; from about Ser-59 to about Asp-70; from about Arg-76 to about Cys-82; from about Asn-88 to about Asn-92; from about Tyr-98 to about Phe-101; from about His-105 to about Cys-110; from about Glu-118 to about Cys-123; from about Met-131 to about Ser-133; from about Cys-138 to about Gly-141; from about Ser-145 to about His-149; from about Gln-153 to about Asn-160; from about Asn-163 to about Gly-167; from about Glu-174 to about Gly-179; from about Cys-184 to about Asp-196; from about Tyr-203 to about Cys-208; from about Cys-212 to about Gly-222; from about Thr-231 to about Lys-234; from about His-229 to about Asn-237; from about Asn-243 to about Asp-248; from about His-252 to about Gly-257; from about Cys-262 to about Val-274; from about Gln-269 to about Asp-279; from about Asn-284 to about Asp-289; from about Asn-294 to about Ser-298; from about Cys-303 to about Tyr-307; from about Glu-313 to about Asl-329; from about Thr-335 to about Ser-338; from about His-344 to about Tyr-347; from about Thr-353 to about Val-359; from about Ile-363 to about Cys-368; from about Asn-374 to about Ser-378; from about Cys-383 to about Gln-387; from about Asn-393 to about Pro-400, from about Gln-404 to about Ser-410; from about Asn-417 to about Asp-423 to about Thr-432 from about Gly-452; from about Cys-447 to about Asn-469; from about Asn-482 to about Thr-486; from about Thr-493 to about Glu-528; from about Asp-539 to about Gly-536; from about Thr-541 to about Lys-545; from about Cys-567 to about Gly-568; from about Gly-602 to about Gly-612; from about Glu-617 to about Thr-634; from about Cys-639 to about Thr-643; from about His-646 to about Gln-648; from about Val-653 to about Gln-566; from about Leu-671 to about Gly-682; from about Ala-690 to about Arg-720; from about Pro-725 to about Leu-730; from about Glu-735 to about Gly-736; from about Asp-742 to about Thr-745; from about Ser-750 to about Ile-759; from about Gly-768 to about Arg-775; from about Arg-771 to about Thr-794; from about 801 to about Glu-810; from about Ser-817 to about Gly-827; from about Ile-834 to about Lys-839; from about Phe-850 to about Gly-858; from about Asn-866 to about Ser-871; from about Acy-878 to about Pro-874; from about Thr-900 to about Gly-908; from about Tyr-916 to about Asp-927; from about Asp-930 to about Arg-931; from about Ser-936 to about Glu-947; from about Ala-956 to about Gln-960; and from about Pro-991 to about Lys-1000. Particularly interesting helix structures are from about His-17 to about Ala-27; from about Leu-554 to about Glu-555; from about Arg-573 to about Leu-580; from about Leu-574 to about Lys-589; from about Tyr-920 to about Arg-929, from about Gln-940 to about Phe-953; and from about Ser-986 to about Leu-981. Particularly interesting strand structures are the following: from about Lys-54 to about Cys-57; from about Cys-95 to about Cys-97; from about Leu-111 to about Val-113; from about Ile-180 to about Cys-182; from about Ile-225 to about Leu-229; from about Cys-240 to about Ala-241; from about His-269 to about Cys-260; from about Cys-281 to about Arg-282; from about His-290 to about Cys-292; from about Leu-309 to about Ile-301; from about His-330 to about Val-333; from about Gln-340 to about Leu-342; from about Leu-358 to about Tyr-350; from about Tyr-379 to about Cys-371; from about Ala-412 to about Leu-414; from about Thr-444 to about Ser-446; from about Val-521 to about Leu-526; from about Cys-636 to about Val-637; from about Val-676 to about Thr-678; from about Thr-621 to about Phe-624; from about Ile-738 to about Phe-738; from about Val-794 to about Ala-797; from about Tyr-814 to about Ile-815; from about Glu-829 to about Trp-832; from about Lys-841 to about Val-845; from about Val-860 to about Arg-864; from about Phe-887 to about Phe-887; and from about Ile-911 to about Val-914. Further encompassed by the invention are contiguous amino acid residue combinations of any of the predicted secondary structures described above. For example, one coil-strand-coil-helix-coil motif of LP344 combines the Cys-212 to about Gly-222 coil; with the Ile-225 to about Leu-229 strand; with the Thr-231 to about Lys-234 coil; to form an interesting fragment of contiguous amino acid residues from Cys-212 to Lys-234. Other combinations of contiguous amino acids are contemplated as can be easily determined.

Interesting segments of LP345, are the following segments: from about Cys-33 to about Cys-68, from about Cys-74 to about Cys-110, from about Cys-116 to about Cys-151, from about Cys-201 to about Cys-236, from about Cys-240 to about Cys-275, from about Cys-281 to about Cys-316, from about Cys-322 to about Cys-355, from about Cys-366 to about Cys-397, from about Cys-55 to about Cys-68, from about Cys-95 to about Cys-110, from about Cys-136 to about Cys-151, from about Cys-182 to about Cys-197, from about Cys-260 to about Cys-275, from about Cys-301 to about Cys-316, from about Cys-341 to about Cys-355, and from about Cys-161 to about Cys-197, which have been discovered to be EGF-like domains. Additionally interesting segments of LP345, are the segments: from about Asp-29 to about Cys-55, from about Asp -70 to about Cys-95, from about Asp-112 to about Cys-136, from about Asp-318 to about Cys-341, and from about Asp-151 to about Arg-166 which are all identified as a calcium-binding EGF-like domains. Additional interesting segments of LP345, are from about Cys-46 to about Cys-57, from about Cys-86 to about Cys-97, from about Cys-127 to about Cys-138, from about Cys-308 to about Cys-319, from about Cys-348 to about Cys-359, and from about Cys-388 to about Cys-399, which have been discovered to be aspartic acid and/or asparagine hydroxylation-like sites. A further interesting segment of LP345, is from about Cys-95 to about Thr-231, which has been discovered to be a keratin B2-like domain. A further interesting segment of LP345, is from about Cys-376 to about Thr-449, which has been discovered to be a metallothionein-like domain. A further interesting segment of LP345, is from about Cys-750 to about Tyr-859, which has been discovered to be a CUB-like domain. Other interesting segments of LP345, are discovered portions of LP345, from about Leu-11 to about Ser-24; from about Lys-55 to about Asn-79; from about Arg-154 to about Pro-176; from about Lys-192 to about Lys-226; from about Asn-238 to about Ala-255; from about Thr-256 to about Thr-274; from about Cys-275 to about Cys-382; from about Gly-337 to about Asp-359; from about Cys-361 to about Cys-383; from about Trp-492 to about Gly-405; from about Lys-41 1 to about Ser-431; from about Ala-433 to about Pro-450; from about Ala-454 to about Ala-483; from about Phe-585 to about Gly-510; from about Pro-511 to about Thr-512; from about Phe-522 to about Val-557; from about Thr-558 to about Gly-568; from about Asn-692 to about Glu-617; from about Gly-658 to about Glu-681; from about Asp-735 to about Cys-750; from about Phe-757 to about Ile-777; from about Asn-779 to about Val-806; from about Gln-856 to about Val-874; from about Pro-905 to about Ile-924; from about Leu-11 to about Gln-22; from about Lys-25 to about Glu-35; from about Thr-37 to about Arg-51; from about Ser-52 to about Gly-65; from about Gly-65 to about Gly-81; from about Gly-81 to about Gly-91; from about His-137 to about Lys-177; from about Gly-179 to about Phe-227; from about Phe-266 to about Glu-300; from about Ser-302 to about Ile-331; from about Cys-381 to about Gly-405; from about Gly-408 to about Arg-434; from about Ala-454 to about Ala-483; from about Lys-487 to about Gly-513; from about Phe-523 to about Val-558; from about Thr-553 to about Asn-592; from about His-609 to about Gln-629; from about His-680 to about Ser-733; from about Ile-705 to about Ser-733; from about Phe-746 to about Gly-751; from about Gly-752 to about Ile-780; from about Lys-811 to about Thr-836; from about Tyr-827 to about Ile-842; from about Asn-850 to about Glu-864; from about Glu-864 to about Leu-869; from about Tyr-880 to about Ala-897; from about Glu-900 to about Lys-925; from about Gly-8 to about Ala-18; from about Arg-19 to about Asp-43; from about Ile-45 to about Ile-56; from about Cys-57 to about His-84; from about Ala-104 to about Asn-129; from about Ser-145 to about His-170; from about Cys-172 to about Glu-189; from about Glu-189 to about Cys-201; from about Asn-202 to about Cys-223; from about Pro-221 to about Cys-212; from about Gln-269 to about His-290; from about Cys-292 to about Lys-308, from about Asn-312 to about Leu-342; from about Thr-353 to about Gly-321; from about Asn-374 to about Leu-390; from about Asn-393 to about Met-413; from about Ser-415 to about Pro-430; from about Ala-457 to about Val-476; from about Ser-478 to about Leu-494; from about Leu-494 to about Val-521; from about Cys-538 to about Arg-549; from about Leu-554 to about Leu-569; from about Leu-572 to about Lys-585; from about Leu-613 to about Cys-636; from about Ser-584 to about Cys-601; from about Ala-603 to about Asp-626; from about Leu-627 to about Ala-639; from about Thr-630 to about Phe-651 from about Lys-650 to about Cys-659; from about Pro-671 to about Ser-68⁵; from about Phe-686 to about Arg-707; from about Ile-656 to about Cys-735; from about Cys-725 to about Ala-743; from about Ala-743 to about Thr-768; from about Gly-764 to about Glu-875; from about Lys-777 to about Leu-797; from about Pro-798 to about Leu-807; from about Met-809 to about Pro-830; from about Lys-845 to about Gln-856; from about Val-860 to about Ile-873; from about Val-874 to about Lys-893; from about Leu-894 to about Ala-903; and from about His-904 to about Pro-920; whose discoveries were based on an analysis of hydrophobicity, hydropathicity, and hydrophilicity plots. Additional interesting sections of LP345, are the discovered portions of LP345, from about Leu-12 to about Val-30; from about Asp-31 to about Ile-45; from about Cys-46 to about Tyr-61; from about Thr-62 to about Asp-72; from about Cys-82 to about Tyr-98; from about Asp-99 to about Cys-110; from about Cys-114 to about Val-128; from about Val-129 to about Leu-144; from about Ser-145 to about Gly-167; from about Ile-171 to about Lys-192: from about Lys-198 to about Asp-214; from about Thr-232 to about Asp-248; from about Ser-249 to about Val-258; from about His-259 to about Asp-271; from about Asp-279 to about His-290; from about Ile-291 to about Cys-303; from about Leu-280 to about Phe-324; from about Arg-325 to about Cys-343; from about Tyr-350 to about Arg-365; from about Ser-362 to about Gly-367; from about Thr-375 to about Arg-389; from about Arg-432 to about Pro-452; from about Asn-469 to about Gln-481; from about Ser-484 to about Gly-500; from about Glu-503 to about Ile-524; from about Glu-562 to about Arg-575; from about Leu-597 to about Val-614; from about Gly-616 to about Gln-629; from about Lys-850 to about Ala-674; from about Ala-664 to about Gly-682; from about Ala-683 to about Val-693; from about Thr-703 to about Arg-721; from about Glu-722 to about Ala-743; from about Lys-746 to about Pro-767; from about Gly-768 to about Arg-786; from about Leu-789 to about Glu-802; from about Glu-802 to about Pro-815; from about Ser-826 to about Phe-833; from about Ala-851 to about Asp-863; from about Glu-878 to about Arg-878; from about His-885 to about Leu-901; from about Pro-904 to about Pro-920; and from about Ile-924 to about Phe-934. These fragments were discovered based on analysis of antigenicity plots. Further, particularly interesting LP 283 segments are LP secondary structures (e.g., such as a helix, a strand, or a coil). Particularly interesting LP345, coil structures are the following: from about Met-1 to about Pro-7; from about Glu-35 to about Cys-40; from about Asn-48 to about Ser-52; from about Ser-59 to about Asp-70; from about Arg-76 to about Cys-82; from about Asn-88 to about Asn-92; from about Tyr-98 to about Phe-101; from about His-105 to about Cys-110; from about Glu-118 to about Cys-123; from about Met-131 to about Ser-133; from about Cys-138 to about Gly-141; from about Ser-145 to about His-149; from about Gln-153 to about Asn-160; from about Asn-163 to about Gly-167; from about Glu-174 to about Gly-179; from about Cys-184 to about Asp-196; from about Tyr-203 to about Cys-208; from about Cys-212 to about Gly-222; from about Thr-231 to about Lys-234; from about His-229 to about Asn-237; from about Asn-243 to about Asp-248; from about His-252 to about Gly-257; from about Cys-262 to about Val-274; from about Gln-269 to about Asp-279; from about Asn-284 to about Asp-289;.from about Asn-294 to about Ser-298; from about Cys-303 to about Tyr-307; from about Glu-313 to about Asl-329; from about Thr-335 to about Ser-338; from about His-344 to about Tyr-347; from about Thr-353 to about Val-359; from about Ile-363 to about Cys-368; from about Asn-374 to about Ser-378; from about Cys-383 to about Gln-387; from about Asn-393 to about Pro-400; from about Gln-404 to about Ser-410; from about Asn-417 to about Asp-423 to about Thr-432 from about Gly-452; from about Cys-447 to about Asn-469; from about Asn-482 to about Thr-486; from about Thr-493 to about Glu-528; from about Asp-539 to about Gly-536; from about Thr-541 to about Lys-545; from about Cys-567 to about Gly-568; from about Gly-602 to about Gly-612; from about Glu-617 to about Thr-634; from about Pro-671 to about Leu-676; from about Glu-681 to about Gly-682; from about Asp-698 to about Thr-691; from about Ser-696 to about Ile-705; from about Gly-714 to about Arg-721; from about Arg-717 to about Thr-720; from about 747 to about Glu-756; from about Ser-763 to about Gly-773; from about Ile-780 to about Lys-785; from about Phe-796 to about Gly-804; from about Asn-812 to about Ser-817; from about Acy-824 to about Pro-820; from about Thr-846 to about Gly-854; from about Tyr-862 to about Asp-873; from about Asp-876 to about Arg-878; from about Ser-872 to about Glu-893; from about Ala-902 to about Gln-906; and from about Pro-937 to about Lys-946. Particularly interesting helix structures are from about His-17 to about Ala-27; from about Leu-554 to about Glu-555; from about Arg-573 to about Leu-580; from about Leu-574 to about Lys-589; from about Tyr-866 to about Arg-875; from about Gln-886 to about Phe-899; and from about Ser-932 to about Leu-937. Particularly interesting strand structures are the following: from about Lys-54 to about Cys-57; from about Cys-95 to about Cys-97; from about Leu-111 to about Val-113; from about Ile-180 to about Cys-182; from about Ile-225 to about Leu-229; from about Cys-240 to about Ala-241; from about His-269 to about Cys-260; from about Cys-281 to about Arg-282; from about His-290 to about Cys-292; from about Leu-309 to about Ile-301; from about His-330 to about Val-333; from about Gln-340 to about Leu-342; from about Leu-358 to about Tyr-350; from about Tyr-379 to about Cys-371; from about Ala-412 to about Leu-414; from about Thr-444 to about Ser-446; from about Val-521 to about Leu-526; from about Val-622 to about Thr-624; from about Thr-567 to about Phe-570; from about Ile-684 to about Phe-684; from about Val-720 to about Ala-743; from about Tyr-760 to about Ile-761; from about Glu-765 to about Trp-778; from about Lys-787 to about Val-791; from about Val-806 to about Arg-810; from about Phe-833 to about Phe-833; and from about Ile-857 to about Val-860. Further encompassed by the invention are contiguous amino acid residue combinations of any of the predicted secondary structures described above. For example, one coil-strand-coil-helix-coil motif of LP345, combines the Cys-212 to about Gly-222 coil; with the Ile-2²⁵ to about Leu-229 strand; with the Thr-231 to about Lys-234 coil; to form an interesting fragment of contiguous amino acid residue, from Cys-212 to Lys-234. Other combinations of contiguous amino acids are contemplated as can be easily determined.

Other interesting segments of LP346 are discovered portions of LP346 from about Pro-7 to about His-17; from about Ala-18 to about Glu-32; from about Cys-33 to about Ile-43; from about Asp-43 to abort Ser-52; from about Lys-58 to about Val-71; from about Asp-72 to about Asn-88; from about Ala-104 to about Val-113; from about Asp-114 to about Asn-129; from about Asp-146 to about Met-159; from about Asn-160 to about His-170; from about Ile-171 to about Pro-186; from about Cys-201 to about His-210; from about Thr-211 to about Cys-218; from about Thr-231 to about Gln-249; from about Pro-250 to about Leu-259; from about Leu-11 to about Ala-20; from about Ala-21 to about Glu-32; from about Cys-33 to about Ile-42; froma about Asp-43 to about Cys-57; from about Lys-58 to about Val-71; from about Asp-72 to about Asp-85; from about Ile-89 to about Asp-99; from about Gly-100 to about Leu-111; from about Asp-112 to about Val-128; from about Asn-129 to about Leu-144; from about Ser-145 to about Glu-157; from about Gly-158 to about Ala-169; from about His-70 to about Glu-183; from about Cys-184 to about Lys- 198; from about Leu-199 to about Cys-212; from about Asp-213 to about Cys-223; from about Thr-231 to about Thr-242; from about Pro-243 to about Cys-251; from about Arg-19 to about Val-30; from about Asp 31 to about Ala-44; from about Ile-45 to about Ile-56; from about Cys-57 to about Val-71; from about Asp-72 to about Asn-88; from about Ile-89 to about Gly-100; from about Phe-101 to about Gly-119; from about Asn-120 to about Asn-129; from about Met-130 to about Phe-142; from about Phe-143 to about Gly-158; from about Met-159 to about His-170; from about Ile-171 to about Cys-184; from about Arg-185 to about Leu-199; from about Thr-100 to about Cys-212; from about Asp-213 to about Lys-226; from about Val-228 to about Ser-240; and from about Gly-241 to about Cys-251, whose discoveries were based on an analysis of hydrophobicity, hydropathicity, and hydrophilicity plots. Additional interesting sections of LP346 are the discovered portions of LP346 from about Leu-11 to about Ala-21; from about Gln-22 to about Asp-31; from about Glu-32 to about Ile-42; from about Asp-43 to about Tyr-53; from about Tyr-53 to about Thr-62; from about Gly-63 to about Cys-74; from about Glu-75 to about Val-87; from about Asn-88 to about Tyr-98; from about Asp-99 to about Asp-112; from about Val-113 to about Gln-124; from about Gln-125 to about Phe-142; from about Leu-144 to about Glu-157; from about Gly-158 to about Cys-168; from about Cys-172 to about Cys-182; from about Glu-183 to about Lys-192; from about Leu-199 to about Cys-208; from about Gln-209 to about His-224; and from about Gln-245 to about Leu-259. These fragments were discovered based on analysis of antigenicity plots. Further, particularly interesting LP346 segments are LP secondary structures (e.g., such as a helix, a strand, or a coil). Particularly interesting LP346 coil structures are the following: from about Met-1 to about Pro-7; from about Glu-35 to about Cys-40; from about Asn-48 to bout Ser-52; from about Ser-59 to about Asp-70; from about Arg-76 to about Cys-82; from about Asn-88 to about Asn-92; from about Tyr-98 to about Phe-101; from about His-105 to about Cys-110; from about Glu-118 to about Cys-123; from about Met-131 to about Ser-133; from about Cys-138 to about Gly-141; from abou Ser-145 to about His-149; from about Gln-153 to about Asn-160; from about Asn-163 to about Cys-168; from about Arg-173 to about Gly-179; from about Cys-184 to about Asp-196; from about Tyr-203 to about Cys-208; from about Cys-212 to about Gly-222; from about Thr-231 to about Lys-234; from about Ala-239 to about Pro-250; from about Thr-255 to about Ser-261; and from about Ile-265 to Ile-265. A particularly interesting helix structure is from about His-17 to about Ala-27. Particularly interesting strands are from about Lys-54 to about Cys-57; from about Cys-95 to about Cys-97; from about Leu-111 to about Val- 113; from about Ile-180 to about Cys-182; and from about Ile-225 to about Leu-229. Further encompassed by the invention are contiguous amino acid residue combinations of any of the predicted secondary structures described above. For example, one coil-strand-coil-coil motif of LP346 combines the His-105 to about Cys-110 coil; with the Leu-111 to about Val-113 strand; with the Glu-118 to about Cys-123 coil; with the Met-131 to about Gly-141 coil to form an interesting fragment (of contiguous amino acid residues from about His-105 to about Gly-141. Other combinations of contiguous amino acids are contemplated as can be easily determined

LP283 and variants Functions: Given the teachings supplied herein, for example, of: LP283 (or variants) primary amino acid, the sequence information and knowledge of the secondary structural features of proteins that exhibit sequence similarity to LP283 or variants, such as, for example, SCUBE1, SCUBE2, Drosophila tolloid, the mammalian tolloid-related genes BMP1 and mTll, fibropellin I and III from sea urchin, and the serum glycoprotein attractin sequence, and how these features map onto LP283 sequence presented herein (e.g., such as the relationship between the primary amino acid sequence of LP283 active regions and higher order structure of similar CUB-like domains such as, the crystal structure of 1SPP (Romero, et al. 1997 Nat Struct Biol. Oct;4(10):783-788 tided, “The crystal structures of two spermadhesins reveal the CUB domain fold.” The three dimensional structure is available from the Protein Data Bank as entry 1SPP)), it is likely that an LP283, an LP283 variant, and/or an LP283 binding agent (e.g., such as an LP283 antibody (or fragment thereof)) plays a similar role/s in a variety of physiological processes. Some non-limiting examples of functions an LP283, LP283 variant, or an LP283 binding agent is likely to participate in are, for example, those such as: a cell adhesion; cell-matrix adhesion; neural development, such as, e.g., brain development, sense organ development, such as, for example, the eye; limb development; protein-protein interactions; protein-extracellular matrix interactions; chemotaxis; metalloproteinase activity, added hair growth/hair replacement, cause breast cancer and embryogenesis. Other combinations of contiguous amino acids are contemplated as can be easily determined.

TABLE 9

| | | | | | | | 5 ′NT of | | | | | |
|---|---|---|---|---|---|---|---|---|---|---|---|---|
| | | | | | 3′ NT | | First | AA | First | | | |
| | | NT SEQ | Total | 5′ NT of | of | | AA of | SEQ | AA of | | First AA | |
| LP | CDNA | ID | NT | Clone | Clone | | signal | ID | Sig | Last AA | Secreted | Last AA of |
| NO. | Clone ID | NO: X | (bp) | SEQ. | SEQ. | ORF | Pep. | NO Y: | Pep | of Sig Pep | Portion | ORF |
| 1 | LP318a | 1 | 685 | 1 | 685 | 41-679 | 41 | 2 | Met-1 | Lys-40 | Ala-41 | Asp-172 |
| 2 | LP318b | 3 | 508 | 1 | 508 | 41-502 | 41 | 4 | — | — | Met-1 | Glu-153 |
| 3 | LP288 | 5 | 7974 | 1 | 7974 | 142-5859 | 142 | 6 | Met-1 | Ala-20 | Ser-21 | Val-1905 |
| 4 | LP289 | 7 | 2653 | 1 | 2653 | 17-1027 | 17 | 8 | Met-1 | Ser-30 | Gln-31 | Met-336 |
| 4 | LP343 | 9 | 754 | 1 | 754 | 19-120 | 19 | 10 | — | — | Met-1 | Thr-33 |
| 5 | LP319a | 11 | 2597 | 1 | 2597 | 62-787 | 62 | 12 | Met-1 | Ser-30 | Gln-31 | Tyr-241 |
| 6 | LP319b | 13 | 2601 | 1 | 2601 | 21-791 | 21 | 14 | Met-1 | Ser-30 | Gln-31 | Tyr-256 |
| 7 | LP321 | 15 | 480 | 1 | 480 | 75-338 | 75 | 16 | Met-1 | Ala-19 | Asp-20 | Ser-87 |
| 8 | LP317 | 17 | 540 | 1 | 540 | 97-345 | 97 | 18 | Met-1 | Gly-25 | Gly-26 | Cys-82 |
| 9 | LP283 | 19 | 3183 | 1 | 3183 | 100-3129 | 100 | 20 | Met-1 | Ala-27 | Ala-28 | Lys-1009 |
| 9 | LP344 | | | 1 | | | | 21 | Met-1 | Ala-27 | Ala-28 | Lys-993 |
| 9 | LP345 | | | 1 | | | | 22 | Met-1 | Ala-27 | Ala-28 | Glu-939 |
| 9 | LP346 | | | 1 | | | | 23 | Met-1 | Ala-27 | Ala-28 | Ile-265 |

Table 9 summarizes information corresponding to each “LP No.” of the invention as described herein. The column labeled, “Total NT Seq.” refers to the total number of nucleotides in a polynucleotide sequence identified by an “LP No.” The nucleotide position of SEQ ID NO: X of the putative start codon (methionine) is identified as “5′ NT of Start Codon.” Similarly, the nucleotide position of SEQ ID NO: X of a predicted signal sequence of an LP protein or polypeptide is identified as “5′ NT of First AA of Signal Pep.”

The corresponding translated amino acid sequence of a particular NT SEQ ID NO:X, typically beginning with the methionine, is identified as “AA SEQ ID NO: Y,” although other reading frames can also be easily translated using techniques known in molecular biology. A polypeptide produced using an alternative open reading frame/s is also specifically encompassed by the present invention. The first and last amino acid position of a SEQ ID NO: Y of the predicted signal peptide is identified as “First AA of Signal Pep” and “Last AA of Signal Pep.” The predicted first amino acid position of SEQ ID NO: Y of the secreted portion is identified as “Predicted First AA of Secreted Portion.” Finally, the amino acid position of SEQ ID NO: Y of the last amino acid in the open reading frame is identified as “Last AA of ORF.”

An LP polypeptide or fragment thereof, identified from SEQ ID NO: Y may be used, e.g., as an immunogen to generate an antibody that specifically and/or selectively binds a protein comprising an LP polypeptide sequence (or fragment thereof) of the invention and/or to a mature LP polypeptide or secreted LP protein, e.g., encoded by a polynucleotide sequence described herein. An LP polypeptide of the invention can be prepared in any manner suitable to those known in the art. Such a polypeptide includes, e.g., naturally occurring polypeptides that are isolated, recombinantly produced polypeptides, synthetically produced polypeptides, or polypeptides produced by any combination of these methods. Means for preparing such polypeptides are well understood in the art. An LP polypeptide (or fragment thereof) may be in the form of, a mature polypeptide, a secreted protein (including the mature form), or it may be a fragment thereof, or it may be a part of a larger polypeptide or protein, such as, e.g., a fusion protein.

It is often advantageous to include with an LP polypeptide (or fragment thereof), e.g., additional amino acid sequence that contains, e.g., secretory or leader sequences, pro-sequences, sequences that aid in purification, such as, e.g., multiple histidine residues, or an additional sequence for stability during recombinant production. Such variants are also encompassed herein. An LP polypeptide (or fragment thereof) is preferably provided in an isolated or recombinant form, or it may be preferably substantially purified. A recombinantly produced version of an LP polypeptide of the invention, including a secreted polypeptide, can be substantially purified using techniques described herein or otherwise known in the art, such as, e.g., the single-step purification method (Smith and Johnson (1988) Gene 67(1):31-40). An LP polypeptide (or fragment thereof) can also be purified from natural, synthetic or recombinant sources using techniques described herein or otherwise known in the art, such as, e.g., using an antibody of the invention raised against a secreted protein. The present invention provides an isolated or recombinant LP polynucleotide comprising, or alternatively consisting of, a nucleic acid molecule having a mature polynucleotide sequence of SEQ ID NO: X wherein said polynucleotide sequence or said cDNA encodes at least 12 contiguous amino acids of a mature polypeptide of SEQ ID NO: Y.

II. Definitions

LP Polynucleotide

As used herein, the term “LP polynucleotide” refers to a molecule comprising a nucleic acid sequence contained in a Table herein or in a sequence of SEQ ID NO:X. For example, the polynucleotide can contain the nucleotide sequence of the full length cDNA sequence, including the 5′ and 3′ untranslated sequences, the coding region, with or without the signal sequence, the secreted protein coding region, as well as fragments, epitopes, domains, and variants of the nucleic acid sequence. An “LP polynucleotide” also encompasses, e.g., those polynucleotides that stably hybridize, under stringent hybridization conditions to an LP sequence of a table herein, or to a sequence contained in SEQ ID NO:X. In specific embodiments, an LP polynucleotide sequence is at least 15, at least 30, at least 50, at least 100, at least 125, at least 500, or at least 1000 contiguous nucleotides but are less than or equal to 300 kb, 200 kb, 100 kb, 50 kb, 15 kb, 10 kb, 7.5 kb, 5 kb, 2.5 kb, 2.0 kb, or 1 kb, in length.

An LP polynucleotide sequence can be composed of any polyribonucleotide or polydeoxribonucleotide, which may be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single-and double-stranded regions. In addition, the polynucleotide can be composed of triple-stranded regions comprising RNA or DNA or both RNA and DNA. A polynucleotide may also contain one or more modified bases or DNA or RNA backbones modified for stability or for other reasons. “Modified” bases can include, e.g., for example, tritylated bases and unusual bases such as inosine. A variety of modifications can be made to DNA and RNA; thus, the term “polynucleotide” embraces chemically, enzymatically, or metabolically modified forms. “Altered” nucleic acid sequences encoding LP include those sequences with deletions, insertions, or substitutions of different nucleotides, resulting in a polypeptide the same as LP or a polypeptide with at least one functional characteristic of LP. Included within this definition are polymorphisms which may or may not be readily detectable using a particular oligonucleotide probe of the polynucleotide encoding LP, and improper or unexpected hybridization to allelic variants, with a locus other than the normal chromosomal locus for the polynucleotide sequence encoding LP.

“Substantial similarity” in a nucleic acid sequence comparison context means either that the segments, or their complementary strands, when compared, are identical when optimally aligned, with appropriate nucleotide insertions or deletions, in at least about 50% of the nucleotides, generally at least 56%, more generally at least 59%, ordinarily at least 62%, more ordinarily at least 65%, often at least 68%, more often at least 71%, typically at least 74%, more typically at least 77%, usually at least 80%, more usually at least about 85%, preferably at least about 90%, more preferably at least about 95 to 98% or more, and in particular embodiments, as high at about 99% or more of the nucleotides. Alternatively, substantial similarity exists when the segments will hybridize under selective hybridization conditions, to a strand, or its complement, typically using a sequence derived from SEQ ID X. Typically, selective hybridization will occur when there is at least about 55% similarity over a stretch of at least about 30 nucleotides, preferably at least about 650% over a stretch of at least about 25 nucleotides, more preferably at least about 75%, and most preferably at least about 90% over about 20 nucleotides. See Kanehisa (1984) Nuc. Acids Res. 12:203-213. The length of similarity comparison, as described, may be over longer stretches, and in certain embodiments will be over a stretch of at least about 17 nucleotides, usually at least about 20 nucleotides, more usually at least about 24 nucleotides, typically at least about 28 nucleotides, more typically at least about 40 nucleotides, preferably at least about 50 nucleotides, and more preferably at least about 75 to 100 or more nucleotides, e.g., 150, 200, etc.

For sequence comparison, typically one sequence acts as a reference sequence, to which test sequences are compared. When using a sequence comparison algorithm, test and reference sequences are input into a computer, subsequent coordinates are designated, if necessary, and sequence algorithm program parameters are designated. The sequence comparison algorithm then calculates the percent sequence identity for the test sequence(s) relative to the reference sequence, based on the designated program parameters. Optical alignment of sequences for comparison can be conducted, e.g., by the local homology algorithm of Smith and Waterman (1981) Adv. Appl. Math. 2:482, by the homology alignment algorithm of Needlman and Wunsch (1970) J. Mol. Biol. 48:443, by the search for similarity method of Pearson and Lipman (1988) Proc. Nat'l Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra). One example of a useful algorithm is PILEUP. Another example of algorithm that is suitable for determining percent sequence identity and sequence similarity is the BLAST algorithm, which is described Altschul, et al. (1990) J. Mol. Biol. 215:403-410. A further indication that two nucleic acid sequences of polypeptides are substantially identical is that the polypeptide encoded by the first nucleic acid is immunologically cross reactive with the polypeptide encoded by the second nucleic acid. Another indication that two nucleic acid sequences are substantially identical is that the two molecules hybridize to each other under stringent conditions.

“Homologous” polynucleotide sequences, when compared, exhibit significant similarity (e.g., sequence identity at the nucleotide level). Generally, standards for determining homology between nucleic acid molecules (or polynucleotide sequences) use art known techniques which examine, e.g., the extent of structural similarity or sequence identity between polynucleotide sequences; and/or that determine a phylogenetic relationship (e.g., whether compared sequences are orthologs or paralogs); and/or that are based on the ability of sequences to form a hybridization complex. Hybridization conditions are described in detail herein.

“Hybridization” refers to the process by which a polynucleotide strand anneals with a complementary strand through base pairing under defined hybridization conditions. Specific hybridization is an indication that two nucleic acid sequences share a high degree of similarity and/or identity. Specific hybridization complexes form under permissive annealing conditions and remain hybridized after “washing.” Washing is particularly important in determining the stringency of the hybridization process, typically, with more stringent conditions allowing less non-specific binding (e.g., binding between polynucleotide sequences that demonstrate less sequence identity or similarity). Permissive conditions for annealing of nucleic acid sequences are routinely determinable by one of ordinary skill in the art and may be consistent among hybridization experiments, whereas wash conditions may be varied among experiments to achieve a desired stringency, and therefore, a particular hybridization specificity.

“Stringent conditions,” when referring to homology or substantial similarity and/or identity in the hybridization context, will be stringent combined conditions of salt, temperature, organic solvents, and other parameters, typically those controlled in hybridization reactions. Stringent temperature conditions will usually include temperatures in excess of about 30° C., more usually in excess of about 37° C., typically in excess of about 40° C., characteristically in excess of about 42° C., routinely in excess of about 45° C., usually in excess of about 47° C., preferably in excess of about 50° C., more typically in excess of about 55° C., characteristically in excess of about 60° C., preferably in excess of about 65° C., and more preferably in excess of about 70° C. In this context, the term “about” includes, e.g., a particularly recited temperature (e.g., 50° C.), and/or a temperature that is greater or lesser than that of the stated temperature by, e.g., one, two, three, four, or five degrees Celsius (e.g., 49° C. or 51° C.). Stringent salt conditions will ordinarily be less than about 500 mM, usually less than about 450 mM, even more usually less than about 400 mM, more usually less than about 350 mM, even more usually less than about 300 mM, typically less than about 250 mM, even more typically less than about 200 mM, preferably less than about 100 mM, and more preferably less than about 80 mM, even down to less than about 20 mM. In this context, the term “about” includes, e.g., a particularly recited molarity (e.g., 400 mM), and/or a molarity that is greater or lesser than that of the stated molarity by, e.g., three, five, seven, nine, eleven or fifteen millimolar (e.g., 389 mM or 415 mM). It is to be remembered that the combination of parameters is more important than the measure of any single parameter (see, e.g., Wetmur and Davidson (1968) J. Mol. Biol. 31:349-370).

A nucleic acid probe that binds to a target nucleic acid under stringent conditions to form a stable hybridization complex is said to be specific for said target nucleic acid. Preferably, hybridization under stringent conditions should give a signal of at least 2-fold over background, more preferably a signal of at least 3 to 5-fold over background or more. Typically, a hybridization probe is more than 11 nucleotides in length and is sufficiently identical (or complementary) to the sequence of the target nucleic acid (over the region determined by the sequence of the probe) to bind the target under stringent hybridization conditions to form a detectable stable hybridization complex. The term “hybridization complex” refers to a complex formed between two nucleic acid molecules by virtue of the formation of hydrogen bonds between complementary bases. A hybridization complex may be formed in solution (e.g., C₀t or R₀t analysis) or formed between one nucleic acid sequence present in solution and another nucleic acid sequence immobilized on a solid support (such as, e.g., without limitation, paper, plastic, a membrane, a filter, a chip, a pin, glass, or any other appropriate substrate to which cells or their nucleic acids can be complexed with either covalently or non-covalently).

An equation for calculating Tₘ and conditions for nucleic acid hybridization are well known (see, e.g., Sambrook, et al. (1990) Molecular Cloning: A Laboratory Manual (cur. ed.) Vol. 1-3, Cold Spring Harbor Laboratory, Cold Spring Harbor Press, NY, which is incorporated herein by reference and hereinafter referred to as “Sambrook, et al.”). A non-limiting example of a high stringency condition of the invention comprises including a wash condition of 68° C. in the presence of about 0.2×SSC and about 0.1% SDS, for 1 hour. Alternatively, temperatures of about 67° C., 63° C., 61° C., 59° C., 57° C., 53° C., 51° C., 49° C., 47° C., 43° C., or 41° C. may be used. SSC concentration may be varied from about 0.1 to 2.0×SSC, with SDS being present at about 0.1%. Typically, blocking reagents are used to block non-specific hybridization. Such blocking reagents include, for instance, sheared, and denatured salmon sperm DNA at about 100-200 ug/ml. Organic solvent, such as, e.g., formamide at a concentration of about 35-50% v/v, may also be used under particular circumstances, such as for a RNA:DNA hybridization. Useful variations on these wash conditions will be readily apparent to those of ordinary skill in the art. Hybridization, particularly under high stringency conditions, may be suggestive of evolutionary similarity between the nucleotides. Such similarity is indicative of a similar functional and/or biological role for the nucleotide sequence and its correspondingly encoded polypeptide sequence.

Another non-limiting example of a stringent hybridization condition comprises, e.g., an overnight incubation at 42° C. in a solution comprising 50% formamide, 5×SSC (750 mM NaCl, 75 mM trisodium citrate), 50 mM sodium phosphate (pH 7.6), 5× Denhardt's solution, 10% dextran sulfate, and 20 pg/ml denatured, sheared salmon sperm DNA, followed by washing the filters in 0.1×SSC at about 65° C. Also contemplated are nucleic acid molecules that hybridize to an LP polynucleotide sequence at lower stringency hybridization conditions. Changes in the stringency of hybridization and signal detection can be accomplished through the manipulation of formamide concentration (lower percentages of formamide result in lowered stringency); salt conditions, or temperature. For example, an alternate stringency condition can comprise, e.g., an overnight incubation at 37° C. in a solution comprising 6×SSPE (20×SSPE=3M NaCl; 0.2M NaH, PO, 0.02M EDTA, pH 7.4), 0.5% SDS, 30% formamide, 100 ml salmon sperm blocking DNA; followed by washes at 50° C. with 1×SSPE, 0.1% SDS. In addition, to achieve another alternate stringency condition, washes are performed following stringent hybridization at higher salt concentrations (e.g. 5×SSC). Note that variations in the above conditions may be accomplished through the inclusion and/or substitution of alternate blocking reagents used to suppress background in hybridization experiments. Typical blocking reagents include, e.g., Denhardt's reagent, BLOTTO, heparin, denatured salmon sperm DNA, and commercially available proprietary formulations. The inclusion of specific blocking reagents may require modification of a hybridization conditions described herein. A polynucleotide that hybridizes only to polyA+ sequences (such as any 3′ terminal polyA+ tract of a cDNA of the invention), or to a complementary stretch of T (or U) residues, is not included, e.g., in the definition of an “LP polynucleotide” since such a polynucleotide would hybridize to any nucleic acid molecule containing a poly (A) stretch or the complement thereof (i.e., practically any double-stranded cDNA clone generated using oligo dT as a primer).

Still another non-limiting example of a stringent hybridization condition is one that employs, e.g.: low ionic strength and high temperature for washing (e.g., 15 mM sodium chloride/1.5 mM sodium citrate/0.1% sodium dodecyl sulfate at 50° C.); a denaturing agent (during hybridization) such as formamide (e.g., 50% (v/v) formamide with 0.1% bovine serum albumin/0.1% ficoll/0.1% polyvinylpyrrolidone/50 mM sodium phosphate buffer at pH 6.5 with 750 mM sodium chloride/75 mM sodium citrate at 42° C.); or 50% formamide, 5×SSC (750 μM sodium chloride, 75 mM sodium citrate), 50 MM sodium phosphate (pH 6.8), 0.1% sodium pyrophosphate, 5× Denhardt's solution, sonicated salmon sperm DNA (50 μg/mL), 0.1% SDS, and 10% dextran sulfate at 42° C. with washes at 42° C. in 0.2×SSC (30 mM sodium chloride/3 mM sodium citrate) and 50% formamide at 55° C., followed by a high-stringency wash consisting of 0.1×SSC containing EDTA at 55° C.

An “isolated” nucleic acid is a nucleic acid molecule or a polynucleotide sequence (e.g., an RNA, DNA, cDNA, genomic DNA, or a mixed polymer) which is substantially separated from other biologic components that naturally accompany a native sequence (e.g., proteins and flanking genomic sequences from the originating species). In a preferable embodiment, the isolated LP sequence is free of association with components that can interfere with diagnostic or therapeutic uses for the sequence including, e.g., enzymes, hormones, and other proteinaceous or non-proteinaceous agents. The term embraces a polynucleotide sequence removed from its naturally occurring environment. For example, an isolated polynucleotide sequence could comprise part of a vector or a composition of matter, or could be contained within a cell, and still be “isolated” because the vector, composition of matter, or cell is not the original environment of the polynucleotide sequence. Moreover, the term encompasses recombinant or cloned DNA isolates, chemically synthesized analogs, or analogs biologically synthesized using heterologous systems. Furthermore, the term includes both double-stranded and single-stranded embodiments. If single-stranded, the polynucleotide sequence may be either the “sense” or the “antisense” strand. A substantially pure molecule includes isolated forms of the molecule.

An isolated nucleic acid molecule will usually contain homogeneous nucleic acid molecules, but, in some embodiments, it will contain nucleic acid molecules having minor sequence heterogeneity. Typically, this heterogeneity is found at the polymer ends or portions of the LP sequence that are not critical to a desired biological function or activity.

The term “isolated” does not refer to genomic or cDNA libraries, whole cell total or mRNA preparations, genomic DNA preparations (including those separated by electrophoresis and transferred onto blots), sheared whole cell genomic DNA preparations, or other compositions where the art demonstrates no distinguishing features of a LP polynucleotide sequence of the present invention.

A “recombinant” nucleic acid or polynucleotide sequence is defined either by its method of production or its structure. In reference to its method of production, e.g., a product made by a process, the process is use of any genetic engineering technique, e.g., products made by transforming cells with any non-naturally occurring vector are encompassed, as are nucleic acids comprising sequence derived using any synthetic oligonucleotide process. A similar concept is intended for a recombinant LP polypeptide. Specifically included are synthetic nucleic acid molecules which, due to the redundancy of the genetic code, encode polypeptides similar to fragments of these antigens, and fusions of sequences from various different species variants.

LP Protein

As used herein, an “LP protein” shall encompass, when used in a protein context, a protein or polypeptide having an amino acid sequence shown in SEQ ID NO: Y or a significant fragment of such a protein or polypeptide, preferably a natural embodiment. The term “protein” or “polypeptide” is meant any chain of contiguous amino acid residues, regardless of length or postranslation modification (e.g., glycosylation, or phosphorylation).

Further, an LP protein or an LP polypeptide encompass polypeptide sequences that are pre- or pro-proteins. Moreover, the present invention encompasses a mature LP protein, including a polypeptide or protein that is capable of being directed to the endoplasmic reticulum (ER), a secretory vesicle, a cellular compartment, or an extracellular space typically, e.g., as a result of a signal sequence, however, a protein released into an extracellular space without necessarily having a signal sequence is also encompassed. Generally, the polypeptide undergoes processing, e.g., cleavage of a signal sequence, modification, folding, etc., resulting in a mature form (see, e.g., Alberts, et al. (1994) Molecular Biology of The Cell, Garland Publishing, New York, N.Y., pp. 557-560, 582-592.).

The invention also embraces polypeptides that exhibit similar structure to an LP polypeptide (e.g., one that interacts with an LP protein specific binding composition). These binding compositions, e.g., antibodies, typically hind an LP protein with high affinity, e.g., at least about 100 nM; usually, better than about 30 nM; preferably, better than about 10 nM; and more preferably, at better than about 3 nM.

Modifications

An LP polypeptide can be composed of amino acids joined to each other by peptide bonds or modified peptide bonds, i.e., peptide isosteres, and may contain amino acids other than the 20 gene-encoded amino acids. The polypeptides may be modified by either natural processes, such as post-translational processing, or by chemical modification techniques that are well known in the art. Such modifications are well described in basic texts and in more detailed monographs, as well as in a voluminous research literature.

Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include, e.g., acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination (see, e.g., Creighton (1993) 2nd ed. Proteins-Structure and Molecular Properties, W. H. Freeman and Company, New York; Johnson (1983) ed. Posttranslational Covalent Modification of Proteins, Academic Press, New York, pp. 1-12; Seifter et al. (1990) Meth Enzymol 182:626-646; Rattan et al. (1992) Ann NY Acad Sci 663:48XX).

The encoded protein may also be “altered,” and may contain deletions, insertions, or substitutions of amino acid residues that produce a silent change and result in a functionally equivalent LP. Deliberate amino acid substitutions may be made based on similarity in polarity, charge, solubility, hydrophobicity, hydrophilicity, and/or the amphipathic nature of the residues, as long as the biological or immunological activity of the LP is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

“Substantially pure” refers to LP nucleic acid or LP protein or polypeptide that are removed from their natural environment and are isolated and/or separated from other contaminating proteins, nucleic acids, and other biologicals. Purity may be assayed by standard methods, and will ordinarily be at least about 50% pure, more ordinarily at least about 60% pure, generally at least about 70% pure, more generally at least about 80% pure, often at least about 85% pure, more often at least about 90% pure, preferably at least about 95% pure, more preferably at least about 98% pure, and in most preferred embodiments, at least 99% pure. Similar concepts apply, e.g., to LP antibodies or nucleic acids of the invention. For example, it may be desirable to purify an LP polypeptide from recombinant cell proteins or polypeptides. Various art known methods of protein purification may be employed (see, e.g., Deutscher, (1990) Methods in Enzymology 182: 83-9 and Scopes, (1982) Protein Purification: Principles and Practice, Springer-Verlag, NY.)

“Solubility” of an LP protein or polypeptide is reflected by sedimentation measured in Svedberg units, which are a measure of the sedimentation velocity of a molecule under particular conditions (see, Freifelder (1982) Physical Biochemistry (2d ed.) W. H. Freeman & Co., San Francisco, Calif.; and Cantor and Schimmel (1980) Biophysical Chemistry parts 1-3, W. H. Freeman & Co., San Francisco, Calif.). A soluble particle or polypeptide will typically be less than about 30 S, more typically less than about 15 S, usually less than about 10 S, more usually less than about 6 S, and, in particular embodiments, preferably less than about 4 S, and more preferably less than about 3 S. Solubility of a polypeptide or fragment depends upon the environment and the polypeptide. Many parameters affect polypeptide solubility, including temperature, electrolyte environment, size and molecular characteristics of the polypeptide, and nature of the solvent. Typically, the temperature at which the polypeptide is used ranges from about 4° C. to about 65° C. Usually the temperature at use is greater than about 18° C. and more usually greater than about 22° C. For diagnostic purposes, the temperature will usually be about room temperature or warmer, but less than the denaturation temperature of components in the assay. For therapeutic purposes, the temperature will usually be body temperature, typically about 37° C. for humans, though under certain situations the temperature may be raised or lowered in situ or in vitro.

The size and structure of the polypeptide should generally be in a substantially stable state, and usually not in a denatured state. The polypeptide may be associated with other polypeptides in a quaternary structure, e.g., to confer solubility, or associated with lipids or detergents in a manner which approximates natural lipid bilayer interactions.

The solvent will usually be a biologically compatible buffer, of a type used for preservation of biological activities, and will usually approximate a physiological solvent. Usually the solvent will have a neutral pH, typically between about 5 and 10, and preferably about 7.5. On some occasions, a detergent will be added, typically a mild non-denaturing one, e.g., CHS (cholesteryl hemisuccinate) or CHAPS (3-[3-cholamidopropyl)-dimethylammonio]-1-propane sulfonate), or a low enough concentration as to avoid significant disruption of structural or physiological properties of the protein.

Signal Sequence

The present invention encompasses “mature” forms of a polypeptide comprising a polypeptide sequence listed in a Table herein, or a polypeptide sequence of SEQ ID NO: Y. Methods for predicting whether a protein has a signal sequence, as well as the cleavage point for that sequence, are known in the art (see, e.g., McGeoch, 1985 Virus Res. 3:271-286 and Henrik Nielsen et al. (1997) Protein Engineering 10: 1-6). Employing such known art methods a signal sequence for an LP polypeptide was made. However, cleavage sites may vary and cannot be predicted with absolute certainty. Accordingly, the present invention provides secreted LP polypeptides having a sequence listed in a Table herein, or a polypeptide sequence of SEQ ID NO: Y, in which a particular N-terminus variant polypeptide sequence can begin within five, four, three, two, or one amino acid residues (e.g., +5, +4, +3, +2, +1, or −5, −4, −3, −2, −1) from a particular cleavage point designated as such herein. Similarly, it is also recognized that in some cases, cleavage of a signal sequence of a secreted protein is not uniform, resulting in more than one secreted species for a given protein (e.g., a cleavage variant). Such cleavage variant LP polypeptides, and the polynucleotides encoding them, are also encompassed by the present invention.

Moreover, the signal sequence identified by the above analysis may not necessarily predict a naturally occurring signal sequence. For example, a naturally occurring signal sequence may be further upstream from a predicted signal sequence. However, it is likely that a predicted signal sequence will be capable of directing the secreted protein to the ER. Nevertheless, the present invention encompasses a mature LP polypeptide or protein produced by expression of a polynucleotide sequence listed in a Table herein or an LP polynucleotide sequence of SEQ ID NO: X. These LP polypeptides (and fragments thereof), and the polynucleotides encoding them, are also encompassed by the present invention.

LP Variants

The present invention encompasses variants of an LP polynucleotide sequence disclosed in a table herein or SEQ ID NO: X and/or the complementary strand thereto. The present invention also encompasses variants of a polypeptide sequence disclosed in a table herein or SEQ ID NO: Y. The term “variant” refers to a polynucleotide or polypeptide differing from an LP polynucleotide sequence or an LP polypeptide of the present invention, but retaining essential properties thereof. Generally, variants are closely similar overall in structural and/or sequence identity, and, in many regions, identical to an LP polynucleotide or polypeptide of the present invention. For example, the present invention encompasses nucleic acid molecules that comprise, or alternatively consist of, a polynucleotide sequence that is at least: 80%, 85%, 90%, 95%, 96%, 97%, 98%, or 99% identical to, e.g., a polynucleotide coding sequence of SEQ ID NO: X (or a strand complementary thereto); a nucleotide sequence encoding a polypeptide of SEQ ID NO: Y; and/or polynucleotide fragments of any of these nucleic acid molecules (e.g., a fragment as defined herein). Polynucleotides, that stably hybridize to a polynucleotide fragment (as defined herein) under stringent hybridization conditions or lower stringency conditions, are also encompassed by the invention, as are polypeptides (or fragments thereof) encoded by these polynucleotides.

The present invention is also directed to polypeptides that comprise, or alternatively consist of, an amino acid sequence that is at least: 80%, 85%, 90%, 95%, 96%, 97%, 98%, 99% identical to, e.g., a polypeptide sequence of SEQ ID NO: Y (or fragments thereof); a polypeptide sequence encoded by a cDNA contained in a deposited clone, and/or a polypeptide fragment of any of these polypeptides (e.g., those fragments as defined herein). A polynucleotide sequence having at least some “percentage identity,” (e.g., 95%) to another polynucleotide sequence, means that the sequence being compared (e.g., the test sequence) may vary from another sequence (e.g. the referent sequence) by a certain number of nucleotide differences (e.g., a test sequence with 95% sequence identity to a reference sequence can have up to five point mutations per each 100 contiguous nucleotides of the referent sequence). In other words, for a test sequence to exhibit at least 95%, identity to a referent sequence, up to 5% of the nucleotides in the referent may differ, e.g., be deleted or substituted with another nucleotide, or a number of nucleotides (up to 5% of the total number of nucleotides in the reference sequence) may be inserted into the reference sequence. The test sequence may be: an entire polynucleotide sequence, e.g., as shown in a Table herein, the ORF (open reading frame), or any fragment, segment, or portion thereof (as described herein). As a practical matter, determining if a particular nucleic acid molecule or polynucleotide sequence exhibits at least about: 80%, 85%, 90%, 95%, 96%, 97%, 98% or 99% identity to an LP polynucleotide sequence can be accomplished using any art known method.

Variants encompassed by the present invention may contain alterations in the coding regions, non-coding regions, or both. Moreover, variants in which 1-2, 1-5, or 5-10 amino acids are substituted, deleted, or added in any combination are also preferred. Of course, in order of ever-increasing preference, it is highly preferable for a peptide or polypeptide to have an amino acid sequence that comprises an amino acid sequence of the present invention, which contains at least: one, but not more than: 10, 9, 8, 7, 6, 5, 4, 3, 2, or 1 amino acid substitutions. In specific embodiments, the number of additions, substitutions, and/or deletions in an polypeptide sequence of the present invention or fragments thereof (e.g., a mature form and/or other fragments described herein), is at least: 1, 2, 3, 4, 5, 6, 7, 8, 9, 10, 11, 12, 13, 14, 15, 16, 17, 18, 19, 20, 21, 22, 23, 24, 25, 10-50, or 50-150; wherein conservative amino acid substitutions are more preferable than non-conservative substitutions.

LP Polynucleotide and LP Polypeptide Fragments

The present invention is also directed to fragments of an LP polynucleotide. An LP polynucleotide “fragment” encompasses a short polynucleotide of a nucleic acid molecule, or a portion of a polynucleotide sequence of SEQ ID NO: X or a complementary strand thereto, or a portion of a polynucleotide sequence encoding a polypeptide of SEQ ID NO: Y (or fragment thereof). Polynucleotide fragments of the invention encompass a polynucleotide sequence that is preferably at least about 15 nucleotides, more preferably at least about: 20, 21, 22, 24, 26, or 29 nucleotides, favorably at least about: 30, 32, 34, 36, 38, or 39 nucleotides, and even more preferably, at least about: 40, 42, 44, 46, 48, or 49 nucleotides, desirably at least about: 50, 52, 54, 56, 58, or 59 nucleotides, particularly at least about 75 nucleotides, or at least about 150 nucleotides in length.

A polynucleotide fragment “at least 20 nucleotides in length,” e.g., is intended to include, e.g., 20 or more contiguous bases from a nucleotide sequence shown in SEQ ID NO: X or in a Table herein. In this context “at least about” includes, e.g., a specifically recited value (e.g., 20 nt), and a value that is larger or smaller by one or more nucleotides (e.g., 5, 4, 3, 2, or 1), at either terminus or at both termini. A polynucleotide fragment has use that includes without limit; e.g., diagnostic probes and primers as discussed herein. Larger fragments (e.g., 50, 150, 500, 600, or 2000 nucleotides) are also useful and preferred.

Representative examples of various lengths of polynucleotide fragments encompassed by the invention, include, e.g., fragments comprising, or alternatively consisting of, a polynucleotide sequence of SEQ ID NO:X from about nucleotide number 1-50, 51-100, 101-150, 151-200, 201-250, 251-300, 301-350, 351-400, 401-450, 451-500, 501-550, 551-600, 651-700, 701-750, 751-800, 800-850, 851-900, 901-950, 951-1000, 1001-1050, 1051-1100, 1101-1150, 1151-1200, 1201-1250, 1251-1300, 1301-1350, 1351-1400, 1401-1450, 1451-1500, 1501-1550, 1551-1600, 1601-1650, 1651-1700, 1701-1750, 1751-1800, 1801-1850, 101851-1900, 1901-1950, 1951-2000, or 2001 to the end of SEQ ID NO:X, or a strand complementary thereto. In this context, the term “about” includes, e.g., a particularly recited polynucleotide fragment range herein, and/or ranges that have lengths that are larger or smaller by several nucleotides (e.g., 5, 4, 3, 2, or 1 nt), at either terminus or at both termini. Preferably, these fragments encode a polypeptide possessing biological activity as defined herein, e.g., immunogenicity, or antigenicity. More preferably, a polynucleotide fragment can be used as a probe or primer as discussed herein. Furthermore, the present invention also encompasses a polynucleotide that stably hybridizes to a polynucleotide fragment described herein under either stringent or lowered stringency hybridization conditions. Additionally incorporated are polypeptides encoded by a polynucleotide fragment or a hybridized polynucleotide stably bound to a polynucleotide fragment of the invention. Additionally encompassed by the invention is a polynucleotide encoding a polypeptide, which is specifically or selectively bound by an antibody directed to/or generated against a mature polypeptide of the invention (or fragment thereof), e.g., a mature polypeptide of SEQ ID NO: Y.

In the present invention, a “polypeptide fragment or segment” encompasses an amino acid sequence that is a portion of SEQ ID NO: Y. Protein and/or polypeptide fragments or segments may be “free-standing,” or they may comprise part of a larger polypeptide or protein, of which the fragment or segment forms a portion or region, e.g., a single continuous region of SEQ ID NO: Y connected in a fusion protein. Representative examples of lengths of polypeptide fragments or segments encompassed by the invention, include, e.g., fragments comprising, or alternatively consisting of, from about amino acid residue number 1-20, 21-40, 41-60, 61-80, 81-100, 102-120, 121-140, 141-160, 161-170, 171-180, 181-190, 191-200, 201-210, etc., to the end of the mature coding region of a polypeptide of the invention (or fragment thereof).

Preferably, a polypeptide segment of the invention can have a length of contiguous amino acids of a polypeptide of the invention (or fragment thereof) that is at least about: 7, 8, 9, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 120, 130, 140, or 150 contiguous amino acids in length. In this context “about” includes, e.g., the specifically recited ranges or values described herein, and it also encompasses values that differ from these recited values by several amino acid residues (e.g., plus or minus 5, plus or minus 4, plus or minus 3, plus or minus 2, or; plus or minus 1 amino acid residues), at either or both ends of the fragment. Further, a polynucleotide encoding such a polypeptide fragment is also encompassed by the invention.

Moreover, a polypeptide comprising more than one of the above polypeptide fragments is encompassed by the invention; including a polypeptide comprising at least: one, two, three, four, five, six, seven, eight, nine, ten, or more fragments, wherein the fragments (or combinations thereof) may be of any length described herein (e.g., a fragment of 12 contiguous amino acids and another fragment of 30 contiguous amino acids, etc.). The invention also encompasses proteins or polypeptides comprising a plurality of distinct, e.g., non-overlapping, segments of specified lengths. Typically, the plurality will be at least two, more usually at least three, and preferably four, five, six, seven, eight, nine, ten, or even more. While length minima are stipulated, longer lengths (of various sizes) may be appropriate (e.g., one of length seven, and two of lengths of twelve). Features of one of the different polynucleotide sequences should not be taken to limit those of another of the polynucleotide sequences. Preferred polypeptide fragments include, e.g., the secreted protein as well as the mature form. Further preferred polypeptide fragments include, e.g., the secreted protein or the mature form having a continuous series of deleted residues from the amino or the carboxy terminus, or both. For example, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids can be deleted from the amino terminus of either the secreted polypeptide or the mature form. Similarly, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, can be deleted from the carboxy terminus of the secreted protein or mature form. Furthermore, any combination of the above amino and carboxy terminus deletions are preferred. Similarly, polynucleotides encoding these polypeptide fragments are also preferred.

Also preferred are polypeptide fragments or segments (and their corresponding polynucleotide fragments) that characterize structural or functional domains, such as, fragments, or combinations thereof, that comprise e.g., alpha-helix, and alpha-helix forming regions, beta-sheet, and beta-sheet-forming regions, turn, and turn-forming regions, coil, and coil-forming regions, hydrophilic regions, hydrophobic regions, alpha amphipathic regions, beta amphipathic regions, flexible regions, loop regions, hairpin domains, beta-alpa-beta motifs, helix bundles, alpha/beta barrels, up and down beta barrels, jelly roll or swiss roll motifs, transmembrane domains, surface-forming regions, substrate binding regions, transmembrane regions, linkers, immunogenic regions, epitopic regions, and high antigenic index regions. Polypeptide fragments of SEQ ID NO: Y falling within conserved domains are specifically encompassed by the present invention. Moreover, polynucleotides encoding these domains are also encompassed. Other preferred polypeptide segments are biologically active fragments. Biologically active fragments are those exhibiting activity similar, but not necessarily identical, to an activity of an LP polypeptide (or fragment thereof). The biological activity of the fragments may include, e.g., an improved desired activity, or a decreased undesirable activity. Polynucleotides encoding these polypeptide fragments are also encompassed by the invention.

Preferably, the polynucleotide fragments of the invention encode a polypeptide that demonstrates a functional activity. The phrase “functional activity” encompasses a polypeptide segment that can accomplish one or more known functional activities associated with a full-length (complete) polypeptide of invention protein. Such functional activities include, e.g., without limitation, biological activity, antigenicity [ability to bind (or compete with a polypeptide of the invention for binding) to an antibody to a polypeptide of the invention], immunogenicity (ability to generate antibody that binds to a polypeptide of the invention), ability to form multimers with a polypeptide of the invention, and the ability to bind to a receptor or ligand of a polypeptide of the invention.

The functional activity of a polypeptide of the invention (including fragments, variants, derivatives, and analogs thereof) can be assayed by various methods. For example, where one is assaying for the ability to bind or compete with a full-length polypeptide of the invention for binding to an antibody of a polypeptide of the invention, various immunoassays known in the art can be used, including, e.g., without limitation, competitive and non-competitive assay systems using techniques such as radioimmunoassays, ELISA (enzyme linked immunosorbent assay), “sandwich” immunoassays, immunoradiometric assays, gel diffusion precipitation reactions, immunodiffusion assays, in situ immunoassays (using colloidal gold, enzyme or radioisotope labels, for example), western blots, precipitation reactions, agglutination assays (e.g., gel agglutination assays, hemagglutination assays, complement fixation assays, immunofluorescence assays, protein A assays, and immunoelectrophoresis assays, etc.)

In another embodiment, antibody binding is accomplished by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

In another embodiment, where a ligand for a polypeptide of the invention is identified, or the ability of a polypeptide fragment, variant or derivative of the invention to multimerize is being evaluated, binding can be assayed, e.g., by using reducing and non-reducing gel chromatography, protein affinity chromatography, and affinity blotting (see generally, Phizicky, et al. (1995) Microbial. Rev. 59:94-123). In another embodiment, physiological correlates of binding of a polypeptide of the invention to its substrates (signal transduction) can be assayed with common techniques. In addition, assays described herein (see, e.g., the “Examples” section of the application), or otherwise known in the art, can routinely be applied to measure the ability of a polypeptide of the invention (its fragments, variants derivatives and analogs thereof) to elicit a related biological activity (either in vitro or in vivo).

Epitopes and Antibodies

The present invention encompasses a polypeptide comprising, or alternatively consisting of, an epitope of SEQ ID NO: Y or a table herein; or encoded by a polynucleotide that stably hybridizes to form a hybridization complex, under stringent hybridization conditions (or lower stringency hybridization conditions) as defined herein, to a complement of a sequence of SEQ ID NO: X.

The present invention further encompasses a polynucleotide sequence encoding an epitope of a polypeptide sequence of the invention (such as, e.g., a sequence disclosed in SEQ ID NO: X or a Table herein), a polynucleotide sequence of the complementary strand of a polynucleotide sequence encoding an epitope of the invention, and a polynucleotide sequence that stably hybridizes to a complementary strand under stringent hybridization conditions or lower stringency hybridization conditions as defined herein.

The term “epitope,” as used herein, refers to a portion of a polypeptide having antigenic or immunogenic activity in an animal, preferably a mammal, and most preferably in a human. In a preferred embodiment, the present invention encompasses a polypeptide comprising an epitope, as well as the polynucleotide encoding this polypeptide.

An “immunogenic epitope,” as used herein, is defined as a portion of a protein or a linearized polypeptide (or fragment thereof) that elicits an antibody response in an animal, as determined by any art known method (e.g., by the methods for generating antibodies described herein or otherwise known, see, e.g., Geysen, et al. (1983) Proc. Natl. Acad. Sci. USA 308 1:3998-4002).

An “antigenic epitope,” as used herein, is defined as a portion of a protein or polypeptide to which a binding composition, e.g., an antibody or antibody binding fragment, selectively binds or is specifically immunoreactive with as determined by any known art method, e.g., by an immunoassay described herein. Selective binding excludes non-specific binding but does not necessarily exclude cross-reactivity with other antigens. An antigenic determinant may compete with the intact antigen (i.e., the immunogen used to elicit the immune response) for binding to an antibody. Antigenic epitopes need not necessarily be immunogenic.

The phrase “specifically binds to” or is “specifically immunoreactive with”, when referring to a protein or peptide, refers to a binding reaction which is determinative of the presence of a protein or fragment (e.g., an LP protein) in the presence of a heterogeneous population of proteins and/or other biological components. Typically, the interaction is dependent upon the presence of a particular structure, e.g., an antigenic determinant (or epitope) recognized by a binding composition. For example, if an antibody is specific for epitope “A,” the presence of a polypeptide comprising the epitope A, or the presence of free unlabeled A, in a reaction containing free labeled A and the antibody will reduce the amount of labeled A that binds to the antibody. Thus, under designated immunoassay conditions, the specified antibodies bind to a particular protein or polypeptide sequence and do not significantly bind other proteins or other polypeptide sequences that are present in the sample. Specific binding to an antibody under such conditions may require an antibody that is selected for its specificity and/or selectivity for a particular protein. For example, antibodies raised to the protein immunogen with an amino acid sequence depicted in SEQ ID NO: Y can be selected to obtain antibodies specifically immunoreactive with LP proteins or LP polypeptides and not with other proteins or polypeptides. These antibodies will also recognize proteins or polypeptide sequences that have an above average degree of similarity or identity to an LP protein or LP polypeptide sequence. Fragments that function as epitopes can be produced by any conventional means such as, e.g., (1985) Houghten, Proc. Natl. Acad. Sci. USA 82:5131-5135, further described in U.S. Pat. No. 4,631,211.

In the present invention, an antigenic or immunogenic epitope preferably contains a polypeptide sequence of at least four, at least five, at least six, at least seven, more preferably at least eight, at least nine, at least 10, at least 11, at least 12, at least 13, at least 14, it least 15, at least 20, at least 25, at least 30, at least 40, at least 50, and, favorably, between about 15 to about 30 contiguous amino acids of a mature polypeptide of SEQ ID NO: Y or a Table herein. Preferred polypeptide fragments of contiguous amino acid residues of SEQ ID NO: Y comprising immunogenic or antigenic epitopes are at least 10, 15, 20, 25, 30, 35, 40, 45, 50, 55, 60, 65, 70, 75, 80, 85, 90, 95, or 100 contiguous amino acid residues in length.

Additional non-exclusive preferred antigenic epitopes include, e.g., the antigenic epitopes disclosed herein, as well as portions thereof. Antigenic epitopes are useful, e.g., to generate antibodies, including monoclonal antibodies that specifically bind the epitope. Preferred antigenic epitopes include, e.g., the antigenic epitopes disclosed herein, as well as any plurality thereof, e.g., at least: two, three, four, five or more of these antigenic epitopes in any combination or structural arrangement. Antigenic epitopes can be used as the target molecules in immunoassays (see, e.g., Wilson, et al. (1984) Cell 37:767-778, Sutcliffe, et al. (1983) Science 219:660-666). Similarly, immunogenic epitopes can be used, e.g., to induce antibodies according to any known art method (see, for instance, Sutcliffe, et al. supra; Wilson, et al. supra; Chow, et al. Proc. Natl. Acad. Sci. USA 82:910-25914: and Bittle, et al. (1985) J. Gen. Virol. 66:2347-2354.

Preferred immunogenic epitopes include, e.g., an immunogenic epitope disclosed herein, as well as a plurality or any combination thereof, e.g., of at least two, three, four, five or more of these immunogenic epitopes including, e.g., repeats of a particular epitope. A polypeptide comprising a plurality of epitopes may be used to elicit an antibody response with a carrier protein, such as, e.g., an albumin, to an animal system (such as, e.g., a rabbit or a mouse), or, if a polypeptide is of sufficient length (e.g., at least about 25 amino acids), the polypeptide may be presented without a carrier. However, immunogenic epitopes comprising as few as 8 to 10 amino acids have also been shown to be sufficient to generate antibodies and to be useful since they are capable of binding to, e.g., linear epitopes in a denatured polypeptide such as in Western blotting.

Polypeptides or proteins bearing an epitope of the present invention may be used to generate antibodies according to known methods including, e.g., without limitation, in vivo immunization, in vitro immunization, and phage display methods (see, e.g., Sutcliffe, et al. supra; Wilson, et al. supra, and Bittle, et al. (1985) J. Gen. Virol. 66:2347-2354.

“Binding Composition”

The term “binding composition” refers to molecules that bind with specificity and/or selectivity to an LP of the invention or fragment thereof (such as, e.g., in an antibody-antigen interaction). However, other compositions (e.g., antibodies, oligonucleotides, proteins (e.g., receptors), peptides, or small molecules) may also specifically and/or selectivity associate (bind) with the LP in contrast to other molecules. Typically, the association will be in a natural physiologically relevant protein-protein interaction (either covalent or non-covalent) and it may include members of a multi-protein complex (including carrier compounds or dimerization partners). The composition may be a polymer or chemical reagent. A functional analog may be a protein with structural modifications or may be a wholly unrelated molecule (such as, e.g., one that has a molecular shape that interacts with the appropriate binding determinants). The proteins may serve as agonists or antagonists of the binding partner, see, e.g., Goodman, et al. (eds.) (1990) Goodman & Gilman's: The Pharmacological Bases of Therapeutics (cur. ed.) Pergamon Press, Tarrytown, N.Y.

The LP may be used to screen for binding compositions that specifically and/or selectively bind an LP of the invention or fragment thereof (e.g., a binding composition can be a molecule, or part of one, that selectively and/or stoichiometrically binds, whether covalently or not, to one or more specific sites of an LP (or fragment thereof) such as, e.g., in an antigen-antibody interaction, a hormone-receptor interaction, a substrate-enzyme interaction, etc.). At least one and up to a plurality of test binding compositions can be screened for specific and/or selective binding with the LP.

In one embodiment, a binding composition thus identified is closely related to a natural ligand of an LP (such as, e.g., a ligand or fragment thereof, a natural substrate, a structural or functional mimetic, or a natural binding partner; see, e.g., Coligan, et al. (1991) Current Protocols in Immunology 1 (2):Chapter 5.)

“Binding Agent:LP Complex”

The term “binding agent:LP complex,” as used herein, refers to a complex of a binding agent and a LP (or fragment thereof) which is formed by specific and/or selective binding of the binding agent to the respective LP (or fragment thereof). Specific and/or selective binding of the binding agent means that the binding agent has a specific and/or selective binding site that recognizes a site on the LP protein (or fragment thereof). For example, antibodies raised against a LP protein (or fragment thereof) that recognize an epitope on the LP protein (or fragment thereof) are capable of forming a binding agent:LP complex by specific and/or selective binding. Typically, the formation of a binding agent:LP complex allows the measurement of LP protein (or fragment thereof) in a mixture of other proteins and/or biologics.

“Antibody:LP Complex”

The phrase “antibody:LP complex” refers to an embodiment in which the binding agent, e.g., is an antibody. The antibody may be monoclonal, polyclonal, or a binding fragment of an antibody (including, without limit, e.g., Fv, Fab, or F(ab)2 fragments; diabodies; linear antibodies (Zapata, et al., (1995) Protein Engin. 8(10): 1057-62); single-chain antibody molecules; and multispecific antibodies formed from antibody fragments). Preferably, for cross-reactivity purposes, the antibody is a polyclonal antibody.

Antibodies

Antibodies can be raised to various LP proteins, including individual, polymorphic, allelic, strain, or species variants, and fragments thereof, both in their naturally occurring (full-length) forms and in their recombinant forms. Additionally, antibodies can be raised to LP proteins in either their active forms or in their inactive forms. Anti-idiotypic antibodies may also be used. Antibodies of the invention include, e.g., without limitation, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and an epitope-binding fragment of any of the above.

As used herein, the phrase “human antibodies” includes, e.g., without limitation, antibodies having an amino acid sequence of a human immunoglobulin including, e.g., without limitation, an antibody isolated from a human immunoglobulin library or from an animal transgenic for one or more human immunoglobulins and that do not express endogenous immunoglobulins as described herein or, as taught, e.g., in U.S. Pat. No. 5,939,598. An antibody of the present invention may be monospecific, bispecific, trispecific or of greater multispecificity. Multispecific antibodies may be specific for different epitopes of an LP polypeptide (or fragment thereof) or may be specific for both a polypeptide of the present invention as well as for a heterologous epitope, such as a heterologous polypeptide or solid support material (see, e.g., WO 2093/17715; WO 92/08802; WO 91/00360; WO 92/05793; Tutt, et al. (1991) J. Immunol. 147:60-69; U.S. Pat. Nos. 4,474,893; 4,714,681; 4,925,648; 5,573,920; or 5,601,819; or Kostelny, et al. (1992) J. Immunol. 148:1547-1553.

Further encompassed by the present invention is an antibody that selectively binds a polypeptide, which is encoded by a polynucleotide that stably hybridizes, under stringent hybridization conditions (as described herein), to an LP polynucleotide sequence. An antibody of the present invention may also be characterized or specified in terms of its binding affinity to a protein or polypeptide (fragment thereof), or epitope of the invention. A preferred binding affinity of a binding composition, e.g., an antibody or antibody binding fragment, includes, e.g., a binding affinity that demonstrates a dissociation constant or Kd of less than about: 5×10⁻²M, 10⁻²M, 5×10⁻³M, 10⁻³M, 5×10⁻⁴M, 10⁻⁴M, 5×10⁻⁵M, 10⁻⁵M, 5×10 ⁻⁶M, 10 ⁻⁶M, 5×10⁻⁷M, 10⁻⁷M, 5×10⁻⁸M, 10⁻⁸M, 5×10⁻⁹M, 10⁻⁹M, 5×10⁻¹⁰M, 10⁻¹⁰M, 5×10⁻¹¹M, 10⁻¹¹M, 5×10⁻¹²M, 10⁻¹²M, 5×10⁻¹³M, 10⁻¹³M, 5×10⁻¹⁴M, 10⁻¹⁴M,5×10⁻¹⁵M, or 10⁻¹⁵M.

The invention also encompasses antibodies that competitively inhibit binding of a binding composition to an epitope of the invention as determined by any known art method for determining competitive binding, e.g., the immunoassays described herein. In preferred embodiments, the antibody competitively inhibits binding to the epitope by at least 95%, at least 90%, at least 85%, at leas 80%, at least 75%, at least 70%, at least 60%, or at least 50%.

Antibodies of the present invention may act as agonists or antagonists of an LP polypeptide (or fragment thereof). Likewise encompassed by the invention, are neutralizing antibodies that bind a ligand and prevent it binding to a receptor. Similarly encompassed are ligand-binding antibodies that inhibit receptor activation without inhibiting receptor binding. Alternatively, ligand-binding antibodies that activate a receptor are also included. Antibodies of the invention may act as receptor agonists, e.g., by potentiating or activating either all or a subset of the biological activities of the ligand-mediated receptor activation, e.g., by inducing dimerization of a receptor. The antibodies may be specified as agonists, antagonists, or inverse agonists for biological activities comprising the specific biological activities of a peptide of the invention disclosed herein. An antibody agonist can be made using known methods art (see, e.g., WO 96/40281; U.S. Pat. No. 5811,097; Deng, et al., Blood 92(6):1981-1988 (1998); Chen, et al., Cancer Res. 58(16):3668-3678 (1998); Harrop, et al., J. Immunol. 161(4):1786-1794 (1998); Zhu, et al., Cancer Res. 58(15):3209-3214 (1998)).

Antibodies of the present invention may be used, e.g., without limitation, to purify, detect, or target a polypeptide (or fragment thereof) of the present invention for, e.g., in vitro and/or in vivo diagnostic and therapeutic methods. For example, the antibodies have use in immunoassays for qualitatively and/or quantitatively measuring levels of a polypeptide (or fragment thereof) of the present invention in a biological sample (see, e.g., Harlow, et al., Antibodies: A Laboratory Manual, Cold Spring Harbor Laboratory Press, cur. ed.; incorporated by reference).

The term “monoclonal antibody” as used herein is not limited to antibodies produced through hybridoma technology. The term “monoclonal antibody” refers to an antibody that is derived from a single clone, including any eucaryotic, prokaryotic, or phage clone, and not the method by which it is produced. Methods for producing and screening for specific antibodies using hybridoma technology are routine and known in the art. For an overview of the technology for producing human antibodies, see, e.g., Lonberg and Huszar, Int. Rev. Immunol. 13:65-93 (1995). In addition, commercial companies such as, e.g., Abgenix, Inc. (Freemont, Calif.) and Genpharm (San Jose, Calif.) can be hired to produce human antibodies.

Completely human antibodies that recognize a selected epitope can be generated by “guided selection” (see, e.g., Jespers, et al. (1988) BioTechnology 12:899-903). Further, antibodies of the invention can, in turn, be used to generate anti-idiotype antibodies that “mimic” a polypeptide (or fragment thereof) of the invention using known techniques (see, e.g., Greenspan & Bona, FASEB J. 7(5):437-444; (1989) and Nissinoff, J. (1991) Immunol. 147(8):2429-2438). The present invention encompasses antibodies recombinantly fused or chemically conjugated (including both covalent and non-covalent conjugations) to a polypeptide (or portion thereof, preferably comprising at least: 10, 20, 30, 40, 50, 60, 70, 80, 90, or 100 contiguous amino acids of a polypeptide of SED ID NO:X) of the present invention to generate fusion proteins. The fusion does not necessarily need to be direct, but may occur through linker sequences.

The antibodies may be specific for antigens other than a polypeptide of the invention (or portion thereof, preferably at least: 10, 20, 30, 40, 50, 60, 70, 80, 90, or 100 contiguous amino acids) of the present invention. For example, antibodies may be used to target an LP polypeptide (or fragment thereof) to particular cell types, either in vitro or in vivo, by fusing or conjugating a polypeptide (or fragment thereof) of the present invention to an antibody specific for a particular cell surface receptor. Antibodies fused or conjugated to a polypeptide of the invention may also be used in in vitro immunoassays and in purification methods using known art methods (see e.g., Harbor, et al., supra, and WO 9312 1232; EP 439,095; Naramura et al. (1994) Immunol. Lett. 39:9 1-99).

The present invention further includes compositions comprising a polypeptide of the invention (or fragment thereof) fused or conjugated to an antibody domain other than a variable region. For example, a polypeptide of the invention (or fragment thereof) may be fused or conjugated to an antibody Fc region, or portion thereof. The antibody portion that is fused to a polypeptide of the invention (or fragment thereof) may comprise a constant region, a hinge region, a CH1 domain, a CH2 domain, and/or a CH3 domain or any combination of whole domains or portions thereof. A polypeptide of the invention (or fragment thereof) may also be fused or conjugated to an antibody portion described herein to form multimers. For example, Fc portions fused to a polypeptide of the invention (or fragment thereof) can form dimers through disulfide bonding between the Fc portions. Higher multimeric forms can be made by fusing the polypeptides to portions of IgA and IgM. Methods for fusing or conjugating a polypeptide of the invention (or fragment thereof) to an antibody portion are known (see, e.g., U.S. Pat. Nos. 5,336,603; 5,622,929; 5,359,046; 5,349,053; 5,447,851; 5,112,946; EP 307,434; EP 367,166; WO 96/04388).

In many cases, the Fc part of a fusion protein is beneficial in therapy and diagnosis, and thus can result in, e.g., improved pharmacokinetic properties (see, e.g., EP A232, 262). Alternatively, deleting the Fc part after the fusion protein has been expressed, detected, and purified, can be favored. Moreover, an antibody of the present invention (or fragment thereof) can be fused to marker sequences, such as a peptide to facilitate purification. Techniques for conjugating a therapeutic moiety to an antibody are known, see, e.g., Amon, et al., “Monoclonal Antibodies For Immunotargeting Of Drugs In Cancer Therapy”, in Monoclonal Antibodies And Cancer Therapy, Reisfeld, et al. (eds.), pp. 243-56 (Alan R. Liss, Inc.1985); Hellstrom, et al., “Antibodies For Drug Delivery”, in Controlled Drug Delivery (2nd Ed.), Robinson, et al. (eds.), pp. 623-53 (Marcel Dekker, Inc. 1987). Alternatively, an antibody can be conjugated to a second antibody to form an antibody heteroconjugate as described U.S. Pat. No. 4,676,980.

An antibody (or fragment thereof) of the invention may be utilized for immunophenotyping of cell lines and biological samples. The translation product of an LP polynucleotide sequence (or fragment thereof) may be useful as a cell specific marker, or more specifically, as a cellular marker (which is differentially expressed at various stages of differentiation and/or maturation of particular cell types). A particular protein can be measured by a variety of immunoassay methods see, e.g., Stites and Terr (eds.) (1991) Basic and Clinical Immunology (7th ed.); Price and Newman (eds.) (1991) Principles and Practice of Immunoassays Stockton Press, NY; and Ngo (ed.) (1988) Non-isotopic Immunoassays Plenum Press, NY.; Stites and Terr (eds.) Basic and Clinical Immunology (7th ed.) supra; Maggio (ed.) Enzyme Immunoassay, supra; and Harlow and Lane Antibodies, A Laboratory Manual, supra. The ability of the antibody of interest to immunoprecipitate a particular antigen can be assessed by, e.g., Western blot analysis. One of skill in the art would be knowledgeable as to the parameters are modifiable to increase binding of an antibody to an antigen and to decrease background (e.g., by pre-clearing the cell lysate with sepharose beads). Further discussion of icninunoprecipitation protocols can be found in, e.g., Ausubel et al, eds., 1994, Current Protocols in Molecular Biology, Vol. 1, John Wiley & Sons, Inc., New York.

Therapeutic Uses

The present invention further encompasses antibody-based therapies that involve administering LP antibody to an animal, preferably a mammal, most preferably a primate (e.g., a human), to modulate, treat, inhibit, effect, or ameliorate one or more of the disclosed diseases, disorders, or conditions. An antibody of the invention can be used to modulate, treat, inhibit, ameliorate, or prevent diseases, disorders, or conditions associated with aberrant expression and/or activity of a polypeptide (or fragment thereof) of the invention, including, e.g., without limitation, any one or more of the diseases, disorders, syndromes or conditions described herein. The treatment, amelioration, and/or prevention of diseases, disorders, or conditions associated with aberrant expression and/or activity of a polypeptide of the invention includes, e.g., without limitation, ameliorating symptoms associated with those diseases, disorders or conditions. Antibodies of the invention may be provided in pharmaceutically acceptable compositions as known in the art or as described herein.

Making LP Proteins; Mimetics

DNAs which encode a LP protein or fragments thereof can be obtained by chemical synthesis, screening cDNA libraries, or by screening genomic libraries prepared from a wide variety of cell lines or tissue samples. Methods for doing so, or making expression vectors are either art known or are described herein.

These DNAs can be expressed in a wide variety of host cells for the synthesis of a full-length protein or fragments which can in turn, e.g., be used to generate polyclonal or monoclonal antibodies; for binding studies; for construction and expression of modified molecules; and for structure/function studies. Each LP protein or its fragments can be expressed in host cells that are transformed or transfected with appropriate expression vectors. By “transformed” is meant a cell into which (or into an ancestor of which) a DNA molecule has been introduced, by means of recombinant techniques, which encodes an LP polypeptide or fragment thereof.

Expression vectors are typically self-replicating DNA or RNA constructs containing the desired antigen gene or its fragments, usually operably linked to appropriate genetic control elements that are recognized in a suitable host cell. The specific type of control elements necessary to effect expression depends on the host cell used. Generally, genetic control elements include a prokaryotic promoter system or a eukaryotic promoter expression control system, and typically include a transcriptional promoter, an optional operator to control the onset of transcription, transcription enhancers to elevate the level of mRNA expression, a sequence that encodes a suitable ribosome binding site, and sequences that terminate transcription and translation. All of the associated elements both necessary and sufficient for the production of LP polypeptide are in operable linkage with the nucleic acid encoding the LP polypeptide (or fragment thereof). Usually, expression vectors also contain an origin of replication that allows the vector to replicate independently of the host cell.

An expression vector will preferably include, e.g., at least one selectable marker. Such markers include, e.g., without limit, dihydrofolate reductase, G418, or neomycin resistance for eukaryotic cell culture and tetracycline, kanamycin or ampicillin resistance genes for culturing in E. coli and other bacteria.

The vectors of this invention contain DNAs which encode an LP protein, or a fragment thereof, typically encoding, e.g., a biologically active polypeptide, or protein. The DNA can be under the control of a viral promoter and can encode a selection marker. This invention further contemplates use of expression vectors capable of expressing eukaryotic cDNA coding for a LP (or fragment) in a prokaryotic or eukaryotic host, where the vector is compatible with the host and where the eukaryotic cDNA coding for the protein is inserted into the vector such that growth of the host containing the vector expresses the cDNA in question. Usually, expression vectors are designed for stable replication in their host cells or for amplification to greatly increase the total number of copies of the desirable gene per cell. It is not always necessary to require that an expression vector replicate in a host cell, e.g., it is possible to effect transient expression of the protein or its fragments in various hosts using vectors that do not contain a replication origin that is recognized by the host cell. It is also possible to use vectors that cause integration of an LP protein gene or its fragments into the host DNA by recombination, or to integrate a promoter that controls expression of an endogenous gene.

Vectors, as used herein, encompass plasmids, viruses, bacteriophage, integratable DNA fragments, and other vehicles that enable the integration of DNA fragments into the genome of the host. Expression vectors are specialized vectors that contain genetic control elements that effect expression of operably linked genes. Plasmids are the most commonly used form of vector, but many other forms of vectors that perform an equivalent function are also suitable for use (see, e.g., Pouwels, et al. (1985 and Supplements) Cloning Vectors: A Laboratory Manual Elsevier, N.Y.; and Rodriquez, et al. (eds.) (1988) Vectors: A Survey of Molecular Cloning Vectors and Their Uses Buttersworth, Boston, Mass.).

Suitable host cells include prokaryotes, lower eukaryotes, and higher eukaryotes. Prokaryotes include both gram negative and gram positive organisms, e.g., E. coli and B. subtilis. Lower eukaryotes include yeasts, e.g., S. cerevisiae and Pichia, and species of the genus Dictyostelium. Higher eukaryotes include established tissue culture cell lines from animal cells, both of non-mammalian origin, e.g., insect cells, and birds, and of mammalian origin, e.g., human, primates, and rodents.

Prokaryotic host-vector systems include a variety of vectors for many different species. As used herein, E. coli and its vectors will be used generically to include equivalent vectors used in other prokaryotes. A representative vector for amplifying DNA is pBR322 or its derivatives. Vectors that can be used to express these proteins or protein fragments include, but are not limited to, such vectors as those containing the lac promoter (pUC-series); trp promoter (pBR322-trp); Ipp promoter (the pIN-series); lambda-pP or pR promoters (pOTS); or hybrid promoters such as ptac,(pDR540). See Brosius, et al. (1988) “Expression Vectors Employing Lambda-, trp-, lac-, and Ipp-derived Promoters,” in Rodriguez and Denhardt (eds.) Vectors: A Survey of Molecular Cloning Vectors and Their Uses 10:205-236 Buttersworth, Boston, Mass. Other representative bacterial vectors include, e.g., without limit, pQE70, pQE60, and pQE-9, (available from QIAGEN, Inc.); pBluescript vectors, Phagescript vectors, pNH8A, pNH16a, pNH18A, pNH46A, (available from Stratagene Cloning Systems, Inc.); and ptrc99a, pKK223-3, pKK233-3, pDR540, pRIT5 (available from Pharmacia Biotech, Inc).

Higher eukaryotic tissue culture cells are typically the preferred host cells for expression of the functionally active LP protein. Non-limiting representative examples of suitable expression vectors include pCDNA1; pCD (Okayama, et al. (1985) Mol. Cell Biol. 5:1136-1142); pMC1neo Poly-A, (Thomas, et al. (1987) Cell 51:503-512); and a baculovirus vector such as pAC 373 or pAC 610. Additional eukaryotic vectors include, e.g., without limit, pWLNE0, pSV2CAT, pOG44, pXT1 and pSG (available from Stratagene); and pSVK3, pBPV, pMSG and pSVL (available from Pharmacia Biotech, Inc.).

A polypeptide (or fragment thereof) of the present invention, and preferably, a mature and/or secreted form, can also be recovered from natural sources, including, e.g., without limit, bodily fluids, tissues, and cells, (whether directly isolated or cultured); products of chemical synthetic procedures; and products produced by recombinant techniques from a prokaryotic or eukaryotic host (including, e.g., bacterial, yeast, higher plant, insect, and mammalian cells).

It is likely that LP proteins need not be glycosylated to elicit biological responses. However, it will occasionally be desirable to express an LP protein or LP polypeptide in a system that provides a specific or defined glycosylation pattern. In this case, the usual pattern will be that provided naturally by the expression system. However, the pattern will be modifiable by exposing the polypeptide, e.g., in unglycosylated form, to appropriate glycosylating proteins introduced into a heterologous expression system. For example, the LP protein gene may be co-transformed with one or more genes encoding mammalian or other glycosylating enzymes. It is further understood that over glycosylation may be detrimental to LP protein biological activity, and that one of skill may perform routine testing to optimize the degree of glycosylation which confers optimal biological activity.

In addition, an LP polypeptide (or fragments thereof) may also include, e.g., an initial modified methionine residue (in some cases because of host-mediated processes). Typically, the N-terminal methionine encoded by the translation initiation codon removed with high efficiency from any protein after translation in all eukaryotic cells. While the N-terminal methionine on most proteins is also efficiently removed in most prokaryotes, for some proteins depending on the nature of the amino acid to which the N-terminal methionine is covalently linked, the removal process is inefficient. In one embodiment, the yeast Pichia pastoris is used to express a polypeptide of the present invention (or fragment thereof) in an eukaryotic system (see, e.g., Ellis, et al., Mol. Cell. Biol. 5:1111-21 (1985); Koutz, et al., Yeast 5: 167-77 (1989); Tschopp, et al., Nucl. Acids Res. 15:3859-76 (1987)). Thus, a heterologous coding sequence, such as, e.g., an LP polynucleotide sequence, (or fragment thereof) under the transcriptional regulation of all or part of the AOX1 regulatory sequence is expressed at exceptionally high levels in Pichia yeast grown in the presence of methanol.

In one example, the plasmid vector pPIC9K is used to express polynucleotide sequence encoding a polypeptide of the invention, (or fragment thereof) as set forth herein, in a Pichea yeast system essentially as described in “Pichia Protocols: Methods in Molecular Biology,” D. R. Higgins and J. Cregg, eds. The Humana Press, Totowa, N.J., 1998. This expression vector allows expression and secretion of a protein of the invention by virtue of the strong AOX1 promoter linked to the Pichia pastoris alkaline phosphatase (PHO) secretory signal peptide located upstream of a multiple cloning site. Many other yeast vectors could be used in place of pPIC9K, such as, e.g., pYES2, pYD1, pTEF1/Zeo, pYES2/GS, pPICZ, pGAPZ, pGAPZalpha, pPIC9, pPIC3.5, PHIL-D2, PHIL-S1, pPIC3.5K, and, PA08, as a skilled in the artisan would appreciate, as long as the proposed expression construct provides appropriately located and operably linked signals for transcription, translation, secretion (if desired), and the like, (including an in-frame stop codon as required).

Furthermore, heterologously expressed proteins or polypeptides can also be expressed in plant cells. For plant cells viral expression vectors (e.g., cauliflower mosaic virus and tobacco mosaic virus) and plasmid expression vectors (e.g., T1 plasmid) are suitable. Such cells are available from a wide range of sources (e.g., the American Tissue Type Culture Collection, Rockland, Md.; also, see for example, Ausubel, et al. (cur. ed. and Supplements; expression vehicles may be chosen from those provided e.g., in Pouwels, et al. (Cur. ed.) Cloning Vectors, A Laboratory Manual).

A LP protein, or a fragment thereof, may be engineered to be phosphatidyl inositol (PI) linked to a cell membrane, but can be removed from membranes by treatment with a phosphatidyl inositol cleaving enzyme, e.g., phosphatidyl inositol phospholipase-C. This releases the antigen in a biologically active form, and allows purification by standard procedures of protein chemistry (see, e.g., Low (1989) Biochem. Biophys. Acta 988:427-454; Tse, et al. (1985) Science 230:1003-1008; and Brunner, et al. (1991) J. Cell Biol. 114:1275-1283).

Now that LP proteins have been characterized, fragments or derivatives thereof can be prepared by conventional processes for synthesizing peptides. These include processes such as are described in Stewart and Young (1984) Solid Phase Peptide Synthesis Pierce Chemical Co., Rockford, Ill.; Bodanszky and Bodanszky (1984) The Practice of Peptide Synthesis Springer-Verlag, New York, N.Y.; and Bodanszky (1984) The Principles of Peptide Synthesis Springer-Verlag, New York, N.Y. The prepared protein and fragments thereof can be isolated and purified from the reaction mixture by means of peptide separation, for example, by extraction, precipitation, electrophoresis and various forms of chromatography, and the like. An LP protein of this invention can be obtained in varying degrees of purity depending upon its desired use. Purification can be accomplished by use of known protein purification techniques or by the use of the antibodies or binding partners herein described (e.g., in immunoabsorbant affinity chromatography).

Recombinant Proteins

An LP polypeptide, or fragment thereof, can be used to generate a fusion protein. For example, when fused to a second polypeptide, an LP polypeptide, or fragment thereof, can be used as an antigenic tag or an immunogen.

Antibodies raised against an LP polypeptide (or fragment thereof) can be used to indirectly detect a second protein by binding thereto. In one embodiment, if an LP protein has amino acid sequence portion that targets a cellular location (e.g., based on trafficking signals), that portion of the polypeptide can be used by fusing it to another protein (or fragment) to target a protein. Examples of domains that can be fused to an LP polypeptide (or fragment thereof) include, e.g., not only heterologous signal sequences, but also other heterologous functional regions. A fusion does not necessarily need to be direct, but may occur, e.g., through linker sequences. Moreover, fusion proteins may also be engineered to improve characteristics of an LP polypeptide.

For instance, a region of additional amino acids, particularly charged amino acids, may be added to the N-terminus of the polypeptide to improve stability and persistence during purification from a host cell or during subsequent handling and storage. In addition, peptide moieties can be added to the polypeptide to facilitate purification. Such regions may be removed before final preparation of the polypeptide. Additions of peptide moieties to facilitate handling are familiar and routine art techniques. Moreover, an LP polypeptide (including any fragment thereof, and specifically an epitope) can be combined with parts of the constant domain of an immunoglobulin e.g., (IgA, IgE, IgG, IgM) portions thereof (CH1, CH2, CH3), and any combination thereof including both entire domains and portions thereof), resulting in a chimeric polypeptide. Such fusion proteins can facilitate purification and often are useful to increase the in vivo half-life of the protein (Fountoulakis, et al. (1995) J. Biochem.15 270:3958-3964). Enhanced delivery of an antigen across an epithelial barrier to the immune system has been demonstrated for antigens (e.g., insulin) conjugated to an FcRn binding partner such as IgG or Fc fragments (see, e.g., WO 96/22024 and WO 99/104813). IgG fusion proteins that have a disulfide-linked dimeric structure due to the IgG portion disulfide bonds have also been found more efficient in binding and neutralizing other molecules than monomeric polypeptides or fragments thereof alone (Fountoulakis, et al. (1995) J. Biochem. 270:3958-3964).

Additionally, a fusion protein can comprise various portions of the constant region of an immunoglobulin molecule together with a human protein (or part thereof) EP-A-O 464 533 (Canadian counterpart 2045869). In many cases, the Fc part in a fusion protein is beneficial in therapy and diagnosis, and thus, can result in, e.g., improved pharmacokinetic properties (EP-A 0232 262.). Alternatively, deleting the Fc part after the fusion protein has been expressed, detected, and purified, may be desired. For example, the Fc portion may hinder therapy and/or diagnosis if the fusion protein is used as an immunogen for immunizations. In drug discovery for example, human proteins, such as hIL-5, have been fused with Fc portions for the purpose of high-throughput screening assays to identify hIL-5 antagonists (Bennett, et al. (1995) 1. Molecular Recognition 8:52-58; and Johanson, et al. (1995) J. Biol. Chem. 270:9459-9471).

Furthermore, new constructs may be made by combining similar functional domains from other proteins. For example, protein-binding or other segments may be “swapped” between different new fusion polypeptides or fragments (see, e.g., Cunningham, et al. (1989) Science 243:1330-1336; and O'Dowd, et al. (1988) J. Biol. Chem. 263:15985-15992).

Moreover, an LP polypeptide (or fragment thereof) can be fused to a marker sequence, such as a peptide, to facilitate purification. In preferred embodiments, the marker amino acid sequence is a hexa-histidine peptide, such as, e.g., the tag provided in a pQE vector (QIAGEN, Inc., Chatsworth, Calif., 91311), which provides for convenient purification of the fusion protein (Gentz, et al. (1989) Proc. Natl. Acad. Sci. USA 86:821 -824). Another useful peptide-purification tag is the “HA” tag, which corresponds to an epitope derived from an influenza hemagglutinin protein (Wilson, et al. (1984) Cell 37:767). Nucleic acid molecules containing LP polynucleotide sequences encoding an LP epitope can also be recombined with a gene of interest as an epitope tag (e.g., the “HA” or flag tag) to aid in detection and purification of the expressed polypeptide. For example, one system purifies non-denatured fusion proteins expressed in human cell lines (Janknecht, et al. (1991) Proc. Natl. Acad. Sci. USA 88:8972-897). In this system, a gene of interest is subcloned into a vaccinia recombination plasmid such that the open reading frame of the sequence of interest is translationally fused to an amino-terminal tag consisting of six histidine residues. The tag serves as a matrix-binding domain for the fusion protein. Extracts from cells infected with the recombinant vaccinia virus are loaded onto Ni2+ nitriloacetic acid-agarose column and histidine-tagged proteins can be selectively eluted with imidazole-containing buffers.

Additionally, LP fusion constructions may be generated through the techniques of gene-shuffling, motif-shuffling, exon shuffling, and/or codon shuffling (collectively referred to as “DNA shuffling”). DNA shuffling may be employed to modulate an activity of an LP polypeptide. Such methods can be used to generate LP polypeptides (or fragments thereof) with altered activity, as well as agonists and antagonists of an LP polypeptide (see, e.g., U.S. Pat. Nos. 5,605,793; 5,811,238; 5,830,721; 5,834,252; and 5,837,458, and Patten, et al. (1997) Cur. Opinion Biotechnol. 8:724-33 30; Harayama, (1998) Trends Biotechnol. 16(2):76-82; Hansson, et al. (1999) J. Mol. Biol. 287:265-76; and Lorenzo and Blasco, (1998) Biotechniques 24(2): 308-13; each of which is incorporated by reference for these DNA shuffling teachings).

VIII. Functional Variants

“Derivatives” of LP protein antigens include amino acid sequence mutants glycosylation variants, and covalent or aggregate conjugates with other chemical moieties. Covalent derivatives can be prepared by linkage of functionalities to groups which are found in LP protein amino acid side chains or at the N- or C-termini, by any art known means. These derivatives can include, without limitation, aliphatic esters or amides of the carboxyl terminus, or of residues containing carboxyl side chains, O-acyl derivatives of hydroxyl group-containing residues, and N-acyl derivatives of the amino terminal amino acid or amino-group containing residues, e.g., lysine or arginine. Acyl groups are selected from the group of alkyl-moieties including C3 to C18 normal alkyl, thereby forming alkanoyl aroyl species. Covalent attachment to carrier proteins may be important when immunogenic moieties are haptens.

Also provided by the invention is a chemically modified derivative of a polypeptide of the invention (or fragment thereof) that may provide additional advantages such as increased solubility, increased stability increased circulating time, or decreased immunogenicity or antigenicity (see U.S. Pat. No. 4,179,337). A chemical moieties for derivatization may be selected from water soluble polymers such as, e.g., polyethyleneglycol, ethylene glycol, propylene glycol, copolymers, carboxymethylcellulose, dextran, polyvinyl alcohol, etc. A polypeptide of the invention, (or fragment thereof) may be modified at random or at predetermined positions within the molecule and may include, e.g., one, two, three, or more attached chemical moieties. The polymer may be of any molecular weight, and may be branched or unbranched. For polyethylene glycol, a preferred molecular weight is between about 1 kDa and about 100 kDa (the term “about” means that in polyethylene glycol preparations, some molecules will weigh more and some will weigh less, than the stated molecular weight).

Other sizes may be used, depending on the desired effect (e.g., the [period of sustained release, the effects, if any, on biological activity, ease in handling, the degree or lack of antigenicity, and other known effects of polyethylene glycol on a protein, polypeptide or an analog). Polyethylene glycol molecules (or other chemical moieties) should be attached with consideration of the effect on functional, immunogenic, and/or antigenic domains of a polypeptide (or fragment thereof). Attachment methods include; e.g., without limit, (coupling PEG to G-CSF); EP 0 401 384, pegylating GM-CSF using tresyl chloride (Malik, et al. (1992) Exp. Hematol. 20:1028-1035). For example, polyethylene glycol may be covalently bound through amino acid residues via a reactive group, such as, e.g., a free amino or carboxyl group. Reactive groups are those to which an activated polyethylene glycol molecule may be bound. Amino acid residues having a free amino group may include, e.g., lysine residues, and N-terminal amino acid residue. Amino acid residues having a free carboxyl group may include, e.g., aspartic acid residues, glutamic acid residues, and C-terminal amino acid residues. Sulfhydryl groups may also be used to attach to a polyethylene glycol molecule. For human, a preferred attachment is at an amino group, such as, e.g., an attachment at the N-terminus or a lysine group.

One may specifically desire a protein, or a polypeptide (or fragment thereof) that is chemically modified at the N-terminus. Using polyethylene glycol as an illustration of the present composition, one may select from a variety of polyethylene glycol molecules (by molecular weight, branching, etc.), the proportion of polyethylene glycol molecules to a protein (polypeptide) molecule in the reaction mix, the type of pegylation reaction to be performed, and the method of obtaining the selected N-terminally pegylated, e.g., polypeptide. The method of obtaining an N-terminally pegylated preparation (by, e.g., separating this moiety from other monopegylated moieties if necessary) may be by purification of the N-terminally pegylated material from a population of pegylated protein molecules. Selective protein chemical modification at the N-terminus may be accomplished by reductive alkylation, which exploits differential reactivity of different types of primary amino groups (lysine versus the N-terminal) available for derivatization in a particular protein. Under appropriate reaction conditions, substantially selective derivatization of a protein or polypeptide (or fragment thereof) at the N-terminus With a carbonyl-group-containing-polymer is achieved.

This invention also encompasses the use of derivatives of an LP protein other than variations in amino acid sequence or glycosylation. Such derivatives may involve covalent or aggregative association with chemical moieties. Generally, these derivatives fall into the three classes: (1) salts, (2) side chain and terminal residue covalent modifications, and (3) adsorption complexes (e.g., with cell membranes). Such covalent or aggregative derivatives are useful as immunogens, as reagents in immunoassays, or in purification methods such as for affinity purification of proteins or other binding proteins. For example, a LP protein antigen can be immobilized by covalent bonding to a solid support such as cyanogen bromide-activated SEPHAROSE, by methods which are well known in the art, or adsorbed onto polyolefin surfaces, with or without glutaraldehyde cross-linking, for use in an assay or purification of anti-LP protein antibodies or its respective binding partner. An LP protein can also be labeled for use in diagnostic assays with a detectable group (such as, e.g., radioiodinated by the chloramine T procedure; covalently bound to rare earth chelates; or conjugated to another fluorescent moiety). Purification of an LP protein may be effected by immobilized antibodies or a binding partner.

A polypeptide of the invention (or fragment thereof) may be as a monomer or a multimer (e.g., a dimer, a trimer, a tetramer, or a higher multimer). Accordingly, the present invention encompasses monomers and multimers of a polypeptide of the invention, (or fragment thereof) including, e.g., their preparation, and compositions (preferably, therapeutic compositions) containing them. In specific embodiments, the polypeptides and/or fragments of the invention are monomers, dimers, trimers, tetramers or higher multimers. In additional embodiments, a multimer of the invention is at least a dimer, at least a trimer, or at least a tetramer. Multimers encompassed by the invention may be homomers or heteromers. As used herein, the term “homomer,” refers to a multimer containing only a specific polypeptide (or fragment thereof) corresponding to an amino acid sequence of SEQ ID NO:Y or in a talbe herein (including fragments, variants, splice variants, and fusion proteins, corresponding to these polypeptides as described herein). A homomer may contain a polypeptide having identical or different amino acid sequences. In a specific embodiment, a homomer of the invention is a multimer containing oily polypeptides (or fragments thereof) having identical amino acid sequences. In another specific embodiment, a homomer of the invention is a multimer containing polypeptides having different amino acid sequences.

In specific embodiments, a multimer of the invention is a homodimer (e.g., containing polypeptides having identical and/or different amino acid sequences) or a homotrimer (e.g., containing polypeptides having identical and/or different amino acid sequences). In additional embodiments, the homomeric multimer of the invention is at least a homodimer, at least a homotrimer, or at least a homotetramer. As used herein, the term “heteromeric,” refers to a multimer containing one or more heterologous polypeptides. In a specific embodiment, a multimer of the invention is a heterodimer, a heterotrimer, or a heterotetramer. In additional embodiments, the heteromeric multimer of the invention is at least a heterodimer, at least a heterotrimer, or at least a heterotetramer. Multimers of the invention may be the result of hydrophobic, hydrophilic, ionic and/or covalent associations and/or may be indirectly linked, by e.g., liposome formation. Thus, in one embodiment, a multimer of the invention, such as, e.g., homodimers or homotrimers, are formed when polypeptides of the invention (or fragments thereof) contact one another in solution.

In another embodiment, a heteromultimer of the invention, such as, e.g., a heterotrimer or a heterotetramer, is formed when, e.g., a polypeptide of the invention contacts an antibody (generated against a polypeptide; or fragment thereof of the invention (including antibodies to the heterologous polypeptide sequence in a fusion protein of the invention)) in solution. In other embodiments, a multimer of the invention is formed by covalent association with and/or between a polypeptide and a binding partner such as mentioned herein (or fragment thereof). Such covalent associations may involve one or more amino acid residues contained in a polypeptide sequence (e.g., as recited in a sequence listing herein, or contained in a polypeptide encoded by a deposited clone specified herein). In one instance, a covalent association is a cross-link, e.g., between cysteine residues. In another instance, the covalent associations are the consequence of chemical or recombinant manipulation. Alternatively, such covalent associations may involve one or more amino acid residues contained in a heterologous polypeptide sequence such as, e.g., a fusion protein of the invention. In one example, covalent associations form with a heterologous sequence contained in a fusion protein of the invention (see, e.g., U.S. Pat. No. 5,478,925). In a specific example, a covalent association is between a heterologous sequence contained in an Fc fusion protein of the invention (as described herein). In another specific example, a covalent association of a fusion protein of the invention is with a heterologous polypeptide sequence such as, e.g., oseteoprotegerin (see, e.g., WO 98149305, incorporated by reference for these teachings).

In another embodiment, two or more polypeptides of the invention (or fragment thereof) are joined through peptide linkers. Examples include, e.g., peptide linkers described in U.S. Pat. No. 5,073,627 (incorporated by reference for these teachings). A protein comprising multiple polypeptides of the invention that are separated by peptide linkers may be produced using conventional recombinant DNA technology.

Recombinant fusion proteins comprising a polypeptide of the invention (or fragment thereof) fused to a polypeptide sequence that dimerizes or trimerizes in solution can be expressed in a suitable host cell. The resulting soluble multimeric fusion protein can be recovered from a supernatant using any art known technique or method described herein. Trimeric polypeptides of the invention may offer an advantage of enhanced biological activity (as defined herein). Preferred leucine zipper moieties and isoleucine moieties are those that preferentially form trimers. An example is a leucine zipper derived from lung surfactant protein D (SPD), as described in Hoppe, et al. FEBS Letters 344: 19 1,15(1994) and in U.S. patent application Ser. No. 08/446,922, (each hereby incorporated by reference for these teachings). Other peptides derived from naturally occurring trimeric proteins may be employed when preparing a trimeric polypeptide of the invention.

In another example, polypeptides or proteins of the invention are associated by interactions with a Flag polypeptide sequence (e.g., contained in a fusion protein of the invention having a Flag sequence). In a further embodiment, a protein or a polypeptide of the invention is associated by an interaction with a heterologous polypeptide sequence (contained in a Flag fusion protein of the invention) and an anti-Flag antibody.

A multimer of the invention may be generated using chemical art known techniques. For example, polypeptides (or fragments thereof) desired to be contained in a multimer of the invention may be chemically cross-linked using a linker molecule e.g., linker molecules and linker molecule length optimization techniques are known in the art; see, e.g., U.S. Pat. No. 5,478,925, which is incorporated by reference for such teachings. Additionally, a multimer of the invention may be generated using techniques known in the art to form one or more inter-molecule cross-links between the cysteine residues (see, e.g., U.S. Pat. No. 5,478,925, incorporated by reference for these teachings). Further, a polypeptide of the invention modified by the addition of cysteine or biotin to the C or N-terminus of a polypeptide can be generated by art known methods (see, e.g., U.S. Pat. No. 5,478,925, incorporated by reference for these teachings).

Additionally, a multimer of the invention can be generated by art known methods (see, e.g., U.S. Pat. No. 5,478,925, incorporated by reference for these teachings). Alternatively, a multimer of the invention can be generated using other commonly known genetic engineering techniques. In one embodiment, a polypeptide contained in a multimer of the invention is produced recombinantly with fusion protein technology described herein or otherwise known in the art (see, e.g., U.S. Pat. No. 5,478,925, incorporated by reference for these teachings). In a specific embodiment, a polynucleotide encoding a homodimer of the invention can be generated by ligating a polynucleotide sequence encoding a polypeptide (or fragment thereof) of the invention to another sequence encoding a linker polypeptide and then subsequently, further to a synthetic polynucleotide encoding the translated product of the polypeptide in the reverse orientation from the original C-terminus to the N-terminus (lacking the leader sequence) (see, e.g., U.S. Pat. No. 5,478,925, incorporated by reference for these teachings).

In another embodiment, recombinant techniques described herein or otherwise known in the art can be applied to generate a recombinant polypeptide of the invention (or fragment thereof) that contains a transmembrane domain (or hyrophobic or signal peptide) and that can be incorporated by membrane reconstitution techniques into a liposome (see, e.g., U.S. Pat. No. 5,478,925, incorporated by reference for these teachings).

X. Uses

The present invention provides reagents that will find use in diagnostic and/or therapeutic applications as described herein, e.g., in the description of kits for diagnosis.

An LP polynucleotide sequence (or fragment thereof) can be used in numerous ways, e.g., such as a reagent. The following descriptions (using known art techniques) are non-limiting examples of ways to use an LP polynucleotide sequence (or fragment thereof). For example, an LP polynucleotide sequence (or fragment thereof) is useful for chromosome identification. There exists an ongoing need to identify new chromosome markers, since few chromosome-marking reagents, based on actual sequence data (repeat polymorphisms), are presently available. Each polynucleotide of the present invention can therefore, be used as a chromosome marker.

In another embodiment, the invention encompasses a kit, e.g., for analyzing a sample for the presence of a polynucleotide associated with a proliferative disease, syndrome, disorder, or condition. In a general embodiment, the kit includes, e.g., at least an LP polynucleotide sequence (or fragment thereof) probe containing a polynucleotide sequence that hybridizes with an LP polynucleotide sequence (or fragment thereof) and directions, e.g., such as for disposal. In another specific embodiment, a kit includes, e.g., two polynucleotide probes defining an internal region of an LP polynucleotide sequence, where each probe has one strand containing a 31 mer-end internal to a region the polynucleotide.

In a further embodiment, a probe may be useful as a primer for amplification using a polymerase chain reaction (PCR). Where a diagnosis of a disease, syndrome, disorder or condition has already been made according to conventional methods, the present invention is useful as a prognostic indicator, for a subject exhibiting an enhanced or diminished expression of an LP polynucleotide sequence (or fragment thereof) by comparison to a subject expressing the polynucleotide of the present invention (or fragment thereof) at a level nearer a standard level.

The phrase, “measuring level of a composition of the present invention” is intended to mean herein measuring or estimating (either qualitatively and/or quantitatively) a level of, e.g., a polypeptide (or fragment thereof), or a polynucleotide (or fragment thereof) including, e.g., mRNA, DNA, or cDNA, in a first sample (e.g., preferably a biological sample) either directly (e.g., by determining or estimating an absolute protein or mRNA level) or relatively (e.g., by comparing to a polypeptide or mRNA level in a second sample). In one embodiment, the level in the first sample is measured or estimated from an individual having, or suspected of having, a disease, syndrome, disorder or condition and comparing that level to a second level, wherein the second level is obtained from an individual not having and/or not being suspected of having a disease, syndrome, disorder or condition. Alternatively, the second level is determined by averaging levels from a population of individuals not having or suspected of having a disease, syndrome, disorder, or condition.

As is appreciated in the art, once a standard level is determined, it can be used repeatedly as a standard for comparison. A “biological sample” is intended to mean herein any sample comprising biological material obtained from, using, or employing, e.g., an organism, body fluid, exudate, lavage product, waste product, cell (or part thereof), cell line, organ, biopsy, tissue culture, or other source originating from, or associated with, a living cell, tissue, organ, or organism, which contains, e.g., a polypeptide (or fragment thereof), a protein (or fragment thereof), a mRNA (or fragment thereof), or polynucleotide sequence (or fragment thereof) of the present invention, including, e.g., without limitation, a sample such as from, e.g., hair, skin, blood, saliva, semen, vomit, synovial fluid, amniotic fluid, breast milk, lymph, pulmonary sputum, urine, fecal matter, a lavage product, etc.

As indicated, a biological sample can include, e.g., without limitation, body fluids (e.g., such as semen, lymph, sera, plasma, urine, synovial fluid and spinal fluid) that contain a polypeptide (or fragment thereof), mRNA (or fragment thereof), a protein (or fragment thereof), or polynucleotide (or fragment thereof) of the present invention, by product, or, waste product; and/or other tissue source found to express a polypeptide (or fragment thereof), mRNA (or fragment thereof), or nucleic acid (or fragment thereof), by product, or, waste product; of the present invention. Methods for obtaining biological samples, e.g., tissue biopsies, body fluids, cells, or waste products from mammals are known in the art. Where the biological sample is to include, e.g., mRNA, a tissue biopsy is a preferred source.

The present invention further encompasses an LP polynucleotide sequence (or fragment thereof) that is chemically synthesized, or reproduced as a peptide nucleic acid (PNA) using art known methods. The use of a PNA is preferred if a polynucleotide (or a fragment thereof) is incorporated, e.g., onto a solid support, or genechip. For the purposes of the present invention, a peptide nucleic acid (PNA) is a polyamide type of polynucleotide analog in which, generally, e.g., the monomeric units for adenine, guanine, thymine and cytosine are available commercially (see, e.g., Perceptive Biosystems). Certain components of a polynucleotide, such as DNA, like phosphorus, phosphorus oxides, or deoxyribose derivatives, are not present in a PNA. Generally, PNAs bind specifically and tightly to complementary DNA strands and are not degraded by nucleases (Nielsen, et al. (1993) Nature 365: 666). In fact, a PNA binds more strongly to DNA than DNA binds to itself, probably, as there is no electrostatic repulsion between PNA/DNA; furthermore, the PNA polyamide backbone is more flexible than DNA. Because of this, PNA/DNA duplexes can bind under a wider range of stringency conditions than DNA/DNA duplexes thus, making it easier to perform multiplex hybridizations. Moreover, smaller probes can be used with PNA than with DNA due to the strong binding.

In addition, it is more likely that single base mismatches can be determined using a PNA/DNA hybridization since, e.g., a single mismatch in a PNA/DNA 15-mer lowers the melting point (Tₘ) by 8-20° C., versus lowering the melting point 4-1 6° C. for the DNA/DNA 15-mer duplex. In addition, the absence of charge groups in a PNA molecule means that hybridizations can be done at low ionic strengths and the absence of charge groups with the DNA reduces possible interference by salt.

An LP polypeptide (or fragment thereof), can be used in numerous ways. The following descriptions are non-limiting, exemplars that use art known techniques.

A polypeptide (or fragment thereof) can be used to assay a protein level, e.g., of a secreted protein, in a sample, e.g., such as a bodily fluid by using antibody-based techniques. For example, protein expression in a tissue can be studied by an immunohistological method (see, e.g., Jalkanen, et al. (1985) J. Cell Biol. 101:976-985; Jalkanen, et al. (1987) J. Cell Biol. 105:3087-303096). Another useful antibody-based method for detecting protein or polypeptide expression includes, e.g., an immunoassay like an enzyme linked immunosorbent assay or a radioimmunoassay (RIA). In addition to assaying, e.g., the level of a secreted protein in a sample, a protein can also be detected by in vivo imaging. Thus, the invention provides a means for detecting, marking, locating or diagnosing a disease, syndrome, syndrome, disorder, and/or condition comprising assaying the expression of a polynucleotide (or fragment thereof), or a polypeptide (or fragment thereof), of the present invention that is in a sample, e.g., cells or body fluid of an individual by comparing one level of expression with another level of expression, e.g., a standard level of expression to indicate, e.g., a disease, syndrome, disorder, and/or condition, (or predilection to the same), or to make a prognosis or prediction.

Furthermore, an LP polypeptide (or fragment thereof) can be used to treat, prevent, modulate, ameliorate, and/or diagnose a disease, syndrome, condition, and/or a disorder. For example, a subject can be administered a polypeptide (or fragment thereof) of the invention to replace absent or decreased levels of a polynucleotide or polypeptide (e.g., insulin); to supplement absent or decreased levels of a different polynucleotide or polypeptide (e.g., hemoglobin S for hemoglobin B; SOD to catalyze DNA repair proteins); to inhibit the activity of a polynucleotide or polypeptide (e.g., an oncogene or tumor suppressor); to activate a polynucleotide or polypeptide (e.g., by binding to a receptor), to reduce activity of a membrane bound receptor by competing with the receptor for free ligand (e.g., soluble TNF receptors can be used to reduce inflammation), or to bring about a desired response (e.g., blood vessel growth inhibition, enhancement of an immune response to proliferating cells or to an infectious agent).

Similarly, an antibody directed to a polypeptide (or fragment thereof) of the present invention can also be used to treat, prevent, modulate, ameliorate, and/or diagnose a condition, syndrome, state, disease or disorder. For example, administration of an antibody directed to an LP polypeptide (or fragment thereof) can bind and reduce the level of the targeted polypeptide. Similarly, administration of an antibody can activate an LP polypeptide (or fragment thereof), such as by binding to the polypeptide that is bound to a membrane (e.g., a receptor).

Diagnosis and Imaging Using an LP Antibody

Antibodies of the invention can be used to assay polypeptide levels in a sample e.g., using classical immunohistological methods known to those of skill in the art (see e.g., Jalkanen, et al., J. Cell. Biol. 101:976-985 (1985); Jalkanen, et al., J. Cell . Biol. 105:3087-3096 (1987)). Other antibody-based methods typically useful for detecting polypeptide expression include, e.g., immunoassays, such as the enzyme linked immunosorbent assay (ELISA) and the radioimmunoassay (RIA).

Sequences encoding an LP polypeptide (or fragment thereof) are used for the diagnosis of disorders associated with LP (such as, e.g., LP misexpression, LP overexpression, LP underexpression, etc.). Examples of such disorders include, without limit, a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, Hamartoma, sarcoma, teratocarcinoma, and, in particular, a cancer of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a cardiovascular disorder such as congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, complications of cardiac transplantation, arteriovenous fistula, atherosclerosis, hypertension, vasculitis, Raynaud's disease, aneurysms, arterial dissections, varicose veins, thrombophlebitis and phlebothrombosis, vascular tumors, and complications of thrombolysis, balloon angioplasty, vascular replacement, and coronary artery bypass graft surgery; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, Amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural edema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tatdive dyskinesia, dystonias, paranoid psychoses, post-therapeutic neuralgia, Tourette's disorder, progressive supranuclear palsy, corticobasal degeneration, and familial frontotemporal dementia; and a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephali, craniorachischisis, congenital glaucoma, cataract, and sensorineural hearing loss. Sequences encoding an LP polypeptide (or fragment thereof) are used in Southern or northern analysis; dot blot or other membrane-based technologies; PCR technologies; in dipstick, pin, and multiformat ELISA-like assays; and in microarrays utilizing fluids or tissues from a subject; to detect an altered LP polypeptide (or fragment thereof) expression. Such qualitative or quantitative methods are well known in the art.

Therapeutic Uses

This invention also provides reagents with significant therapeutic value. An LP protein or polypeptide (naturally occurring or recombinant), fragments thereof, and antibodies thereto, along with compounds identified as having binding affinity to an LP, are useful in the treatment of conditions associated with abnormal physiology or development, including abnormal proliferation, e.g., cancerous conditions, or degenerative conditions. Abnormal proliferation, regeneration, degeneration, and atrophy may be modulated by appropriate therapeutic treatment using a composition(s) provided herein. For example, a disease or disorder associated with abnormal expression or abnormal signaling by an LP protein is a target for an agonist or antagonist of the protein.

Other abnormal developmental conditions are known in cell types shown to possess LP mRNA by northern blot analysis (see, e.g., Berkow (ed.) The Merck Manual of Diagnosis and Therapy, Merck & Co., Rahway, N.J.; Thorn et al. Harrison's Principles of Internal Medicine, McGraw-Hill, N.Y.; and Rich (ed.) Clinical Immunology. Principles and Practice, Mosby, St. Louis (cur. ed.); and below). Developmental or functional abnormalities, (e.g., of the neuronal, immune, or hematopoetic system) cause significant medical abnormalities and conditions which may be susceptible to prevention or treatment using compositions provided herein.

Recombinant LP or LP antibodies can be purified and administered to a subject for treatment. These reagents can be combined for use with additional active or inert ingredients, e.g., in conventional pharmaceutically acceptable carriers or diluents, e.g., immunogenic adjuvants, along with physiologically innocuous stabilizers and excipients. These combinations can be sterile filtered and placed into dosage forms as by lyophilization in dosage vials or storage in stabilized aqueous preparations. This invention also contemplates use of antibodies or binding fragments thereof, including forms which are not complement binding. Another therapeutic approach included within the invention involves direct administration of reagents, formulations, or compositions by any conventional administration techniques (such as, e.g., without limit, local injection, inhalation, or systemic administration) to a subject. The reagents, formulations, or compositions included within the bounds and metes of the invention may also be targeted to a cell by any of the methods described herein (e.g., polynucleotide delivery techniques). The actual dosage of reagent, formulation, or composition that modulates a disease, disorder, condition, syndrome, etc., depends on many factors, including the size and health of an organism, however one of one of ordinary skill in the art can use the following teachings describing methods and techniques for determining clinical dosages (sec, e.g., Spilker (1984) Guide to Clinical Studies and Developing Protocols. Raven Press Books, Ltd., New York, pp. 7-13, 54-60; Spilker (1991) Guide to Clinical Trials, Raven Press, Ltd., New York, pp. 93-101; Craig and Stitzel (eds. 1986) Modern Pharmacology. 2d ed., Little, Brown and Co., Boston, pp. 127-33; Speight (ed. 1987) Avery's Drug Treatment: Principles and Practice of Clinical Pharmacology and Therapeutics, 3d ed., Williams and Wilkins, Baltimore, pp. 50-56; Tallarida, et al. (1988) Principles in General Pharmacology, Springer-Verlag, New York, pp. 18-20; and U.S. Pat. Nos. 4,657,760; 5,206,344; and 5,225,212.). Generally, in the range of about between 0.5 fg/ml and 500 μg/ml inclusive final concentration are administered per day to a human adult in any pharmaceutically acceptable carrier. Furthermore, animal experiments provide reliable guidance for the determination of effective does for human therapy. Interspecies scaling of effective doses can be performed following art known principles (e.g., see, Mordenti and Chappell (1989) “The Use of Interspecies Scaling in Toxicokinetics,” in Toxicokinetics and New Drug Development; Yacobi, et al. (eds.) Pergamon Press, NY).

Effective doses can also be extrapolated using dose-response curves derived from in vitro or animal-model test systems. For example, for antibodies a dosage is typically 0.1 mg/kg to 100 mg/kg of a recipients body weight. Preferably, a dosage is between 0.1 mg/kg and 20 mg/kg of a recipients body weight, more preferably 1 mg/kg to 10 mg/kg of a recipients body weight. Generally, homo-specific antibodies have a longer half-life than hetero-specific antibodies, (e.g., human antibodies last longer within a human host than antibodies from another species, e.g., such as a mouse, probably, due to the immune response of the host to the foreign composition). Thus, lower dosage of human antibodies and less frequent administration is often possible if the antibodies are administered to a human subject. Furthermore, the dosage and frequency of administration of antibodies of the invention may be reduced by enhancing uptake and tissue penetration (e.g., into the brain) by using modifications such as, e.g., lipidation.

The invention also provides a pharmaceutical pack or kit comprising one or more containers filled with one or more of the ingredients of the compositions of the invention and instructions such as, e.g., for disposal (typically, in a form prescribed by a governmental agency regulating the manufacture, use or sale of pharmaceuticals or biological products). The quantities of reagents necessary for effective treatment will depend upon many different factors, including means of administration, target site, physiological state of the patient, and other medicaments administered. Thus, treatment dosages should be titrated to optimize safety and efficacy. Typically, dosages used in vitro may provide useful guidance in the amounts useful for in situ administration of these reagents. Animal testing of effective doses for treatment of particular disorders will provide further predictive indication of human dosage. Various considerations are described, e.g., in Gilman, et al. (eds.) (1990) Goodman and Gilman's: The Pharmacological Bases of Therapeutics (8th ed.) Pergamon Press; and (1990) Remington's Pharmaceutical Sciences (17th ed.) Mack Publishing Co., Easton, Pa. Methods for administration are discussed therein and below, e.g., for oral, intravenous, intraperitoneal, or intramuscular administration, transdermal diffusion, and others. Pharmaceutically acceptable carriers will include water, saline, buffers, and other compounds described, e.g., in the Merck Index, Merck & Co., Rahway, N.J. Dosage ranges would ordinarily be expected to be in amounts lower than 1 mM concentrations, typically less than about 10 μM concentrations, usually less than about 100 nM, preferably less than about 10 pM (picomolar), and most preferably less than about 1 fM (femtomolar), with an appropriate carrier. Slow release formulations, or a slow release apparatus will often be utilized for continuous administration.

LP protein, fragments thereof, and antibodies to it or its fragments, antagonists, and agonists, may be administered directly to the host to be treated or, depending on the size of the compounds, it may be desirable to conjugate them to carrier proteins such as ovalbumin or serum albumin prior to their administration. Therapeutic formulations may be administered in any conventional dosage formulation. While it is possible for the active ingredient to be administered alone, it is preferable to present it as a pharmaceutical formulation. Formulations typically comprise at least one active ingredient, as defined above, together with one or more acceptable carriers thereof. Each carrier should be both pharmaceutically and physiologically acceptable in the sense of being compatible with the other ingredients and not injurious to the patient. Formulations include those suitable for oral, rectal, nasal, or parenteral (including subcutaneous, intramuscular, intravenous and intradermal) administration. The formulations may conveniently be presented in unit dosage form and may be prepared by any methods well known in the art of pharmacy. See, e.g., Gilman, et al. (eds.) (1990) Goodman and Gilman's: The Pharmacological Bases of Therapeutics (8th ed.) Pergamon Press; and (1990) Remington's Pharmaceutical Sciences (17th ed.) Mack Publishing Co., Easton, Pa.; Avis, et al. (eds.) (1993) Pharmaceutical Dosage Forms: Parenteral Medications Dekker, N.Y.; Lieberman, et al. (eds.) (1990) Pharmaceutical Dosage Forms: Tablets Dekker, N.Y.; and Lieberman, et al. (eds.) (1990) Pharmaceutical Dosage Forms: Disperse Systems Dekker, N.Y. The treatment of this invention may be combined with or used in association with other therapeutic agents.

The present invention also provides a pharmaceutical composition. Such a composition comprises, e.g., a therapeutically effective amount of a composition of the invention in a pharmaceutically acceptable carrier. As used herein, the term “pharmaceutically acceptable carrier” means a carrier approved by a federal regulatory agency of the United States of America, or a regulatory/administrative agency of a state government of the United States or a carrier that is listed in the U.S. Pharmacopeia or other pharmacopeia; which is generally recognized by those in the art for use in an animal, e.g., a mammal, and, more particularly, in a primate, e.g., a human primate.

Various delivery systems are known and can be used to administer, e.g., a composition, formulation, antibody polypeptide (or fragment thereof), or polynucleotide (or fragment thereof) of the invention. For example, delivery can use liposomes, microparticles, microcapsules, recombinant cells, receptor-mediated endocytosis (see, e.g., Wu and Wu (1987) J. Biol. Chem. 262:4429-4432), inclusion of a nucleic acid molecule as part of a retroviral or other vector, etc. Methods of administration include, e.g., without limit, intradermal, intramuscular, intraperitoneal, intravenous, subcutaneous, intranasal, epidural, and oral routes.

An LP can be useful in ameliorating, treating, preventing, modulating, and/or diagnosing a disease, disorder, syndrome, or condition of the immune system, by, e.g., activating or inhibiting the proliferation, differentiation, or mobilization (chemotaxis or directed movement) of an immune cell. Typically, immune cells develop through a process called hematopoiesis, producing myeloid (platelets, red blood cells, neutrophils, and macrophages) and lymphoid (B and T lymphocytes) cells from pluripotent stem cells. The etiology of an immune disease, disorder, syndrome, or condition may be genetic and/or somatic, (e.g., such as some forms of cancer or some autoimmune conditions acquired by e.g., chemotherapy or toxins or an infectious agent, e.g., a virus or prion-like entity. Moreover, an LP can be used to mark or detect a particular immune system disease, syndrome, disorder, state, or condition.

An LP can be useful in ameliorating, treating, preventing, modulating, and/or diagnosing a disease, disorder, syndrome, and/or a condition of a hematopoietic cell. An LP could be used to increase or inhibit the differentiation or proliferation of a hematopoietic cell, including a pluripotent stem cell such an effect can be implemented to treat, prevent, modulate, or ameliorate a disease, disorder, syndrome, and/or a condition associated with a decrease in a specific type of hematopoietic cell. An example of such an immunologic deficiency, disease, disorder, syndrome, and/or condition includes, e.g., without limitation, a blood condition (e.g. agammaglobulinemia, digammaglobulinemia), ataxia telangiectasia, common variable immunodeficiency, Digeorge Syndrome, HIV infection, HTLV-BLV infection, leukocyte adhesion deficiency syndrome, lymphopenia, phagocyte bactericidal dysfunction, severe combined immunodeficiency (SCIDs), Wiskott-Aldrich Disorder, anemia, thrombocytopenia, or hemoglobinuria.

Moreover, an LP can be used to modulate hemostatic or thrombolytic activity. For example, increasing hemostatic or thrombolytic activity can treat or prevent a blood coagulation condition such as e.g., afibrinogenemia, a factor deficiency, a blood platelet disease (e.g. thrombocytopenia), or a wound resulting from e.g., trauma, surgery, etc. Alternatively, a composition of the invention can be used to decrease hemostatic or thrombolytic activity or to inhibit or dissolve a clotting condition. Such compositions can be important in a treatment or prevention of a heart condition, e.g., an attack infarction, stroke, or mycardial scarring.

An LP may also be useful in ameliorating, treating, preventing, modulating and/or diagnosing an autoimmune disease, disorder, syndrome, and/or condition such as results, e.g., from the inappropriate recognition by a cell of the immune system of the self as a foreign material. Such an inappropriate recognition results in an immune response leading to detrimental effect destruction on the host, e.g., on a host cell, tissue, protein, or moiety, e.g., a carbohydrate side chain. Therefore, administration of an LP which inhibits a detrimental immune response, particularly, e.g., a proliferation, differentiation, or chemotaxis of a T-cell, may be effective in detecting, diagnosing, ameliorating, or preventing such an autoimmune disease, disorder, syndrome, and/or condition. Examples of autoimmune conditions that can be affected by the present invention include, e.g., without limit Addison's Disease syndrome hemolytic anemia, anti-phospholipid syndrome, rheumatoid arthritis, dermatitis, allergic encephalomyelitis, glomerulonephritis, Goodpasture's Syndrome, Graves' Disease syndrome, Multiple Sclerosis, Myasthenia Gravis, Neuritis, Ophthalmia, Bullous Pemphigoid, Pemphigus, Polyendocrinopathies, Purpura, Reiter's Disease syndrome, Stiff-Man Syndrome, Autoimmune Thyroiditis, Systemic Lupus Erythematosus, Autoimmune Pulmonary inflammation, Guillain-BarreSyndrome, insulin dependent diabetes mellitis, and autoimmune inflammatory eye disease.

Similarly, allergic reactions and conditions, such as asthma (e.g., allergic asthma) or other respiratory problems, may also be ameliorated, treated, modulated or prevented, and/or diagnosed by an LP polynucleotide or polypeptide (or fragment thereof), or an agonist or antagonist thereto. Moreover, such inventive compositions can be used to effect, e.g., anaphylaxis, hypersensitivity to an antigenic molecule, or blood group incompatibility. An LP may also be used to modulate, ameliorate, treat, prevent, and/or diagnose organ rejection or graft-versus-host disease (GVHD). Generally speaking, organ rejection occurs by a host's, immune-cell destruction of a transplanted tissue or cell. A similarly destructive immune response is involved in GVHD, however, in this case, transplanted foreign immune cells destroy host tissues and/or cells. Administration of a composition of the invention, which ameliorates or modulates such a deleterious immune response (e.g., a deleterious proliferation, differentiation, or chemotaxis of a T cell), can be effective in modulating, ameliorating, diagnosing, and/or preventing organ rejection or GVHD.

Similarly, an LP may also be used to detect, treat, modulate, ameliorate, prevent, and/or diagnose an inflammation, e.g., by inhibiting the proliferation and/or differentiation of a cell involved in an inflammatory response, or an inflammatory condition (either chronic or acute), including, e.g., without limitation, chronic prostatitis, granulomatous prostatitis and malacoplakia, an inflammation associated with an infection (such as, e.g., septic shock, sepsis, or systemic inflammatory response syndrome (SIRS)), ischemia-reperfusion injury, endotoxin lethality, arthritis, complement-mediated hyperacute rejection, nephritis, cytokine or chemokine induced lung injury, inflammatory bowel disease syndrome, Crohn's disease syndrome, or a condition resulting from an over production of a cytokine(s) (e.g., TNF or IL-1.)

An LP can be used to modulate, ameliorate, treat, prevent, and/or diagnose a hyperproliferative disease, condition, disorder, or syndrome (such as, e.g., a neoplasm) via direct or indirect interactions. For example, such as by initiating the proliferation of cells that, in turn, modulate a hyperproliferative state; or by increasing an immune response (e.g., by increasing the antigenicity of a protein involved in a hyperproliferative condition); or by causing the proliferation, differentiation, or mobilization of a specific cell type (e.g., a T-cell). A desired effect using a composition of the invention may also be accomplished either by e.g., enhancing an existing immune response, or by initiating a new immune response. Alternatively, the desired result may be effected either by, e.g., diminishing or blocking an existing immune response, or by preventing the initiation of a new immune response.

Examples of such hyperproliferative states, diseases, disorders, syndromes, and/or conditions include, e.g., without limitation, a neoplasm of the colon, abdomen, bone, breast, digestive system, liver, pancreas, peritoneum, endocrine system (e.g., an adrenal gland, a parathyroid gland, the pituitary, the testicles, the ovary, the thymus, or the thyroid), eye, head, neck, nervous system (central or peripheral), the lymphatic system, pelvis, skin, spleen, thorax, and urogenital system. Similarly, other hyperproliferative conditions, include, e.g., without limit hypergammaglobulinemia, lymphoproliferative conditions, paraproteinemias, purpura, sarcoidosis, Hamartoma, Sezary Syndrome, Waldenstron's Macroglobulinemia, Gaucher's Disease syndrome, histiocytosis, and other hyperproliferative states.

One preferred embodiment utilizes an LP to inhibit aberrant cellular division, through a polynucleotide delivery technique. Thus, the present invention provides a method for treating, preventing, modulating, ameliorating, preventing, inhibiting, and/or diagnosing cell proliferative diseases, disorders, syndromes, and/or conditions described herein by inserting into an abnormally proliferating cell a composition of the present invention, wherein said composition beneficially modulates an excessive condition of cell proliferation, e.g., by inhibiting transcription and/or translation. Another embodiment comprises administering one or more active copies of an LP polynucleotide sequence to an abnormally proliferating cell. For example in one embodiment, an LP polynucleotide sequence is operably linked in a construct comprising a recombinant expression vector that is effective in expressing a polypeptide (or fragment thereof) corresponding to the polynucleotide of interest. In another preferred embodiment, the construct encoding a polypeptide or fragment thereof, is inserted into a targeted cell utilizing a retrovirus or an adenoviral vector (see, e.g., Nabel, et al. (1999) Proc. Natl. Acad. Sci. USA 96: 324-326). In a still preferred embodiment, the viral vector is defective and only transforms or transfects a proliferating cell but does not transform or transfects a non-proliferating cell. Moreover, in a still further preferred embodiment, an LP polynucleotide sequence is inserted into a proliferating cell either alone, (or in combination with, or fused to, another polynucleotide sequence, which can subsequently be modulated via an external stimulus (e.g., a magnetic signal, a specific small molecule, a chemical moiety or a drug administration, etc.) that acts on an upstream promoter to induce expression of the LP polypeptide (or fragment thereof). As such, a desired effect of the present invention (e.g., selectively increasing, decreasing, or inhibiting expression of an LP polynucleotide sequence) may be accomplished based on using an external stimulus.

An LP sequence may be useful in repressing the expression of a gene or an antigenic composition, e.g., an oncogenic retrovirus. By “repressing the expression of a gene” is meant, e.g., the suppression of the transcription of a ‘gene’, the degradation of a ‘gene’ transcript (pre-message RNA), the inhibition of splicing of a ‘gene’, the destruction of mRNA, the prevention of a post-translational modification of a polypeptide, the destruction of a polypeptide, or the inhibition of a normal function of a protein.

Local administration to an abnormally proliferating cell may be achieved by any art known method or technique discussed herein including, e.g., without limit to transfection, electroporation, microinjection of cells, or in vehicles (such as a liposome, lipofectin, or a naked polynucleotide). Encompassed delivery systems include, without limit, retroviral vectors (Gilboa, J. Virology 44:845 (1982), Hocke, Nature 320:275 (1986); Wilson, et al., Proc. Natl. Acad. Sci. U.S.A. 85:3014); vaccinia virus systems (Chakrabarty, et al., Mol. Cell Biol. 5:3403 (1985); Yates, et al., Nature 3 13:8 12 (1985). Preferably a retroviral, or adenoviral delivery system (as known in the art or described herein) is used to specifically deliver a recombinant construct or to transfect a cell that is abnormally proliferating. An LP polynucleotide sequence may be delivered directly to the site of a cell proliferation, e.g., in an internal organ, body cavity, and the like by use of, e.g., an imaging device used to guide the recombinant construct. Alternatively, administration to an appropriate location may be carried out at a time of surgical intervention.

By “cell proliferative condition” is meant any human or animal disease, syndrome, disorder, condition, or state, affecting any cell, tissue, any site or any combination of organs, tissues, or body parts, which is characterized by a single or multiple local abnormal proliferation of cells, groups of cells, or tissues, whether benign or malignant. Any amount of LP may be administered as long as it has a desired effect on the treated cell, e.g., a biologically inhibiting effect on an abnormally proliferating cell. Moreover, it is possible to administer more than one LP polynucleotide or polypeptide (or fragment thereof), or an agonist or antagonist thereto, simultaneously to the same site.

By “biologically inhibiting” is meant a partial or total inhibition of mitotic activity and/or a decrease in the rate of mitotic activity or metastatic activity of a targeted cell. A biologically inhibitory dose can be determined by assessing the effects of an LP on abnormally proliferating cell division in a cell or tissue culture, tumor growth in an animal or any other art known method. In another embodiment, an LP can be useful to inhibit angiogenesis associated with abnormally proliferative cells or tissues, either alone, or as a protein fusion, or in combination with another LP polynucleotide or polypeptide (or fragment thereof), or an agonist or antagonist, thereto. In a preferred embodiment, a desired anti-angiogenic effect may be achieved indirectly, e.g., through the inhibition of hematopoietic, tumor-specific cells, such as, e.g., tumor-associated macrophages (see e.g., Joseph, et al. (1998) J Natl. Cancer Inst. 90(21): 1648-53). Alternatively, in a desired anti-angiogenic effect may be achieved directly, (e.g., see Witte, et al., (1998) Cancer Metastasis Rev. 17(2): 155-61).

An LP, including a protein fusion, may be useful in inhibiting an abnormally proliferative cell or tissue, via an induction of apoptosis. An LP may act either directly, or indirectly to induce apoptosis in a proliferative cell or tissue, e.g., by activating the death-domain FA receptor, such as, e.g., tumor necrosis factor (TNF) receptor-1, CD95 (F&APO-I), TNF-receptor-related apoptosis-mediated protein (TRAMP) and TNF-related apoptosis-inducing ligand (TRAIL) receptor-1 and -2 (see, e.g., Schulze-Osthoff, et al., Eur J Biochem 254 (3): 439-59 (1998), which is hereby incorporated by reference fur teachings on apoptotic cell death). Moreover, in another preferred embodiment, an LP may induce apoptosis via other mechanisms, such as, e.g., through the activation of a pathway that subsequently activates apoptosis, or through stimulating the expression of a protein(s) that activates an apoptotic pathway, either alone or in combination with small molecule drugs or adjuvants, such as apoptonin, galectins, thioredoxins, anti-inflammatory proteins (see e.g., Mutat Res 400 (1-2):447-55 (1998), Med Hypotheses. 50(5): 423-33 (1998), Chem Biol Interact. Apr. 24; III-112:23-34 (1998), J Mol Med. 76(6): 402-12(1998), Int J Tissue React; 20 (1):3-15 (1998), which are all hereby incorporated by reference for these teachings).

An LP is useful in inhibiting cell metastasis either directly as a result of administering a polynucleotide or polypeptide (or fragment thereof), or an agonist or antagonist thereto, (as described elsewhere herein), or indirectly, such as, e.g., by activating or increasing the expression of a protein known to inhibit metastasis, such as, e.g., an alpha integrin, (see, e.g., Cur. Top Microbial Immunol 1998; 23 1: 125-4 1, which is hereby incorporated by reference for these teachings). Such a desired effect can be achieved either alone using an LP or in combination with e.g., a small molecule drug or an adjuvant.

An LP, or a protein fusion thereto, is useful in enhancing the immunogenicity and/or antigenicity of a proliferating cell or tissue, either directly, (such as would occur if e.g., an LP polypeptide (or fragment thereof) ‘vaccinated’ the immune system to respond to a proliferative antigen or immunogen), or indirectly, (such as in activating, e.g., the expression a of protein known to enhance an immune response (e.g. a chemokine), to an antigen on an abnormally proliferating cell).

An LP may be used to, modulate, ameliorate, effect, treat, prevent, and/or diagnose a cardiovascular disease, disorder, syndrome, and/or condition. As described herein, including, e.g., without limitation, cardiovascular abnormalities, such as arterio-arterial fistula, arteriovenous fistula, cerebral arteriovenous malformations, congenital heart defects, pulmonary atresia, and Scimitar Syndrome peripheral artery disease, syndrome, such as limb ischemia. Additional cardiovascular disorders encompass, e.g., congenital heart defects which include, e.g., aortic coarctation, car triatriatum, coronary vessel anomalies, crisscross heart, dextrocardia, patent ductus arteriosus, Ebstein's anomaly, Eisenmenger complex, hypoplastic left heart syndrome, levocardia, tetralogy of fallot, transposition of great vessels, double outlet right ventricle, tricuspid atresia, persistent truncus arteriosus, and heart septal defects, such as e.g., aortopulmonary septal defect, endocardial cushion defects, Lutembacher's Syndrome, trilogy of Fallot, and ventricular heart septal defects. Further cardiovascular conditions include, e.g., heart disease syndrome, such as, e.g., arrhythmias, carcinoid heart disease syndrome, high cardiac output, low cardiac output, cardiac tamponade, endocarditis (including bacterial endocarditis), heart aneurysm, cardiac arrest, congestive heart failure, congestive cardiomyopathy, paroxysmal dyspnea, cardiac edema, heart hypertrophy, congestive cardiomyopathy, left ventricular hypertrophy, right ventricular hypertrophy, post-infarction heart rupture, ventricular septal rupture, heart valve disease, myocardial disease, myocardial ischemia, pericardial effusion, pericarditis (including constrictive and tuberculous pericarditis), pneumopericardium, post-pericardiotomy syndrome, pulmonary heart disease syndrome, rheumatic heart disease syndrome, ventricular dysfunction, hyperemia, cardiovascular pregnancy complications, Scimitar Syndrome, cardiovascular syphilis, and cardiovascular tuberculosis. Further cardiovascular disorders include, e.g., arrhythmias including, e.g., sinus arrhythmia, atrial fibrillation, atrial flutter, bradycardia, extra systole, Adams-Stokes Syndrome, bundle-branch block, sinoatrial block, long QT syndrome, parasystole, Lown-Ganong-Levine Syndrome, Mahaim-type pre-excitation syndrome, Wolff-Parkinson-White syndrome, sick sinus syndrome, and ventricular fibrillation tachycardias. Tachycardias encompassed with the cardiovascular condition described herein include, e.g., paroxysmal tachycardia, supraventricular tachycardia, accelerated idioventricular rhythm, atrioventricular nodal re-entry tachycardia, ectopic atrial tachycardia, ectopic junctional tachycardia, sinoatrial nodal re-entry tachycardia, sinus tachycardia, Torsades de Pointes Syndrome, and ventricular tachycardia. Additional cardiovascular disorders include, e.g., heart valve disease such as, e.g., aortic valve insufficiency, aortic valve stenosis, heart murmurs, aortic valve prolapse, mitral valve prolapse, tricuspid valve prolapse, mitral valve insufficiency, mitral valve stenosis, pulmonary atresia, pulmonary valve insufficiency, pulmonary valve stenosis, tricuspid atresia, tricuspid valve insufficiency, and tricuspid valve stenosis. Myocardial conditions associated with cardiovascular disease include, e.g., myocardial diseases such as, e.g., alcoholic cardiomyopathy, congestive cardiomyopathy, hypertrophic cardiomyopathy, aortic subvalvular stenosis, pulmonary subvalvular stenosis, restrictive cardiomyopathy, Chagas cardiomyopathy, endocardial fibroelastosis, endomyocardial fibrosis, Kearns Syndrome, myocardial reperfusion injury, and myocarditis.

Cardiovascular conditions include, e.g., myocardial ischemias such as, e.g., coronary disease syndrome, such as e.g., angina pectoris, coronary aneurysm, coronary arteriosclerosis, coronary thrombosis, coronary vasispasm, myocardial infarction, and myocardial stunning. Cardiovascular diseases also encompassed herein include, e.g., vascular diseases such as e.g., aneurysms, angiodysplasia, angiomatosis, bacillary angiomatosis, Hippel-Lindau Disease syndrome, Klippel-Trenaunay-Weber Syndrome, Sturge-Weber Syndrome, angioneurotic edema, aortic disease, Takayasu's Arteritis, aortitis, Leriche's Syndrome, arterial occlusive disease, arteritis, enarteritis, polyarteritis nodosa, cerebrovascular disease, diabetic angiopathies, diabetic retinopathy, embolism, thrombosis, erythromeialgia, hemorrhoids, hepatic veno-occlusive disease syndrome, hypertension, hypotension, ischemia, peripheral vascular diseases, phlebitis, pulmonary veno-occlusive disease syndrome, Raynaud's disease syndrome, CREST syndrome, retinal vein occlusion, Scimitar syndrome, superior vena cava syndrome, telangiectasia, ataxia telangiectasia, hereditary hemorrhagic telangiectasia, varicocele, varicose veins, varicose ulcer, vasculitis, and venous insufficiency. Cardiovascular conditions further include, e.g., aneurysms such as, e.g., dissecting aneurysms, false aneurysms, infected aneurysms, ruptured aneurysms, aortic aneurysms, cerebral aneurysms, coronary aneurysms, heart aneurysms, and iliac aneurysms. Arterial occlusive cardiovascular conditions include, e.g., arteriosclerosis, intermittent claudication, carotid stenosis, fibromuscular dysplasias, mesenteric vascular occlusion, Moyamoya disease syndrome, renal artery obstruction, retinal artery occlusion, and thromboangiitis obliterans.

Cerebrovascular cardiovascular conditions include, e.g., carotid artery disease, cerebral amyloid angiopathy, cerebral aneurysm, cerebral anoxia, cerebral arteriosclerosis, cerebral arteriovenous malformation, cerebral artery disease, cerebral embolism and thrombosis, carotid artery thrombosis, sinus thrombosis, Wallenberg's syndrome, cerebral hemorrhage, epidural hematoma, subdural hematoma, subarachnoid hemorrhage, cerebral infarction, cerebral ischemia (including transient cerebral ischemia), subclavian steal syndrome, periventricular leukomalacia, vascular headache, cluster headache, migraine, and vertebrobasilar insufficiency. Embolic cardiovascular conditions include, e.g., air embolisms, amniotic fluid embolisms, cholesterol embolisms, blue toe syndrome, fat embolisms, pulmonary embolisms, and thromboembolisms. Thrombotic cardiovascular conditions include, e.g., coronary thrombosis, hepatic vein thrombosis, retinal vein occlusion, carotid artery thrombosis, sinus thrombosis, Wallenberg's syndrome, and thrombophlebitis. Ischemic conditions include, e.g., cerebral ischemia, ischemic colitis, compartment syndromes, anterior compartment syndrome, myocardial ischemia, reperfusion injuries, and peripheral limb ischemia. Vasculitic conditions include, e.g., aortitis, arteritis, Behcet's Syndrome, Churg-Strauss Syndrome, mucocutaneous lymph node syndrome, thromboangiitis obliterans, hypersensitivity vasculitis, Schoenlein-Henoch purpura, allergic cutaneous vasculitis, and Wegener's granulomatosis. An LP can be beneficial in ameliorating critical limb ischemia and coronary disease. An LP may be administered using any art known method, described herein An LP may administered as part of a therapeutic composition or formulation, as described in detail herein. Methods of delivering an LP are also described in detail herein.

Anti-Hemopoietic Activity

The naturally occurring balance between endogenous stimulators and inhibitors of angiogenesis is one in which inhibitory influences typically predominate (see, e.g., Rastnejad, et al., Cell 56345-355 (1989)). When neovascularization occurs under normal physiological conditions, such as wound healing, organ regeneration, embryonic development, and female reproductive processes, angiogenesis is stringently regulated, and delimited spatially and temporally. In pathological angiogenesis such as, e.g., during solid tumor formation, these regulatory controls fail and unregulated angiogenesis can become pathologic by sustaining progression of many neoplastic and non-neoplastic diseases. A number of serious diseases are dominated by abnormal neovascularization (including, e.g., solid tumor growth and metastases, arthritis, some types of eye conditions, and psoriasis; see, e.g., reviews by Moses, et al., Biotech. 9630-634 (1991); Folkman, et al., N. Engl. J. Med., 333: 1757-1763 (1995); Auerbach, et al., J. Microvasc. Res. 29:401-4 11 (1985); Folkman, “Advances in Cancer Research”, eds. Klein and Weinhouse, Academic Press, New York, pp. 175-203 (1985); Patz, Am. J. Opthalmol. 94:7 15-743 (1982); and Folkman, et al., Science 221:7 19-725 (1983).

In a number of pathological conditions, angiogenesis contributes to a disease-state, e.g., for example, significant data have accumulated suggesting that solid tumor formation is dependent on angiogenesis (see, e.g., Folkman and Klagsbrun, Science 235:442-447 (1987)). In another embodiment of the invention, administration of an LP provides for the treatment, amelioration, modulation, diagnosis, and/or inhibition of a disease, disorder, syndrome, and/or condition associated with neovascularization. Malignant and metastatic conditions that can be effected in a desired fashion using an LP include, e.g., without limitation, a malignancy, solid tumor, and a cancer as described herein or as otherwise known in the art (for a review of such disorders, syndromes, etc. see, e.g., Fishman, et al., Medicine, 2d Ed., J. B. Lippincott Co., Philadelphia (1985)). Thus, the present invention provides a method of ameliorating, modulating, treating, preventing, and/or diagnosing an angiogenesis-related disease and/or disorder, comprising administering to a subject in need thereof a beneficially effective amount of an LP. For example, cancers that may be so affected using a composition of the invention includes, e.g., without limit a solid tumor, including e.g., prostate, lung, breast, ovarian, stomach, pancreas, larynx, esophagus, testes, liver, parotid, biliary tract, colon, rectum, cervix, uterus, endometrium, kidney, bladder, thyroid cancer; primary tumors and metastases; melanomas; glioblastoma; Kaposi's sarcoma; leiomyosarcoma; non-small cell lung cancer; colorectal cancer; advanced malignancies; and blood born tumors such as e.g., leukemia.

Moreover, an LP may be delivered topically, to treat or prevent cancers such as, e.g., skin cancer, head and neck tumors, breast tumors, and Kaposi's sarcoma. Within yet another aspect, an LP may be utilized to treat superficial forms of bladder cancer by, e.g., intravesical administration into the tumor, or near the tumor site; via injection or a catheter. Of course, the appropriate mode of administration will vary according to the cancer to be treated. Other modes of delivery are discussed herein. An LP may also be useful in modulating, ameliorating, treating, preventing, and/or diagnosing another disease, disorder, syndrome, and/or condition, besides a cell proliferative condition (e.g., a cancer) that is assisted by abnormal angiogenic activity. Such close group conditions include, e.g., without limitation, benign tumors, e.g., such as hemangiomas, acoustic neuromas, neurofibromas, trachomas, and pyogenic granulomas; atherosclerotic plaques; ocular angiogenic diseases, e.g., diabetic retinopathy, retinopathy of prematurity, macular degeneration, cornea graft rejection, neovascular glaucoma, retrolental fibroplasia, rubeosis, retinoblastoma, uvietis and Pterygia (abnormal blood vessel growth) of the eye; rheumatoid arthritis; psoriasis; delayed wound healing; endometriosis; vasculogenesis; granulations; hypertrophic scars (keloids); nonunion fractures; scleroderma; trachoma; vascular adhesions; myocardial angiogenesis; coronary collaterals; cerebral collaterals; arteriovenous malformations; ischemic limb angiogenesis; Osler-Webber Syndrome; plaque neovascularization; telangiectasia; hemophiliac joints; angiofibroma; fibromuscular dysplasia; wound granulation; Crohn's disease; and atherosclerosis.

For example, within another aspect of the present invention methods are provided for modulating, ameliorating, treating, preventing, and/or diagnosing hypertrophic scars and keloids, comprising administering an LP to a site of hypertrophic scar or keloid formation. Within one embodiment, the method involves a direct injection into a hypertrophic scar or keloid, to provide a beneficial effect, e.g., by preventing progression of such a lesion. This method is of particular value to a prophylactic treatment of a condition known to result in the development of a hypertrophic scar or a keloid (e.g., burns), and is preferably initiated after the proliferative phase of scar formation has had time to progress (approximately, e.g., 14 days after the initial injury), but before hypertrophic scar or keloid development. As noted above, the present invention also provides methods for ameliorating, treating, preventing, and/or diagnosing neovascular diseases of the eye, including e.g., corneal graft neovascularization, neovascular glaucoma, proliferative diabetic retinopathy, retrolental fibroplasia and macular degeneration. Moreover, ocular diseases, disorders, syndromes, and/or conditions associated with neovascularization that can be modulated ameliorated, treated, prevented, and/or diagnosed with an LP include, e.g., without limit; neovascular glaucoma, diabetic retinopathy, retinoblastoma, retrolental fibroplasia, uveitis, retinopathy of premature macular degeneration, corneal graft neovascularization, as well as other inflammatory eye diseases, ocular tumors, and diseases associated with choroidal or iris neovascularization (see, e.g., reviews by Waltman, et al., (1978) Am. J. Ophthal. 8.51704-710 and Gartner, et al., (1978) Sun. Ophthd. 22:291-3 12). Thus, within one aspect of the present invention methods are provided for treating or preventing neovascular diseases of the eye such as corneal neovascularization (including corneal graft neovascularization), comprising administering to a patient a therapeutically effective amount of an LP composition to the cornea, such that the formation of blood vessels is inhibited or delayed. Briefly, the cornea is a tissue that normally lacks blood vessels. In certain pathological conditions however, capillaries may extend into the cornea from the pericorneal vascular plexus of the limbus. When the cornea becomes vascularized, it also becomes clouded, resulting in a decline in the patient's visual acuity. Visual loss may become complete if the cornea completely opacifies. A wide variety of diseases, disorders, syndromes, and/or conditions can result in corneal neovascularization, including e.g., corneal infections (e.g., trachoma, herpes simplex keratitis, leishmaniasis and onchocerciasis), immunological processes (e.g., graft rejection and Stevens-Johnson's syndrome), alkali burns, trauma, inflammation (of any cause), toxic and nutritional deficiency states, and as a complication of using contact lenses.

Within particularly preferred embodiments, an LP composition may be prepared for topical administration in saline (combined with any of the preservatives and anti-microbial agents commonly used in ocular preparations), and administered in drop form to the eye. The solution or suspension may be prepared in its pure form and administered several times daily. Alternatively, anti-angiogenic compositions, prepared as described herein, may also be administered directly to the cornea. Within preferred embodiments, an anti-angiogenic composition is prepared with a muco-adhesive polymer, which binds to the cornea.

Within further embodiments, an anti-angiogenic factor or anti-angiogenic LP composition may be utilized as an adjunct to conventional steroid therapy. Topical therapy may also be useful prophylactically in corneal lesions that are known to have a high probability of inducing an angiogenic response (such as, e.g., a chemical burn). In these instances, the treatment (likely in combination with steroids) may be instituted immediately to help prevent subsequent complications. Within other embodiments, an LP composition may be injected directly into the corneal stroma using microscopic guidance by an ophthalmologist. The preferred site of injection may vary with the morphology of the individual lesion, but the goal of the administration is to place a composition of the invention at the advancing front of the vasculature (i.e., interspersed between the blood vessels and the normal cornea). In most instances, this would involve perilimbic corneal injection to “protect” the cornea from advancing blood vessels. This method may also be utilized shortly after a corneal insult to prophylactically prevent corneal neovascularization. In such a situation, the composition could be injected into the perilimbic cornea interspersed between the corneal lesion and its undesired potential limbic blood supply. Such methods may also be utilized in a similar fashion to prevent capillary invasion of transplanted corneas. In a sustained-release form, injections might only be required 2-3 times per year. A steroid could also be added to the injection solution to reduce inflammation resulting from the injection itself.

Within another aspect, methods are provided for treating or preventing neovascular glaucoma, comprising administering to a patient a therapeutically effective amount of an LP to the eye, such that the formation of blood vessels is inhibited. In one embodiment, the composition may be administered topically to the eye to treat or prevent early forms of neovascular glaucoma. Within other embodiments, the composition may be implanted by injection into the region of the anterior chamber angle. Within other embodiments, the composition may also be placed in any location such that the composition is continuously released into the aqueous humor. Within another aspect, methods are provided for treating or preventing proliferative diabetic retinopathy, comprising administering to a patient a therapeutically effective amount of an LP to the eyes, such that the formation of blood vessels is inhibited. Within a particularly preferred embodiment, proliferative diabetic retinopathy may be treated by injection into the aqueous or the vitreous humor, to increase the local concentration of a composition of the invention in the retina. Preferably, this treatment should be initiated before the acquisition of severe disease requiring photocoagulation. Within another aspect of the present invention, methods are provided for treating or preventing retrolental fibroplasia, comprising administering to a patient a beneficially effective amount of an LP to the eye, such that the formation of blood vessels is inhibited. The composition may be administered topically, via intravitreous injection and/or via intraocular implants. Additional, diseases, disorders, syndromes, and/or conditions that can be modulated, ameliorated, treated, prevented, and/or diagnosed with an LP include, e.g., without limitation, hemangioma, arthritis, psoriasis, angiofibroma, atherosclerotic plaques, delayed wound healing, granulations, hemophilic joints, hypertrophic scars, nonunion fractures, Osler-Weber syndrome, pyogenic granuloma, scleroderma, trachoma, and vascular adhesions.

Moreover, diseases, disorders, states, syndromes, and/or conditions that can be modulated, ameliorated, treated, prevented, and/or diagnosed with an LP include, e.g., without limitation, solid tumors, blood born tumors such as leukemias, tumor metastasis, Kaposi's sarcoma, benign rumors (e.g., hemangiomas), acoustic neuromas, neurofibromas, trachomas, and pyogenic granulomas, rheumatoid arthritis, psoriasis, ocular angiogenic diseases, e.g., diabetic retinopathy, retinopathy of prematurity, macular degeneration, corneal graft rejection, neovascular glaucoma, retrolental fibroplasia, rubeosis, retinoblastoma, and uvietis, delayed wound healing, endometriosis, vasculogenesis, granulations, hypertrophic scars (keloids), nonunion fractures, scleroderma, trachoma, vascular adhesions, myocardial angiogenesis, coronary collaterals, cerebral collaterals, arteriovenous malformations, ischemic limb angiogenesis, Osler-Webber Syndrome, plaque neovascularization, telangiectasia, hemophiliac joints, angiofibroma fibromuscular dysplasia, wound granulation, Crohn's disease, syndrome, atherosclerosis, birth-control inhibition of vascularization necessary for embryo implantation during the control of menstruation, and diseases that have angiogenesis as a pathologic consequence such as, e.g., cat scratch disease (Rochele minalia quintosa), ulcers (Helicobacter pylori), Bartonellosis and bacillary angiomatosis.

In another embodiment as a birth control method, an amount of an LP sufficient to block embryo implantation is administered before or after intercourse and fertilization have occurred, thus providing an effective method of birth control, possibly a “morning after” method. An LP may also be used in controlling menstruation or administered either as a peritoneal lavage fluid or for peritoneal implantation in the treatment of endometriosis.

An LP may be utilized in a wide-variety of surgical procedures. For example, within one aspect of the present invention a compositions (in the form of, e.g., a spray or film) may be utilized to coat or spray an area before removal of a tumor, to isolate normal surrounding tissues from malignant tissue, and/or to prevent the spread of disease to surrounding tissues. Within other aspects, an LP composition (e.g., in the form of a spray) may be delivered via endoscopic procedures to coat tumors, or inhibit angiogenesis in a desired locale. Within yet another aspect, surgical meshes that have been coated with an anti-angiogenic composition of the invention may be utilized in a procedure in which a surgical mesh might be utilized. For example, a surgical mesh laden with an anti-angiogenic composition may be utilized during cancer resection surgery (e.g., abdominal surgery subsequent to colon resection) to provide support to the structure, and to release an amount of the anti-angiogenic factor. Within further aspects of the present invention, methods are provided for treating tumor excision sites, comprising administering an LP to the resection margins of a tumor after excision, such that the local recurrence of cancer and the formation of new blood vessels at the site is inhibited.

Within one embodiment, an anti-angiogenic composition of the invention is administered directly to a tumor excision site (e.g., applied by swabbing, brushing or otherwise coating the resection margins of the tumor with the anti-angiogenic composition). Alternatively, an anti-angiogenic composition may be incorporated into a known surgical paste before administration. Within a particularly preferred embodiment, an anti-angiogenic composition of the invention is applied after hepatic resections for malignancy, and after neurosurgical operations. Within another aspect, administration can be to a resection margin of a wide variety of tumors, including e.g., breast, colon, brain, and hepatic rumors. For example, within one embodiment, anti-angiogenic compositions may be administered to the site of a neurological tumor after excision, such that the formation of new blood vessels at the site is inhibited.

Diseases at the Cellular Level

Diseases associated with increased cell survival or the inhibition of apoptosis that could be modulated, ameliorated, treated, prevented, and/or diagnosed by an LP include, e.g., cancers (such as, e.g., follicular lymphomas, carcinomas with p53 mutations, and hormone-dependent tumors, including, e.g., but without limit, colon cancer, cardiac tumors, pancreatic cancer, melanoma, retinoblastoma, glioblastoma, lung cancer, intestinal cancer, testicular cancer, stomach cancer, neuroblastoma, myxoma, myoma, lymphoma, endothelioma, osteoblastoma, osteoclastoma, osteosarcoma, chondrosarcoma, adenoma, breast cancer, prostate cancer, Kaposi's sarcoma and ovarian cancer); autoimmune conditions (such as, e.g., multiple sclerosis, Sjogren's syndrome, Hashimoto's thyroiditis, biliary cirrhosis, Behcet's disease syndrome, Crohn's disease syndrome, polymyositis, systemic lupus erythematosus, immune-related glomerulonephritis, and rheumatoid arthritis); viral infections (such as, e.g., herpes viruses, pox viruses, and adenoviruses); inflammation; graft v. host disease syndrome, acute graft rejection, and chronic graft rejection.

In preferred embodiments, an LP is used to inhibit growth, progression, and/or metastases of cancers such as, in particular, those listed herein. Additional diseases, states, syndromes, or conditions associated with increased cell survival that could be modulated, ameliorated, treated, prevented, or diagnosed by an LP include, e.g., without limitation, progression, and/or metastases of malignancies and related disorders such as leukemia including acute leukemias (such as, e.g., acute lymphocytic leukemia, acute myelocytic leukemia, including myeloblastic, promyelocytic, myelomonocytic, monocytic, and erythroleukemia) and chronic leukemias (e.g., chronic myelocytic, chronic granulocytic, leukemia, and chronic lymphocytic leukemia)), polycythemia Vera, lymphomas (e.g., Hodgkin's disease, and non-Hodgkin's disease), multiple myeloma, Waldenstrom's macroglobulinemia, heavy chain disease, syndrome, and solid tumors including, e.g., without limitation, sarcomas and carcinomas (such as, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma).

Diseases associated with increased apoptosis that could be modulated, ameliorated, treated, prevented, and/or diagnosed by an LP include, e.g., AIDS, conditions (such as, e.g., Alzheimer's disease syndrome, Parkinson's disease syndrome, Amyotrophic lateral sclerosis, Retinitis pigmentosa, Cerebellar degeneration and brain tumor, or prion associated disease); autoimmune conditions (such as, e.g., multiple sclerosis, Sjogren's syndrome, Hashimoto's thyroiditis, biliary cirrhosis, Behcet's disease syndrome, Crohn's disease syndrome, polymyositis, systemic lupus erythematosus, immune-related glomerulonephritis, and rheumatoid arthritis); myelodysplastic syndromes (such as aplastic anemia), graft v. host disease syndrome; ischemic injury (such as that caused by myocardial infarction, stroke and reperfusion injury); liver injury (such as, e.g., hepatitis related liver injury, ischemia reperfusion injury, cholestosis (bile duct injury), and liver cancer); toxin-induced liver disease (such as, e.g., that caused by alcohol), septic shock, cachexia, and anorexia.

Wound Healing and Epithelial Cell Proliferation

In accordance with yet a further aspect of the invention, there is provided a process for using an LP to stimulate epithelial cell proliferation and basal keratinocytes for the purpose of, e.g., wound healing, to stimulate hair follicle production, and to heal a dermal wound. An LP composition may be clinically useful in stimulating wound healing including e.g., surgical wounds, excisional wounds, deep wounds involving damage of the dermis and epidermis, eye tissue wounds, dental tissue wounds, oral cavity wounds, diabetic ulcers, dermal ulcers, cubitus ulcers, arterial ulcers, venous stasis ulcers, burns resulting from exposure heat or chemicals, abnormal wound healing conditions associated with e.g., uremia, malnutrition, vitamin deficiency and wound healing complications associated with systemic treatment with steroids, radiation therapy, anti-neoplastic drugs, and anti-metabolites. An LP could be used to promote dermal reestablishment after dermal loss.

An LP could be used to increase the adherence of skin grafts to a wound bed and to stimulate re-epithelialization from the wound bed. The following is a non-exhaustive list of grafts that an LP could be used to increase adherence to: a wound bed, autografts, artificial skin, allografts, autodermic grafts, autoepidermic grafts, avascular grafts, Blair-Brown grafts, bone grafts, brephoplastic grafts, cutis grafts, delayed grafts, dermic grafts, epidermic grafts, fascia grafts, full thickness grafts, heterologous grafts, xenografts, homologous grafts, hyperplastic grafts, lamellar grafts, mesh grafts, mucosal grafts, Ollier-Thiersch grafts, omenpal grafts, patch grafts, pedicle grafts, penetrating grafts, split skin grafts, and thick split grafts. An LP can be used to promote skin strength and to improve the appearance of aged skin. It is believed that an LP will also produce changes in hepatocyte proliferation, and epithelial cell proliferation in, for example, the lung, breast, pancreas, stomach, small intestine, and large intestine. Epithelial cell proliferation can be effected in epithelial cells such as, e.g., sebocytes, hair follicles, hepatocytes, type II pneumocytes, mucin-producing goblet cells, and other epithelial cells or their progenitors which are contained within the skin, lung, liver, and gastrointestinal tract.

An LP may: promote proliferation of endothelial cells, keratinocytes, and basal keratinocytes; it could also be used to reduce the side effects of gut toxicity that result from radiation, chemotherapy treatments or viral infections, it may have a cytoprotective effect on the small intestine mucosa; it may also stimulate healing of mucositis (mouth ulcers) that result from chemotherapy and viral infections, it could further be used in full regeneration of skin in full and partial thickness skin defects, including burns, (i.e., re-population of hair follicles, sweat glands; and sebaceous glands), treatment of other skin defects such as psoriasis, it also could be used to treat epidermolysis bullosa, a defect in adherence of the epidermis to the underlying dermis which results in frequent, open and painful blisters by accelerating re-epithelialization of these lesions; it could also be used to treat gastric and doudenal ulcers and help heal by scar formation of the mucosal lining and regeneration of glandular mucosa and duodenal mucosal lining more rapidly. Inflammatory bowel diseases, such as Crohn's disease and ulcerative colitis, are diseases that result in destruction of the mucosal surface of the small or large intestine, respectively. Thus, an LP could be used to promote resurfacing of a mucosal surface to aid more rapid healing and to prevent progression of inflammatory bowel disease resulting in a desired effect, e.g., such as on the production of mucus throughout the gastrointestinal tract and the protection of intestinal mucosa from injurious substances that are ingested or following surgery. An LP could be used to treat a condition associated with the under expression of an LP polynucleotide sequence or an LP polypeptide of the present invention (or fragment thereof), or an agonist or antagonist thereto.

Moreover, an LP could be used to prevent and heal damage to the lungs due to various pathological states, such as, e.g., stimulating proliferation and differentiation to promote repair of alveoli and bronchiolar epithelium. For example, emphysema, inhalation injuries, that (e.g., from smoke inhalation) and burns, which cause necrosis of the bronchiolar epithelium and alveoli could be effectively ameliorated, treated, prevented, and/or diagnosed using a polynucleotide or polypeptide of the invention (or fragment thereof), or an agonist or antagonist thereto. Also, an LP could be used to stimulate the proliferation of and differentiation of type II pneumocytes, to help treat or prevent hyaline membrane diseases, such as e.g., infant respiratory distress syndrome and bronchopulmonary displasia, (in premature infants). An LP could stimulate the proliferation and/or differentiation of a hepatocyte and, thus, could be used to alleviate or treat a liver condition such as e.g., fulminant liver failure (caused, e.g., by cirrhosis), liver damage caused by viral hepatitis and toxic substances (e.g., acetaminophen, carbon tetrachloride, and other known hepatotoxins). In addition, an LP could be used treat or prevent the onset of diabetes mellitus. In patients with newly diagnosed Types I and II diabetes, where some islet cell function remains, an LP could be used to maintain the islet function so as to alleviate, modulate, ameliorate, delay, or prevent permanent manifestation of the disease. In addition, an LP could be used as an auxiliary in islet cell transplantation to improve or promote islet cell function.

Neurological Diseases

Nervous system diseases, disorders, syndromes, states, and/or conditions that can be modulated, ameliorated, treated, prevented, and/or diagnosed with an LP composition include, e.g., without limitation, nervous system injuries diseases, disorders, states, syndromes, and/or conditions that result in either a disconnection or misconnection of an axon or dendrite; a diminution or degeneration of a cell (or part of a cell) of the nervous system (such as, e.g., without limitation, neurons, astrocytes, microglia, macroglia, oligodendroglia, Schwann cells, and ependymal cells); demyelination or improper mylenation; neural cell dysfunction (such as, e.g., failure of neurotransmitter release or uptake); or interference with mylenization. Nervous system lesions that may be modulated, ameliorated, treated, prevented, and/or diagnosed in a subject using an LP composition of the invention, include, e.g., without limitation, the following lesions of either the central (including spinal cord and brain) or peripheral nervous system: (1) ischemic lesions, in which a lack of oxygen in a portion of the nervous system results in neuronal injury or death, including e.g., cerebral infarction (or ischemia), or spinal cord infarction (or ischemia); (2) traumatic lesions, including, e.g., lesions caused by physical injury or associated with surgery (e.g., lesions that sever a portion of the nervous system), or compression injuries; (3) malignant lesions, in which a portion of the nervous system is comprised by malignant tissue, which is either a nervous system associated malignancy or a malignancy derived from non-nervous-system tissue; (4) infectious lesions, in which a portion of the nervous system is comprised because of infection (e.g., by) an abscess or associated with infection by human immunodeficiency virus, herpes zoster, or herpes simplex virus or with Lyme disease, syndrome, tuberculosis, syphilis); (5) degenerative lesions, in which a portion of the nervous system is comprised because of a degenerative process including, without limit, degeneration associated with Parkinson's disease syndrome, Alzheimer's disease syndrome, Huntington's chorea, or Amyotrophic lateral sclerosis (ALS); (6) lesions associated with a nutritional condition, in which a portion of the nervous system is comprised by a nutritional disorder (or a disorder of metabolism including, without limit, vitamin B 12 deficiency, folic acid deficiency, Wernicke disease, syndrome, tobacco-alcohol amblyopia, Marchiafava-Bignami disease (primary degeneration of the corpus callosum), and alcoholic cerebellar degeneration; (7) neurological lesions associated with systemic diseases including, e.g., without limitation, diabetes (diabetic neuropathy, Bell's palsy), systemic lupus erythematosus, carcinoma, or sarcoidosis; (8) lesions caused by toxic substances including e.g., alcohol, lead, or a neurotoxin; and (9) demyelinating lesions in which a portion of the nervous system is comprised by a demyelinating cause (including, e.g., without limitation, multiple sclerosis, human immunodeficiency virus-associated myelopathy, transverse myelopathy or various etiologies, progressive multifocal leukoencephalopathy, and central pontine myelinolysis).

In a preferred embodiment, an LP can be used to protect a neuronal cell from the damaging effects of cerebral hypoxia; cerebral ischemia, cerebral infarction; stroke; or a neural cell injury associated with a heart attack. An LP, which is useful for producing a desired effect in a nervous system condition, may be selected by testing for biological activity in promoting survival and/or differentiation of neural cell. For example, an LP that elicits any of the following effects may be useful according to the invention: (1) increased survival time of neurons in culture; (2) increased or decreased sprouting of a neural in culture or in vivo; (3) increased or decreased production of a neuron-associated molecule e.g., such as a neurotransmitter in culture or in vivo, e.g., choline acetyltransferase or acetylcholinesterase with respect to a motor neuron; or (4) decreasing a symptom of neuronal dysfunction in vivo or in a model system, e.g., such as a mouse model for Parkinsons Syndrome. Such an effect may be measured by any known art method.

In a preferred, non-limiting embodiment any art known method can be used to: measure increased neuronal survival (such as, e.g., described in Arakawa, et al. (1990) J. Neurosci. 10:3507-3515); detect increased or decreased sprouting (such as, e.g., described in Pestronk, et al. (1980) Exp. Neurol. 70:65-82; Brown, et al. (1981) Ann. Rev. Neurosci. 4:17-42); measure increased production of a neuron-associated molecule (e.g., by bioassay, enzymatic assay, antibody binding, Northern blot assay, etc., depending on the molecule to be measured); and measure motor neuron dysfunction (by, e.g., assessing the physical manifestation of motor neuron disorder, e.g., weakness, motor neuron conduction velocity, or functional disability in a model system). In specific embodiments, motor neuron diseases, disorders, syndromes, and/or conditions that may be modulated, ameliorated, treated, prevented, and/or diagnosed using an LP composition include, e.g., without limitation, infarction, infection, exposure to toxin, trauma, surgical damage, degenerative disease or malignancy (that may affect motor neurons as well as other components of the nervous system), as well as conditions that selectively affect neurons such as, e.g., without limitation, Amyotrophic lateral sclerosis progressive spinal muscular atrophy, progressive bulbar palsy, primary lateral sclerosis, infantile and juvenile muscular atrophy, progressive bulbar paralysis of childhood (Fazio-Londe syndrome), poliomyelitis post polio syndrome, and Hereditary Motorsensory Neuropathy (Charcot-Marie-Tooth Disease).

Infectious Disease

An LP composition can be used to modulate, ameliorate, treat, prevent, and/or diagnose an effect of an infectious agent in a subject or associated with a condition. For example, by increasing an immune response e.g., particularly increasing the proliferation and differentiation a of B and/or a T cell, infectious diseases may be modulated, ameliorated, treated, prevented, and/or diagnosed. The immune response may be increased either by enhancing an existing immune response, or by initiating a new immune response. Alternatively, an LP may also directly inhibit an infectious agent, without necessarily eliciting an immune response. Viruses are a type of an infectious agent that can cause diseases, disorders, syndromes, and/or conditions that may be modulated, ameliorated, treated, prevented, and/or diagnosed using an LP composition of the invention. Examples of such viruses, include, e.g., without limitation, the following DNA and RNA viruses and viral families: Arbovirus, Adenoviridae, Arenaviridae, Arterivirus, Birnaviridae, Bunyaviridae, Caliciviridae, Circoviridae, Coronaviridae, Dengue, EBTV, HIV, Flaviviridae, Hepadnaviridae (Hepatitis), Herpesviridae (such as, e.g., Cytomegalovirus, Herpes Simplex, Herpes Zoster), Mononegavirus (e.g., Paramyxoviridae, Morbillivirus, Rhabdoviridae), Orthomyxoviridae (e.g., Influenza A, Influenza B, and parainfluenza), Papilomavirus, Papovaviridae, Parvoviridae, Picornaviridae, Poxviridae (such as, e.g., Smallpox or Vaccinia), Reoviridae (e.g., Rotavirus), Retroviridae (such as, e.g., HTLV-I, HTLV-II, Lentivirus),and Togaviridae (e.g., Rubivirus). Typically, viruses of these families can cause a variety of undesired conditions, including, but not limited to: e.g., arthritis, bronchiollitis, respiratory syncytial virus, encephalitis, eye infections (e.g., conjunctivitis, keratitis), chronic fatigue syndrome, hepatitis (e.g., of type A, B, C, E, Chronic Active, or Delta), Japanese Bencephalitis, Junin, Chikungunya, Rift Valley fever, yellow fever, meningitis, opportunistic infections (e.g., AIDS), pneumonia, Burkitt's Lymphoma, chickenpox, hemorrhagic fever, Measles, Mumps, Parainfluenza, Rabies, a common cold, Polio, leukemia, Rubella, sexually transmitted diseases, skin diseases (e.g., Kaposi's, warts), and viremia. An LP can be used to modulate, ameliorate, treat, prevent, and/or diagnose any of these symptoms or diseases.

In specific embodiments, an LP composition is used to modulate, ameliorate, treat, prevent, and/or diagnose e.g., meningitis, Dengue, EBV, and/or hepatitis (e.g., hepatitis B). In a further specific embodiment, an LP is administered to a subject that is non-responsive to one or more currently established commercially available, hepatitis vaccines. In a further specific embodiment an LP can be used to modulate, ameliorate, treat, prevent, and/or diagnose AIDS or an AIDS-related syndrome or condition. Similarly, bacterial or fungal agents that can cause a disease, disorder, condition, syndrome, or symptom and that can be ameliorated, treated, prevented, and/or diagnosed by an LP composition of the invention include, e.g., but without limitation, the following: Gram-Negative and Gram-positive bacteria and bacterial families and fungi such as: Actinomycetales (e.g., Corynebacterium, Mycobacterium, Norcardia), Cryptococcus neoformans, Aspergillosis, Bacillaceae (e.g., Anthrax, Clostridium), Bacteroidaceae, Blastomycosis, Bordetella, Borrelia (e.g., Borrelia burgdorferi), Brucellosis, Candidiasis, Campylobacter, Coccidioidomycosis, Cryptococcosis, Dermatocycoses, E. coli (e.g., Enterotoxigenic E. coli and Enterohemorrhagic E. coli), Enterobacteriaceae (Klebsiella, Salmonella (e.g., Salmonella typhi, and Salmonella paratyphi), Serratia, Yersinia), Erysipelothrix, Helicobacter, Legionellosis, Leptospirosis, Listeria, Mycoplasmatales, Mycobacterium leprae, Vibrio cholerae, Neisseriaceae (e.g., Acinetobacter, Gonorrhea, Menigococcal), Meisseria meningitidis, Pasteurellacea Infections (e.g., Actinobacillus, Heamophilus (e.g., Heamophilus influenza type B), Pasteurella), Pseudomonas, Rickettsiaceae, Chlamydiaceae, Syphilis, Shigella spp., Staphylococcal, Meningiococcal, Pneumococcal and Streptococcal (e.g., Streptococcus pneumoniae and Group B Streptococcus).

These bacterial or fungal families can cause the following diseases, disorders, conditions, syndromes, or symptoms including, e.g., without limitation, bacteremia, endocarditis, eye infections (conjunctivitis, tuberculosis, uveitis), gingivitis, opportunistic infections (e.g., AIDS related infections), paronychia, prosthesis-related infections, Reiter's Disease syndrome, respiratory tract infections, such as Whooping Cough or Empyema, sepsis, Lyme Disease syndrome, Cat-Scratch Disease syndrome, Dysentery, Paratyphoid Fever, food poisoning, Typhoid, pneumonia, Gonorrhea, meningitis (e.g., meningitis types A and B), Chlamydia, Syphilis, Diphtheria, Leprosy, Paratuberculosis, Tuberculosis, Lupus, Botulism, gangrene, tetanus, impetigo, Rheumatic Fever, Scarlet Fever, sexually transmitted diseases, skin diseases (e.g., cellulitis, dermatocycoses), toxemia, urinary tract infections and wound infections. An LP can be used to modulate, ameliorate, treat, prevent, and/or diagnose any of these diseases, disorders, conditions, syndromes, or symptoms.

In specific embodiments, an LP composition can be used to modulate, ameliorate, treat, prevent, and/or diagnose: tetanus, Diptheria, botulism, and/or meningitis type B. Moreover, parasitic agents causing diseases, disorders, conditions, syndromes, or symptoms that can be modulated, ameliorated, treated, prevented, and/or diagnosed by an LP include, e.g., without limitation, a parasitic agent from any of the following groupings: Amebiasis, Babesiosis, Coccidiosis, Cryptosporidiosis, Dientamoebiasis, Dourine, Ectoparasitic, Giardiasis, Helminthiasis, Leishmaniasis, Theileriasis, Toxoplasmosis, Trypanosomiasis, Trichomona, Sporozoans (e.g., Plasmodium virax, Plasmodium falciparium, Plasmodium malariae, and Plasmodium ovale). These parasites can cause a variety of diseases or symptoms, including, e.g., without limitaton: Scabies, Tromibiculiasis, eye infections, intestinal disease (e.g., dysentery, giardiasis), liver disease syndrome, lung disease syndrome, opportunistic infections (e.g., AIDS related conditions), malaria, complications of pregnancy, and toxoplasmosis. An LP composition of the invention can be used to modulate, ameliorate, treat, prevent, and/or diagnose any of these diseases, disorders, conditions, syndromes, or symptoms. In specific embodiments, an LP can be used to modulate, ameliorate, treat, prevent, and/or diagnose malaria.

Preferably, treatment or prevention using an LP is accomplished either by administering an effective amount of an LP composition to a subject, or by removing cells from a subject, delivering an LP then returning the resulting engineered cell to the patient (ex vivo therapy). Furthermore, an LP sequence can be used as an antigen in a vaccine to raise an immune response against an infectious disease.

Regeneration

An LP composition of the invention can be used e.g., to differentiate a cell, tissue; or biological structure, de-differentiate a cell, tissue; or biological structure; cause proliferation in cell or a zone (similar to a ZPA in a limb bud), have an effect on chemotaxis, remodel a tissue (e.g., basement membrane, extra cell matrix, connective tissue, muscle, epithelia), or initiate the regeneration of a tissue, organ, or biological structure (see, e.g., Science (1997) 276:59-87). Regeneration using an LP composition of the invention could be used to repair, replace, remodel, or protect tissue damaged by, e.g., congenital defects, trauma (such as, e.g., wounds, burns, incisions, or ulcers); age; disease (such as, e.g., osteoporosis, osteoarthritis, periodontal disease syndrome, or liver failure), surgery, (including, e.g., cosmetic plastic surgery); fibrosis; re-perfusion injury; or cytokine damage. Tissues that can be regenerated include, e.g., without limitation, organs (e.g., pancreas, liver, intestine, kidney, epithelia, endothelium), muscle (smooth, skeletal, or cardiac), vasculature (including vascular and lymphatics), nervous system tissue, cells, or structures; hematopoietic tissue; and skeletal (bone, cartilage, tendon, and ligament) tissue. Preferably, regeneration occurs with little or no scarring. Regeneration also may include, e.g., angiogenesis.

Moreover, an LP composition may increase the regeneration of an aggregation of special cell types, a tissue, or a matrix that typically is difficult to heal. For example, by increasing the rate at which a tendon/ligament heals after damage. Also encompassed is using an LP prophylactically to avoid damage (e.g., in an interstitial space of a joint or on the cartalagenous capsule of a bone). Specific diseases that could be modulated, ameliorated, treated, prevented, and/or diagnosed using an LP composition include, e.g., without limitation, tendinitis, carpal tunnel syndrome, and other tendon or ligament defects. Examples of non-healing wounds include, wounds that would benefit form regeneration treatment, e.g., without limit pressure ulcers, ulcers associated with vascular insufficiency, surgical wounds, and traumatic wounds.

Similarly, nerve and brain tissue also could be regenerated using an LP. Such nervous system conditions that could be modulated, ameliorated, treated, prevented, and/or diagnosed using an LP composition include, e.g., without limitation, central and peripheral nervous system diseases, neuropathies, or mechanical and traumatic conditions (e.g., spinal cord disorders or syndromes, head trauma, cerebrovascular disease syndrome, and stoke). Specifically, diseases associated with peripheral nerve injuries include, e.g., without limitation, peripheral neuropathy (e.g., resulting from chemotherapy or other medical therapies), localized neuropathies, and central nervous system diseases (e.g., Alzheimer's disease syndrome, Parkinson's disease syndrome, Huntington's disease syndrome, Amyotrophic lateral sclerosis, and Shy-Drager syndrome). All could be ameliorated, treated, prevented, and/or diagnosed using an LP.

An LP may have an effect on a chemotaxis activity. Briefly, chemotactic molecules can attract or mobilize (but may also repeal) cells (e.g., monocytes, fibroblasts, neutrophils, T-cells, mast cells, cosinophils, epithelial and/or endothelial cells) or cell processes (e.g., filopodia, psuedopodia, lamellapodia, dendrites, axons, etc.) to a particular site (e.g., such as inflammation, infection, site of hyperproliferation, the floor plate of the developing spinal cord, etc.). In some instances, such mobilized cells can then fight off and/or modulate a particular trauma, abnormality, condition, syndrome, or disease. An LP may have an effect on a chemotactic activity of a cell (such as, e.g., an attractive or repulsive effect).

A chemotactic molecule can be used to modulate, ameliorate, treat, prevent, and/or diagnose inflammation, infection, hyperproliferative diseases, disorders, syndromes, and/or conditions, or an immune system disorder by increasing the number of cells targeted to a particular location in the body. For example, a chemotactic molecule can be used to attract an immune cell to an injured location in a subject. An LP that had an effect on a chemotactant could also attract a fibroblast, which can be used to modulate, ameliorate, and/or treat a wound. It is also contemplated that an LP may inhibit a chemotactic activity to modulate, ameliorate, treat, prevent, and/or diagnose a disease, disorder, syndrome, and/or a condition.

XI. Kits

This invention also contemplates use of LP proteins, fragments thereof, peptides, and their fusion products in a variety of diagnostic kits and methods for detecting the presence of LP protein or a binding partner. Typically, the kit will have a compartment containing either a defined LP protein peptide or gene segment or a reagent, which recognizes one or the other, e.g., binding partner fragments or antibodies.

A preferred kit for determining the concentration of, e.g., a LP protein in a sample would typically comprise a labeled compound, e.g., binding partner or antibody, having known binding affinity for the LP protein, a source of LP protein (naturally occurring or recombinant), and a means for separating the bound from free labeled compound, for example, a solid phase for immobilizing the LP protein. Compartments containing reagents, and instructions, will normally be provided. Another diagnostic aspect of this invention involves use of oligonucleotide or polynucleotide sequences taken from the sequence of a LP protein. These sequences are used as probes for detecting levels of the LP protein message in samples from natural sources, or patients suspected of having an abnormal condition, e.g., cancer or developmental problem. The preparation of both RNA and DNA nucleotide sequences, the labeling of the sequences, and the preferred size of the sequences has received ample description and discussion in the literature.

In specific embodiments, a kit may include, e.g., a recombinantly produced or chemically synthesized polypeptide antigen. The polypeptide antigen of the kit may also be attached to a solid support. In a more specific embodiment the detecting means of the above-described kit includes, e.g., a solid support to which said polypeptide antigen is attached. Such a kit may also include, e.g., a non-attached reporter-labeled anti-human antibody. In this embodiment, binding of the antibody to the polypeptide antigen is detected by binding of the reporter-labeled antibody.

Other Preferred Embodiments

Other preferred embodiments of the claimed invention include an isolated or recombinant nucleic acid molecule comprising a polynucleotide sequence that is at least 95% identical to a polynucleotide sequence of at least about: 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 120, 130, 140, or 150 contiguous nucleotides of a sequence of SEQ ID NO:X wherein X is any integer as defined in a Table herein. Other preferred embodiments of the claimed invention include an isolated or recombinant nucleic acid molecule comprising a polynucleotide sequence that is at least 95% identical to a polynucleotide sequence of at least about: 10, 12, 14, 16, 18, 20, 22, 24, 26, 28, 30, 32, 34. 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 120, 130, 140, or 150 contiguous nucleotides of a mature coding portion of SEQ ID NO:X wherein X is any integer as defined in a Table herein. Also preferred is a nucleic acid molecule wherein said sequence of contiguous nucleotides is include, e.g. in the nucleotide sequence of SEQ ID NO:X in the range of positions beginning with the nucleotide at about the position of the 5′ nucleotide of the Clone Sequence and ending with the nucleotide at about the position of the 3′ nucleotide of the Clone Sequence as defined for SEQ ID NO:X in a Table herein. Also preferred is a nucleic acid molecule wherein said sequence of contiguous nucleotides is included, e.g., in the nucleotide sequence of SEQ ID NO:X in the range of positions beginning with the nucleotide at about the position of the 5′ nucleotide of the Start Codon and ending with the nucleotide at about the position of the 3′ nucleotide of the Clone Sequence as defined for SEQ ID NO:X in a Table herein. Similarly preferred is a nucleic acid molecule comprising polynucleotide sequence of SEQ ID NO:X in the range of positions beginning with the nucleotide at about the position of the 5′ nucleotide of a correspondingly encoded First Amino Acid of a Signal Peptide and ending with the nucleotide at about the position of the 3′ nucleotide of a Clone Sequence as defined for SEQ ID NO:X in a Table herein. Also preferred is an isolated or recombinant nucleic acid molecule comprising a polynucleotide sequence that is at least 9⁵% identical to a polynucleotide sequence of at least about: 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 120, 130, 140, or 150 contiguous nucleotides in at least one polynucleotide sequence fragment of SEQ ID NO:X. More preferably said polynucleotide sequence that is at least 95% identical to one, exhibits 95% sequence identity to at least: 2, 3, 4, 5, 6, 7, 8, 9, 10, or more polynucleotide fragments 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 120, 130, 140, or 150 contiguous nucleotides in length of the mature coding portion of SEQ ID NO:X., wherein any one such fragment is at least 21 contiguous nucleotides in length. Further preferred is an isolated or recombinant nucleic acid molecule comprising a polynucleotide sequence that is at least 95% identical to a polynucleotide sequence of at least about: 200, 250, 300, 350, 400, 450, or 500 contiguous nucleotides of the mature coding portion of SEQ ID NO:X. Also preferred is an isolated or recombinant nucleic acid molecule comprising a polynucleotide sequence that is at least 95% identical to a sequence of at least about: 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 120, 130, 140, or 150 contiguous nucleotides in at least one nucleotide sequence fragment of SEQ ID NO:X, wherein the length of at least one such fragment is about 200, 250, 300, 350, 400, 450, or 500 contiguous nucleotides of SEQ ID NO:X. Another preferred embodiment is an isolated or recombinant nucleic acid molecule comprising a polynucleotide sequence that is at least 95% identical to a sequence of SEQ ID NO:X beginning with the nucleotide at about the position of the 5′ Nucleotide of the First Amino Acid of the Signal Peptide and ending with the nucleotide at about the position of the 3′ Nucleotide of a Clone Sequence as defined for SEQ ID NO:X in a Table herein. A further preferred embodiment is an isolated or recombinant nucleic acid molecule comprising a polynucleotide sequence, which is at least 95% identical to the complete mature coding portion of SEQ ID NO:X or a species variant thereof. Also preferred is an isolated or recombinant nucleic acid molecule comprising polynucleotide sequence that hybridizes under stringent hybridization conditions to a mature coding portion of a polynucleotide of the invention (or fragment thereof), wherein the nucleic acid molecule that hybridizes does not hybridize under stringent hybridization conditions to a nucleic acid molecule having a nucleotide sequence consisting of only A residues or of only T residues. Thus, the invention provides an assay system or kit for carrying out a diagnostic method. The kit generally includes, e.g., a support with surface-bound recombinant antigens, and a reporter-labeled anti-human antibody for detecting surface-bound anti-antigen antibody.

The broad scope of this invention is best understood with reference to the following examples, which are not intended to limit the invention to specific embodiments.

### EXAMPLES

General Methods

Many of the standard methods described herein are described or referenced, e.g., in Maniatis, et al. (Cur. ed.) Molecular Cloning A Laboratory Manual Cold Spring Harbor Laboratory, Cold Spring Harbor Press, NY; Sambrook, et al.; Ausubel, et al., Biology Greene Publishing Associates, Brooklyn, N.Y.; or Ausubel, et al. (1987 and Supplements) Current Protocols in Molecular Biology Wiley/Greene, NY; Innis, et al. (eds.) (1990) PCR Protocols: A Guide to Methods and Applications Academic Press, NY. Methods for protein purification include such methods as ammonium sulfate precipitation, column chromatography, electrophoresis, centrifugation, crystallization, and others. See, e.g., Ausubel, et al. (1987 and periodic supplements); Deutscher (1990) “Guide to Protein Purification,” Methods in Enzymology vol. 182, and other volumes in this series; Coligan, et al. (1995 and supplements) Current Protocols in Protein Science John Wiley and Sons, New York, N.Y.; P. Matsudaira (ed.) (1993) A Practical Guide to Protein and Peptide Purification for Microsequencing, Academic Press, San Diego, Calif.; and manufacturer's literature on use of protein purification products, e.g., Pharmacia, Piscataway, N.J., or Bio-Rad, Richmond, Calif. Combination with recombinant techniques allow fusion to appropriate segments (epitope tags), e.g., to a FLAG sequence or an equivalent which can be fused, e.g., via a protease-removable sequence. See, e.g., Hochuli (1989) Chemische Industrie 12:69-70; Hochuli (1990) “Purification of Recombinant Proteins with Metal Chelate Absorbent” in Setlow (ed.) Genetic Engineering, Principle and Methods 12:87-98, Plenum Press, NY; and Crowe, et al. (1992) QIAexpress: The High Level Expression and Protein Purification System QUIAGEN, Inc., Chatsworth, Calif. Standard immunological techniques are described, e.g., in Hertzenberg, et al. (eds. 1996) Weir's Hanbook of Experimental Immunology vols. 1-4, Blackwell Science; Coligan (1991) Current Protocols in Immunology Wiley/Greene, NY; and Methods in Enzymology volumes. 70, 73, 74, 84, 92, 93, 108, 116, 121, 132, 150, 162, and 163. Assays for neural cell biological activities are described, e.g., in Wouterlood (ed. 1995) Neuroscience Protocols modules 10, Elsevier; Methods in Neurosciences Academic Press; and Neuromethods Humana Press, Totowa, N.J. Methodology of developmental systems is described, e.g., in Meisami (ed.) Handbook of Human Growth and Developmental Biology CRC Press; and Chrispeels (ed.) Molecular Techniques and Approaches in Developmental Biology Interscience. FACS analyses are described in Melamed, et al. (1990) Flow Cytometry and Sorting Wiley-Liss, Inc., New York, N.Y.; Shapiro (1988) Practical Flow Cytometry Liss, New York, N.Y.; and Robinson, et al. (1993) Handbook of Flow Cytometry Methods Wiley-Liss, New York, N.Y.

### Example 1

### Isolation of LP clones

Standard methods are used to isolate full length genes from a cDNA library made from an appropriate source, e.g., human cells. The appropriate sequence is selected, and hybridization at high stringency conditions is performed to find a full length corresponding gene using standard techniques. The full length, or appropriate fragments, of human genes are used to isolate a corresponding monkey or other primate gene. Preferably, a full length coding sequence is used for hybridization. Similar source materials as indicated above are used to isolate natural genes, including genetic, polymorphic, allelic, or strain variants. Other species variants are also isolated using similar methods. With a positive clone, the coding sequence is inserted into an appropriate expression vector. This may be in a vector specifically selected for a prokaryote, yeast, insect, or higher vertebrate, e.g., mammalian expression system. Standard methods are applied to produce the gene product, preferably as a soluble secreted molecule, but will, in certain instances, also be made as an intracellular protein. Intracellular proteins typically require cell lysis to recover the protein, and insoluble inclusion bodies are a common starting material for further purification. With a clone encoding a vertebrate LP protein, recombinant production means are used, although natural forms may be purified from appropriate sources. The protein product is purified by standard methods of protein purification, in certain cases, e.g., coupled with immunoaffinity methods. Immunoaffinity methods are used either as a purification step, as described above, or as a detection assay to determine the separation properties of the protein.

Preferably, the protein is secreted into the medium, and the soluble product is purified from the medium in a soluble form. Alternatively, as described above, inclusion bodies from prokaryotic expression systems are a useful source of material. Typically, the insoluble protein is solubilized from the inclusion bodies and refolded using standard methods. Purification methods are developed as described herein. The product of the purification method described above is characterized to determine many structural features. Standard physical methods are applied, e.g., amino acid analysis and protein sequencing. The resulting protein is subjected to CD spectroscopy and other spectroscopic methods, e.g., NMR, ESR, mass spectroscopy, etc. The product is characterized to determine its molecular form and size, e.g., using gel chromatography and similar techniques. Understanding of the chromatographic properties will lead to more gentle or efficient purification methods. Prediction of glycosylation sites may be made, e.g., as reported in Hansen, et al. (1995) Biochem. J. 308:801-813. The purified protein is also be used to identify other binding partners of an LP of the invention as described, e.g., in Fields and Song (1989) Nature 340:245-246.

### Example 2

### Tissue Distribution of an LP Polynucleotide

Tissue distribution of mRNA expression of a polynucleotide of the present invention (or fragment thereof) is determined using protocols for Northern blot analysis, described (among others) by, e.g., Sambrook, et al. For example, a cDNA probe produced using common techniques is labeled with p³² using the Rediprime™ DNA labeling system (Amersham Life Science), according to manufacturer's instructions. After labeling, the probe is purified using CHROMA SPIN-100™ column (Clontech Laboratories, Inc.), according to manufacturer's protocol number PT1200-1. The purified, labeled probe is then used to examine various human tissues for mRNA expression. Multiple Tissue Northern (MTN) blots containing various human tissues (H) or human immune system tissues (IM) (Clontech) are examined with the labeled probe using Express Hyb™ hybridization solution (Clontech) according to manufacturer's protocol number PT1190-1. After hybridization and washing, blots are mounted and exposed to film (overnight at −70° C.), and the films are subsequently developed according to standard procedures.

### Example 3

### Chromosomal Mapping of an LP Polynucleotide

An oligonucleotide primer set is designed according to the sequence at the 5′ end of a SEQ ID NO:X identified sequence. This primer preferably spans about 100 nucleotides. This primer set is then used in a polymerase chain reaction under the following set of conditions: 30 seconds, 95° C.; 1 minute, 56° C.; 1 minute, 70° C. This cycle is repeated 32 times followed by one 5-minute cycle at 70° C. Human, mouse, and hamster DNA is used as template in addition to a somatic cell hybrid panel containing individual chromosomes or chromosome fragments (Bios, Inc). The reaction is analyzed on either 8% polyacrylamide gels or 3.5 % agarose gels. Chromosome mapping is determined by the presence of an approximately 100 bp PCR fragment in a particular somatic cell hybrid.

### Example 4

### Production of a Secreted LP Protein for a High-Throughput Screening Assay

The following protocol produces a supernatant containing an LP polypeptide (or fragment thereof) to be tested. This supernatant can then be used in a variety of screening assays (such as, e.g., those taught herein). First, dilute Poly-D-Lysine (644 587 Boehringer-Mannheim) stock solution (1 mg/ml in PBS) 1:20 in PBS (w/o calcium or magnesium 17-5 16F Biowhittaker) to obtain a working stock solution of 50 ug/ml. Add 200 ul of this solution to each well (24-well plates) and incubate (RT for 20 min). Distribute the solution over each well (a 12-channel pipetter may be used with tips on every other channel). Aspirate off the Poly-D-Lysine solution and rinse with 1 ml PBS (Phosphate Buffered Saline). The PBS should remain in the well until just before plating the cells and plates may be coated (up to two weeks in advance) with poly-lysine. Plate 2933: cells (do not carry cells past P+20) at 2×10⁵ cells/well in 0.5 ml DMEM (Dulbecco's Modified Eagle Medium)(with 4.5 G/L glucose and L-glutamine5 (12-604F Biowhittaker))/10% heat inactivated FBS (14-503F Biowhittaker)/1× Pinstripe (17-602E Biowhittaker). Let the cells grow overnight.

The next day, mix in a sterile solution basin: 300 ul Lipofectamine (18324-012 Gibco; BRL) and 5ml Optimem I (31985070 Gibco; BRL) per 96-well plate. With a small volume multi-channel pipetter, aliquot approximately 2 ug of an expression vector containing an LP polynucleotide insert of the invention, produced by any art known methods or as taught herein, into an appropriately labeled 96-well round-bottom plate. With a multi-channel pipetter, add 50 μl of the Lipofectamine/Optimem I mixture to each well. Pipette up and down gently to mix. Incubate at RT for 15-45 minutes. After about 20 minutes, use a multi-channel pipetter to add 150 μl of Optimem I to each well. As a control, transfect one plate of vector DNA lacking an insert with each set of transfections.

Preferably, transfections should be performed by splitting the following tasks between two individuals to reduce the time, and to insure that the cells do not spend too much time in PBS. First, person A aspirates off the media from four 24-well plates of cells, and then person B rinses each well with 0.5-l ml of PBS. Person A then aspirates off the PBS rinse, and person B (using a 12-channel pipetter with tips on every other channel) adds 200 μl of DNA/Lipofectamine/Optimem I complex first to the odd wells, then to the even wells (of each row on the 24-well plates). Incubate at 37° C. for 6 hours. While cells are incubating, prepare appropriate media, either 1% BSA in DMEM with 1× penstrep, or CHO-5 media (116.6 mg/L of CaCl₂ (anhyd); 0.00130 mg/L CuSO₄-5H₂O; 0.050 mg/L of Fe(NO₃)₃-9H₂O ; 0.417 mg/L of FeSO₄-7H₂O; 311.80 mg/L of KCl; 28.64 mg/L of MgCl₂; 48.84 mg/L of MgSO₄; 6995.50 mg/L of NaCl; 2400.0 mg/L of NaHCO₃; 62.50 mg/L of NaH₂PO₄—H₂O; 71.02 mg/L of Na₂HPO₄; 0.4320 mg/L of ZnSO₄-7H₂0; 0.002 mg/L of Arachidonic Acid; 1.022 mg/L of Cholesterol; 0.070 mg/L of DL-alpha-Tocopherol-Acetate; 0.0520 mg/L of Linoleic Acid; 0.010 mg/L of Linolenic Acid; 0.010 mg/L of Myristic Acid; 0.010 mg/L of Oleic Acid; 0.010 mg/L of Palmitric Acid; 0.010 mg/L of Palmitic Acid; 100 mg/L of Pluronic F-68; 0.010 mg/L of Stearic Acid; 2.20 mg/L of Tween 80; 4551 mg/L of D-Glucose; 130.85 mg/ml of L-Alanine; 147.50 mg/ml of L-Arginine-HCL; 7.50 mg/ml of L-Asparagine-H₂O; 6.65 mg/ml of L-Aspartic Acid; 29.56 mg/ml of L-Cystine-2HCL-H₂O; 31.29 mg/ml of L-Cystine-2HCL; 7.35 mg/ml of L-Glutamic Acid; 365.0 mg/ml of L-Glutamine; 18.75 mg/ml of Glycine; 52.48 mg/ml of L-Histidine-HCL-H₂O; 106.97 mg/ml of L-Isoleucine; 111.45 mg/ml of L-Leucine; 163.75 mg/ml of L-Lysine HCL; 32.34 mg/ml of L-Methionine; 68.48 mg/ml of L-Phenylalanine; 40.0 mg/ml of L-Proline; 26.25 mg/ml of L-Serine; 101.05 mg/ml of L-Threonine; 19.22 mg/ml of L-Tryptophan; 91.79 mg/ml of L-Tryrosine-2Na-2H₂O; 99.65 mg/ml of L-Valine; 0.0035 mg/L of Biotin; 3.24 mg/L of D-Ca Pantothenate; 11.78 mg/L of Choline Chloride; 4.65 mg/L of Folic Acid; 15.60 mg/L of i-Inositol; 3.02 mg/L of Niacinamide; 3.0 mg/L of Pyridoxal HCL; 0.031 mg/L of Pyridoxine HCL; 0.319 mg/L of Riboflavin; 3.17 mg/L of Thiamine HCL; 0.365 mg/L of Thymidine; and 0.680 mg/L of Vitamin B12; 25 mM of HEPES Buffer; 2.39 mg/L of Na Hypoxanthine; 0.105 mg/L of Lipoic Acid; 0.081 mg/L of Sodium Putrescine-2HCL; 55.0 mg/L of Sodium Pyruvate; 0.0067 mg/L of Sodium Selenite; 20 uM of Ethanolamine; 0.122 mg/L of Ferric Citrate; 41.70 mg/L of Methyl-B-Cyclodextrin complexed with Linoleic Acid; 33.33 mg/L of Methyl-B-Cyclodextrin complexed with Oleic Acid; and 10 mg/L of Methyl-B-Cyclodextrin complexed with Retinal) with 2 mm glutamine, and 1× penstrep (BSA (81-068-3 Bayer) 100 gm dissolved in 1 L DMEM for a 10% BSA stock solution). Filter the media and collect 50 ul for endotoxin assay in 15 ml polystyrene conical.

The transfection reaction is terminated, preferably by splitting tasks (as above) at the end of the incubation period. Person A aspirates off the transfection media, while person B adds 1.5 ml appropriate media to each well. Incubate at 37° C. for 45 or 72 hours depending on the media used (1% BSA for 45 hours or CHO-5 for 72 hours). On day four, using a 300 ul multichannel pipetter, aliquot 600 μl in one 1 ml deep well plate and the remaining supernatant into a 2 ml deep well. The supernatants from each well can then be used in an assay taught herein. It is specifically understood that when activity is obtained in an assay described herein using a supernatant, the activity originates either from the polypeptide (or fragment thereof) directly (such as, e.g., from a secreted protein or fragment thereof) or by the polypeptide (or fragment thereof) inducing expression of another protein(s), which is/are then released into the supernatant. Thus, the invention provides a method of identifying a polypeptide (or fragment thereof) in a supernatant characterized by an activity in a particular assay taught herein.

### Example 5

### Construction of a GAS Reporter Construct

One signal transduction pathway involved in cellular differentiation and proliferation is a Jaks-STATS pathway. Activated proteins in a Jaks-STATS pathway have been shown to bind to gamma activation site “GAS” elements or interferon-sensitive responsive element (“ISRE”), which are located, e.g., in the promoter region of many genes. Typically, binding, e.g., by a protein, to such an element alters expression of an associated gene. GAS and ISRE elements are recognized by a class of transcription factors called Signal Transducers and Activators of Transcription, or “STATS.” The Stat1 and Stat3 members of the STATS family are present in many cell types, (as is Stat2) probably, because the response to IFN-alpha is widespread. Stat4, however, is more restricted to particular cell types though, it has been found in T helper class I cells after their treatment with IL-12. Stat 5 (originally designated mammary growth factor) has been found at higher concentrations in cells besides breast cells, e.g., myeloid cells. Stat 5 is activated in tissue culture cells by many cytokines.

After tyrosine phosphorylation (by kinases known as the Janus Kinase Family or “Jaks”), members of the STATS family typically translocate from the cytoplasm to the nucleus of the cell. Jaks represent a distinct family of soluble tyrosine kinases and include, e.g., Tyk2, Jak1, Jak2, and Jak3. These Jak kinases display significant sequence similarity to each other and, generally, are catalytically inactive in resting cells. However, Jaks are catalytically activated by a wide range of receptors (summarized in the Table below, adapted from Schidler and Darnell (1995) Ann. Rev. Biochem. 64:621-51). One cytokine receptor family, which is capable of activating a Jak, is divided into two groups (Class 1 and 2). Class 1 includes, e.g., receptors for IL-2, IL-3, IL-4, IL-6, IL-7, IL-9, IL-11, IL-12, IL-15, Epo, PRL, GH, G-CSF, GM-CSF, LIF, CNTF, and thrombopoietin; while Class 2 includes, e.g., IFN-a, IFN-g, and IL-10. The Class 1 receptors share a conserved cysteine motif (a set of four conserved cysteines and one tryptophan) and a WSXWS motif (a membrane proximal region encoding Trp-Ser-Xxx-Trp-Ser). Thus, after a ligand binds a receptor, Jaks are typically activated and, in turn, subsequently activate STATS, which translocate and bind to GAS transcriptional elements (located in the nucleus of the cell). This entire process of sequential activation is encompassed in a typical Jaks-STATS signal transduction pathway. Therefore, activation of a Jaks-STATS pathway (reflected by binding of a GAS or 1SRE element) is used to indicate that an LP polypeptide (or fragment thereof) is involved in the proliferation and/or differentiation of a cell. For instance, growth factors and cytokines are examples of proteins that are known to activate a Jaks-STATS pathway. Consequently, by using a GAS element linked to a reporter molecule, an activator of a Jaks-STATS pathway is identified.

| | | JAKS | | | | |
|---|---|---|---|---|---|---|
| Ligand | tyk2 | Jak1 | Jak2 | Jak3 | STATS | GAS(elements) or ISRE |
| IFN family | | | | | | |
| IFN-a/B | + | + | − | − | 1, 2, 3 | ISRE |
| IFN-G | | + | + | − | 1 | GAS (IRF1>Lys6>IFP) |
| IL-10 | + | ? | ? | − | 1, 3 | |
| gp130 family | | | | | | |
| IL-6 (Pleiotrophic) | + | + | + | ? | 1, 3 | GAS (IRFl>Lys6>IFP) |
| IL-11 (Pleiotrophic) | ? | + | ? | ? | 1, 3 | |
| OnM (Pleiotrophic) | ? | + | + | ? | 1, 3 | |
| LIF (Pleiotrophic) | ? | + | + | ? | 1, 3 | |
| CNTF (Pleiotrophic) | −/+ | + | + | ? | 1, 3 | |
| G-CSF (Pleiotrophic) | ? | + | ? | ? | 1, 3 | |
| IL-12 (Pleiotrophic) | + | − | + | + | 1, 3 | |
| g-C family | | | | | | |
| IL-2 (lymphocytes) | − | + | − | + | 1, 3, 5 | GAS |
| IL-4 (lymph/myeloid) | − | + | − | + | 6 | GAS (IRF1=IFP>>Ly6) (IgH) |
| IL-7 (lymphocytes) | − | + | − | + | 5 | GAS |
| IL-9 (lymphocytes) | − | + | − | + | 5 | GAS |
| IL-13 (lymphocyte) | − | + | ? | ? | 6 | GAS |
| IL-15 | ? | + | ? | + | 5 | GAS |
| gp140 family | | | | | | |
| IL-3 (myeloid) | − | − | + | − | 5 | GAS (IRF1>IFP>>Ly6) |
| IL-5 (myeloid) | − | − | + | − | 5 | GAS |
| GM-CSF (myeloid) | − | − | + | − | 5 | GAS |
| Growth hormone family | | | | | | |
| GH | ? | − | + | − | 5 | |
| PRL | ? | +/− | + | − | 1, 3, 5 | |
| EPO | ? | − | + | − | 5 | GAS |
| | | | | | | (B-CAS>IRF1=IFP>>Ly6) |
| Receptor Tyrosine | | | | | | |
| Kinases | | | | | | |
| EGF | ? | + | + | − | 1, 3 | GAS (IRF1) |
| PDGF | ? | + | + | − | 1, 3 | |
| CSF-1 | ? | + | + | − | 1, 3 | GAS (not IRF1) |

To construct a synthetic GAS containing promoter element, like that described in an assays taught herein, a PCR based strategy is employed to generate a GAS-SV40 promoter sequence. The 5′ primer contains four tandem copies of the GAS binding site found in the IRF1 promoter, which has previously been shown to bind STATS after induction by a range of cytokines (see, e.g., Rothman, et al. (1994) Immunity 1:457-468). Although, however, it is possible to use other GAS or ISRE elements. The 5′ primer also contains 18 bp of sequence complementary to the SV40 early promoter sequence and is flanked with an XhoI site. The sequence of the 5′ primer is:

| | |
|---|---|
| 5′: GCGCCTCGACATTTCCCCGAAATCTAGATTTCCCCGAAATGATTTCCCCGAAATGATTTCCCCGAAATA | (SEQ ID NO:24) |
| TCTGCCATCTCAATTAG:3′ | |

The downstream primer, which is complementary to the SV40 promoter and is flanked with a Hind III site, is: 5′:GCGGCAAGCTTTTTGCAAAGCCTAGGC:3′ (SEQ ID NO:25). PCR amplification is performed using the SV40 promoter template present in a B-gal:promoter plasmid (Clontech). The resulting PCR fragment is digested with xhoI/Hind III and subcloned into BLSK2- (Stratagene). Sequencing with forward and reverse primers confirms that the insert contains the following sequence:

| | |
|---|---|
| 5′:CTCGAGATTTCCCCGAAATCTAGATTTCCCCGAAATGATTCCCCGAAATGATTTCCCCGAAATATCTGC | (SEQ ID NO:26) |
| CATCTCAATTAGTCAGCAACCATAGTCCCGCCCCTAACTCCGCCCATCCCGCCCCTAACTCCGCCCAGTTCC | |
| GCCCATTCTCCGCCCCATGGCTGACTAATTTTTTTTATTTATGCAGAGGCCGAGGCCGCCTCGGCCTCTGAG | |
| CTATTCCAGAAGTAGTGAGGAGGCTTTTTTGGAGGCCTAGGCTTTTGCAAAAAGCTT: 3′ | |

With this GAS promoter element linked to the SV40 promoter, a GAS:SEAP2 reporter construct is next engineered. Here, the reporter molecule is a secreted alkaline phosphatase (SEAP). Clearly, in this or in any of the other assays described herein, any applicable reporter molecule is used instead of SEAP without undue experimentation. For example, using art known methods, such as, e.g., without limitation, chloramphenicol acetyltransferase (CAT), luciferase, alkaline phosphatase, B-galactosidase, green fluorescent protein (GFP), or any protein (detectable by an antibody or detectable binding partner) could be substituted for SEAP. Once the above sequence is confirmed, the synthetic GAS-SV40 promoter element is subcloned into a pSEAP-Promoter vector (Clontech) using HindIII and XhoI. This, effectively, replaces the SV40 promoter with the amplified GAS:SV40 promoter element to create a GAS-SEAP vector. However, since the resulting GAS-SEAP vector does not contain a neomycin resistance gene it is not a preferred embodiment for use in mammalian expression systems. To generate stable mammalian cell lines that express a GAS-SEAP reporter, the GAS-SEAP cassette is removed (using Sal1 and NotI) from the GAS-SEAP vector and inserted into a backbone vector containing a neomycin resistance gene, such as, e.g., pGFP-1 (Clontech), using these restriction sites in the multiple cloning site, to create a GAS-SEAP/Neo vector. Once the GAS-SEAP/Neo vector is transfected into a mammalian cell, it can also be used as a reporter molecule for GAS binding as taught in an assay as described herein.

Similar constructs is made using the above description and replacing GAS with a different promoter sequence. For example, construction of reporter-molecules containing NFK-B and EGR promoter sequences are applicable. Additionally, however, many other promoters is substituted using a protocols described herein, e.g., SRE, IL-2, NFAT, or Osteocalcin promoters is substituted, alone or in combination with another (e.g., GAS/NF-KB/EGR, GAS/NF-KB, IL-2/NFAT, or NF-KB/GAS). Similarly, other cell lines is used to test reporter construct activity, such as, e.g., without limitation, HELA (epithelial), HUVEC (endothelial), Reh (B-cell), Saos-2 (osteoblast), HUVAC (aortic), or Cardiomyocyte cell lines. Alternatively, testing whether an LP polypeptide (or fragment thereof) is involved in a JAK/STATs signal transduction pathway can be performed (without undue experimentation) by adopting a method as described, e.g., in Ho, et al. (1995) Mol. Cell. Biol. 15:5043-5-53. Furthermore, it nay be possible to test the JAK/STATs signal transduction pathway for blockage using an LP composition of the invention. Additionally, standard methods exist for testing whether an LP polypeptide (or fragment thereof) of the invention is involved in a STAT signaling pathway (e.g., such methods are described, e.g., in Starr, et al. (1997) Nature 387:917-921; Endo, et al. (1997) Nature 387:921-924; and Naka, et al. Nature 387:924-929 and can be employed here without undue experimentation).

### Example 6

### High-Throughput Screening Assay for T-Cell Activity

The following protocol is used to assess T-cell activity by identifying factors and/or determining whether a supernate (described herein) containing an LP polypeptide (or fragment thereof) modulates the proliferation and/or differentiation of a T-cell. T-cell activity is assessed using a GAS/SEAP/Neo construct. Thus, a factor that increases SEAP activity indicates an ability to activate a Jaks-STATS signal transduction pathway. One type of T-cell used in this assay is, e g., a Jurkat T-cell (ATCC Accession No. TIB-152), although other cells can also be used such as, e.g., without limitation, Molt-3 cells (ATCC Accession No. CRL-1552) or Molt-4 cells (ATCC Accession No. CRL-1582).

Jurkat T-cells are lymphoblastic CD4+ Th1 helper cells. To generate stable cell lines, approximately 2 million Jurkat cells are transfected with a GAS-SEAP/Neo vector using DMRIE-C (Life Technologies, in a transfection procedure as described below. Transfected cells are seeded to a density of approximately 20,000 cells per well and any resulting transfectant (resistant to 1 mg/ml genticin) is subsequently selected. Resistant colonies are then expanded and tested for their response to increasing concentrations of interferon gamma. The dose response of a selected clone is then established. Typically, the following method yields a number of cells sufficient for 75 wells (each containing approximately 200 ul of cells). The method can be modified easily (e.g., it can either be scaled up or performed in multiples to generate sufficient numbers of cells for multiple 96 well plates). Jurkat cells are maintained in RPM1+10% serum with 1% Pen-Strep. Combine 2.5 mls of OPTI-MEM (LifeTechnologies) with 10 ug of plasmid DNA in a T25 flask. Add 2.5 ml OPTI-MEM containing 50 μl of DMRIE-O and incubate (RT) for 15-45 min. During incubation, determine the cell concentration, spin down the required number of cells ( 107 per transfection), and resuspend in OPTI-MEM to a final concentration of 10⁷ cells/ml. Then add 1 ml of 1×10⁷ cells in OPTI-MEM to a T25 flask and incubate at 37° C. for 6 hrs. After incubation, add 10 ml of RPM1+15% serum.

The Jurkat:GAS-SEAP table reporter lines are maintained in RPMI+10% serum, 1 mg/ml Genticin, and 1% Pen-Strep. These cells are treated with supernatants containing an LP polypeptide (or fragment thereof) and/or an induced polypeptide of the invention (or fragment thereof) as produced by a protocol taught herein. On the day of treatment with the supernatant, the cells should be washed, and re-suspended in fresh RPM1+10% serum to a density of 500,000 cells per ml. The exact number of cells required depends on the number of supernatants being screened. For one 96 well plate, approximately 10 million cells are required (for 10 plates, 100 million cells). Transfer the cells to a triangular reservoir boat, to dispense the cells into a 96 well dish, using a 12 channel pipette to transfer 200 ul of cells into each well (therefore adding 100,000 cells per well). After all the plates have been seeded, 50 ul of the supernatants are transferred directly from the 96 well plate containing the supernatants into each well using a 12 channel pipette. In addition, a dose of exogenous interferon gamma (0.1 ng, 1.0 ng, 10.0 ng) is added to wells H9, H10, and H11 to serve as additional positive controls for the assay. The 96 well dishes containing Jurkat cells treated with supernatants are placed in an incubator for 48 hrs (note: this time is variable between 48-72 hrs). Then, 35 ul samples from each well are transferred to an opaque 96 well plate using a 12-channel pipette. The opaque plates should be covered (using cellophane), and stored at −20° C. until SEAP assays are performed as described herein or known in the art. Plates containing the remaining treated cells are placed at 4° C., and can serve as a source of material for repeated assays on a specific well if so desired. As a positive control, 100 Unit/ml interferon gamma is used to activate Jurkat T cells. Typically, a 30-fold induction or greater is observed in positive control wells. As will be apparent to those of ordinary skill in the art, the above protocol may be used in the generation of both transient, as well as, stably transfected cells.

### Example 7

### High-Throughout Screening Assay to Identify Myeloid Activity

The following protocol is used to assess myeloid activity by determining whether an LP polypeptide (or fragment thereof) mediates the proliferation, and/or differentiation of a myeloid cell. Myeloid cell activity is assessed using a GAS/SEAP/Neo construct as described herein. Thus, a factor that increases SEAP activity indicates the ability to activate a Jaks-STATS signal transduction pathway. A typical myeloid cell used in such an assay is U937 (a pre-monocyte cell line) although, other myeloid cells can be used, such as, e.g., without limitation, TF-1, HL60, or KG1.

To transiently transfect U937 cells with a GAS/SEAP/Neo construct a DEAE-Dextran method is used (Kharbanda, et al. (1994) Cell Growth & Differentiation, 5: 259-265). First, 2×10⁷ U937 cells are harvested and then washed with PBS. Typically, U937 cells are grown in RPMI 1640 medium containing 10% heat-inactivated fetal bovine serum (FBS) supplemented with 100 units/ml penicillin, and 100 mg/ml streptomycin. Next, suspend the cells in 1 ml of 20 mM Tris-HCl (pH 7.4) buffer containing 0.5 mg/ml DEAE-Dextran, 8 ug GAS-SEAP2 plasmid DNA, 140 mM NaCl, 5 mM KCl, 375 uM Na₂HPO₄-7H20, 1 mM MgCl₂, and 675 uM CaCl₂. Incubate at 37° C. for 45 min. Wash the cells with RPMI 1640 medium containing, 10% FBS and then resuspend in 10 ml complete medium and incubate at 37° C. for 36 hr. The GAS-SEAP/U937 stable cells are obtained by growing the cells in 400 ug/ml G418. The G418-free medium is used for routine growth but periodically (every one to two months), the cells should be re-grown in 400 ug/ml G418 for several passages. These cells are tested by harvesting 1×10⁸ cells (approximately enough for ten 96-well plate assays) and then washing with PBS. Suspend the cells in 200 ml of the above described growth medium to a final density of 5×10⁵ cells/ml. Plate 200 ul cells/well in a 96-well plate (or 1×10 ⁵ cells/well). Add 50 ul of supernatant as described herein then, incubate at 37° C. for 48 to 72 hr. As a positive control, 100 Unit/ml interferon gamma is used to activate U937 cells. Typically, a 30-fold induction is observed in wells containing the positive controls. Assay a supernatant according to a SEAP protocol taught herein or art-known.

### Example 8

### High-Throughput Screening Assay to Identify Neuronal Activity

When cells undergo differentiation and proliferation, genes are activated through man) different signal transduction pathways. One such gene, EGR1 (early growth response gene 1), is induced in various tissues and cell types upon activation. The promoter of EGRI is responsible for such induction. The activation of particular cells is assessed using the EGRI promoter linked to a reporter molecule. Specifically, the following protocol is used to assess neuronal activity in a PC12 cell (rat phenochromocytoma cell). PC12 cells show proliferative and/or differentiative responses (e.g., EGRI expression) upon activation by a number of stimulators, such as, e.g., TPA (tetradecanoyl phorbol acetate), NGF (nerve growth factor), and EGF (epidermal growth factor). Thus, PC12 cells (stably transfected with a construct comprising an EGR promoter operably linked to SEAP reporter) are used in an assay to determine activation of a neuronal cell by an LP polypeptide (or fragment thereof). A EGR/SEAP reporter construct is created as follows: the EGR-1 promoter sequence (−633 to +1; Sakamoto, et al. (1991) Oncogene 6:867-871) is PCR amplified from human genomic DNA using the following primers:

(SEQ ID NO:27)

5′ GCGCTCGAGGGATGACAGCGATAGAACCCCGG-3′

(SEQ ID NO:28)

5′ GCGAAGCTTCGCGACTCCCCGGATCCGCCTC-3′

Using a GAS:SEAP/Neo vector (described herein), the EGR1 amplified product is inserted into this vector by linearizing the GAS:SEAP/Neo vector (XhoI/HindIII) and removing the GAS/SV40 stuffer. The EGRI amplified product is restricted using those same enzymes (XhoI/HindIII). Then, the EGR1 promoter is ligated to the vector. To prepare 96 well-plates for cell culture, add two mls of a coating solution (dilute (1:30) collagen type I (Upstate Biotech Inc. Cat#08-115) in filter sterilized 30% ethanol) per one 10 cm plate or 50 ml per well of the 96-well plate, and then air dry for 2 hr. Routinely grow PC12 cells on pre-coated 10 cm tissue culture dishes using RPMI-1640 medium (Bio Whittaker) containing 10% horse serum (JRH BIOSCIENCES, Cat. # 12449-78P), 5% heat-inactivated fetal bovine serum (FBS) supplemented with 100 units/ml penicillin and 100 ug/ml streptomycin. Every three to four days, perform a one to four split of the cells. Cells are removed from a plate by scraping and re-suspending (typically, by pipetting up and down more than 15 times). To transfect an EGR/SEAP/Neo construct into PC12 cells use the Lipofectamine protocol taught herein. Produce stable EGR-SEAP/PC12 cells by growing transfected cells in 300 ug/ml G418. The G418-free medium is used for routine growth but periodically (every one to two months), the PC12 cells should be re-grown in 300 ug/ml G41830 for several passages.

To assay a PC12 cell for neuronal activity, a 10 cm plate (containing cells that are around 70 to 80% confluent) is screened by removing the old medium and washing the cells once with PBS. Then, starve the cells overnight in low serum medium (RPMI-1640 containing 1% horse serum, and 0.5% FBS with antibiotics). The next morning, remove the medium, and wash the cells with PBS. Scrape off the cells from the plate and suspend them thoroughly in 2 ml low serum medium. Count the cell number, and add more low serum medium to achieve a final cell density of approximately 5×10⁵ cells/ml. Add 200 ul of the cell suspension to each well of 96-well plate (equivalent to 1×10⁵ cells/well). Add 50 ul of supernatant and store at 37° C. for 48 to 72 hr. As a positive control, use a growth factor known to activate PC12 cells through EGR, such as, e.g., 50 ng/ul of Neuronal Growth Factor (NGF). Typically, a fifty-fold or greater induction of SEAP is achieved with a positive control. Assay the supernatant according to a SEAP method described herein.

### Example 9

### High-Throughput Screening Assay to Identify T-Cell Activity

NF-KB (Nuclear Factor kappa B) is a transcription factor activated by a wide variety of agents including, e.g., inflammatory cytokines (such as, e.g., IL-1, TNF, CD30, CD40, lymphotoxin-alpha, and lymphotoxin-beta); LPS, thrombin; and by expression of certain viral gene products. As a transcription factor, NF-KB typically regulates: the expression of genes involved in immune cell activation; the control of apoptosis (NF-KB appears to shield cells from apoptosis); the development of B-cells or T-cells; anti-viral or antmicrobial responses; and multiple stress responses. Under non-stimulating conditions, NF-KB is retained in the cytoplasm with I-KB (Inhibitor KB). However, upon proper stimulation, I-KB is phosphorylated and degraded, leading to NF-KB translocating into the nucleus of the cell, thereby activating transcription of specific target genes, such as, e.g., IL-2, IL-6, GM-CSF, ICAM-I, and Class 1 MHC. Due to NF-KB's role in transcriptional activation and its ability to respond to a range of stimuli, reporter constructs utilizing the NF-KB promoter element are useful in screening a supernatant produced as described herein. Activators or inhibitors of NF-KB are useful in treating diseases, e.g., inhibitors of NF-KB is used to treat diseases, syndromes, conditions, etc., related to the acute or chronic activation of NF-KB, such as, e.g., rheumatoid arthritis. To construct a vector comprising a NF-KB promoter element, a PCR based strategy is employed. The upstream primer should contain four tandem copies of the NF-KB binding site (GGGGACTTTCCC; SEQ ID NO:29), 18 bp of sequence that is complementary to the 5′ end of the SV40 early promoter sequence, and that is flanked by the XhoI site:

| | |
|---|---|
| 5′: GCGGCCTCGAGGGGACTTTCCCGGGGACTTTCCGGGGATCCGGGACTTTCCATCCTGCCATC | (SEQ ID NO:30) |
| TCAATTAG:3′ | |

The downstream primer is complementary to the 3′ end of the SV40 promoter and is flanked by the Hind III site:

| | |
|---|---|
| 5′:GCGGCAAGCTTTTTGCAAAGCCTAGGC:3′. | (SEQ ID NO:31) |

A PCR amplification is performed using the SV40 promoter template present in a pB-gal promoter plasmid (Clontech). The resulting PCR fragment is digested with XhoI, and Hind III, then subcloned into BLSK2 (Stratagene). Sequencing with the T7, and T3 primers should confirm that the insert contains the following sequence:

(SEQ ID NO: 32)

5′: CTCGAGGGGACTTTCCCGGGGACTTTCCGGGGACTTTCCGGGACTT
TCCATCTGCCATCTCAATTAGTCAGCAACCATAGTCCCGCCCCTAACTCC
GCCCATCCCGCCCCTAACTCCGCCCAGTTCCGCCCATTCTCCGCCCCATG
GCTGACTAATTTTTTTTATTTATGCAGAGGCCGAGGCCGCCTCGGCCTCT
GAGCTATTCCAGAAGTAGTGAGGAGGCTTTTTTGGAGGCCTAGGCTTTTG
CAAAAAGCTT:3′

Next, replace the SV40 minimal promoter element present in the pSEAP2-promoter plasmid (Clontech) with the NF-KB/SV40 fragment using XhoI, and HindIII (note, this vector does not contain a neomycin resistance gene, and therefore, is not preferred for use in a mammalian expression system). To generate a stable mammalian cell line, the NF-KB/SV40/SEAP construct is removed from the above NF-KB/SEAP vector using restriction enzymes SaII, and NotI, and then inserted into a vector having neomycin resistance. For example, the NF-KB/SV40/SEAP construct is inserted into pGFP-1 (Clontech), replacing the GFP gene, after restricting pGFP-1 with SaII, and NotI. After a NF-KB/SV40/SEAP/Neo vector is established, then stable Jurkat T-cells are created and maintained as described herein. Similarly, a method for assaying supernatants with these stable Jurkat T-cells is used as previously described herein. As a positive control, exogenous TNF alpha (at, e.g., concentration of 0.1 ng, 1.0 ng, and 10 ng) is added to a control well (e.g., wells H9, H10, and H11). Typically, a 5- to 10-fold activation is observed in the control.

### Example 10

### Assay for Reporter Activity (e.g., SEAP)

As a reporter molecule for the assays taught herein, SEAP activity is assessed using the Tropix Phospho-light Kit (Cat. BP-400) according to the following general procedure. The Tropix Phospho-light Kit supplies the dilution, assay, and reaction buffers described below. Prime a dispenser with the 2.5× dilution buffer and dispense 15 ul of 2.5× dilution buffer into Optiplates containing 35 ul of a supernatant. Seal the plates with a plastic sealer and incubate at 65° C. for 30 min. Separate the Optiplates to avoid uneven heating. Cool the samples, until they are maintained at RT for 15 minutes. Empty the dispenser and prime with the assay buffer. Add 50 ml assay buffer and incubate (5 min. at RT). Empty the dispenser and prime with the reaction buffer (see the table below). Add 50 ul reaction buffer and incubate (20 min. at RT). Since the intensity of the chemiluminescent signal is time dependent, and it takes about 10 minutes to read five plates on luminometer, treat five plates at each time and start the second set 10 minutes later. Read the relative light unit in the luminometer using the H12 location on the plate as blank, and print the results. An increase in chemiluminescence indicates reporter activity.

Reaction Buffer Formulation:

| # of plates | Rxn buffer diluent (ml) | CSPD (ml) |
|---|---|---|
| 10 | 60 | 3 |
| 11 | 65 | 3.25 |
| 12 | 70 | 3.5 |
| 13 | 75 | 3.75 |
| 14 | 80 | 4 |
| 15 | 85 | 4.25 |
| 16 | 90 | 4.5 |
| 17 | 95 | 4.75 |
| 18 | 100 | 5 |
| 19 | 105 | 5.25 |
| 20 | 110 | 5.5 |
| 21 | 115 | 5.75 |
| 22 | 120 | 6 |
| 23 | 125 | 6.25 |
| 24 | 130 | 6.5 |
| 25 | 135 | 6.75 |
| 26 | 140 | 7 |
| 27 | 145 | 7.25 |
| 28 | 150 | 7.5 |
| 29 | 155 | 7.75 |
| 30 | 160 | 8 |
| 31 | 165 | 8.25 |
| 32 | 170 | 8.5 |
| 33 | 175 | 8.75 |
| 34 | 180 | 9 |
| 35 | 185 | 9.25 |
| 36 | 190 | 9.5 |
| 37 | 195 | 9.75 |
| 38 | 200 | 10 |
| 39 | 205 | 10.25 |
| 40 | 210 | 10.5 |
| 41 | 215 | 10.75 |
| 42 | 220 | 11 |
| 43 | 225 | 11.25 |
| 44 | 230 | 11.5 |
| 45 | 235 | 11.75 |
| 46 | 240 | 12 |
| 47 | 245 | 12.25 |
| 48 | 250 | 12.5 |
| 49 | 255 | 12.75 |
| 50 | 260 | 13 |

### Example 11

### High-Throughput Screening Assay Identifying Changes in Small Molecule Concentration and Membrane Permeability

Binding by a ligand to a receptor can affect: intracellular levels of small molecules (such as, e.g., without limitation, calcium, potassium, and sodium); pH, and a membrane potential of the cell. These alterations are measured in an assay to identify supernatants that bind to a receptor. The following protocol is a non-limiting exemplar for assaying the effects on calcium ions in a cell (such as, e.g., without limitation, Ca⁺⁺ sequestration, removal, uptake, release, etc.) however, this assay can easily be modified to detect other cellular changes (such as, e.g., potassium, sodium, pH, membrane potential) effected by binding of a ligand with a receptor.

The following assay uses Fluorometric Imaging Plate Reader (“FLIPR”) to measure changes in fluorescent molecules (Molecular Probes) that bind small molecules, such as, e.g., Ca⁺⁺. Clearly, as would be recognized by the skilled artisan, other fluorescent molecules that can detect a small composition (such as, e.g., a small molecule) can be employed instead of the calcium fluorescent molecule, fluo-4 (Molecular Probes, Inc.; No. F-14202), used here. For adherent cells, seed the cells at 10,000-20,000 cells/well in a Co-starblack 96-well plate with a clear bottom. Incubate the plate in a CO₂ incubator for 20 hours. The adherent cells are washed twice in a Biotek washer with 200 ul of HBSS (Hank's Balanced Salt Solution) leaving 100 ul of buffer after the final wash. A stock solution of 1 mg/ml fluo-4 is made in 10% pluronic acid DMSO. To load the cells with fluo-4, 50 ul of 12 ug/ml fluo-4 is added to each well. The plate is incubated at 37° C. in a CO₂ incubator for 60 min. Wash the plate four times in a Biotek washer with 200 ul of HBSS leaving 100 ul of buffer (as described above). For non-adherent cells, the cells are spun down from culture media. Cells are re-suspended in a 50-ml conical tube to 2-5×10⁶ cells/ml with HBSS. Then, 4 ul of 1 mg/ml fluo-4 solution in 10% pluronic acid DMSO is added to each ml of cell suspension. Subsequently, the tube is placed in a 37° C. water bath for 30-60 min. The cells are washed twice with HBSS, re-suspended to 1×10⁶ cells/ml, and dispensed into a microplate (100 ul/well). The plate is centrifuged at 1000 rpm×g (times gravity) for 5 min. The plate is then washed once in 200 ul Denley Cell Wash followed by an aspiration step to 100 ul final volume. For a non-cell based assay, each well contains a fluorescent molecule, such as, e.g., fluo-4. The supernatant is added to the well, and a change in fluorescence is detected. To measure the fluorescence of intracellular calcium, the FLIPR is set for the following parameters: (1) System gain is 300-800 mW; (2) Exposure time is 0.4 second; (3) Camera F/stop is F/2; (4) Excitation is 488 nm; (5) Emission is 530 nm; and (6) Sample addition is 50 ul. Observance of an increased emission at 530 nm indicates an extracellular signaling event, which has resulted in an increase in the concentration of intracellular Ca⁺⁺.

### Example 12

### High-Throughput Screening Assay to Identify Tyrosine Kinase Activity

The Protein Tyrosine Kinases (PTK) represent a diverse group of transmembrane and cytoplasmic kinases. Within the Receptor Protein Tyrosine Kinase (RPTK) group are receptors for a range of mitogenic and metabolic growth factors including, e.g., the PDGF, FGF, EGF, NGF, HGF, and Insulin receptor subfamilies. In addition, a large number of RPTKs have no known corresponding ligand. Ligands for RPTKs include, e.g., mainly secreted small proteins, but also can include membrane-bound proteins, and extracellular matrix proteins.

Activation of an RPTK by a ligand typically involves dimerization of a ligand-mediated receptor resulting in the transphosphorylation of a receptor subunit(s) and subsequent activation of a cytoplasmic tyrosine kinase. Typically, cytoplasmic tyrosine kinases include, e.g., receptor associated tyrosine kinases of the src-family (such as, e.g., src, yes, lck, lyn, and fyn); non-receptor linked tyrosine kinases, and cytosolic protein tyrosine kinases (such as, e.g., Jaks, which mediate, e.g., signal transduction triggered by the cytokine superfamily of receptors such as, e.g., the Interleukins, Interferons, GM-CSF, and Leptin). Because of the wide range of factors that stimulate tyrosine kinase activity, the identification of a novel human secreted protein capable of activating tyrosine kinase signal transduction pathways would be useful. Therefore, the following protocol is designed to identify a novel human secreted protein (or fragments thereof) that activates a tyrosine kinase signal transduction pathway. Seed target cells (e.g., primary keratinocytes) at a density of approximately 25,000 cells per well in a 96 well Loprodyne Silent Screen Plates purchased (Nalge Nunc, Naperville, Ill.). Sterilize the plates using two 30-minute rinses with 100% ethanol, then rinse with doubly deionized water, and dry overnight. Coat some plates for 2 hr with 100 ml of cell culture grade type I collagen (50 mg/ml), gelatin (2%), polylysine (50 mg/ml) (Sigma Chemicals, St. Louis, Mo.); 10% Matrigel (Becton Dickinson, Bedford, Mass.); or calf serum. Then rinse the plates (PBS) and store at 4° C. Seed 5,000 cells/well in growth medium on a plate and then (after 48 hrs) assay cell growth by estimating the resulting cell number using the Alamar Blue method (Alamar Biosciences, Inc., Sacramento, Calif.). Use Falcon plate covers (#3071 from Becton Dickinson, Bedford, Mass.) to cover the Loprodyne Silent Screen Plates. Falcon Microtest III cell culture plates can also be used in some proliferation experiments.

To prepare extracts, seed A431 cells onto nylon membranes of Loprodyne plates (20,000/200 ml/well) and culture overnight in complete medium. Quiesce the cells by incubation in serum-free basal medium for 24 hr. Treat the cells with EGF (60 ng/ml) or 50 ul of a supernatant described herein, for 5-20 minutes. After removing the medium, add 100 ml of extraction buffer to each well (20 mM HEPES pH 7.5, 0.15M NaCl, 1% Triton X-100, 0.1% SDS, 2 mM Na₃VO₄, 2 mM Na₄P₂O₇ and a cocktail of protease inhibitors (Boeheringer Mannheim, Cat No. 1836170; Indianapolis, Tenn.) and shake the plate on a rotating shaker for 5 minutes at 4° C. Then place the plate in a vacuum transfer manifold and extract filter through the 0.45 mm membrane bottom of each well (using house vacuum). Collect the extracts of a 96-well catch/assay plate in the bottom of the vacuum manifold and immediately place on ice. To clarify an extract by centrifugation, remove the content of a well (after detergent solubilization for 5 min) and centrifuge (15 min at 16000×G at 4° C). Test the filtered extracts for levels of tyrosine kinase activity. Although many methods of detecting tyrosine kinase activity are known and can be used without undue experimentation, a non-limiting method is described here for exemplar purposes. Generally, the tyrosine kinase activity of a supernatant is evaluated by determining its ability to phosphorylate a tyrosine residue on a specific substrate (e.g., a biotinylated peptide). An example of a biotinylated peptide useful for this purpose includes, e.g., without limitation, PSK1 (corresponding to amino acid residue numbers 6-20 of the cell division kinase cdc2-p34) and PSK2 (corresponding to amino acid residue numbers 1-17 of gastrin). Both of these biotinylated peptides are substrates for a number of tyrosine kinases and are commercially available (Boehringer Mannheim, Indianapolis, Ind.).

The tyrosine kinase reaction is set up by adding the following components as follows: First, add 10 μl of 5 uM biotinylated peptide, then 10 μ1 ATP/Mg⁺² (5 mM ATP/50 mM MgCl₂, then 10 μl of 5× Assay Buffer (40 mM imidazole hydrochloride, pH7.3, 40 mM beta-glycerophosphate, 1 mM EGTA, 100 mM MgCl₂, 5 mM MnCl₂, 0.5 mg/ml BSA), then 5 μl of Sodium Vanadate (1 mM), and then 5 μl of water. Mix the components gently and pre-incubate the reaction mix at 30° C. for 2 min. Initialize the reaction by adding 10 μl of the control enzyme or the filtered supernatant. Stop the tyrosine kinase assay reaction by adding 10 ul of 120 mm EDTA and place the reactions on ice. Determine tyrosine kinase activity by transferring 50 ul of the reaction mixture to a microtiter plate (MTP) module and incubating at 37° C. for 20 min. This allows the streptavadin coated 96 well plate to associate with the biotinylated peptide. Wash the MTP module four times with 300 ul of PBS per well. Next add 75 ul of anti-phosphotyrosine antibody conjugated to horseradish peroxidase (anti-P-Tyr-POD (0.5 μl/ml)) to each well and incubate for one hour at 37° C. Wash each well as described above. Next, add 100 μl of peroxidase substrate solution (Boehringer Mannheim, Indianapolis, Ind.) and incubate for a minimum of five minutes (up to 30 min) at RT. Measure the absorbance of the sample at 405 nm using an ELISA reader (the level of bound peroxidase activity reflects the level of tyrosine kinase activity and is quantitated using an ELISA reader).

LP-induced tyrosine phosphorylation is determined as follows using any appropriate cell line (such as, e.g., Saos, GH4C1, LNCAP, LLC-PK1, L6, GT1-7, SK-N-MC, U373MG, MCF-7, Ishikawa, PA1, HEP-G2, ECV304, GLUTag, BTC6, HuVEC, TF-1, Balb/C 373, HDF, M07E, T1165, THP-1, or Jurkat). On day 1, approximately 2.0×10⁴ cells per are plated onto poly-D-lysine-coated wells (96 well plates) containing 100 μL cell propagation media (DMEM:F12 at a 3:1 ratio, 20 mM Hepes at pH 7.5, 5% FBS, and 50 μg/ml Gentamicin) then incubated overnight. On day 2, the propagation media is replaced with 100 μL starvation medium (DMEM:F12 at a 3:1, 20 mM Hepes at pH 7.5, 0.5% FBS, and 50 μg/ml Gentamicin) and incubated overnight. On day 3, a 100× stock of pervanadate solution is prepared (100 μL of 100 mM sodium orthovanadate and 3.4 μL of H₂O₂). Cells are stimulated with varying concentrations of an LP of the invention (e.g., 0.1, 0.5, 1.0, 5, and 10 μL of an LP stock solution) and incubated (10 min. at RT). After stimulation, the medium is aspirated and 75 μL lysis buffer (50 mM Hepes at pH 7.5, 150 mM NaCl, 10% glycerol, 1% TRITON X-100, 1 mM EDTA, 1 mM pervanadate, and BM protease inhibitors) is added to each well (4° C. for 15 minutes). Subsequently, 25 μL of 4× loading buffer is added to the cell lysates and the resulting solution is mixed and then heated to 95° C.

Detection of tyrosine phosphorylation is accomplished by Western immunoblotting. Samples of the treated cells (20 μl) are separated using SDS-PAGE 8-16% AA ready gels (Bio-Rad). Separated proteins are subsequently electrotransferred (˜1 hr at 250 mA) in transfer buffer (25 mM Tris base at pH 8.3, 0.2 M glycine, 20% methanol) to a nitrocellulose membrane that is incubated (1 hr at RT) in a blocking buffer (20 mM Tris HCl at pH 7.5, 150 mM NaCl, 0.1% TWEEN-20; 1% BSA). To detect the presence of LP-induced phosphorylated proteins any appropriate commercially available anti-phosphotyrosine antibody is added to a membrane (such as, e.g., a monoclonal antibody that can detect, e.g., Erk-1, Erk-2 kinase, Raf, JNK, p38 MAP, Map kinase kinase (MEK), MEK kinase, Src, Muscle specific kinase (MUSK), IRAK, Tee, and Janus, etc.). The membrane is incubated overnight (4° C. with gentle rocking) in a first solution (primary antibody, TBST, and 1% BSA), followed by TBST washing (×3 for 5 min/wash at RT) and incubation (1 hr at RT with gentle rocking) with a second solution (secondary antibody, TBST, and 1% BSA). After the secondary incubation, another series of TBST washes is carried out (×4 for 10 min/wash at RT) and detection of the immuno-identified proteins is visualized by incubating the membranes (10-30 ml of SuperSignal Solution for approximately 1 min at RT). After excess developing solution is removed, the membrane is wrapped (plastic wrap) and exposed to X-ray film (20 sec., 1 min., and 2 min. or longer if needed). LP-induced tyrosine phosphorylation is determined by comparing the number and intensity of immunostained protein bands from treated cells (visual inspection) with the number and intensity of immunostained protein bands from negative control cells buffer only without LP solution).

### Example 13

### High-Throughput Screening Assay to Identify Phosphorylation Activity

An alternative and/or complimentary tyrosine kinase assay, which can also be used detects activation (e.g., phosphorylation) of intracellular signal transduction intermediates. For example, as described herein, such an assay detects tyrosine phosphorylation of an Erk-1 and/or Erk-2 kinase. However, detecting phosphorylation of other molecules, such as, e.g., Raf, JNK, p38 MAP, Map kinase kinase (MEK), MEK kinase, Src, Muscle specific kinase (MUSK), IRAK, Tee, and Janus; as well as any other phosphoserine, phosphotyrosine, or phosphothreonine molecule, can be determined by substituting one of these molecules for an Erk-1 or Erk-2 molecule used as follows. Specifically, assay plates are made by coating the wells of a 96-well ELISA plate with 0.1 ml of protein G (1 ug/ml) for 2 hr at RT. Then, the plates are rinsed with PBS and blocked with 3% BSA/PBS for 1 hr at RT. The protein G plates are subsequently treated for one hour at RT (100 ng/well) using a commercial monoclonal antibody directed against Erk-1 and/or Erk-2 (Santa Cruz Biotechnology). After 3-5 rinses with PBS, the plates are stored at 4° C. until further use. To detect phosphorylation of another molecule (as stated above) modify this step of the method by substituting an appropriate monoclonal antibody, which can detect one of the above-described molecules (such as, e.g., Raf, JNK, p38 MAP, Map kinase kinase (MEK), MEK kinase, Src, Muscle specific kinase (MUSK), IRAK, Tee, Janus, etc.)). Seed A431 cells at 20,000 cells/well in a 96-well Loprodyne filterplate and culture in an appropriate growth medium overnight. Then starve the cells for 48 hr in basal medium (DMEM) and treat for 5-20 minutes with EGF (6.0 ng/well) or with 50 ul of a supernatant described herein. Then, solubilize the cells and filter the cell extract directly into the assay plate. After incubation with the filtered extract for 1 hr at RT, rinse the wells again. As a positive control, use a commercial preparation of MAP kinase (10 ng/well) in place of the extract. Treat the plates (1 hr at RT) with a commercial polyclonal antibody (rabbit; 1 ug/ml) that recognizes a phosphorylated epitope of an Erk-1 and/or an Erk-2 kinase. Biotinylate the antibody using any standard, art-known procedure. Quantitate the amount of bound polyclonal antibody by successive incubations with Europium-streptavidin and Europium fluorescence enhancing reagent in a Wallac DELFIA instrument (using time-resolved fluorescence). Observance of an increased fluorescent signal over background indicates that phosphorylation has occurred.

### Example 14

### Method of Detecting Abnormal Levels of an LP Polypeptide in a Sample

An LP polypeptide (or fragment thereof) can be detected in a sample (such as, e.g., a biological sample as described herein). Generally, if an increased or decreased level of the LP polypeptide (compared to a normal level) is detected, then this level of the polypeptide (or fragment thereof) is a useful marker such as, e.g., for a particular cellular phenotype. Methods to detect the level of a polypeptide (or fragment thereof) are numerous, and thus, it is to be understood that one skilled in the art can modify the following exemplar assay to fit a particular need without incurring undue experimentation.

For example, an antibody-sandwich ELISA is used to detect an LP polypeptide (or fragment thereof) in a sample. Wells of a microtiter plate are coated with specific antibodies, at a final concentration of 0.2 to 10 ug/ml. The antibodies (either monoclonal or polyclonal) are produced by any art known method (or as described herein). The wells are treated with an appropriate blocking reagent so that non-specific binding of the LP polypeptide (or fragment thereof) to the well is reduced and/or prevented. The coated wells are then incubated for greater than 2 hours at RT with the sample containing the LP polypeptide (or fragment thereof). Preferably, serial dilutions of the sample containing the suspected polypeptide (or fragment thereof) should be used to validate results. The plates are then washed three times with doubly deionized or distilled water to remove unbound polypeptide. Next, 50 ul of specific antibody-alkaline phosphatase conjugate (at a concentration of 25-400 ng) is added and incubated (2 hours at RT). The plates are again washed three times with doubly deionized or distilled water to remove unbound conjugate. Subsequently, 75 ul of 4-methylumbelliferyl phosphate (MUP) or p-nitrophenylphosphate (NPP) substrate solution is added to each well and incubated (approximately one hour at RT). The reaction is then measured by a microtiter plate reader. A standard curve is prepared, using serial dilutions of a control sample, and the polypeptide concentration is plotted on the X-axis (log scale) with fluorescence or absorbance plotted on the Y-axis (linear scale). The concentration of the polypeptide in the sample can then be interpolated using the standard curve.

### Example 15

### Detecting Stimulation or Inhibition of B cell Proliferation and Differentiation

Generation of functional humoral immune responses requires both soluble and cognate signaling between B-lineage cells and their microenvironment a signal may impart a positive stimulus that allows a B-lineage cell to continue its programmed development, or a negative stimulus that instructs the cell to arrest its current developmental pathway. To date, numerous stimulatory and inhibitory signals have been found that influence B cell responsiveness (including, e.g., signals from: IL-2, IL-4, IL-5, IL-6, IL-7, IL-10, IL-4, IL-13, IL-14, and IL-15). Interestingly, a signal by itself can be a weak effector but, in combination with various co-stimulatory proteins, the signal can induce, e.g., activation, proliferation, differentiation, homing, tolerance, and death among B cell populations.

One of the best-studied examples of a B-cell co-stimulatory protein is the class of molecules represented by the TNF-superfamily. Within this family, it has been demonstrated that CD40, CD27, and CD30 along with their respective ligands (CD154, CD70, and CD 153) regulate a variety of immune responses. Assays which allow for the detection and/or observation of the proliferation and/or differentiation of a B-cell population and/or its precursors are useful in determining the effect of a composition of the invention on a B-cell population (e.g., in terms of proliferation and differentiation). Taught herein below are two assays designed to detect the effect of a composition of the invention on the differentiation, proliferation, and/or inhibition of a B-cell population or its precursor.

In vitro Assay: An LP polypeptide of the invention (or fragment thereof), is assessed for its ability to induce activation, proliferation, differentiation, inhibition, and/or death in a B-cell and its precursors. The activity of the LP polypeptide on purified human tonsillar B cells (measured qualitatively over the dose range from 0.1 to 10,000 ng/mL) is assessed using a standard B-lymphocyte co-stimulation assay in which purified, tonsillar B cells are cultured in the presence a priming agent (such as, e.g., either formalin-fixed Staphylococcus aureus Cowan I (SAC) or immobilized anti-human IgM antibody). A second signal (such as, e.g., IL-2, and IL-15) synergizes with SAC and IgM crosslinking to elicit B cell proliferation (measured by tritiated-thymidine incorporation). A novel synergizing agent can readily be identified using this assay. The assay involves isolating human tonsillar B cells by magnetic-bead-depletion (MACS) of CD3-positive cells The resulting cell population is greater than 95% B cells as assessed by expression of CD45R(B220). Various dilutions of each sample are placed into individual wells of a 96-well plate to which are added 10⁵ B-cells suspended in culture medium (RPM1 1640 containing 10% 5FBS, 5×10⁻⁵M 2ME, 100U/ml penicillin, 10 ug/ml streptomycin, and 10⁻⁵ dilution of SAC) in a total volume of 150 μl. Proliferation or inhibition is quantitated by a 20 h pulse (1 uCi/well) with 3H-thymidine (6.7 Ci/mM) beginning 72 h post factor addition. The positive and negative controls are respectively, IL2 and medium.

In vivo Assay: BALB/C mice are injected (i.p.) twice daily either with buffer alone or with 10 mg/Kg of an LP polypeptide of the invention (or fragment thereof). Mice receive this treatment for four consecutive days, at which time they are sacrificed and various tissues and serum collected for analyses. Comparison of sections (hemotoxylin and eosin stained) from normals and spleens treated with an LP polypeptide (or fragment thereof) are assessed to identify an effect of the activity of the LP polypeptide (or fragment thereof) on spleen cells (such as, e.g., the diffusion of peri-arterial lymphatic sheaths, and/or significant increases in the nucleated cellularity of the red pulp regions, which may indicate activation of differentiation and proliferation of a B-cell population). Any immunohistochemical technique using any appropriate B cell marker (such as, e.g., anti-CD45R) is used to determine whether a physiological change to a splenic cell (such as, e.g., splenic disorganization) is due to an increased B-cell representation within a loosely defined B-cell zone that infiltrates an established T-cell region. Flow cytometric analyses of spleens from treated mice are used to indicate whether the tested LP polypeptide (or fragment) specifically increases the proportion of ThB+, CD45R dull B cells over control levels. Similarly, an indication of an increased representation of mature B-cells in vivo is the detection in a relative increase in serum titers of Ig. Furthermore, determining whether increased B-cell maturation has occurred can also be achieved by comparing serum IgM and IgA levels between LP polypeptide-treated mice and mice treated with buffer only. cl Example 16

### T-Cell Proliferation Assay

To assess the effect of an LP polypeptide (or fragment thereof) of the invention on T-cell proliferation (e.g., by measuring CD3-induced proliferation), an assay is performed on PBMCs to measure ³H-thymidine uptake. Ninety-six well plates are coated with 100 μl/well of monoclonal antibody to CD3 (such as, e.g., HIT3a, Pharmingen) or an isotype-matched control mAb (e.g., B33.1) overnight at 4° C. (1 μg/ml in 0.05M bicarbonate buffer, pH 9.5), then washed ×3 (PBS). PBMC are isolated by F/H gradient centrifugation from human peripheral blood and added to quadruplicate wells (5×10⁴/well) of mAb coated plates in RPM1 containing 10% FCS and P/S in the presence of varying concentrations of an LP polypeptide (or fragment thereof) (total volume 200 ul). Relevant protein buffer (or medium only) is used as a control. After 48 hr culture at 37° C., plates are spun for 2 min. at 1000 rpm and 100 μl of supernatant is removed and stored at −20° C. for measurement of IL-2 (or other cytokines) if an effect on proliferation is observed. Wells are supplemented with 100 μl of medium containing 0.5 uCi of ³H-thymidine and cultured at 37° C. for 18-24 hr. Wells are harvested and the amount of incorporation of ³H-thymidine is used as a measure of proliferation. Anti-CD3 by itself is used as a positive control for proliferation. IL-2 (100 U/ml) is also used as a control that enhances proliferation. A control antibody that does not induce proliferation of T cells is used as a negative control for the effect of an LP polypeptide (or fragment thereof).

### Example 17

### Effect of an LP polypeptide (or Fragment Thereof) on the Expression of MHC Class II, Co-stimulatory and Adhesion Molecules and Cell Differentiation of Monocytes and Monocyte-Derived Human Dendritic Cells

Dendritic cells are generated by the expansion of proliferating precursors found in the peripheral blood: adherent PBMC or elutriated monocytic fractions are cultured for 7-10 days with GM-CSF (50 ng/ml) and IL-4 (20 ng/ml). These dendritic cells have the characteristic phenotype of immature cells (e.g., expression of CD1, CD80, CD86, CD40, and MHC class II antigens). Treatment with an activating factor (such as, e.g., TNF-alpha) causes a rapid change in surface phenotype (e.g., an increased expression of MHC class I and II, co-stimulatory and adhesion molecules, down regulation of FQRII, and/or an up regulation of CD83). Typically, these changes correlate with an increased antigen-presenting capacity and/or with a functional maturation of a dendritic cell. A FACS analysis of surface antigens is performed as follows: cells are treated 1-3 days with increasing concentrations of an LP polypeptide (or fragment thereof) or LPS as a positive control, washed with PBS containing 1% BSA and 0.02 mM NaN₃, and then incubated with 1:20 dilution of appropriate FITC- or PE-labeled monoclonal antibodies for 30 minutes at 4° C. After an additional wash, the labeled cells are analyzed by flow cytometry on a FACScan (Becton Dickinson).

Effect on the Production of Cytokines

Cytokines generated by dendritic cells, in particular IL-12, are important in the initiation of T-cell dependent immune responses. IL-12 strongly influences the development of Th-1 helper T-cell immune response, and induces cytotoxic T and NK cell function. An ELISA is used to measure IL-12 release in a dendritic cell that has been exposed to an LP polypeptide of the invention (or fragment thereof) as follows: dendritic cells (10⁶/ml) are treated with increasing concentrations of an LP polypeptide (or fragment thereof) for 24 hours. LPS (100 ng/ml) is added to a cell culture as a positive control. Supernatants from the cell cultures are then collected and analyzed for IL-12 using a commercial ELISA kit (e.g., R & D Systems; Minneapolis, Minn.). The standard protocol provided with the kit is used to measure IL-12 expression.

Effect on the Expression of MHC Class II, Co-stimulatory, and Adhesion Molecules.

Three major families of cell surface antigens is identified on monocytes: adhesion molecules, molecules involved in antigen presentation, and Fc receptor. Modulation of the expression of MHC class II antigens and other co-stimulatory molecules (such as, e.g., B7 and ICAM- 1) may result in changes in the antigen presenting capacity of a monocyte and in an ability to induce T cell activation. Increased expression of Fc receptors may correlate with improved monocyte cytotoxic activity, cytokine release, and phagocytosis. A FACS analysis is used to examine surface antigens as follows: monocytes are treated for 1-5 days with increasing concentrations of an LP polypeptide (or fragment thereof) or LPS (as a positive control), washed with PBS containing 1% BSA and 0.02 mM sodium azide (NaN₃), and then incubated with a 1:20 dilution of appropriate FITC- or PE-labeled monoclonal antibodies for 30 minutes at 4° C. After an additional wash, the labeled cells are analyzed by flow cytometry on a FACS scanner (Becton Dickinson).

Monocyte Activation and/or Increased Survival

Assays for molecules that: activate (or, alternatively, inactivate) monocytes; and/or increase monocyte survival (or, alternatively, decrease monocyte survival) are known in the art and may routinely be applied to determine whether a composition of the invention (such as, e.g., a polypeptide or fragment thereof) functions as an inhibitor or activator of a monocyte. Polypeptides (fragments thereof), agonists, or antagonists of the invention is screened using any of the assays described below. For each of these assays, peripheral blood mononuclear cells (PBMC) are purified from single donor leukopacks (American Red Cross, Baltimore, Md.) by centrifugation through a Histopaque gradient (Sigma). Monocytes are isolated from PBMC by counterflow centrifugal elutriation.

Monocyte Survival Assay

Human, peripheral-blood monocytes progressively lose viability when cultured in the absence of serum or other stimuli. Their death typically results from internally regulated processes (such as, e.g., apoptosis). Addition to a culture of activating factors, such as, e.g., TNF-alpha dramatically improves PBMC survival and prevents DNA fragmentation. Propidium iodide (PI) staining is used to measure apoptosis as follows: monocytes are cultured for 48 hours in polypropylene tubes in serum-free medium (positive control), in the presence of 100 ng/ml TNF-alpha (negative control), and in the presence of varying concentrations of a composition of the invention (such as, e.g., an LP polypeptide or fragment thereof). Cells are suspended at a concentration of 2×10⁶/ml in PBS containing PI at a final concentration of 5 μg/ml, and then incubated at RT for 5 minutes before FACS scan analysis. PI uptake has been demonstrated to correlate with DNA fragmentation in this method.

Effect on Cytokine Release

An important function of monocytes/macrophages is their regulatory activity on other cellular populations of the immune system (e.g., through the release of cytokines after appropriate stimulation). An ELISA assay to measure cytokine release is performed as follows: human monocytes are incubated at a density of 5×10⁵ cells/mi with increasing additions of varying concentrations of an LP polypeptide (or fragment thereof) of the invention (controls employ the same conditions without the LP polypeptide). For IL-12 production, the cells are primed overnight with IFN (100 U/ml) in presence of an LP polypeptide (or fragment thereof). LPS (10 ng/ml) is then added. Conditioned media are collected after 24 h and kept frozen until use. Measurement of TNF-alpha, IL-1, MCP-1, and IL-8 is then performed using any commercially available ELISA kit (e.g., R & D Systems; Minneapolis, Minn.) according to a standard protocol provided with the kit.

Oxidative Burst

Purified monocytes are plated in 96-w plate at approximately 1×10⁵ cells/well. Increasing concentrations of a polypeptide of the invention (or fragment thereof) are added to the wells in a total volume of 0.2 ml culture medium (RPM1 1640+10% FCS, glutamine and antibiotics). After 3 days incubation, the plates are centrifuged and the medium is removed from the wells. To the macrophage monolayers, 0.2 ml per well of phenol red solution (140 mM NaCl, 10 mM potassium phosphate buffer pH 7.0, 5.5 mM dextrose, 0.56 mM phenol red and 19 U/ml of HRPO) is added, together with a stimulant (200 nM PMA). The plates are incubated at 37° C. for 2 hours and the reaction is stopped by adding 20 μl (1N NaOH) per well. The absorbance is read at 610 nm. To calculate the amount of H2O2 produced by the macrophages, a standard curve of a H₂O₂ solution of known molarity is performed for each experiment.

### Example 18

### Biological Effects of an LP Polypeptide (or Fragment Thereof)

Astrocyte and Neuronal Cell Assays

An LP polypeptide of the invention (or fragment thereof) is tested for its capacity to promote survival, neurite outgrowth, and/or phenotypic differentiation of a cell of the nervous system (such as, e.g., a cortical neuronal cell) and/or for it capacity to induce the proliferation of a cell of the nervous system (such as, e.g., a glial fibrillary acidic protein inmnunopositive cell like, e.g., an astrocyte). The use of a cortical cell for this assay is based on the prevalent expression of FGF-1 and FGF-2 (basic FGF) in cortical structures and on reported enhancement of cortical neuronal survival after FGF-2 treatment. A thymidine incorporation assay, e.g., is used to assess the effect of the LP on the nervous system cell.

An in vitro effect of FGF-2 on cortical or hippocampal neurons shows increased neuronal survival and neurite outgrowth (see, e.g., Walicke, et al. (1986) Proc. Natl. Acad. Sci. USA 83:3012-3016). However, reports from experiments on PC-12 cells suggest that neuronal survival and neurite outgrowth are not necessarily synonymous and that a specific effect may depend not only on which FGF is tested but also on the particular receptor(s) that are expressed on a target cell. Using a primary cortical neuronal culture paradigm, the ability of an LP polypeptide (or fragment thereof) to induce neurite outgrowth and effect neuronal survival compared to FGF-2 is assessed using, e.g., a thymidine incorporation assay.

Fibroblast and Endothelial Cell Assays.

For proliferation assays, human lung fibroblasts (Clonetics; San Diego, Calif.) and/or dermal microvascular endothelial cells (Cell Applications; San Diego, Calif.) are cultured at 5,000 cells/well in a 96-well plate for one day in growth medium. The cells are then incubated for one day in 0.1% BSA basal medium. After replacing the medium with fresh 0.1% BSA medium, the cells are incubated (72 hr) with varying concentrations of an LP polypeptide of the invention (or fragment thereof). Then, Alamar Blue (Alamar Biosciences, Sacramento, Calif.) is added to each well to a final concentration of 10% and the cells are incubated for 4 hr. Cell viability is measured using a CytoFluorfluorescence reader. For a PGE assay, the human lung fibroblasts are cultured at 5,000 cells/well in a 96-well plate for one day. After a medium change to 0.1% BSA basal medium, the cells are incubated with FGF-2 or an LP polypeptide (or fragment thereof) with (or without) IL-1 alpha for 24 hours. Then supernatants are collected and assayed for PGE, by EIA (Cayman; Ann Arbor, Mich.). For an IL-6 assay, the human lung fibroblasts are cultured at 5,000 cells/well in a 96-well plate for 24 hrs. After a medium change to 0.1% BSA basal medium, the cells are incubated with FGF-2 or an LP polypeptide (or fragment thereof) with (or without) IL-1 alpha for 24 hours. The supernatants are collected and assayed for IL-6 by ELISA kit (Endogen; Cambridge, Mass.). Human lung fibroblasts are cultured with FGF-2 or an LP polypeptide (or fragment thereof) for 3 days in basal medium before the addition of Alamar Blue to assess any effect on growth of the fibroblasts. FGF-2 should show a stimulatory effect at about 10-2500 ng/ml, which can then be used to compare any stimulatory effect of an LP polypeptide (or fragment thereof).

Parkinson Models

The loss of motor function in Parkinson's syndrome is attributed to a deficiency of striatal dopamine due to the degeneration of nigrostriatal dopaminergic projection neurons. A Parkinsonian animal model involves systemic administration of 1-methyl-4 phenyl 1,2,3,6-tetrahydropyridine (MPTP). In the central nervous system, MPTP is taken-up by astrocytes and catabolized to 1-methyl-4-phenyl pyridine (MPP⁺), which is subsequently released. Released MPP⁺ is accumulated in dopaminergic neurons by the high-affinity re-uptake transporter for dopamine. MPP⁺ is then concentrated in mitochondria via an electrochemical gradient where it selectively inhibits nicotinamide adenine disphosphate: ubiquinone oxidoreductionase (complex I) thereby, interfering with electron transport and eventually generating oxygen radicals. In tissue culture, FGF-2 (basic FGF) has trophic activity towards nigral dopaminergic neurons (Ferrari, et al. (1989) Dev. Biol. 133(1):140-147), and administering a striatal gel foam implant containing FGF-2 protects nigral dopaminergic neurons from MPTP toxicity (Otto and Unsicker, (1990) J. Neuroscience 10(6):1912-1921). Based on these reported data for the effect of FGF-2, an LP polypeptide (or fragment thereof) of the invention is evaluated to determine whether it has a similar effect as FGF-2 (such as, e.g., by modulating dopaminergic neuronal survival (either in vitro or in vivo) from an effect of MPTP treatment). An in vitro dopaminergic neuronal cell culture is prepared by dissecting the midbrain floor plate from gestation day 14 Wistar rat embryos. The tissue is dissociated with trypsin and seeded at a density of 200,000 cells/cm² on polyorthinine-laminin coated glass coverslips. The cells are maintained in Dulbecco's Modified Eagle's medium and F12 medium containing hormonal supplement (N 1). After 8 days in vitro, cultures are fixed with paraformaldehyde and processed for immunohistochemical staining of tyrosine hydroxylase (a specific marker for dopaminergic neurons). Dissociated cell cultures are prepared from embryonic rats. The culture medium is changed every third day and the factors are added at that time. Typically, dopaminergic neurons isolated from gestation-day-14 animals are past a point when dopaminergic precursor cells are believed to be proliferating, therefore, an increase in the number of tyrosine hydroxylase immunopositive neurons is interpreted to suggest that a similar increase in the number of surviving dopaminergic neurons would occur if the treatment had occurred in vitro. Therefore, if an LP polypeptide (or fragment thereof) prolongs the survival of dopaminergic neurons in an assay as taught herein, it suggests that the polypeptide (or fragment) is used to ameliorate, modulate, treat, or effect a Parkinson's disease, syndrome, condition, or state.

### Example 19

### The Effect of an LP Polypeptide on Endothelial Cells

An LP polypeptide (or fragment thereof) is tested for its effect on an endothelial cell (such as, e.g., the effect on the growth of vascular endothelial cells) using the following assay: on day 1, human umbilical vein endothelial cells (HUVEC) are seeded at 2-5×10² cells/35 mm dish density in M199 medium containing 4% fetal bovine serum (FBS), 16 units/ml heparin, and 50 units/ml endothelial cell growth supplements (ECGS, Biotechnique, Inc.). On the following day, the medium is replaced with M199 containing 10% FBS, 8 units/ml heparin. An LP polypeptide (or fragment thereof, and positive controls (such as, e.g., VEGF, and basic FGF (bFGF)) are added to the cells at varying concentrations. On days 4, and 6, the medium is replaced. On day 8, cell number is determined with a Coulter Counter. An increase in the number of HUVEC cells indicates that the polypeptide (or fragment thereof) mediates proliferation of vascular endothelial cells.

### Example 20

### Stimulatory Effect of an LP Polypeptide on the Proliferation of Vascular Endothelial Cells

An LP polypeptide (or fragment thereof) is tested for its stimulatory effect on an endothelial cell (such as, e.g., a vascular endothelial cell) to evaluate a mitogenic effect. A calorimetric MTS (3-(4,5-dimethylthiazol-2-yl)-5-(3-carboxymethoxyphenyl)-2-(4-sulfophenyl) 2H-tetrazolium) assay with the electron coupling reagent PMS (phenazine methosulfate) is performed (Cell Titer 96 AQ, Promega) based on Leak, et al. (1994) In vitro Cell. Dev. Biol. 30A:512-518 (incorporated herein for its assay teachings). Briefly, cells are seeded in a 96-well plate (5,000 cells/well) in 0.1 mL serum-supplemented medium and allowed to attach overnight. After serum-starvation for 12 hours (in 0.5% FBS conditions), bFGF, VEGF, or an LP polypeptide (or fragment thereof), in 0.5% FBS (either with or without Heparin (8 U/ml), is added to a well of the plate. After 48 hours, 20 mg of MTS/PMS mixture (1:0.05) is added per well and incubated (1 hour at 37° C.) before measuring the absorbance (490 nm in an ELISA plate reader). Background absorbance from control wells (some media, no cells) is subtracted, and seven wells are performed in parallel for each condition to test for the presence of mitogenic activity (Leak, et al. supra).

### Example 21

### Inhibition of PDGF-induced Vascular Smooth Muscle Cell Proliferation

An LP polypeptide (or fragment thereof) is tested for its effect on vascular smooth muscle cell proliferation (e.g., by measuring BrdUrd incorporation) according to an assay of Hayashida, et al. (1996) J. Biol. Chem. 6:271(36): 21985-21992 (incorporated herein for its assay teachings).

Briefly, subconfluent, quiescent HAoSMC cells grown on 4-chamber slides are transfected with CRP or FITC-labeled AT2-3LP. Then, the cells are pulsed with 10% calf serum and 6 mg/ml BrdUrd. After 24 h, immunocytochemistry is performed using BrdUrd Staining Kit (Zymed Laboratories). In brief, after being exposed to denaturing solution, the cells are incubated with biotinylated mouse anti-BrdUrd antibody (4° C. for 2 h) and then incubated with streptavidin-peroxidase and diaminobenzidine. After counterstaining with hematoxylin, cells are mounted for microscopic examination, and BrdUrd-positive cells are counted. A BrdUrd index is calculated as a percentage of the number of BrdUrd-positive cells per number of total cells. Additionally, simultaneous detection of BrdUrd staining (nucleus) and FITC uptake (cytoplasm) is performed for an individual cell by the concomitant use of bright field illumination and dark field, UV fluorescent illumination (see, Hayashida, et al., supra, for details).

### Example 22

### Stimulation of Endothelial Migration by an LP

An LP polypeptide (or fragment thereof) is tested for its effect on lymphatic endothelial cell migration. Endothelial cell migration assays are performed using a 48 well micro-chemotaxis chamber (Neuroprobe Inc.; Falk, et al. (1980) J. Immunological Methods: 33:239-247). Polyvinylpyrrolidone-free polycarbonate filters with a pore size of 8 μm (Nucleopore Corp.; Cambridge, Mass.) are coated with 0.1% gelatin (at least 6 hours at RT) and dried under sterile air. Test substances are diluted to appropriate concentrations in M199 supplemented with 0.25% bovine serum albumin (BSA), and 10 ul of the final dilution is placed in the lower chamber of a modified Boyden apparatus. Subconfluent, early passage (2-6) HUVEC or BMEC cultures are washed and trypsinized for the minimum time required to achieve cell detachment. After placing the filter between lower and upper chamber, 2.5×10⁵ cells (suspended in 50 ul M199 containing 1% FBS) are seeded to the upper compartment. The apparatus is then incubated (5 hrs 37° C. in a humidified chamber (5% CO₂)) to allow cell migration. After the incubation period, the filter is removed and the upper side of the filter (containing non-migrated cells) is scraped to remove cells. Then the filters are fixed with methanol and stained with Giemsa solution (Diff-Quick, Baxter, McGraw Park, Ill.). Migration is assessed by counting the number of cells occupying three random high-power fields (40×) in each well (measurements in all groups are performed in quadruplicate).

### Example 23

### LP Stimulation of Nitric Oxide Production by Endothelial Cells

An LP polypeptide (or fragment thereof) is tested for its effect on nitric oxide production by an endothelial cell according to the following assay.

Nitric oxide released by the vascular endothelium is believed to be a mediator of vascular endothelium relaxation. Nitric oxide is measured in 96-well plates of confluent microvascular endothelial cells after 24 hours starvation and a subsequent 4 hr exposure to various levels of an LP polypeptide (or fragment thereof) or a positive control (such as, e.g., VEGF-1). The presence of nitric oxide in the medium is determined by use of the Griess reagent to measure total nitrite after reduction of nitric oxide-derived nitrate by nitrate reductase. The effect of an LP polypeptide (or fragment thereof) on nitric oxide release is examined on HUVEC cells. Briefly, NO release from a cultured HUVEC monolayer is measured with a NO-specific polarographic electrode connected to a NO meter (Iso-NO, World Precision Instruments Inc.) (1049). Calibration of the electrodes is performed with air-saturated distilled water (ISO) or acidified nitrite (Iso-NO) according to the procedure recommended by the manufacturer. The Iso-NO is prepared by the addition of KNO to a helium-gassed solution of 0.14 M KSO and 0.1 M KI in 0.1 M HSO. The standard calibration curve is obtained by adding graded concentrations of KNO₂ (e.g., 0, 5.0, 10.0, 25, 50, 100, 250, and 500 nmol/L) into the calibration solution containing KI and H₂SO₄. The specificity of the Iso-NO electrode to NO is previously determined by measurement of NO from authentic NO gas (1050). The culture medium is removed and HUVECs are washed twice with Dulbecco's phosphate buffered saline. The cells are then bathed in 5 ml of filtered Krebs-Henseleit solution in 6-well plates, and the cell plates are kept on a slide warmer (Lab Line Instruments Inc.) To maintain the temperature at 37° C., the NO sensor probe is inserted vertically into the wells, keeping the tip of the electrode 2 mm under the surface of the solution, before addition of the different conditions. S-nitroso acetyl penicillamin (SNAP) is used as a positive control. The amount of released NO is expressed as picomoles per 1×10⁶ endothelial cells. All values should be established from the means of four to six measurements in each group (number of cell culture wells). See, e.g., Leak, et al. (1995) Biochem. and Biophys. Res. Comm. 217:96-105 (incorporated by reference for teachings on NO assays).

### Example 24

### Effect of an LP Polypeptide on Cord Formation/Hematopoiesis

An LP polypeptide (or fragment thereof) is tested in the following assay for its effect on angiogenesis (such as, e.g., endothelial cell differentiation during cord formation such as, e.g., the ability of microvascular endothelial cells to form capillary-like hollow structures when cultured in vitro). Microvascular endothelial cells (CADMEC; Cell Applications, Inc.) purchased as proliferating cells (passage 2) are cultured in CADMEC growth medium (Cell Applications, Inc.) and used at passage 5. For an in vitro angiogenesis assay, the wells of a 4% cell culture plate are coated (200 ml/well) with attachment factor medium (Cell Applications, Inc.) for 30 min. at 37° C. CADMEC cells are seeded onto the coated wells at 7,500 cells/well and cultured overnight in the growth medium. The growth medium is then templed with 300 mg chord formation medium (Cell Applications, Inc.) containing either a control buffer or an LP polypeptide (or fragment thereof) (ranging from 0.to 100 ng/ml). Commercial VEGF (50 ng/ml; R&D) is used as a positive control. Beta-esteradiol (1 ng/ml) is used as a negative control. An appropriate buffer (without the polypeptide) is also utilized as a control. Treated cells are then cultured for 48 hr. Any resulting capillary-like chords are quantitated (numbers and lengths) using a video image analyzer (e.g., Boeckeler VIA-170). All assays are done in triplicate.

### Example 25

### Effect of an LP Polypeptide on Angiogenesis in a Chick Chorioallantoic Membrane

An LP polypeptide (or fragment thereof) is tested in the following assay for its effect on angiogenesis (such as, e.g., the formation of blood vessels on a chick chorioallantoic membrane (CAM)). The chick chorioallantoic membrane (CAM) is a well-established system to examine angiogenesis. Blood vessel formation on CAM is easily visible and quantifiable.

Fertilized eggs of the White Leghorn chick (Gallus gallus) and the Japanese quail (Cotumix cotumix) are incubated (37.8° C. and 80% humidity). Differentiated CAM of 16-day-old chick and 13-day-old quail embryos is studied as follows: On day 4 of development, a window is made on the shell of a chick egg. The embryos are checked for normal development and the eggs sealed with cellotape. The eggs are further incubated until development day 13 (using standard development stages). Thermanox coverslips (Nunc, Naperville, Ill.) are cut into disks of about 5 mm in diameter. Sterile and salt-free growth factors and an LP polypeptide (or fragment thereof) (ranging from 0.1 to 100 ng/ml) are dissolved in distilled water and about 3.3 mg/5 ml of the mixture are pipetted on the disks. After air-drying, the inverted disks are applied on a CAM. After 3 days, the specimens are fixed in 3% glutaraldehyde and 2% formaldehyde and rinsed in 0.12 M sodium cacodylate buffer. They are then photographed with a stereo microscope [Wild M8] and embedded for semi- and ultra-thin sectioning using any art known method. Controls are performed with carrier disks alone. The extent of angiogenesis due to a growth factor only, an LP polypeptide only, or a combination of a growth factor and an LP is measured with respect to the degree of angiogenesis found on the untreated controls.

### Example 26

### An In Vivo Angiogenesis Assay Using a Matrigel Implant

An LP polypeptide (or fragment thereof) is tested in the following assay for its effect on angiogenesis (such as, e.g., its effect on the ability of an existing capillary network to form new vessels in a capsule of extracellular matrix material (Matrigel) which is implanted in a living rodent). Briefly, varying concentrations of an LP polypeptide (or fragment thereof) are mixed with liquid Matrigel (Becton Dickinson Labware; Kollaborative Biomedical Products) at 4° C. and then injected subcutaneously into a rodent (e.g., a mouse) where it subsequently solidifies into a plug. After 7 days, the plug is removed and examined for the presence of new blood vessels. More specifically, an LP polypeptide (or fragment thereof), preferably a secreted protein, (e.g., such as, 150 ng/ml) is mixed with Matrigel at 4° C. (the Matrigel material is liquid at 4° C.) and then drawn into a cold 3 ml syringe. A female C57BY6 mouse (approximately 8 weeks old) is then injected with approximately 0.5 ml of the mixture at two separate locations (preferably, around the midventral aspect of the abdomen). After 7 days, all injected mice are sacrificed, the Matrigel plugs are removed and cleaned (i.e., all clinging membranes and fibrous tissue is removed). The plugs are then fixed in neutral buffered formaldehyde (10%), embedded in paraffin, sectioned for histological examination, and stained (e.g., Masson's Trichrome). Cross sections from three different regions of each plug are so processed while other elected sections are stained for the presence of vWF. A positive control for this assay is bovine basic FGF (150 ng/ml). Matrigel alone (without an LP polypeptide or FGF) is used as a control to determine basal levels of angiogenesis.

### Example 27

### Effect of LP on Ischemia in a Rabbit Lower Limb Model

An LP polypeptide (or fragment thereof) is tested in the following assay for its effect on ischemia using a rabbit hindlimb ischemia model (created by surgical removal of a femoral artery as described by Takeshita, et al. (1995) Am J. Patho 147:1649-16605 and Howell et al., (2000) Nonviral Delivery of the Developmentally Regulated Endothelial Locus-1 (del-1) Gene Increases Collateral Vessel Formation to the Same Extent as hVEGF165 in a Rabbit Hindlimb Ischemia Model, Program No.: 536, Third Annual Meeting of the American Society of Gene Therapy; each of which are incorporated by reference herein for the teachings of this assay).

### Example 28

### Effect of an LP Polypeptide on Vasodialation

An LP polypeptide (or fragment thereof) is tested in the following assay for its ability to affect blood pressure in spontaneously hypertensive rats (SHR), such as, e.g., by modulating dilation of the vascular endothelium. In one embodiment, a retrovirally-mediated recombinant construct comprising an LP polypeptide (or fragment thereof) at varying dosages (e.g., 0.5, 1, 10, 30, 100, 300, and 900 mg/kg) is delivered intracardiacally to determine the affect on the development of high blood pressure in a spontaneously hypertensive (SH) rat model of human essential hypertension to determine whether attenuation of high BP is associated with prevention of other pathophysiological changes induced by a hypertensive state. Intracardiac delivery of a polypeptide (or fragment thereof) is administered to 13-14 week old spontaneously hypertensive rats (SHR) according to a method of Martens, et al. (1998) Proc Natl Acad. Sci USA 95(5):2664-9 (incorporated herein for the teachings of this method). Control SHR and Wister-Kyoto rats (WKY) receive a placebo for the same period. The duration and initiation of treatment, site of administration, among other factors, can influence the reversal of pathophysiological alterations associated with hypertension. At the end of treatment, the effect on arterial systolic blood pressure and the level of perivascular collagen concentration is compared to controls. In addition, the medial cross-sectional area of the aorta is compared to that of untreated SHR. Data on vasuclar lumen changes is expressed as the mean (+/−) of a SEM. Other measurements used to determine treatment outcome are: (1) coronary flow (using the Langendorff-perfused heart model at baseline) after maximum vasodilation in response to adenosine (10(−5) M), after endothelium-dependent vasodilation in response to bradykinin (10(−8) M), and after ecNOS inhibition by nitro-L-arginine methyl ester (L-NAME) (10(−4) M); (2) medial thickening of coronary microvessels and perivascular collagen on histological heart sections; and (3) ecNOS expression by immunohistochemical staining in appropriate vessels using 20-week-old spontaneously hypertensive (SHR) and Wistar-Kyoto control rats (WKY). These measurements are determined by computer-directed color analysis. Statistical analysis are performed with a paired t-test and statistical significance is defined as p<0.05 vs. the response to buffer alone.

### Example 29

### Effect of an LP Polypeptide in a Rat Ischemic Skin Flap Model

Current estimates indicate that over 2,000,000 US citizens have chronic wounds each year, and the problem is increasing as the population ages. The cost of caring for chronic wounds reaches into the billions of dollars a year. Clearly, there is a need for better treatment to promote healing of chronic wounds. Ischemia is a major factor contributing to the failure of most chronic wounds to heal. Wound healing involves, e.g., soluble factors that control a series of processes including inflammation, cellular proliferation, and maturation (see, e.g., Robson, M. C. (1997) Wound Repair and Regeneration 5:12-17). Pro-inflammatory cytokines such as tumor necrosis factor (TNF) and Interleukin-1 (IL-1), proteases, protease inhibitors, and growth factors play important roles in normal wound healing. Excessive production of these proteins can impede wound healing (see, e.g., Mast, & Schultz (1996) Wound Repair and Regeneration 4:411-420). Ischemia of wound tissues occurs frequently in subjects having vascular disease (such as, e.g., venous hypertension, arterial insufficiency, or diabetes). Also, extended periods of pressure can cause ischemia in tissue pressure points in persons without nerve function who have lost nerve functions but are otherwise healthy (such as, e.g., quadriplegics or paraplegics). Thus, methods to restore reverse local tissue ischemia would promote healing of many chronic wounds. Delivery of an LP polypeptide (or fragment thereof) to wound cells (e.g., in a recombinant construct encoding the polypeptide or fragment) is used to test a polypeptide of the invention for its ability to treat ischemic, non-healing wounds. In one embodiment an LP polypeptide (or fragment thereof) is used in a rodent single pedicle dorsal skin flap method based on a technique of McFarlane, et al. (1965) Plastic and Reconstructive Surgery 35:177-182 to test angiogenesis.

### Example 30

### Effect of an LP Polypeptide in a Peripheral Arterial Disease Model

Angiogenic treatment using an LP polypeptide (or fragment thereof) is a novel therapeutic strategy to obtain restoration of blood flow around an ischemia (e.g., in a case of peripheral arterial disease). To test the ability of an LP polypeptide (or fragment thereof) to modulate such a peripheral arterial disease, the following experimental protocol is used: a) Using a rodent (as in the above described method) one side of the femoral artery is ligated to create ischemic damage to a muscle of the hindlimb (the other non-damaged hindlimb functions as the control); b) an LP polypeptide (or fragment thereof) is delivered to the animal either intravenously and/or intramuscularly (at the damaged limb) at least ×3 times per week for 2-3 weeks at a range of dosages (20 mg-500 mg); and c) the ischemic muscle tissue is collected after at 1, 2, and 3 weeks post-ligation for an analysis of expression of an LP polypeptide (or fragment thereof) and histology. Generally, (as above) parameters for evaluation include determining viability and vascularization of tissue surrounding the ischemia, while more specific evaluation parameters may include, e.g., measuring skin blood flow, skin temperature, and factor VIII immunohistochemistry, and/or endothelial alkaline phosphatase reaction. Polypeptide expression during the ischemia, is studied using any art known in situ hybridization technique. Biopsy is also performed on the other side of normal muscle of the contralateral hindlimb for analysis as a control.

### Example 31

### Effect of an LP Polypeptide in an Ischemic Myocardial Disease Mouse Model

An LP polypeptide (or fragment thereof) is evaluated as a treatment capable of stimulating the development of collateral vessels, and/or restructuring new vessels after coronary artery occlusion. The model is based on Guo, et al. (1999) Proc Natl Acad. Sci USA. 96:11507-11512 (incorporated herein for these teachings) demonstrating that a robust infarct-sparing effect occurs during the early and the late phases of preconditioning in the mouse and that the quantitative aspects of this effect are consistent with previous experience in other species. The model is useful to elucidate the molecular basis of ischemic preconditioning by making it possible to apply molecular biology techniques to intact animal preparations to dissect the precise role of a specific LP during ischemic events.

### Example 32

### Effect of an LP Polypeptide in a Rat Corneal Wound Healing Model

This animal model examines effects of an LP polypeptide (or fragment thereof) for angiogenic or anti-angiogenic activity on the normally avascular cornea. Briefly, the protocol comprises making a 1-1.5 mm long incision from the center of the corneal epithelium of an anesthetized mouse (e.g., a C57BL mouse strain) into the stromal layer then inserting a spatula below the lip of the incision facing the outer corner of the eye to make a pocket (whose base is 1-1.5 mm form the edge of the eye). Next, a pellet comprising an LP polypeptide or fragment thereof, (in a dosage range of about 50 ng-5 ug) is positioned within the pocket (being immobilized in a slow release form, e.g., in an inert hydron pellet of approximately 1-2 ml volume). Alternatively, treatment with an LP polypeptide (or fragment thereof) can also be applied topically to the corneal wound in a dosage range of 20 mg-500 mg (daily treatment for five days). Over a 5 to 7 day post-operative period any angiogenic effect (e.g., stimulating the in growth of vessels from the adjacent vascularized corneal limbus) is determined. A photographic record is created by slit lamp photography. The appearance, density and extent of these vessels are evaluated and scored. In some instances, the time course of the progression is followed in anesthetized animals, before sacrifice. Vessels are evaluated for length, density and the radial surface of the limbus from which they emanate (expressed as clock-faced hours). Corneal wound healing is also assessed using any other art known technique.

### Example 33

### Effect of an LP Polypeptide in a Diabetic Mouse and Glucocorticoid-Impaired Wound Healing Models

Diabetic Mouse (db-+/db+) as a Model

A genetically-induced diabetic mouse is used to examine the effect of an LP polypeptide (or fragment thereof) on wound healing. Mutant diabetic (db+/db+) mice have a single autosomal recessive mutation on chromosome 4 (db+) are used (Coleman et al. (1982) Proc. Natl. Acad. Sci. USA 72283-293). Typically, homozygous (db−/db−) mice are obese in comparison to their normal heterozygous (db+/db+) littermates. The mutant mice (db+/db+) have unique behavioral characteristics (such as, e.g., polyphagia, polydipsia, and polyuria); characteristic physiology (e.g., elevated blood glucose, increased or normal insulin levels, and suppressed cell-mediated immunity); and specific pathologies (such as, e.g., peripheral neuropathy, myocardial complications, and microvascular lesions, basement membrane thickening, and glomerular filtration abnormalities (see, e.g., Mandel, et al. (1978) J. Immunol. 120: 1375; Debray-Sachs, et al. (1983) Clin. Exp. Immunol. 51(1):1-7; Leiter, et al. (1985) Am. J. of Pathol. 114:46-55; Norido, et al. (1984) Exp. Neural. 83(2):221-232; Robertson, et al. (1980) Diabetes 29(1):60-67; Giacomelli, et al. (1979) Lab Invest. 40(4):460-473; Coleman, (1982) Diabetes 31 (Suppl): 1-6). These homozygous diabetic mice also develop a form of insulin-resistant hyperglycemia that is analogous to human type II diabetes (Mandel, et al. (1978) J. Immunol. 120: 1375-1377). All things considered, healing in the db+/db+ mouse may model the healing observed in humans with diabetes (see, Greenhalgh, et al. (1990) Am. J. of Pathol. 136:1235-1246). Thus, full-thickness, wound-healing using the db+/db+ mouse is a useful well-characterized, clinically relevant, and reproducible model of impaired wound healing in humans. Generally, it is agreed that healing of the diabetic wound is dependent on formation of granulation tissue and re-epithelialization rather than simply by contraction (see, e.g., Gartner, et al. (1992) J. Surg. Res. 52:389; Greenhalgh, et al (1990) Am. J. Pathol. 136:1235). Moreover, the diabetic db+/db+ animals have many of the characteristic features observed in Type II diabetes mellitus. Therefore, the genetically-induced db+/db+ diabetic mouse is useful to examine the effect of an LP polypeptide (or fragment thereof) on wound healing according to the following method. Genetically, diabetic female C57BWKsJ mice and their non-diabetic heterozygous littermates are purchased at 6 weeks of age (Jackson Laboratories) and are 8 weeks old at the start of testing. Animals are individually housed and received food and water ad libitum. All manipulations are performed using standard aseptic techniques. The wounding protocol is performed generally according to the method of Tsuboi & Rifkin, (1990) Exp. Med. 172:245-251.

Steroid Impaired Rat Model

The following method is designed to investigate the effect of a topical treatment of varying concentrations of an LP polypeptide (or fragment thereof) on the wound of a healing-impaired rat (methylprednisolone impairment of a full thickness excisional skin wound). The inhibition of wound healing by steroids (such as, e.g., the glucocorticoid methylprednisolone) is well documented both in vitro and in vivo (see, e.g., Wahl, (1989) Glucocorticoids and Wound healing. In: Anti-Inflammatory Steroid Action: Basic and Clinical Aspects pp. 280-302; Wahlet, al. (1975) J. Immunol. 115: 476-481; and Werb, et al. (1978) J. Exp. Med. 147:1684-1694). Glucocorticoids (such as methylprednisolone) are believed to retard wound healing by inhibiting angiogenesis, decreasing vascular permeability, fibroblast proliferation, collagen synthesis, and by transiently reducing the level of circulating monocytes. Furthermore, the systemic administration of steroids (such as glucocorticoids) to impair wound healing is a well established method used in rodents, such as, e.g., the rat (see, e.g., Ebert, et al. (1952) An. Intern. Med. 37:701-705; Beck, et al. (1991) Growth Factors. 5: 295-304; Haynes, et al. (1978) J. Clin. Invest. 61: 703-797; Haynes, et al. (1978) J. Clin. Invest. 61: 703-797; and Wahl, (1989), supra); and Pierce, et al. (1989) Proc. Natl. Acad. Sci. USA 86: 2229-2233). Thus, such a model is useful in assessing the effect of an LP polypeptide (or fragment thereof) of the invention on wound healing.

The assays, methods, or examples described herein test the activity of an LP polynucleotide sequence or an LP polypeptide (or fragment thereof). However, an ordinarily skilled artisan could easily modify (without undue experimentation) any exemplar taught herein using a different composition and/or concentration (such as, e.g., an agonist and/or an antagonist of an LP polynucleotide sequence or an LP polypeptide (or fragment thereof) of the invention. It will be clear that the invention may be practiced otherwise than as specifically described in the foregoing description and examples. Numerous modifications and variations of the present invention are possible in light of the above teachings and, therefore, are within the scope of the appended claims. The entire disclosure of each document cited (including patents, patent applications, journal articles, abstracts, laboratory manuals, books, or other disclosures) in the Background of the Invention, Detailed Description, and Examples is hereby incorporated herein by reference for the teachings they were intended to convey. Moreover, the hard copy of the sequence listing submitted herewith and the corresponding computer readable form are both incorporated herein by reference in their entireties, including without reservation, all corresponding drawings, pictures, graphs, diagrams, figures, figure legends, and http sites (including all corresponding information contained therein). The foregoing written specification is considered sufficient to enable a person of ordinary skill in the art to practice the invention. Indeed, various modifications of the invention in addition to those shown and described herein will become apparent from the foregoing description and these modifications also fall within the scope of the appended claims. All references cited herein are incorporated herein by reference to the same extent as if each individual publication or patent application was specifically and individually indicated to be incorporated by reference in its entirety for all purposes. Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

What is claimed is:

1. An isolated or recombinant polynucleotide comprising sequence encoding an antigenic polypeptide comprising at least 17 contiguous amino acids from a mature coding portion of SEQ ID NO: Y (LP318a, LP318b, LP288, LP289, LP343, LP319a, LP319b, LP321, LP317, LP283, LP344, LP345, or LP346).

2. The polynucleotide of claim 1, encoding:
a) a full length polypeptide of SEQ ID NO: Y or table 1-8;
b) a mature polypeptide of SEQ ID NO: Y or table 1-8;
c) an antigenic fragment at least 12 contiguous amino acid residues in length of SEQ ID NO: Y from an LP of Table 1, 2, 3, 4, 5, 6, 7 or 8;
d) at least two fragments of SEQ ID NO: Y from an LP of Table 1, 2, 3, 4, 5, 6, 7 or 8, wherein said fragment do not overlap;
e) a plurality of fragments of SEQ ID NO: Y from an LP of Table 1, 2, 3, 4, 5, 6, 7 or 8, wherein said fragment do not overlap; or
f) a mature polypeptide of SEQ ID NO:Y with less than five amino acid substitutions.

3. The polynucleotide of claim 1, which hybridizes at 55° C., less than 500 mM salt, to:
a) the mature coding portion of SEQ ID NO: 1;
b) the mature coding portion of SEQ ID NO: 3;
c) the mature coding portion of SEQ ID NO: 5;
d) the mature coding portion of SEQ ID NO: 7;
e) the mature coding portion of SEQ ID NO: 9;
f) the mature coding portion of SEQ ID NO: 11;
g) the mature coding portion of SEQ ID NO: 13;
h) the mature coding portion of SEQ ID NO: 15.
i) the mature coding portion of SEQ ID NO: 17;
j) the mature coding portion of SEQ ID NO: 19;
k) the mature coding portion of SEQ ID NO: 21;
l) the mature coding portion of SEQ ID NO: 22
m) the mature coding portion of SEQ ID NO: 23;

4. The polynucleotide of claim 3, wherein said temperature is at least 65° C., and said salt is less than 300 mM.

5. The polypeptide of claim 3, comprising at least 30, 32, 34, 36, 38, 39, 40, 42, 44, 46, 48, 49, 50, 52, 54, 56, 58, 59, 75, or at least about 150 contiguous nucleotides to a nucleotide sequence of LP(LP318a, LP318b, LP288, LP289, LP343, LP319a, LP319b, LP321, LP317, LP283, LP344, LP345, or LP346) of:
a) the mature coding portion of SEQ ID NO: 1;
b) the mature coding portion of SEQ ID NO: 3;
c) the mature coding portion of SEQ ID NO: 5;
d) the mature coding portion of SEQ ID NO: 7;
e) the mature coding portion of SEQ ID NO: 9;
f) the mature coding portion of SEQ ID NO: 11;
g) the mature coding portion of SEQ ID NO: 13;
h) the mature coding portion of SEQ ID NO: 15.
i) the mature coding portion of SEQ ID NO: 17;
j) the mature coding portion of SEQ ID NO: 19;
k) the mature coding portion of SEQ ID NO: 21;
l) the mature coding portion of SEQ ID NO: 22 or
m) the mature coding portion of SEQ ID NO: 23.

6. An expression vector comprising a polynucleotide of claim 1, wherein said temperature is at least 65° C., and said salt is less than 300 mM.

7. The expression vector of claim 6, which further comprises a plurality of 23 nucleotide segments with identity to the coding portion of SEQ ID NO: X.

8. A host cell containing the expression vector of claim 6, including a eukaryotic cell.

9. A method of making an antigenic polypeptide comprising expressing a recombinant polynucleotide of claim 1.

10. A method for detecting a polynucleotide of claim 1, comprising contacting said polynucleotide with a probe that hybridizes, under stringent conditions, to at least 25 contiguous nucleotides of:
a) the mature coding portion of SEQ ID NO: 1;
b) the mature coding portion of SEQ ID NO: 3;
c) the mature coding portion of SEQ ID NO: 5;
d) the mature coding portion of SEQ ID NO: 7;
e) the mature coding portion of SEQ ID NO: 9;
f) the mature coding portion of SEQ ID NO: 11;
g) the mature coding portion of SEQ ID NO: 13;
h) the mature coding portion of SEQ ID NO: 15.
i) the mature coding portion of SEQ ID NO: 17;
j) the mature coding portion of SEQ ID NO: 19;
k) the mature coding portion of SEQ ID NO: 21;
l) the mature coding portion of SEQ ID NO: 22 or
m) the mature coding portion of SEQ ID NO: 23.
to form a duplex, wherein detection of said duplex indicates the presence of said polynucleotide.

11. A kit for the detection of a polynucleotide of claim 1, comprising a compartment containing a probe that hybridizes, under stringent hybridization conditions, to at least 34 contiguous nucleotides of a polynucleotide of claim 1 to form a duplex.

12. The kit of claim 11, wherein said probe is detectably labeled.

13. A binding compound comprising an antibody which specifically binds to at least a 17 contiguous amino acid antigen binding site region of:
a) primate LP318a (SEQ ID NO: 2);
b) primate LP318b (SEQ ID NO: 4);
c) primate LP288 (SEQ ID NO: 6);
d) primate LP289 (SEQ ID NO: 8);
e) primate LP343 (SEQ ID NO: 10);
f) primate LP319a (SEQ ID NO: 12);
g) primate LP319b (SEQ ID NO: 14);
h) primate LP321 (SEQ ID NO: 16);
i) primate LP317 (SEQ ID NO: 18);
j) primate LP283 (SEQ ID NO: 20);
k) primate LP344 (SEQ ID NO: 21);
l) primate LP345 (SEQ ID NO: 22) or
m) primate LP346 (SEQ ID NO: 23).

14. The binding compound of claim 13, wherein:
a) said antibody binding site is:
i) specifically immunoreactive with a polypeptide of SEQ ID NO: Y;
ii) specifically immunoreactive with a polypeptide of SEQ ID NO: 2;
iii) specifically immunoreactive with a polypeptide of SEQ ID NO: 4;
iv) specifically immunoreactive with a polypeptide of SEQ ID NO: 6;
v) specifically immunoreactive with a polypeptide of SEQ ID NO: 8;
vi) specifically immunoreactive with a polypeptide of SEQ ID NO: 10;
vii) specifically immunoreactive with a polypeptide of SEQ ID NO: 12:
viii) specifically immunoreactive with a polypeptide of SEQ ID NO: 14;
ix) specifically immunoreactive with a polypeptide of SEQ ID NO: 16;
x) specifically immunoreactive with a polypeptide of SEQ ID NO: 18;
xi) specifically immunoreactive with a polypeptide of SEQ ID NO: 20;
xii) specifically immunoreactive with a polypeptide of SEQ ID NO: 21;
xiii) specifically immunoreactive with a polypeptide of SEQ ID NO: 22;
xiv) specifically immunoreactive with a polypeptide of SEQ ID NO: 23;
xv) raised against a purified or recombinantly produced human LP protein selected from: LP318a, LP318b, LP288, LP289, LP343, LP319a, LP319b, LP321, LP317, LP283, LP344, LP345, or LP346;
xvi) in a monoclonal antibody, Fab, or F(ab)2; or
b) said binding compound is:
i) an antibody molecule;
ii) a polyclonal antiserum;
iii) detectably labeled;
iv) sterile; or
v) in a buffered composition.

15. A method using the binding compound of claim 13, comprising contacting said binding compound with a biological sample comprising an antigen, thereby forming an LP binding compound:antigen complex.

16. The method of claim 15, wherein said biological sample is from a human, and wherein said binding compound is an antibody.

17. A detection kit comprising said binding compound of claim 14, and:
a) instructional material for the use of said binding compound for said detection; or
b) a compartment providing segregation of said binding compound.

18. A substantially pure or solated antigenic polypeptide, which binds to said binding composition of claim 13, and further comprises at least 25 contiguous amino acids from:
a) primate LP318a (SEQ ID NO: 2);
b) primate LP318b (SEQ ID NO: 4);
c) primate LP288 (SEQ ID NO: 6);
d) primate LP2894 (SEQ ID NO: 8);
e) primate LP343 (SEQ ID NO: 10);
f) primate LP319a (SEQ ID NO: 12);
g) primate LP319b (SEQ ID NO: 14);
h) primate LP321 (SEQ ID NO: 16);
i) primate LP317 (SEQ ID NO: 18);
j) primate LP283 (SEQ ID NO: 20);
k) primate LP344 (SEQ ID NO: 21);
l) primate LP345 (SEQ ID NO: 22) or
m) primate LP346 (SEQ ID NO: 23);

19. The polypeptide of claim 18, which:
a) comprises it least a fragment of at least 29 contiguous amino acid residues from a primate LP protein selected from: LP318a, LP318b, LP288, LP289, LP343, LP319a, LP319b, LP321, LP317, LP283, LP344, LP345, or LP346;
b) is a soluble polypeptide;
c) is detectably labeled;
d) is in a sterile composition;
e) is in a buffered composition;
f) is recombinantly produced, or
g) has a naturally occurring polypeptide sequence.

20. The binding compound of claim 14, where said compound is an antibody that:
a) is raised against a peptide sequence of a mature polypeptide of Table 1, 2, 3, 4, 5, 6, 7 or 8;
b) is produced in a mammal, or a plant;
c) is immunoselected; or
d) binds to a denatured polypeptide of Table 1, 2, 3, 4, 5, 6, 7 or8.


SEQUENCE LISTING (32 sequences)

SEQ ID NO: 1 - DNA, 685 bp, Primate
  CDS (41)..(679)
tagggataaaagtacaacattggactcagatcatgacacaatgaggcttcccccaaaggttattttcctgcttcgttccattagcaaagcagttgctgctacagactgggcacattcgggccatcggtgggttactgggtcaagaacttttgacaggaaggctatgggctgccagtggcctctgtgtctttgggtgagccccggagtccaggtgaccctcaaccttcacggcgaggcctcctacctcctccaggcactgggctccctctgctccccgtgggccgctccccgcgtggggccactgcccccggcccccgccatggtgcggatttcaaagcccaagacgtttcaggcctacttggatgattgtcaccggaggtatagctgtgcccactgccgcgctcacctggccaaccacgacgacctcatctccaagtccttccagggcagtcaggggcgtgcctacctcttcaactcagtggtgaacgtgggctgcgggccagccgaggagcgggtgctgctgaccggcctccatgctgtcgccgacatccactgcgagaactgcaagaccactttgggctggaaatatgaacaggcctttgagagcagccagaagtacaaagaggggaagtacatcattgaactcaaccacatgatcaaagacaacggctgggactgaaataaa

SEQ ID NO: 2 - protein, 212 aa, Primate
  misc_feature (1)..(685): LP318a
MRLPPKVIFLLRSISKAVAATDWAHSGHRWVTGSRTFDRKAMGCQWPLCLWVSPGVQVTLNLHGEASYLLQALGSLCSPWAAPRVGPLPPAPAMVRISKPKTFQAYLDDCHRRYSCAHCRAHLANHDDLISKSFQGSQGRAYLFNSVVNVGCGPAEERVLLTGLHAVADIHCENCKTTLGWKYEQAFESSQKYKEGKYIIELNHMIKDNGWD

SEQ ID NO: 3 - DNA, 508 bp, Primate
  misc_feature (1)..(508): LP318b
agaatatatagagaatatagatacataggtcctttatccaatggaggagctgatgctcagtcggaatgacagtgttctccacccatcctcaggtcatgacatccccccagcctcaggtcatgaactccccgcatcctcatacgtcatgaccaccgatcctcagctaacagccgcggggctggactcccctggttctgggcagcaccgcttcgttccagaaagcactcgcagccaatctcacgggaccttcttctttcagtcctttcaggggagccagggacgcgcctacctcttcaattccgtggtgaacgtgggctgcggccctgcagaggagagggtccttctcaccgggctgcatgcggttgccgacatctactgcgagaactgcaagaccacgctcgggtggaaatacgagcatgcctttgagagcagtcagaaatataaggaaggaaaattcatcattgagcttgctcatatgatcaaagacaatggctgggagtaaaaaaaa

SEQ ID NO: 4 - protein, 153 aa, Primate
  misc_feature (1)..(508): LP318b
MEELMLSRNDSVLHPSSGHDIPPASGHELPASSYVMTTDPQLTAAGLDSPGSGQHRFVPESTRSQSHGTFFFQSFQGSQGRAYLFNSVVNVGCGPAEERVLLTGLHAVADIYCENCKTTLGWKYEHAFESSQKYKEGKFIIELAHMIKDNGWE

SEQ ID NO: 5 - DNA, 7974 bp, Primate
  misc_feature (1)..(7974): LP288
ggcggcccgagggcgacttgcggggcgcgcaggccgccgtgcacccgggacgcttccccctcggggaccctccgcgggcttctccgccgcgccgtccggcgggagccggcgggaccccgggcgagcggcgcgggcggcaccatgaggcggcagtggggcgcgctgctgcttggcgccctgctctgcgcacacggcctggccagcagccccgagtgtgcttgtggtcggagccacttcacatgtgcagtgagtgctcttggagagtgtacctgcatccctgcccagtggcagtgtgatggagacaatgactgcggggaccacagcgatgaggatggatgtatactacctacctgttcccctcttgactttcactgtgacaatggcaagtgcatccgccgctcctgggtgtgtgacggggacaacgactgtgaggatgactcggatgagcaggactgtcccccccgggagtgtgaggaggacgagtttccctgccagaatggctactgcatccggagtctgtggcactgcgatggtgacaatgactgtggcgacaacagcgatgagcagtgtgacatgcgcaagtgctccgacaaggagttccgctgtagtgacggaagctgcattgctgagcattggtactgcgacggtgacaccgactgcaaagatggctccgatgaggagaactgtccctcagcagtgccagcgcccccctgcaacctggaggagttccagtgtgcctatggacgctgcatcctcgacatctaccactgcgatggcgacgatgactgtggagactggtcagacgagtctgactgctcctcccaccagccctgccgctctggggagttcatgtgtgacagtggcctgtgcatcaatgcaggctggcgctgcgatggtgacgcggactgtgatgaccagtctgatgagcgcaactgcaccacctccatgtgtacggcagaacagttccgctgtcactcaggccgctgtgtccgcctgtcctggcgctgtgatggggaggacgactgtgcagacaacagcgatgaagagaactgtgagaatacaggaagcccccaatgtgccttggaccagttcctgtgttggaatgggcgctgcattgggcagaggaagctgtgcaacggggtcaacgactgtggtgacaacagcgacgaaagcccacagcagaattgccggccccggacgggtgaggagaactgcaatgttaacaacggtggctgtgcccagaagtgccagatggtgcggggggcagtgcagtgtacctgccacacaggctaccggctcacagaggatgggcacacgtgccaagatgtgaatgaatgtgccgaggaggggtattgcagccagggctgcaccaacagcgaaggggctttccaatgctggtgtgaaacaggctatgaactacggcccgaccggcgcagctgcaaggctctggggccagagcctgtgctgctgttcgccaatcgcatcgacatccggcaggtgctgccacaccgctctgagtacacactgctgcttaacaacctggagaatgccattgcccttgatttccaccaccgccgcgagcttgtcttctggtcagatgtcaccctggaccggatcctccgtgccaacctcaacggcagcaacgtggaggaggttgtgtctactgggctggagagcccagggggcctggctgtggattgggtccatgacaaactctactggaccgactcaggcacctcgaggattgaggtggccaatctggacggggcccaccggaaggtgttgctgtggcagaacctggagaagccccgggccattgccttgcatcccatggagggtaccatttactggacagactggggcaacaccccccgtattgaggcctccagcatggatggctctggacgccgcatcattgccgatacccatctcttctggcccaatggcctcaccatcgactatgccgggcgccgtatgtactgggtggatgctaagcaccatgtcatcgagagggccaatctggatgggagtcaccgtaaggctgtcattagccagggcctcccgcatcccttcgccatcacagtgtttgaagacagcctgtactggacagactggcacaccaagagcatcaatagcgctaacaaatttacggggaagaaccaggaaatcattcgcaacaaactccacttccctatggacatccacaccttgcacccccagcgccaacctgcagggaaaaaccgctgtggggacaacaacggaggctgcacgcacctgtgtctgcccagtggccagaactacacctgtgcctgccccactggcttccgcaagatcagcagccacgcctgtgcccagagtcttgacaagttcctgctttttgcccgaaggatggacatccgtcgaatcagctttgacacagaggacctgtctgatgatgtcatcccactggctgacgtgcgcagtgctgtggcccttgactgggactcccgggatgaccacgtgtactggacagatgtcagcactgataccatcagcagggccaagtgggatggaacaggacaggaggtggtagtggataccagtttggagagcccagctggcctggccattgattgggtcaccaacaaactgtactggacagatgcaggtacagaccggattgaagtagccaacacagatggcagcatgagaacagtactcatctgggagaaccttgatcgtcctcgggacatcgtggtggaacccatgggcgggtacatgtattggactgactggggtgcgagccccaagattgaacgagctggcatggatgcctcaggccgccaagtcattatctcttctaatctgacctggcctaatgggttagctattgattatgggtcccagcgtctatactgggctgacgccggcatgaagacaattgaatttgctggactggatggcagtaagaggaaggtgctgattggaagccagctcccccacccatttgggctgaccctctatggagagcgcatctattggactgactggcagaccaagagcatacagagcgctgaccggctgacagggctggaccgggagactctgcaggagaacctggaaaacctaatggacatccatgtcttccaccgccgccggcccccagtgtctacaccatgtgctatggagaatggcggctgtagccacctgtgtcttaggtccccaaatccaagcggattcagctgtacctgccccacaggcatcaacctgctgtctgatggcaagacctgctcaccaggcatgaacagtttcctcatcttcgccaggaggatagacattcgcatggtctccctggacatcccttattttgctgatgtggtggtaccaatcaacattaccatgaagaacaccattgccgttggagtagacccccaggaaggaaaggtgtactggtctgacagcacactgcacaggatcagtcgtgccaatctggatggctcacagcatgaggacatcatcaccacagggctacagaccacagatgggctcgcggttgatgccattggccggaaagtatactggacagacacgggaacaaaccggattgaagtgggcaacctggacgggtccatgcggaaagtgttggtgtggcagaaccttgacagtccccgggccatcgtactgtaccatgagatggggtttatgtactggacagactggggggagaatgccaagttagagcggtccggaatggatggctcagaccgcgcggtgctcatcaacaacaacctaggatggcccaatggactgactgtggacaaggccagctcccaactgctatgggccgatgcccacaccgagcgaattgaggctgctgacctgaatggtgccaatcggcatacattggtgtcaccggtgcagcacccatatggcctcaccctgctcgactcctatatctactggactgactggcagactcggagcatccaccgtgctgacaagggtactggcagcaatgtcatcctcgtgaggtccaacctgccaggcctcatggacatgcaggctgtggaccgggcacagccactaggttttaacaagtgcggctcgagaaatggcggctgctcccacctctgcttgcctcggccttctggcttctcctgtgcctgccccactggcatccagctgaagggagatgggaagacctgtgatccctctcctgagacctacctgctcttctccagccgtggctccatccggcgtatctcactggacaccagtgaccacaccgatgtgcatgtccctgttcctgagctcaacaatgtcatctccctggactatgacagcgtggatggaaaggtctattacacagatgtgttcctggatgttatcaggcgagcagacctgaacggcagcaacatggagacagtgatcgggcgagggctgaagaccactgacgggctggcagtggactgggtggccaggaacctgtactggacagacacaggtcgaaataccattgaggcgtccaggctggatggttcctgccgcaaagtactgatcaacaatagcctggatgagccccgggccattgctgttttccccaggaaggggtacctcttctggacagactggggccacattgccaagatcgaacgggcaaacttggatggttctgagcggaaggtcctcatcaacacagacctgggttggcccaatggccttaccctggactatgatacccgcaggatctactgggtggatgcgcatctggaccggatcgagagtgctgacctcaatgggaaactgcggcaggtcttggtcggccatgtgtcccacccctttgccctcacacagcaagacaggtggatctactggacagactggcagaccaagtcaatccagcgtgttgacaaatactcaggccggaacaaggagacagtgctggcaaatgtggaaggactcatggatatcatcgtggtttcccctcagcggcagacagggaccaatgcctgtggtgtgaacaatggtggctgcacccacctctgctttgccagagcctcggacttcgtatgtgcctgtcctgacgaacctgatagccagccctgctcccttgtgcctggcctggtaccaccagctcctagggctactggcatgagtgaaaagagcccagtgctacccaacacaccacctaccaccttgtattcttcaaccacccggacccgcacgtctctggaggaggtggaaggaagatgctctgaaagggatgccaggctgggcctctgtgcacgttccaatgacgctgttcctgctgctccaggggaaggacttcatatcagctacgccattggtggactcctcagtattctgctgattttggtggtgattgcagctttgatgctgtacagacacaaaaaatccaagttcactgatcctggaatggggaacctcacctacagcaacccctcctaccgaacatccacacaggaagtgaagattgaagcaatccccaaaccagccatgtacaaccagctgtgctataagaaagagggagggcctgaccataactacaccaaggagaagatcaagatcgtagagggaatctgcctcctgtctggggatgatgctgagtgggatgacctcaagcaactgcgaagctcacgggggggcctcctccgggatcatgtatgcatgaagacagacacggtgtccatccaggccagctctggctccctggatgacacagagatggagcagctgttacaggaagagcagtctgagtgtagcagcgtccatactgcagccactccagaaagacgaggctctctgccagacacgggctggaaacatgaacgcaagctctcctcagagagccaggtctaaatgcccacattctcttccctgcctgcctgttccttctcctttatggacgtctagtccttgtgctcgcttacaccgcaggccccgcttctgtgtgcttgtcctcctcctcctcccaccccataactgttcctaagccttcaccggagctgtttaccacgtgagtccataactacctgtgcacaagaaatgatggcacatcacgagaatttagacctggattttaccatgaacctcacatcttgtactccatcctgggccccctgaaactgcttattcgtgattcctcaccagcgtagagctccacctcccctttccccagtaccctcagtgcctgcttctcagtgctgatgcagctgatgacccaggactgcgctctgccccatcacagccagcatgactgcttctctgagagaacttgcccatcaggggctgggacatgggggtgtgggtaaagacagggatgaaggatagaggctgagagaagaaggaagaatcagcccagcaggtatgggcatctgggaaacctccagcctcaagtgtgttggtaacatgaaaaagctctggggggtagttggatctgggtgtctggtccattgctggcagtggacattattcttgccctaagagacactgccttttcagcagcagatactggtgagatgggggtggctcaggctgttcttcctcctcctagaatgtctggagctgtttctacattcagataactgggtcccctatcacaaggctactggctaataggaattccctcctggtgccaccactggccagtacctttcctaagtctttgctcaaattaaccaggttgtgagccagtggcttgagtgaatgttaggccttgggggctgagtctctgaaaagtctaagaagctctgcctagaccaaatatggtatacctcctgacccctctctccctcatgtcctgggattctggggaagagacctagaaacaagctttcaaagaaaaaccagaagttgtcataaatggtcagaaagaacgatcaggttggagacttgggaaacccagggcctaaagagaagtatccatgagggtcaaacttcctgttgaacttcctatgttctttctcaagtgctcagggatctaagttagtggacagcaagcctgtggctacggggtggtgatgttcctcttccagctgtcccctcagctaaggggcttagtttccatgtgggatgccatcacttggttcatgctcattcacacaaagggcacgtgtctcagcctggtatcagggaaattgagacttatttttgccctaaaacgtctccctagctgttcttcgtggggtttttttgtttgtttttttgcctaatttgctttttctgaccaagccttgtggcaccagcaatctccaaagtcctgtggtgggagggctgaataaataaaaatacaaagaggtgggtaaggagtaggaaggtagagagcaccactgatgaggccctcctagcccatggcagacccagacctcttctcccccaggaattagaagtggcaggagagaacaacaggggctgggaatggaggggagaatttctaggggaagtttcctgagttgaaacttctcctgtggttactggtattgagaaatcagctaccaaagtgaaaaaggacaagatcaattcttttctagtcagttctaagactgctagagagagataccaggcccttagccttgctctcagtagcgtcagccccagttctgagcctccccacattacacttaacaagcagtaaaggagtgagcactttgggtccttagactcatgtctggggaggaagagcaagtagaaaagtggcattttcttgattggaaagggggaaggatcttattgcacttgggctgttcagaatgtagaaaggacatatttgaggaagtatctatttgagcactgatttactctgtaaaaagcaaaatctctctgtcctaaactaatggaagcgattctcccatgctcatgtgtaatggttttaacgttactcactggagagattggactttctggagttatttaaccactatgttcagtattttaggactttatgataatttaatataaatttagcttttcttaatc

SEQ ID NO: 6 - protein, 1905 aa, Primate
  misc_feature (1)..(7974): LP288
MRRQWGALLLGALLCAHGLASSPECACGRSHFTCAVSALGECTCIPAQWQCDGDNDCGDHSDEDGCILPTCSPLDFHCDNGKCIRRSWVCDGDNDCEDDSDEQDCPPRECEEDEFPCQNGYCIRSLWHCDGDNDCGDNSDEQCDMRKCSDKEFRCSDGSCIAEHWYCDGDTDCKDGSDEENCPSAVPAPPCNLEEFQCAYGRCILDIYHCDGDDDCGDWSDESDCSSHQPCRSGEFMCDSGLCINAGWRCDGDADCDDQSDERNCTTSMCTAEQFRCHSGRCVRLSWRCDGEDDCADNSDEENCENTGSPQCALDQFLCWNGRCIGQRKLCNGVNDCGDNSDESPQQNCRPRTGEENCNVNNGGCAQKCQMVRGAVQCTCHTGYRLTEDGHTCQDVNECAEEGYCSQGCTNSEGAFQCWCETGYELRPDRRSCKALGPEPVLLFANRIDIRQVLPHRSEYTLLLNNLENAIALDFHHRRELVFWSDVTLDRILRANLNGSNVEEVVSTGLESPGGLAVDWVHDKLYWTDSGTSRIEVANLDGAHRKVLLWQNLEKPRAIALHPMEGTIYWTDWGNTPRIEASSMDGSGRRIIADTHLFWPNGLTIDYAGRRMYWVDAKHHVIERANLDGSHRKAVISQGLPHPFAITVFEDSLYWTDWHTKSINSANKFTGKNQEIIRNKLHFPMDIHTLHPQRQPAGKNRCGDNNGGCTHLCLPSGQNYTCACPTGFRKISSHACAQSLDKFLLFARRMDIRRISFDTEDLSDDVIPLADVRSAVALDWDSRDDHVYWTDVSTDTISRAKWDGTGQEVVVDTSLESPAGLAIDWVTNKLYWTDAGTDRIEVANTDGSMRTVLIWENLDRPRDIVVEPMGGYMYWTDWGASPKIERAGMDASGRQVIISSNLTWPNGLAIDYGSQRLYWADAGMKTIEFAGLDGSKRKVLIGSQLPHPFGLTLYGERIYWTDWQTKSIQSADRLTGLDRETLQENLENLMDIHVFHRRRPPVSTPCAMENGGCSHLCLRSPNPSGFSCTCPTGINLLSDGKTCSPGMNSFLIFARRIDIRMVSLDIPYFADVVVPINITMKNTIAVGVDPQEGKVYWSDSTLHRISRANLDGSQHEDIITTGLQTTDGLAVDAIGRKVYWTDTGTNRIEVGNLDGSMRKVLVWQNLDSPRAIVLYHEMGFMYWTDWGENAKLERSGMDGSDRAVLINNNLGWPNGLTVDKASSQLLWADAHTERIEAADLNGANRHTLVSPVQHPYGLTLLDSYIYWTDWQTRSIHRADKGTGSNVILVRSNLPGLMDMQAVDRAQPLGFNKCGSRNGGCSHLCLPRPSGFSCACPTGIQLKGDGKTCDPSPETYLLFSSRGSIRRISLDTSDHTDVHVPVPELNNVISLDYDSVDGKVYYTDVFLDVIRRADLNGSNMETVIGRGLKTTDGLAVDWVARNLYWTDTGRNTIEASRLDGSCRKVLINNSLDEPRAIAVFPRKGYLFWTDWGHIAKIERANLDGSERKVLINTDLGWPNGLTLDYDTRRIYWVDAHLDRIESADLNGKLRQVLVGHVSHPFALTQQDRWIYWTDWQTKSIQRVDKYSGRNKETVLANVEGLMDIIVVSPQRQTGTNACGVNNGGCTHLCFARASDFVCACPDEPDSQPCSLVPGLVPPAPRATGMSEKSPVLPNTPPTTLYSSTTRTRTSLEEVEGRCSERDARLGLCARSNDAVPAAPGEGLHISYAIGGLLSILLILVVIAALMLYRHKKSKFTDPGMGNLTYSNPSYRTSTQEVKIEAIPKPAMYNQLCYKKEGGPDHNYTKEKIKIVEGICLLSGDDAEWDDLKQLRSSRGGLLRDHVCMKTDTVSIQASSGSLDDTEMEQLLQEEQSECSSVHTAATPERRGSLPDTGWKHERKLSSESQV

SEQ ID NO: 7 - DNA, 2653 bp, Primate
  misc_feature (1)..(2653): LP289
cgccgcctctgccgcgatgcccccccctgcgcccggggcccggctccggcttctcgccgccgccgccctggccggcttggccgtcatcagccgagggctgctctcccagagcctggagttcaactctcctgccgacaactacacagtgtgtgaaggtgacaacgccaccctcagctgcttcatcgacgagcacgtgacccgcgtggcctggctgaaccgctccaacatcctgtatgccggcaatgaccgctggaccagcgacccgcgggtgcggctgctcatcaacacccccgaggagttctccatcctcatcaccgaggtggggctcggcgacgagggcctctacacctgctccttccagacccgccaccagccgtacaccactcaggtctacctcattgtccacgtccctgcccgcattgtgaacatctcgtcgcctgtgacggtgaatgaggggggcaatgtgaacctgctttgcctggccgtggggcggccagagcccacggtcacctggagacagctccgagacggcttcacctcggagggagagatcctggagatctctgacatccagcggggccaggccggggagtatgagtgcgtgactcacaacggggttaactcggcgcccgacagccgccgcgtgctggtcacagtcaactatcctccgaccatcacggacgtgaccagcgcccgcaccgcgctgggccgggccgccctcctgcgctgcgaagccatggcggttccccccgcggatttccagtggtacaaggatgacagactgctgagcagcggcacggccgaaggcctgaaggtgcagacggagcgcacccgctcgatgcttctctttgccaacgtgagcgcccggcattacggcaactatacgtgtcgcgccgccaaccgactgggagcgtccagcgcctccatgcggctcctgcgcccaggatccctggagaactcagccccgaggcccccagggctcctggccctcctctccgccctgggctggctgtggtggagaatgtaggcgcaacccagtggagctcacctccccctgcagggggcctcaggccaagagtgagagaaacgggggagcaagagccgtgggtctcgtgggggcagaagagctctcggccaccaaggaagaagagagaggagaagaggaggaggcagaggaagaaagatcttcagagaacccatcactgtgagggataacgcaaaattatgcatctttctacagccattctcgccacccgttcacgtttccgattgtgacccactcccgccaccccatacccctctctcttagctcaggctgtcaactggcttgtgtgggtgtgggtgtgtgagtgtgagcctgcatgcatgtgtaggtgtctgtgtctctgtttgtgtgtgtgtgggggggtgggctgggggaagggactcagccgtcctcccgcccccaaaacccccatgtccactgtccccaaccctattgcctctcacctctggctggaggtaggtatgaggctctgcagtggaagctgtgaagagaggcttaccaggctccctgcttccccaatatatgcacgcacactacccctccccctgtcgaaagggcaactcctggcaggttgggagtggggaggggcatcgcactcacctgtcaagctgtcattcagcctttgtgagtaagtgggggacctccatctaggctctgggcttccctgctgtcaaccaccagcttcctgtagccagaggccccatcgctggctcagtggcctgagaccttcccacttttcctgtccctccacaccctggattttctggccccctcctgggccaatcggtgcttccatccaactgtcagactggtggcaggagtcacctgtctgtttcagtgctttgccctacccttgccaccactccccttggccttgatttccccacctgcggcaccagccagctgcccttggggcctttgcagctggcccacaggtgtgagcttgctggtctatcacttcctggcctctccaccaactgtacctcccaaaggctacctgaccgccctcacagacactcagacgcaccacacacacatacacacacacagacacacacacacacacacacacacacaccagcgactgtgaccagcaatagccccctggctccagctgtccggtagctggtgactagctgtatttccccctgggtaggcatctggtgtcaggaagggacgctgagaggccgcagcctcagctctgactgtaggaacactccaccctggtacaccacaccacacaaagtgggggacagagaggcatcacacagacatcctgagcccagacaaagatgccatgtcaccaactccgactcccaggagaccctctatataaacacccaccccaaaccacaccacccagagtctggtggagaaaaggagagggcaaggcgcagtgactgtacctcagacgggggcatgggccccatcccacattgtcttccattcccagggtccaggggatggggtgggacaggggagggatgctagggagggggtggggggccctgggggccatgtgttccaattcatggggagtgttgagaccaccacaccaataaacgcctttttccaaaaaaaaaaaaaaagggcggccgccgactagtg

SEQ ID NO: 8 - protein, 336 aa, Primate
  misc_feature (1)..(2653): LP289
MPPPAPGARLRLLAAAALAGLAVISRGLLSQSLEFNSPADNYTVCEGDNATLSCFIDEHVTRVAWLNRSNILYAGNDRWTSDPRVRLLINTPEEFSILITEVGLGDEGLYTCSFQTRHQPYTTQVYLIVHVPARIVNISSPVTVNEGGNVNLLCLAVGRPEPTVTWRQLRDGFTSEGEILEISDIQRGQAGEYECVTHNGVNSAPDSRRVLVTVNYPPTITDVTSARTALGRAALLRCEAMAVPPADFQWYKDDRLLSSGTAEGLKVQTERTRSMLLFANVSARHYGNYTCRAANRLGASSASMRLLRPGSLENSAPRPPGLLALLSALGWLWWRM

SEQ ID NO: 9 - DNA, 754 bp, Primate
  misc_feature (1)..(754): LP343
cgtgccgcctctgccgcgatgcccccccctgcgcccggggcccggctccggcttctcgccgccgccgccctggccggcttggccgtcatcagccgagctgcttcatcgacgagcacgtgacccgcgtggcctggctgaaccgctccaacatcctgtatgccggcaatgaccgctggaccagcgacccgcgggtgcggctgctcatcaacacccccgaggagttctccatcctcatcaccgaggtggggctcggcgacgagggcctctacacctgctccttccagacccgccaccagccgtacaccactcaggtctacctcattgtccacgtccctgcccgcattgtgaacatctcgtcgcctgtgacggtgaatgaggggggcaatgtgaacctgctttgcctggccgtggggcggccagagcccacggtcacctggagacagctccgagacggcttcacctcggagggagagatcctggagatctctgacatccagcggggccaggccggggagtatgagtgcgtgactcacaacggggttaactcggcgcccgacagccgccgcgtgctggtcacagtcaactatcctccgaccatcacggacgtgaccagcgcccgcaccgcgctgggccgggccgccctcctgcgctgcgaagccatggcggttccccccgcggatttccagtggtacaaggatgacagactgctgagcagcggcacggccgaaggcctgaaggtgcagatggagcgcac

SEQ ID NO: 10 - protein, 33 aa, Primate
  misc_feature (1)..(754): LP343
MPPPAPGARLRLLAAAALAGLAVISRAASSTST

SEQ ID NO: 11 - DNA, 2597 bp, Primate
  misc_feature (1)..(2597): LP319a
gcctcgcgcgccccggaccggccccccctttcccctccccctccgtgccgcctctgccgcgatgccccccgctgcgcccggggcccggctccggcttctcgccgccgccgccctggccggcttggccgtcatcagccgggggctgctctcccagaggctggagttcaactctcctgccgacaactacacacatgtgacccgcgtggcctggctgaaccgctccaacatcctgtacgccggcaacgaccgcaggaccagggacccgcgggtgcggctgctcatcaacacctccgaggagttctccatcctcgtcaccgaggtggggctcggcgacgagggcctctacacctgctccttccagacccgccaccagccgtacaccactcaggtctacctcattgtccacgtccctgcccgcgttgtgaacatctcgtcgcctgtgatggtgaatgagggaggtaatgtgaacctgctttgcctggccgtggggcggccagagcccacggtcacctggagacagctccgagacggcttcacctcggagggagagatcctggagatctctgacatcctgcggggccaggccggggagtatgagtgcgtgactcacaacggggttaactcggcgcccgacagccgccgcgtgctggtcacagtcaactatcctccgaccatcacggacgtgaccagcgcccgcaccgcgctgggccgggccgcctactgcgctgcgaagccatggcggtttcccccgcggatttccagtggtataaggatgacagactactgagcagcggcacggccgagggcctgaaggtgcagatggagcgcactcgctcgatgcttctctttgccaacatgagcgcccggcattacggcaactatacgtgttgcgccgccaaccggctgggagcgtccagcgcctccatgcggctcctgtgcccaggatccctggagaactcagccccgaggcccccagggcccctggccctcctctccgccctgggctggctgtggtggagaatgtaggcgcaacccagtggagctcgcctccccctgcagggggcctcaggccaagagtgagagaaaagggggagcaagagccctgggtctcgtgggggcagaagggctctcggccaccaaggaagaagagaagaggagaaagaggaggaggcagaggaagaaagatctttagagaacccatcactgtgaggggtaacgcaaaattatgtatctttctacagccattctcgctacccgttcacgtttccgattgtgacccactcccgccaccccatacccctctctcttagctcaggctgtcaacaggcttgtgtgggtgtgggtgtgtgagtgtaagcctgcatgcgtgtgtaggtgtctgtgtctctgtgtgtgtgtgtgtgggtgggctgggggaagggactcaaccgtcctcccacccccgaaacccccatgtccactgtccccaaccctactgcctctcacctctggctggaggtaggtatgaggctctgcagtggaagctgtgaagagaggcttaccaggctccctgcttccccaatatatgcaggcacactgacccctccccctgtcgaaaggacaactcctggcaggttgggagtggggaggggcatcgcactcacctgtcaagctgtcattcagcctttgcgagtaagtgggggacctccatctgggttctgggcttccctgctgtcaaccaccagcttcctgtagccagaggctccatcgctggctcagtggcctgagaccttcccacttttcctgtccctacccaccctggattttctggccccctcttgggccaatcggtgcttccatccaactgtcagactggtggcaggagtcacctgtctgtttcagtgctttgccttacccctgccaccactccccttggccttgatttccccacctgcggaaccagccagctgcccctggggcctttgcagctggcccacaggtgtgggcttgctggtctatcacttcctggcctctccaccaactgtacttcccaaaggctacctgaccgccctcacacacactcagacacaccacacacacacacaccagcgactgtgaccagcaatagccccctggctccagctgtcctgtagctggtgactagctgtatttccccctgggtaggcatctgttgtcaggaagggacgctgagaggctgcagcctcagctctgattgtaggaatactccgccctggtacaccacaccacacaaagtgggggacagagaggcatcacacagacatcctgagcccagataaagatgccatgtcaccaactccgactcccacgagaccctctatataaacacccaccccaaaccacaccactcagagtctggtggagaagaggagagggcaaggctcagtgactgtacctcagacgggggcatgggccccatcccacgttgtcttccattcccagggtccaggggatggggtgggactggggagggattctagggagggggtgggaggccctgggggctgtgtgttccaattcatggggattgttgagaccaccacaccaataaatgcctttttccaaaaaaaaaaaaaaaa

SEQ ID NO: 12 - protein, 241 aa, Primate
  misc_feature (1)..(2597): LP319a
MPPAAPGARLRLLAAAALAGLAVISRGLLSQRLEFNSPADNYTHVTRVAWLNRSNILYAGNDRRTRDPRVRLLINTSEEFSILVTEVGLGDEGLYTCSFQTRHQPYTTQVYLIVHVPARVVNISSPVMVNEGGNVNLLCLAVGRPEPTVTWRQLRDGFTSEGEILEISDILRGQAGEYECVTHNGVNSAPDSRRVLVTVNYPPTITDVTSARTALGRAAYCAAKPWRFPPRISSGIRMTDY

SEQ ID NO: 13 - DNA, 2601 bp, Primate
  misc_feature (1)..(2601): LP319b
tccgtgccgcctctgccgcgatgccccccgctgcgcccggggcccggctccggcttctcgccgccgccgccctggccggcttggccgtcatcagccgggggctgctctcccagaggctggagttcaactctcctgccgacaactacacagtgtgtgaaggtgacaacgccaccctcagctgcttcatggacgagcatgtgacccgcgtggcctggctgaaccgctccaacatcctgtacgccggcaacgaccgcaggaccagggacccgcgggtgcggctgctcatcaacacctccgaggagttctccatcctcgtcaccgaggtggggctcggcgacgagggcctctacacctgctccttccagacccgccaccagccgtacaccactcaggtctacctcattgtccacgtccctgcccgcgttgtgaacatctcgtcgcctgtgatggtgaatgagggaggtaatgtgaacctgctttgcctggccgtggggcggccagagcccacggtcacctggagacagctccgagacggcttcacctcggagggagagatcctggagatctctgacatcctgcggggccaggccggggagtatgagtgcgtgactcacaacggggttaactcggcgcccgacagccgccgcgtgctggtcacagtcaactatcctccgaccatcacggacgtgaccagcgcccgcaccgcgctgggccgggccgcctactgcgctgcgaagccatggcggtttcccccgcggatttccagtggtataaggatgacagactactgagcagcggcacggccgagggcctgaaggtgcagatggagcgcactcgctcgatgcttctctttgccaacatgagcgcccggcattacggcaactatacgtgttgcgccgccaaccggctgggagcgtccagcgcctccatgcggctcctgtgcccaggatccctggagaactcagccccgaggcccccagggcccctggccctcctctccgccctgggctggctgtggtggagaatgtaggcgcaacccagtggagctcgcctccccctgcagggggcctcaggccaagagtgagagaaaagggggagcaagagccctgggtctcgtgggggcagaagggctctcggccaccaaggaagaagagaagaggagaaagaggaggaggcagaggaagaaagatctttagagaacccatcactgtgaggggtaacgcaaaattatgtatctttctacagccattctcgctacccgttcacgtttccgattgtgacccactcccgccaccccatacccctctctcttagctcaggctgtcaacaggcttgtgtgggtgtgggtgtgtgagtgtaagcctgcatgcgtgtgtaggtgtctgtgtctctgtgtgtgtgtgtgtgggtgggctgggggaagggactcaaccgtcctcccacccccgaaacccccatgtccactgtccccaaccctactgcctctcacctctggctggaggtaggtatgaggctctgcagtggaagctgtgaagagaggcttaccaggctccctgcttccccaatatatgcaggcacactgacccctccccctgtcgaaaggacaactcctggcaggttgggagtggggaggggcatcgcactcacctgtcaagctgtcattcagcctttgcgagtaagtgggggacctccatctgggttctgggcttccctgctgtcaaccaccagcttcctgtagccagaggctccatcgctggctcagtggcctgagaccttcccacttttcctgtccctacccaccctggattttctggccccctcttgggccaatcggtgcttccatccaactgtcagactggtggcaggagtcacctgtctgtttcagtgctttgccttacccctgccaccactccccttggccttgatttccccacctgcggaaccagccagctgcccctggggcctttgcagctggcccacaggtgtgggcttgctggtctatcacttcctggcctctccaccaactgtacttcccaaaggctacctgaccgccctcacacacactcagacacaccacacacacacacaccagcgactgtgaccagcaatagccccctggctccagctgtcctgtagctggtgactagctgtatttccccctgggtaggcatctgttgtcaggaagggacgctgagaggctgcagcctcagctctgattgtaggaatactccgccctggtacaccacaccacacaaagtgggggacagagaggcatcacacagacatcctgagcccagataaagatgccatgtcaccaactccgactcccacgagaccctctatataaacacccaccccaaaccacaccactcagagtctggtggagaagaggagagggcaaggctcagtgactgtacctcagacgggggcatgggccccatcccacgttgtcttccattcccagggtccaggggatggggtgggactggggagggattctagggagggggtgggaggccctgggggctgtgtgttccaattcatggggattgttgagaccaccacaccaataaatgcctttttccaaaaaaaaaaaaaaaa

SEQ ID NO: 14 - protein, 256 aa, Primate
  misc_feature (1)..(2601): LP319b
MPPAAPGARLRLLAAAALAGLAVISRGLLSQRLEFNSPADNYTVCEGDNATLSCFMDEHVTRVAWLNRSNILYAGNDRRTRDPRVRLLINTSEEFSILVTEVGLGDEGLYTCSFQTRHQPYTTQVYLIVHVPARVVNISSPVMVNEGGNVNLLCLAVGRPEPTVTWRQLRDGFTSEGEILEISDILRGQAGEYECVTHNGVNSAPDSRRVLVTVNYPPTITDVTSARTALGRAAYCAAKPWRFPPRISSGIRMTDY

SEQ ID NO: 15 - DNA, 480 bp, Primate
  misc_feature (1)..(480): LP321
tataaatgcaggctggatattcactgtccacacattgagctcctgctcaccaatcctccaggtgactcccagccatgaagacactagtcctcctctctgctcttgtcctgctggccttgcaggtccaggctgatcccattcaagaggcagaggaagagactaaaactgaggagcagccagcagatgaggaccaggatgtgtctgtctcctttgaaggcccagaagcctctgctgttcaagatttacgcgtaagaaggaccttgcagtgcagttgcagaagagtctgcagaaatacgtgtagctgcattcggctatcaaggtccacatatgcatcataaattctgctgcagctgaacatggaccccaaggatgagataaccacatgccctggggcctcacagtcggtcattacccttgtgcttggcctcgactgtttttccttctccaataaactccttgcagataaaaaaaaaaaaaaaa

SEQ ID NO: 16 - protein, 87 aa, Primate
  misc_feature (1)..(480): LP321
MKTLVLLSALVLLALQVQADPIQEAEEETKTEEQPADEDQDVSVSFEGPEASAVQDLRVRRTLQCSCRRVCRNTCSCIRLSRSTYAS

SEQ ID NO: 17 - DNA, 535 bp, Primate
  misc_feature (1)..(535): LP317
gtcacctgacctcatagataactctagggcggacggacggacgcgcgtctccggtcccgtccgtaatagcactgatccgatccacgccggccggcgatggagctcatcaagtccagggcgaccgtgtgcgcgctcctcctggcgctgctcctgctctcgcactacgacggcgggacgacgacgacgatggtggcggaggcccgggtgtgcatgggcaagagccagcaccactcgttcccctgcatctccgaccgcctctgcagcaacgagtgcgtcaaggaggacggcgggtggaccgccggctactgccacctccgctactgcaggtgccagaaggcgtgctaagcaaagctcttgaaacacccttggcttgccagaactgaactgtggtagtactaagtaacacccttggctagctgtgcacaacctacgtaccgtgcatgcatgtaatgtggtgtcatgtaacgtgacagcaataaatattaataacaataataacacggcatgtagcctttgcatgcttccaaaaaaaaaa

SEQ ID NO: 18 - protein, 82 aa, Primate
  misc_feature (1)..(535): LP317
MELIKSRATVCALLLALLLLSHYDGGTTTTMVAEARVCMGKSQHHSFPCISDRLCSNECVKEDGGWTAGYCHLRYCRCQKAC

SEQ ID NO: 19 - DNA, 3183 bp, Primate
  misc_feature (1)..(3183): LP283
ccctcccctccccctcctgcgagctgggatccggccggcttccgccctcccctggccgcgagaccggccccggcggctgggccgccagtagctccagccatgggctcggggcgcgtacccgggctctgcctgcttgtcctgctggtccacgcccgcgccgcccagtacagcaaagccgcgcaagatgtggatgagtgtgtggaggggactgacaactgccacatcgatgctatctgccagaacaccccgaggtcatacaagtgcatctgcaagtctggctacacaggggacggcaaacactgcaaagacgtggatgagtgcgagcgagaggataatgcaggttgtgtgcatgactgtgtcaacatccctggcaattaccggtgtacctgctatgatggattccacctggcacatgacggacacaactgtctggatgtggacgagtgtgccgagggcaacggcggctgtcagcagagctgtgtcaacatgatgggcagctatgagtgccactgccgggaaggcttcttcctcagcgacaaccagcatacctgtatccagcggccagaagaaggaatgaattgcatgaacaagaaccacggctgtgcccacatttgccgggagacacccaaggggggtattgcctgtgaatgccgtcctggctttgagcttaccaagaaccaacgggactgtaaattgacatgcaactatggtaacggcggctgccagcacacgtgtgatgacacagagcagggtccccggtgcggctgccatatcaagtttgtgctccataccgacgggaagacatgcatcggggaaaggcggctagagcagcacatccccactcaagccgtttctaatgagacctgtgctgtcaacaacgggggctgtgacagtaagtgccatgatgcagcgactggtgtccactgcacctgccctgtgggcttcatgctgcagccagacaggaagacgtgcaaagatatagatgagtgccgcttaaacaacgggggctgtgaccatatttgccgcaacacagtgggcagcttcgaatgcagttgcaagaaaggctataagcttctcatcaatgagaggaactgccaggatatagacgagtgttcctttgatcgaacctgtgaccacatatgtgtcaacacaccaggaagcttccagtgtctctgccatcgtggctacctgttgtatggtatcacccactgtggggatgtggatgaatgcagcatcaaccggggaggttgccgctttggctgcatcaacactcctggcagctaccagtgtacctgcccagcaggccagggtcggctgcactggaatggcaaagattgcacagagccactgaagtgtcagggcagtcctggggcctcgaaagccatgctcagctgcaaccggtctggcaagaaggacacctgtgccctgacctgtccctccagggcccgatttttgccagagtctgagaatggcttcacggtgagctgtgggacccccagccccagggctgctccagcccgagctggccacaatgggaacagcaccaactccaaccactgccatgaggctgcagtgctgtccattaaacaacgggcctccttcaagatcaaggatgccaaatgccgtttgcacctgcgaaacaaaggcaaaacagaggaggctggcagaatcacagggccaggtggtgccccctgctctgaatgccaggtcaccttcatccaccttaagtgtgactcctctcggaagggcaagggccgacgggcccggacccctccaggcaaagaggtcacaaggctcaccctggaactggaggcagaggtcagagccgaagaaaccacagccagctgtgggctgccctgcctccgacagcgaatggaacggcggctgaaaggatccctgaagatgctcagaaagtccatcaaccaggaccgcttcctgctgcgcctggcaggccttgattatgagctggcccacaagccgggcctggtagccggggagcgagcagagccgatggagtcctgtaggcccgggcagcaccgtgctgggaccaagtgtgtcagctgcccgcagggaacgtattaccacggccagacggagcagtgtgtgccatgcccagcgggcaccttccaggagagagaagggcagctctcctgcgacctttgccctgggagtgatgcccacgggcctcttggagccaccaacgtcaccacgtgtgcaggtcagtgcccacctggccaacactctgtagatgggttcaagccctgtcagccatgcccacgtggcacctaccaacctgaagcaggacggaccctatgcttcccttgtggtgggggcctcaccaccaagcatgaaggggccatttccttccaagactgtgacaccaaagtccagtgctccccagggcactactacaacaccagcatccaccgctgtattcgctgtgccatgggctcctatcagcccgacttccgtcagaacttctgcagccgctgtccaggaaacacaagcacagactttgatggctctaccagtgtggcccaatgcaagaatcgtcagtgtggtggggagctgggtgagttcactggctatattgagtcccccaactacccgggcaactacccagctggtgtggagtgcatctggaacatcaaccccccacccaagcgcaagatccttatcgtggtaccagagatcttcctgccatctgaggatgagtgtggggacgtcctcgtcatgagaaagaactcatccccatcctccattaccacttatgagacctgccagacctacgagcgtcccattgccttcactgcccgttccaggaagctctggatcaacttcaagacaagcgaggccaacagcgcccgtggcttccagattccctatgttacctatgatgaggactatgagcagctggtagaagacattgtgcgagatggccggctctatgcctctgaaaaccaccaggagattttaaaggacaagaagctcatcaaggccttctttgaggtgctagcccacccccagaactacttcaagtacacagagaaacacaaggagatgctgccaaaatccttcatcaagctgctccgctccaaagtttccagcttcctgaggccctacaaatagtaaccctaggctcagagacccaattttttaagcccccagactccttagccctca

SEQ ID NO: 20 - protein, 1009 aa, Primate
  misc_feature (1)..(3183): LP283
MGSGRVPGLCLLVLLVHARAAQYSKAAQDVDECVEGTDNCHIDAICQNTPRSYKCICKSGYTGDGKHCKDVDECEREDNAGCVHDCVNIPGNYRCTCYDGFHLAHDGHNCLDVDECAEGNGGCQQSCVNMMGSYECHCREGFFLSDNQHTCIQRPEEGMNCMNKNHGCAHICRETPKGGIACECRPGFELTKNQRDCKLTCNYGNGGCQHTCDDTEQGPRCGCHIKFVLHTDGKTCIGERRLEQHIPTQAVSNETCAVNNGGCDSKCHDAATGVHCTCPVGFMLQPDRKTCKDIDECRLNNGGCDHICRNTVGSFECSCKKGYKLLINERNCQDIDECSFDRTCDHICVNTPGSFQCLCHRGYLLYGITHCGDVDECSINRGGCRFGCINTPGSYQCTCPAGQGRLHWNGKDCTEPLKCQGSPGASKAMLSCNRSGKKDTCALTCPSRARFLPESENGFTVSCGTPSPRAAPARAGHNGNSTNSNHCHEAAVLSIKQRASFKIKDAKCRLHLRNKGKTEEAGRITGPGGAPCSECQVTFIHLKCDSSRKGKGRRARTPPGKEVTRLTLELEAEVRAEETTASCGLPCLRQRMERRLKGSLKMLRKSINQDRFLLRLAGLDYELAHKPGLVAGERAEPMESCRPGQHRAGTKCVSCPQGTYYHGQTEQCVPCPAGTFQEREGQLSCDLCPGSDAHGPLGATNVTTCAGQCPPGQHSVDGFKPCQPCPRGTYQPEAGRTLCFPCGGGLTTKHEGAISFQDCDTKVQCSPGHYYNTSIHRCIRCAMGSYQPDFRQNFCSRCPGNTSTDFDGSTSVAQCKNRQCGGELGEFTGYIESPNYPGNYPAGVECIWNINPPPKRKILIVVPEIFLPSEDECGDVLVMRKNSSPSSITTYETCQTYERPIAFTARSRKLWINFKTSEANSARGFQIPYVTYDEDYEQLVEDIVRDGRLYASENHQEILKDKKLIKAFFEVLAHPQNYFKYTEKHKEMLPKSFIKLLRSKVSSFLRPYK

SEQ ID NO: 21 - protein, 993 aa, Primate
  MISC_FEATURE (1)..(993): LP344
MGSGRVPGLCLLVLLVHARAAQYSKAAQDVDECVEGTDNCHIDAICQNTPRSYKCICKSGYTGDGKHCKDVDECEREDNAGCVHDCVNIPGNYRCTCYDGFHLAHDGHNCLDVDECAEGNGGCQQSCVNMMGSYECHCREGFFLSDNQHTCIQRPEEGMNCMNKNHGCAHICRETPKGGIACECRPGFELTKNQRDCKLTCNYGNGGCQHTCDDTEQGPRCGCHIKFVLHTDGKTCIETCAVNNGGCDSKCHDAATGVHCTCPVGFMLQPDRKTCKDIDECRLNNGGCDHICRNTVGSFECSCKKGYKLLINERNCQDIDECSFDRTCDHICVNTPGSFQCLCHRGYLLYGITHCGDVDECSINRGGCRFGCINTPGSYQCTCPAGQGRLHWNGKDCTEPLKCQGSPGASKAMLSCNRSGKKDTCALTCPSRARFLPESENGFTVSCGTPSPRAAPARAGHNGNSTNSNHCHEAAVLSIKQRASFKIKDAKCRLHLRNKGKTEEAGRITGPGGAPCSECQVTFIHLKCDSSRKGKGRRARTPPGKEVTRLTLELEAEVRAEETTASCGLPCLRQRMERRLKGSLKMLRKSINQDRFLLRLAGLDYELAHKPGLVAGERAEPMESCRPGQHRAGTKCVSCPQGTYYHGQTEQCVPCPAGTFQEREGQLSCDLCPGSDAHGPLGATNVTTCAGQCPPGQHSVDGFKPCQPCPRGTYQPEAGRTLCFPCGGGLTTKHEGAISFQDCDTKVQCSPGHYYNTSIHRCIRCAMGSYQPDFRQNFCSRCPGNTSTDFDGSTSVAQCKNRQCGGELGEFTGYIESPNYPGNYPAGVECIWNINPPPKRKILIVVPEIFLPSEDECGDVLVMRKNSSPSSITTYETCQTYERPIAFTARSRKLWINFKTSEANSARGFQIPYVTYDEDYEQLVEDIVRDGRLYASENHQEILKDKKLIKAFFEVLAHPQNYFKYTEKHKEMLPKSFIKLLRSKVSSFLRPYK

SEQ ID NO: 22 - protein, 939 aa, Primate
  MISC_FEATURE (1)..(939): LP345
MGSGRVPGLCLLVLLVHARAAQYSKAAQDVDECVEGTDNCHIDAICQNTPRSYKCICKSGYTGDGKHCKDVDECEREDNAGCVHDCVNIPGNYRCTCYDGFHLAHDGHNCLDVDECAEGNGGCQQSCVNMMGSYECHCREGFFLSDNQHTCIQRPEEGMNCMNKNHGCAHICRETPKGGIACECRPGFELTKNQRDCKLTCNYGNGGCQHTCDDTEQGPRCGCHIKFVLHTDGKTCIETCAVNNGGCDSKCHDAATGVHCTCPVGFMLQPDRKTCKDIDECRLNNGGCDHICRNTVGSFECSCKKGYKLLINERNCQDIDECSFDRTCDHICVNTPGSFQCLCHRGYLLYGITHCGDVDECSINRGGCRFGCINTPGSYQCTCPAGQGRLHWNGKDCTEPLKCQGSPGASKAMLSCNRSGKKDTCALTCPSRARFLPESENGFTVSCGTPSPRAAPARAGHNGNSTNSNHCHEAAVLSIKQRASFKIKDAKCRLHLRNKGKTEEAGRITGPGGAPCSECQVTFIHLKCDSSRKGKGRRARTPPGKEVTRLTLELEAEVRAEETTASCGLPCLRQRMERRLKGSLKMLRKSINQDRFLLRLAGLDYELAHKPGLVAGERAEPMESCRPGQHRAGTKCGQCPPGQHSVDGFKPCQPCPRGTYQPEAGRTLCFPCGGGLTTKHEGAISFQDCDTKVQCSPGHYYNTSIHRCIRCAMGSYQPDFRQNFCSRCPGNTSTDFDGSTSVAQCKNRQCGGELGEFTGYIESPNYPGNYPAGVECIWNINPPPKRKILIVVPEIFLPSEDECGDVLVMRKNSSPSSITTYETCQTYERPIAFTARSRKLWINFKTSEANSARGFQIPYVTYDEDYEQLVEDIVRDGRLYASENHQEILKDKKLIKAFFEVLAHPQNYFKYTEKHKEMLPKSFIKLLRSKVSSFLRPYK

SEQ ID NO: 23 - protein, 265 aa, Primate
  MISC_FEATURE (1)..(265): LP346
MGSGRVPGLCLLVLLVHARAAQYSKAAQDVDECVEGTDNCHIDAICQNTPRSYKCICKSGYTGDGKHCKDVDECEREDNAGCVHDCVNIPGNYRCTCYDGFHLAHDGHNCLDVDECAEGNGGCQQSCVNMMGSYECHCREGFFLSDNQHTCIQRPEEGMNCMNKNHGCAHICRETPKGGIACECRPGFELTKNQRDCKLTCNYGNGGCQHTCDDTEQGPRCGCHIKFVLHTDGKTCIDASGTPSQLHQQPCFFLTNSSLPSLTLI

SEQ ID NO: 24 - DNA, 86 bp, artificial sequence
  synthetic construct
gcgcctcgagatttccccgaaatctagatttccccgaaatgatttccccgaaatgatttccccgaaatatctgccatctcaattag

SEQ ID NO: 25 - DNA, 27 bp, artificial sequence
  synthetic construct
gcggcaagctttttgcaaagcctaggc

SEQ ID NO: 26 - DNA, 270 bp, artificial sequence
  synthetic construct
ctcgagatttccccgaaatctagatttccccgaaatgattccccgaaatgatttccccgaaatatctgccatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctt

SEQ ID NO: 27 - DNA, 32 bp, artificial sequence
  synthetic construct
gcgctcgagggatgacagcgatagaaccccgg

SEQ ID NO: 28 - DNA, 30 bp, artificial sequence
  synthetic construct
gcgaagcttcgcgactccccggatccgcct

SEQ ID NO: 29 - DNA, 12 bp, artificial sequence
  synthetic construct
ggggactttccc

SEQ ID NO: 30 - DNA, 70 bp, artificial sequence
  synthetic construct
gcggcctcgaggggactttcccggggactttccggggatccgggactttccatcctgccatctcaattag

SEQ ID NO: 31 - DNA, 27 bp, artificial sequence
  synthetic construct
gcggcaagctttttgcaaagcctaggc

SEQ ID NO: 32 - DNA, 256 bp, artificial sequence
  synthetic construct
ctcgaggggactttcccggggactttccggggactttccgggactttccatctgccatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctt